SEQUENCE LISTING (125 sequences)

SEQ ID NO: 1 - DNA, 31 bp, synthetic construct
  source 1..31: mol_type = other DNA
gnhggnggtgbhgcrtttggytayaaaatgg

SEQ ID NO: 2 - DNA, 37 bp, synthetic construct
  source 1..37: mol_type = other DNA
gytayaaaatggahgahatyagdgttgahdttgaagg

SEQ ID NO: 3 - DNA, 35 bp, synthetic construct
  source 1..35: mol_type = other DNA
gahgahatyagdgttgahdttgaaggnvthtaytc

SEQ ID NO: 4 - DNA, 31 bp, synthetic construct
  source 1..31: mol_type = other DNA
crtadctvacaccagcyyttdcttgayvagc

SEQ ID NO: 5 - DNA, 34 bp, synthetic construct
  source 1..34: mol_type = other DNA
gyttgayttcnggngttaydtcrtadctvacacc

SEQ ID NO: 6 - DNA, 32 bp, synthetic construct
  source 1..32: mol_type = other DNA
gttaydtcrtadctvacaccagcyyttdcttg

SEQ ID NO: 7 - DNA, 46 bp, synthetic construct
  source 1..46: mol_type = other DNA
  modified_base 31: mod_base = OTHER; note = abasic site
ttgaagatatgcctatcactccatayrttgntgttggygttggtgc

SEQ ID NO: 8 - DNA, 47 bp, synthetic construct
  source 1..47: mol_type = other DNA
  modified_base 32: mod_base = OTHER; note = abasic site
gtkaaygtntattacgatdtagcnattgaagntatgcctatcactcc

SEQ ID NO: 9 - DNA, 30 bp, synthetic construct
  source 1..30: mol_type = other DNA
cactgtgcagctcattcaagggctttcttg

SEQ ID NO: 10 - DNA, 30 bp, synthetic construct
  source 1..30: mol_type = other DNA
cgcttcccactgtgcagctcattcaagggc

SEQ ID NO: 11 - DNA, 32 bp, synthetic construct
  source 1..32: mol_type = other DNA
cttcccactgtgcagctcattcaagggctttc

SEQ ID NO: 12 - DNA, 31 bp, synthetic construct
  source 1..31: mol_type = other DNA
ccactgtgcagctcattcaagggctttcttg

SEQ ID NO: 13 - DNA, 29 bp, synthetic construct
  source 1..29: mol_type = other DNA
cttttgagaggttttgttactttgagtaa

SEQ ID NO: 14 - DNA, 27 bp, synthetic construct
  source 1..27: mol_type = other DNA
tattccatgctgtagtattcaaacaca

SEQ ID NO: 15 - DNA, 31 bp, synthetic construct
  source 1..31: mol_type = other DNA
cactgtgcagctcattcaagggctttcttgc

SEQ ID NO: 16 - DNA, 48 bp, synthetic construct
  source 1..48: mol_type = other DNA
  modified_base 33: mod_base = OTHER; note = abasic site
ccggcttgctggacacacccatccgttacggcnaaaatgcgctggttc

SEQ ID NO: 17 - DNA, 47 bp, synthetic construct
  source 1..47: mol_type = other DNA
  modified_base 32: mod_base = OTHER; note = abasic site
cggcttgctggacacacccatccgttacggcnaaaatgcgctggttc

SEQ ID NO: 18 - DNA, 31 bp, synthetic construct
  source 1..31: mol_type = other DNA
catgcctgttgtcagatatttttccgagatg

SEQ ID NO: 19 - DNA, 30 bp, synthetic construct
  source 1..30: mol_type = other DNA
caaattggcggcggcgttatcactgtattg

SEQ ID NO: 20 - DNA, 30 bp, synthetic construct
  source 1..30: mol_type = other DNA
cttcagcaacaaattggcggcggcgttatc

SEQ ID NO: 21 - DNA, 32 bp, synthetic construct
  source 1..32: mol_type = other DNA
cagcacgggcggaatagagtggcttaattctc

SEQ ID NO: 22 - DNA, 30 bp, synthetic construct
  source 1..30: mol_type = other DNA
catagcgacagcacgggcggaatagagtgg

SEQ ID NO: 23 - DNA, 31 bp, synthetic construct
  source 1..31: mol_type = other DNA
catagcgacagcacgggcggaatagagtggc

SEQ ID NO: 24 - DNA, 34 bp, synthetic construct
  source 1..34: mol_type = other DNA
ctcaatctatccccacgtatgcatctgaattaac

SEQ ID NO: 25 - DNA, 35 bp, synthetic construct
  source 1..35: mol_type = other DNA
tgttcataacagacgggtagtcatgattgagttca

SEQ ID NO: 26 - DNA, 37 bp, synthetic construct
  source 1..37: mol_type = other DNA
atagagtggcttaattctcaatctatccccacgtatg

SEQ ID NO: 27 - DNA, 31 bp, synthetic construct
  source 1..31: mol_type = other DNA
cattttcatagcgacagcacgggcggaatag

SEQ ID NO: 28 - DNA, 50 bp, synthetic construct
  source 1..50: mol_type = other DNA
  modified_base 33: mod_base = OTHER; note = abasic site
cggtaaggttcaagctaaaaattcatttagcgnagttagctattggctag

SEQ ID NO: 29 - DNA, 55 bp, synthetic construct
  source 1..55: mol_type = other DNA
  modified_base 36: mod_base = OTHER; note = abasic site
caagctaaaaattcatttagcggagttagctattgnctagttaaaaataaaattg

SEQ ID NO: 30 - DNA, 24 bp, synthetic construct
  source 1..24: mol_type = other DNA
ctactccaatggaaacttctgagc

SEQ ID NO: 31 - DNA, 20 bp, synthetic construct
  source 1..20: mol_type = other DNA
gtggcgttggaaggcacttc

SEQ ID NO: 32 - DNA, 55 bp, synthetic construct
  source 1..55: mol_type = other DNA
  modified_base 37: mod_base = OTHER; note = abasic site
caagctaaaaattcatttagcggagttagctattggntagttaaaaataaaattg

SEQ ID NO: 33 - DNA, 30 bp, synthetic construct
  source 1..30: mol_type = other DNA
ccaaaccactacgttatctggagtgtgccc

SEQ ID NO: 34 - DNA, 29 bp, synthetic construct
  source 1..29: mol_type = other DNA
aggcaaccaaaccactacgttatctggag

SEQ ID NO: 35 - DNA, 30 bp, synthetic construct
  source 1..30: mol_type = other DNA
caggcaaccaaaccactacgttatctggag

SEQ ID NO: 36 - DNA, 36 bp, synthetic construct
  source 1..36: mol_type = other DNA
atgcgtgtattagccttatcggctgtgtttttggtg

SEQ ID NO: 37 - DNA, 33 bp, synthetic construct
  source 1..33: mol_type = other DNA
cgtgtattagccttatcggctgtgtttttggtg

SEQ ID NO: 38 - DNA, 31 bp, synthetic construct
  source 1..31: mol_type = other DNA
agccttatcggctgtgtttttggtggcatcg

SEQ ID NO: 39 - DNA, 35 bp, synthetic construct
  source 1..35: mol_type = other DNA
cgattatcggaatgcctgcggtagcaaaggaatgg

SEQ ID NO: 40 - DNA, 53 bp, synthetic construct
  source 1..53: mol_type = other DNA
  modified_base 36: mod_base = OTHER; note = abasic site
cgattatcggaatgcctgcggtagcaaaggaatggnaagaaaacaaaagttgg

SEQ ID NO: 41 - DNA, 58 bp, synthetic construct
  source 1..58: mol_type = other DNA
  modified_base 39: mod_base = OTHER; note = abasic site
atgcctgcggtagcaaaggaatggcaagaaaacaaaagntggaatgctcactttactg

SEQ ID NO: 42 - DNA, 51 bp, synthetic construct
  source 1..51: mol_type = other DNA
  modified_base 36: mod_base = OTHER; note = abasic site
cggtagcaaaggaatggcaagaaaacaaaagttggnatgctcactttactg

SEQ ID NO: 43 - DNA, 30 bp, synthetic construct
  source 1..30: mol_type = other DNA
ccgtttaagattattggtaaatccttgctg

SEQ ID NO: 44 - DNA, 34 bp, synthetic construct
  source 1..34: mol_type = other DNA
cttggttcgcccgtttaagattattggtaaatcc

SEQ ID NO: 45 - DNA, 34 bp, synthetic construct
  source 1..34: mol_type = other DNA
atgcgggtaaaaatgcttggttcgcccgtttaag

SEQ ID NO: 46 - DNA, 30 bp, synthetic construct
  source 1..30: mol_type = other DNA
ccagagcacaactacgccctgtgatttatg

SEQ ID NO: 47 - DNA, 35 bp, synthetic construct
  source 1..35: mol_type = other DNA
catggatcatatgaggtcaarccatcaggatcagc

SEQ ID NO: 48 - DNA, 35 bp, synthetic construct
  source 1..35: mol_type = other DNA
catcaggatcagcctcatchatggtsaatgghgtg

SEQ ID NO: 49 - DNA, 34 bp, synthetic construct
  source 1..34: mol_type = other DNA
gghgtggtgagaytgctmaccaaaccatgggatg

SEQ ID NO: 50 - DNA, 35 bp, synthetic construct
  source 1..35: mol_type = other DNA
catratttgtgctgtgcctcgtttygctyttggtg

SEQ ID NO: 51 - DNA, 35 bp, synthetic construct
  source 1..35: mol_type = other DNA
gtcacctccatratttgtgctgtgcctcgtttygc

SEQ ID NO: 52 - DNA, 34 bp, synthetic construct
  source 1..34: mol_type = other DNA
gctgtcacctccatratttgtgctgtgcctcgtt

SEQ ID NO: 53 - DNA, 46 bp, synthetic construct
  source 1..46: mol_type = other DNA
  modified_base 31: mod_base = OTHER; note = abasic site
catycccatggtcacacaaatagcyatgacngayachacacccttt

SEQ ID NO: 54 - DNA, 47 bp, synthetic construct
  source 1..47: mol_type = other DNA
  modified_base 31: mod_base = OTHER; note = abasic site
catggtcacacaaatagcyatgactgayacnacacccttyggacaac

SEQ ID NO: 55 - DNA, 37 bp, synthetic construct
  source 1..37: mol_type = other DNA
htahgahcaagaccacccatacaaaacntgggcntac

SEQ ID NO: 56 - DNA, 32 bp, synthetic construct
  source 1..32: mol_type = other DNA
aaaacntgggcntaccanggnagctangaaac

SEQ ID NO: 57 - DNA, 33 bp, synthetic construct
  source 1..33: mol_type = other DNA
ggnagctangaaacaaaacanactggatcagca

SEQ ID NO: 58 - DNA, 35 bp, synthetic construct
  source 1..35: mol_type = other DNA
cayggyagytaygaaacaaaacaracnggatcagc

SEQ ID NO: 59 - DNA, 40 bp, synthetic construct
  source 1..40: mol_type = other DNA
crtgggchtaccayggyagytaygaaacaaaacaracngg

SEQ ID NO: 60 - DNA, 35 bp, synthetic construct
  source 1..35: mol_type = other DNA
gtrtchactttytctttraavacrcgytgytgtcc

SEQ ID NO: 61 - DNA, 35 bp, synthetic construct
  source 1..35: mol_type = other DNA
gggttctcgtgtccactttytctttraaaacrcgc

SEQ ID NO: 62 - DNA, 48 bp, synthetic construct
  source 1..48: mol_type = other DNA
  modified_base 32: mod_base = OTHER; note = abasic site
grytgctracmaaaccthgggaygtyvtyccnayggtracacaratgg

SEQ ID NO: 63 - DNA, 37 bp, synthetic construct
  source 1..37: mol_type = other DNA
aacatggcactatgatgahgaaaayccytayaaaacg

SEQ ID NO: 64 - DNA, 34 bp, synthetic construct
  source 1..34: mol_type = other DNA
aaaayccytayaaaacgtgggcttaccatggatc

SEQ ID NO: 65 - DNA, 31 bp, synthetic construct
  source 1..31: mol_type = other DNA
tgahgaaaayccytayaaaacgtgggcttac

SEQ ID NO: 66 - DNA, 40 bp, synthetic construct
  source 1..40: mol_type = other DNA
garaayccytacaaaacgtgggcttaccayggatcytatg

SEQ ID NO: 67 - DNA, 35 bp, synthetic construct
  source 1..35: mol_type = other DNA
cttaccayggatcbtatgaagthaargccacaggc

SEQ ID NO: 68 - DNA, 35 bp, synthetic construct
  source 1..35: mol_type = other DNA
gtgtccactttctctttraaracyctytgctgscc

SEQ ID NO: 69 - DNA, 48 bp, synthetic construct
  source 1..48: mol_type = other DNA
  modified_base 31: mod_base = OTHER; note = abasic site
gataaayggagtygtgaaactyctcacnaanccrtgggatgtggtkcc

SEQ ID NO: 70 - DNA, 48 bp, synthetic construct; identical to SEQ ID NO: 69
  source 1..48: mol_type = other DNA
  modified_base 31: mod_base = OTHER; note = abasic site

SEQ ID NO: 71 - DNA, 36 bp, synthetic construct
  source 1..36: mol_type = other DNA
agaaachtggcaytatgaycadgaaaayccatacag

SEQ ID NO: 72 - DNA, 37 bp, synthetic construct
  source 1..37: mol_type = other DNA
gaagctatgaagcyccytcgacaggctcdgcntcytc

SEQ ID NO: 73 - DNA, 37 bp, synthetic construct
  source 1..37: mol_type = other DNA
tcgacaggctctgcatcctccatggtgaacggggtvg

SEQ ID NO: 74 - DNA, 35 bp, synthetic construct
  source 1..35: mol_type = other DNA
ttcgacaggctcwgcrtcctccatggtgaacgggg

SEQ ID NO: 75 - DNA, 40 bp, synthetic construct
  source 1..40: mol_type = other DNA
ccttcgacaggctcwgcrtcctccatggtgaacggggtrg

SEQ ID NO: 76 - DNA, 35 bp, synthetic construct
  source 1..35: mol_type = other DNA
catggragctatgaagcyccttcgacaggctcwgc

SEQ ID NO: 77 - DNA, 35 bp, synthetic construct
  source 1..35: mol_type = other DNA
agctatgaagctccttcgacaggctcwgcrtcctc

SEQ ID NO: 78 - DNA, 35 bp, synthetic construct
  source 1..35: mol_type = other DNA
gtrtcnaccttctcyttraacactctytgttgccc

SEQ ID NO: 79 - DNA, 48 bp, synthetic construct
  source 1..48: mol_type = other DNA
  modified_base 32: mod_base = OTHER; note = abasic site
gggaygtrrttccratggtgacycagttrgcnatgacagayacaaccc

SEQ ID NO: 80 - DNA, 48 bp, synthetic construct
  source 1..48: mol_type = other DNA
  modified_base 31: mod_base = OTHER; note = abasic site
aaaaccytgggaygtrattccratggtgacncagttrgccatgacaga

SEQ ID NO: 81 - DNA, 32 bp, synthetic construct
  source 1..32: mol_type = other DNA
gttaagggagtgaagacgatcagataccgtcg

SEQ ID NO: 82 - DNA, 35 bp, synthetic construct
  source 1..35: mol_type = other DNA
gagtgaagacgatcagataccgtcgtaatcttaac

SEQ ID NO: 83 - DNA, 31 bp, synthetic construct
  source 1..31: mol_type = other DNA
ggagtgaagacgatcagataccgtcgtaatc

SEQ ID NO: 84 - DNA, 33 bp, synthetic construct
  source 1..33: mol_type = other DNA
gactttgatttctcataaggtactgaaggaagc

SEQ ID NO: 85 - DNA, 35 bp, synthetic construct
  source 1..35: mol_type = other DNA
ctttgatttctcataaggtactgaaggaagcaatc

SEQ ID NO: 86 - DNA, 35 bp, synthetic construct
  source 1..35: mol_type = other DNA
cccaaagactttgatttctcataaggtactgaagg

SEQ ID NO: 87 - DNA, 35 bp, synthetic construct; identical to SEQ ID NO: 86
  source 1..35: mol_type = other DNA

SEQ ID NO: 88 - DNA, 47 bp, synthetic construct
  source 1..47: mol_type = other DNA
  modified_base 32: mod_base = OTHER; note = abasic site
ggtgttggatgaaagtgttaaaaataaaagtnatctttcgaggtgac

SEQ ID NO: 89 - DNA, 46 bp, synthetic construct
  source 1..46: mol_type = other DNA
  modified_base 31: mod_base = OTHER; note = abasic site
ctaggtgttggatgaaagtgttaaaaataanagtcatctttcgagg

SEQ ID NO: 90 - DNA, 49 bp, synthetic construct
  source 1..49: mol_type = other DNA
  modified_base 34: mod_base = OTHER; note = abasic site
gactaggctttggatgaaagattttaaaataagngttttctcttcggag

SEQ ID NO: 91 - DNA, 47 bp, synthetic construct
  source 1..47: mol_type = other DNA
  modified_base 31: mod_base = OTHER; note = abasic site
ctatgccgactaggctttggatgaaagattntaaaataagagttttc

SEQ ID NO: 92 - DNA, 30 bp, synthetic construct
  source 1..30: mol_type = other DNA
ccgccgcvccgcavcavmdakctgcdncrg

SEQ ID NO: 93 - DNA, 30 bp, synthetic construct
  source 1..30: mol_type = other DNA
cgccgcvccgcavcavmdakctgcdncrgg

SEQ ID NO: 94 - DNA, 32 bp, synthetic construct
  source 1..32: mol_type = other DNA
accgccgcvccgcavcavmdakctgcdncrgg

SEQ ID NO: 95 - DNA, 31 bp, synthetic construct
  source 1..31: mol_type = other DNA
ccgccgcvccgcavcavmdakctgcdncrgg

SEQ ID NO: 96 - DNA, 31 bp, synthetic construct
  source 1..31: mol_type = other DNA
accgccgcvccgcavcavmdakctgcdncrg

SEQ ID NO: 97 - DNA, 35 bp, synthetic construct
  source 1..35: mol_type = other DNA
ggaaacytctgarycmggrvyyaccgccgcrccsc

SEQ ID NO: 98 - DNA, 30 bp, synthetic construct
  source 1..30: mol_type = other DNA
agrtcraydggytkrttgcggkggcgytgg

SEQ ID NO: 99 - DNA, 31 bp, synthetic construct
  source 1..31: mol_type = other DNA
gttkbagrtcraydggytkrttgcggkggcg

SEQ ID NO: 100 - DNA, 33 bp, synthetic construct
  source 1..33: mol_type = other DNA
ccratgtgttkbagrtcraydggytkrttgcgg

SEQ ID NO: 101 - DNA, 32 bp, synthetic construct
  source 1..32: mol_type = other DNA
gtgttkbagrtcraydggytkrttgcggkggc

SEQ ID NO: 102 - DNA, 28 bp, synthetic construct
  source 1..28: mol_type = other DNA
gtgttkbagrtcraydggytkrttgcgg

SEQ ID NO: 103 - DNA, 31 bp, synthetic construct
  source 1..31: mol_type = other DNA
ccratgtgttkbagrtcraydggytkrttgc

SEQ ID NO: 104 - DNA, 49 bp, synthetic construct
  source 1..49: mol_type = other DNA
  modified_base 32: mod_base = OTHER; note = abasic site
ggcgytggaakgmayykyydgdrtttythccntaccctgcdgwytcdcc

SEQ ID NO: 105 - DNA, 50 bp, synthetic construct
  source 1..50: mol_type = other DNA
  modified_base 33: mod_base = OTHER; note = abasic site
cgytggaakgmayykyydgdrtttythccrtancctgcdgwytcdcctcc

SEQ ID NO: 106 - DNA, 31 bp, synthetic construct
  source 1..31: mol_type = other DNA
aggattcttcgcaaccatcagattygggttg

SEQ ID NO: 107 - DNA, 31 bp, synthetic construct
  source 1..31: mol_type = other DNA
attcttcgcaaccatcagattygggttggag

SEQ ID NO: 108 - DNA, 30 bp, synthetic construct
  source 1..30: mol_type = other DNA
gcaaccatcagattygggttggagacaagg

SEQ ID NO: 109 - DNA, 33 bp, synthetic construct
  source 1..33: mol_type = other DNA
attcttcgcaaccatcagattygggttggagac

SEQ ID NO: 110 - DNA, 35 bp, synthetic construct
  source 1..35: mol_type = other DNA
ttcttcgcaaccatcagattygggttggagacaag

SEQ ID NO: 111 - DNA, 35 bp, synthetic construct
  source 1..35: mol_type = other DNA
ggattcttcgcaaccatcagattygggttggagac

SEQ ID NO: 112 - DNA, 35 bp, synthetic construct
  source 1..35: mol_type = other DNA
caggattcttcgcaaccatcagattygggttggag

SEQ ID NO: 113 - DNA, 32 bp, synthetic construct
  source 1..32: mol_type = other DNA
gaattgattcttcaagaagcaccatataaggg

SEQ ID NO: 114 - DNA, 30 bp, synthetic construct
  source 1..30: mol_type = other DNA
tcaagaagcaccatataaggggctcttggg

SEQ ID NO: 115 - DNA, 30 bp, synthetic construct
  source 1..30: mol_type = other DNA
ttagtctgaattgattcttcaagaagcacc

SEQ ID NO: 116 - DNA, 48 bp, synthetic construct
  source 1..48: mol_type = other DNA
  modified_base 31: mod_base = OTHER; note = abasic site
gcactcaaygaattccagagtgacctcaacnccatcaaragcttgatg

SEQ ID NO: 117 - DNA, 48 bp, synthetic construct
  source 1..48: mol_type = other DNA
  modified_base 32: mod_base = OTHER; note = abasic site
attccagagtgacctcaacaccatcaaragcntgatgctactctacag

SEQ ID NO: 118 - DNA, 30 bp, synthetic construct
  source 1..30: mol_type = other DNA
ccggcttctttgcgactatcagattcggtc

SEQ ID NO: 119 - DNA, 30 bp, synthetic construct
  source 1..30: mol_type = other DNA
gaggaaacaggaatggccggcttctttgcg

SEQ ID NO: 120 - DNA, 30 bp, synthetic construct
  source 1..30: mol_type = other DNA
aattatgtcgaggaaacaggaatggccggc

SEQ ID NO: 121 - DNA, 30 bp, synthetic construct
  source 1..30: mol_type = other DNA
cctggtgcaaactttgtctgaatrgattcc

SEQ ID NO: 122 - DNA, 30 bp, synthetic construct
  source 1..30: mol_type = other DNA
tggataaccacctggtgcaaactttgtctg

SEQ ID NO: 123 - DNA, 30 bp, synthetic construct
  source 1..30: mol_type = other DNA
aaactttgtctgaatrgattcctcaaggag

SEQ ID NO: 124 - DNA, 47 bp, synthetic construct
  source 1..47: mol_type = other DNA
  modified_base 31: mod_base = OTHER; note = abasic site
agttccagagygatctcaataccatcaaagngctgatgctgctctac

SEQ ID NO: 125 - DNA, 46 bp, synthetic construct
  source 1..46: mol_type = other DNA
  modified_base 31: mod_base = OTHER; note = abasic site
gatctcaataccatcaaagggctgatgctgntctacagagaaatag

(19) United States
(12) Patent Application Publication
(10) Pub. No.: US 2025/0340924 A1
(43) Pub. Date: Nov. 6, 2025

(54) Sample Preparation Reagents

(71) Applicants: BIOCIFER PTY LTD, Birtinya (AU); University of the Sunshine Coast, Sippy Downs (AU); DMTC Limited, Kew (AU)

(72) Inventors: Joanne MacDonald, Sippy Downs (AU); Nina Pollak, Sippy Downs (AU); Nicole Ertl, Herston (AU)

(21) Appl. No.: 18/719,802

(22) PCT Filed: Dec. 14, 2022
(86) PCT No.: PCT/AU2022/051506

§ 371 Date: Jun. 13, 2024

(30) Foreign Application Priority Data
Dec. 14, 2021 (AU) 2021904060
Apr. 19, 2022 (AU) 2022901035
Nov. 8, 2022 (AU) 2022903342

(51) Int. Cl. C12Q 1/6806; C12Q 1/6844

(52) U.S. Cl. CPC C12Q 1/6806; C12Q 1/6844

(57) ABSTRACT

This invention relates to compositions, methods and kits for preparing samples for the detection of nucleic acids in the samples.

### CROSS-REFERENCE TO RELATED APPLICATIONS

This application is a U.S. National Phase of International Patent Application No. PCT/AU2022/051506, filed on Dec. 14, 2022, incorporated by reference, which claims priority under 35 USC § 119 from Australian Patent Application Nos. 2022903342, filed on Nov. 8, 2022; 2022901035, filed on Apr. 19, 2022; and 2021904060, filed on Dec. 14, 2021. The entire contents of these applications are incorporated herein by reference in their entirety.

### INCORPORATION BY REFERENCE OF SUBJECT MATTER SUBMITTED ELECTRONICALLY

Incorporated herein by reference is an electronic sequence listing: file name: 1250499 ST26 sequence listing.xml; creation date: Jun. 4, 2025; file size: 127,514 bytes.

### TECHNICAL FIELD

This invention relates to compositions, methods and kits for preparing samples for the detection of nucleic acids in the samples.

### BACKGROUND OF INVENTION

Molecular diagnostic approaches for the identification of nucleic acid, for example pathogen nucleic acid, in clinical samples offers a rapid alternative to traditional approaches involving culturing any pathogen or visualising any pathogen, for example, using microscopy.

However, compounds which are commonly added to clinical samples, have an inhibitory effect on nucleic acid amplification techniques, such as polymerase chain reaction (PCR).

Nucleic acid amplification inhibitors are a very heterogeneous group of chemical substances. A single sample may contain one or more different inhibitory substances and the same inhibitors can be found in many different matrices. Organic, as well as inorganic substances, which may be dissolved or solid, can act as nucleic acid amplification inhibitors. Calcium ions are an example of an inorganic substance with inhibitory effects on amplification. Inhibitors can also include organic compounds, for example, bile salts, urea, phenol, ethanol, polysaccharides, sodium dodecyl sulphate (SDS), humic acids, tannic acid, melanin as well as different proteins, such as collagen, myoglobin, haemoglobin, lactoferrin, immunoglobin G (IgG) and proteinases. Anticoagulants, such as heparin, and other additives may also inhibit nucleic acid amplification from blood samples. Inhibitors in stool may include polysaccharides or chlorophyll originating from herbs and vegetables, bile salts, urea, glycolipids, haemoglobin and heparin.

Furthermore, the concentration of the one or more nucleic acid amplification inhibitors in a sample is important for its inhibitory effect.

Nucleic acid amplification inhibitors are often added to a sample during sample processing and/or during nucleic acid extraction.

For example, salts (e.g. sodium chloride or potassium chloride), detergents or organic molecules [ethylenediaminetetraacetic acid (EDTA), sarkosyl, ethanol, isopropyl alcohol or phenol] may be added for efficient cell lysis or for the preparation of pure nucleic acids, but also cause PCR inhibition at certain concentrations. Ionic detergents (e.g. sodium deoxycholate, sarkosyl and SDS) are highly inhibitory for the PCR, whereas non-ionic detergents (e.g. Nonidet P-40, Tween 20, Triton X-100 and N-octyl glucoside) cause PCR inhibition only at relatively high concentrations. EDTA may deplete magnesium ions and thus inhibit DNA polymerase activity. Additives of the PCR mixture, such as dithiothreitol, dimethyl sulfoxide or mercaptoethanol, may also be inhibitory at certain concentrations.

As a consequence, sample preparation for amplification of pathogen nucleic acid almost always involves the inactivation (e.g. killing) of the pathogen, followed by extraction of nucleic acids in a sample into an extraction buffer system, followed by purification of the nucleic acids away from the sample they were extracted from, and further purification of the nucleic acids.

There remains a need for simple methods of extracting nucleic acids in a sample that can be used for downstream molecular applications.

### SUMMARY OF INVENTION

The present invention is based in part on the development of a Sample Preparation Reagent that can be used in methods of preparing samples and nucleic acid amplification. The Sample Preparation Reagent and methods of using the reagent can avoid the use of additional method steps to inactivate the pathogen, avoid the use of additional method steps to purify nucleic acid from the sample, and/or avoid the use of additional method steps to purify nucleic acid from any extraction buffer system. Desirable or advantageous characteristics of the Sample Preparation Reagent and methods of using the Sample Preparation Reagent include the completion of sample preparation in a short time frame and inactivation of pathogen in the sample with minimal processing, with minimal equipment required, removing nucleic acid sample preparation bottlenecks, improving safety of testing for laboratory personnel and reducing need for extended Biohazard Hood use in laboratories, improving laboratory throughput and/or providing significant savings in personnel hours.

In one embodiment the present invention provides a method of preparing nucleic acids for nucleic acid amplification from a sample, said method comprising; contacting a sample comprising nucleic acids with a composition comprising a quaternary ammonium compound, a denaturing agent and a precipitating agent to form a reaction sample.

In one embodiment, the present invention provides a method as described herein, wherein contacting the sample with the composition renders an infectious agent in the sample non-infectious.

In another embodiment, the present invention provides a method as described herein, wherein the quaternary ammonium compound is betaine.

In a further embodiment, the present invention provides a method as described herein, wherein the denaturing agent is selected from the group consisting of NaOH and KOH.

In a further embodiment, the present invention provides a method as described herein, wherein the precipitating agent is selected from the group consisting of isopropanol, ethanol, and methanol.

In a further embodiment, the present invention provides a method as described herein, wherein the composition does not comprise a detergent.

In a further embodiment, the present invention provides a method as described herein, wherein the composition does not comprise SDS.

In a further embodiment, the present invention provides a method as described herein, wherein the composition does not comprise EDTA.

In a further embodiment, the present invention provides a method as described herein, wherein the composition does not comprise DMSO.

In a further embodiment, the present invention provides a method as described herein, wherein the composition comprises from about 0.078 M to about 2.5 M betaine.

In a further embodiment, the present invention provides a method as described herein, wherein the composition comprises from about 15 mM to about 1000 mM denaturing agent.

In a further embodiment, the present invention provides a method as described herein, wherein the composition comprises from about 10% to about 80% precipitating agent.

In a further embodiment, the present invention provides a method as described herein, wherein the composition comprises up to 80% isopropanol, up to 125 mM NaOH, and up to 0.16 M betaine.

In a further embodiment, the present invention provides a method as described herein, wherein the composition comprises up to 80% isopropanol, up to 31 mM NaOH, and up to 0.31 M betaine.

In a further embodiment, the present invention provides a method as described herein, wherein the composition comprises up to 1000 mM NaOH, up to 0.31 M betaine, and up to 60% isopropanol.

In a further embodiment, the present invention provides a method as described herein, wherein the composition comprises up to 1000 mM NaOH, up to 0.625 M betaine, and up to 40% isopropanol.

In a further embodiment, the present invention provides a method as described herein, wherein the composition comprises up to 2.5 M Betaine, up to 500 mM NaOH, and up to 20% isopropanol.

In a further embodiment, the present invention provides a method as described herein, wherein the composition comprises 1.25 M Betaine, 200 mM NaOH, and 40% isopropanol.

In a further embodiment, the present invention provides a method as described herein, wherein the composition comprises 1.125 M Betaine, 180 mM NaOH, and 36% isopropanol.

In a further embodiment, the present invention provides a method as described herein, wherein the composition comprises RNAse and DNase-free water.

In a further embodiment, the present invention provides a method as described herein, wherein the nucleic acids are not separated from reaction sample the prior to nucleic acid amplification.

In a further embodiment, the present invention provides a method as described herein, wherein the contacting is performed at a sample:composition ratio of 1:1 or greater.

In a further embodiment, the present invention provides a method as described herein, wherein the contacting is performed at a sample:composition ratio of 1:1 or less.

In a further embodiment, the present invention provides a method as described herein, wherein the sample comprises nucleic acids from an infectious agent.

In a further embodiment, the present invention provides a method as described herein,, wherein the sample is selected from the group consisting of cells, tissues, autopsy samples, bone marrow aspirates, blood, serum, plasma, urine, cerebrospinal fluid, middle ear fluids, breast milk, bronchoalveolar lavage, tracheal aspirates, sputum, oral fluids, nasopharyngeal aspirates, oropharyngeal aspirates, saliva, nasal swabs, nasopharyngeal swabs, oropharyngeal swabs, oral swabs, eye swabs, cervical swabs, vaginal swabs, rectal swabs, stool, stool suspensions, wastewater, soil, and plant material.

In a further embodiment, the present invention provides a method as described herein, wherein the sample is from a subject infected with or at risk of infection with an infectious agent.

In a further embodiment, the present invention provides a method as described herein, wherein the infectious agent is selected from the group consisting of a bacterium, a fungus, a yeast, a protozoan, a parasite, or a virus.

In a further embodiment, the present invention provides a method as described herein, wherein contacting the sample with the composition renders the infectious agent non-infectious.

In a further embodiment, the present invention provides a method as described herein, further comprising performing nucleic acid amplification on the reaction sample.

In a further embodiment, the present invention provides a method as described herein, wherein the contacting is performed for less than 15 minutes.

In a further embodiment, the present invention provides a method as described herein, wherein the reaction sample is diluted prior to nucleic acid amplification.

In a further embodiment, the present invention provides a method as described herein, wherein the nucleic acids are not extracted from the reaction sample prior to nucleic acid amplification.

In a further embodiment, the present invention provides a method as described herein, wherein the nucleic acid amplification is selected from the group consisting of real-time PCR, real-time quantitative PCR, reverse transcription real-time PCR, reverse transcription real-time quantitative PCR, droplet digital PCR, ligase chain reaction, Recombinase-Polymerase Amplification, loop-mediated isothermal amplification, reverse-transcription LAMP, strand displacement amplification, transcription-mediated amplification, transcription-free isothermal amplification, repair chain reaction amplification; ligase chain reaction amplification, gap filling ligase chain reaction amplification, coupled ligase detection and PCR, and Nucleic Acid Sequenced Based Amplification and RNA transcription-free amplification.

In a further embodiment, the present invention provides a nucleic acid sample prepared by a method as described herein.

In another embodiment, the present invention provides a method of detecting nucleic acids in a sample comprising: contacting a sample comprising nucleic acids with a composition comprising a quaternary ammonium compound, a denaturing agent and a precipitating agent to form a reaction sample; optionally diluting the reaction sample; and performing nucleic acid amplification on the sample.

In a further embodiment, the present invention provides a method as described herein, wherein contacting the sample with the composition renders an infectious agent in the sample non-infectious.

In another embodiment, the present invention provides a sample preparation composition comprising a quaternary ammonium compound, a denaturing agent and a precipitating agent.

In another embodiment, the present invention provides a sample preparation composition as described herein, wherein the quaternary ammonium compound is betaine.

In another embodiment, the present invention provides a sample preparation composition as described herein, wherein the denaturing agent is selected from the group consisting of NaOH, and KOH.

In another embodiment, the present invention provides a sample preparation composition as described herein, wherein the precipitating agent is selected from the group consisting of isopropanol, ethanol, and methanol.

In another embodiment, the present invention provides a sample preparation composition as described herein, wherein the composition does not comprise a detergent.

In another embodiment, the present invention provides a sample preparation composition as described herein, wherein the composition does not comprise SDS.

In another embodiment, the present invention provides a sample preparation composition as described herein, wherein the composition does not comprise EDTA.

In another embodiment, the present invention provides a sample preparation composition as described herein, wherein the composition does not comprise DMSO.

In another embodiment, the present invention provides a sample preparation composition as described herein, wherein the composition comprises from about 0.078 M to about 2.5 M betaine.

In another embodiment, the present invention provides a sample preparation composition as described herein, wherein the composition comprises from about 10% to about 80% precipitating agent.

In another embodiment, the present invention provides a sample preparation composition as described herein, wherein the composition comprises up to 80% precipitating agent.

In another embodiment, the present invention provides a sample preparation composition as described herein, wherein the composition comprises up to 80% isopropanol, up to 125 mM NaOH, and up to 0.16 M betaine.

In another embodiment, the present invention provides a sample preparation composition as described herein, wherein the composition comprises up to 80% isopropanol, up to 31 mM NaOH, and up to 0.31 M betaine.

In another embodiment, the present invention provides a sample preparation composition as described herein, wherein the composition comprises up to 1000 mM NaOH, up to 0.31 M betaine, and up to 60% isopropanol.

In another embodiment, the present invention provides a sample preparation composition as described herein, wherein the composition comprises up to 1000 mM NaOH, up to 0.625 M betaine, and up to 40% isopropanol.

In another embodiment, the present invention provides a sample preparation composition as described herein, wherein the composition comprises up to 2.5 M Betaine, up to 500 mM NaOH, and up to 20% isopropanol.

In another embodiment, the present invention provides a sample preparation composition as described herein, wherein the composition comprises 1.25 M Betaine, 200 mM NaOH, and 40% isopropanol.

In another embodiment, the present invention provides a sample preparation composition as described herein, wherein the composition comprises 1.125 M Betaine, 180 mM NaOH, and 36% isopropanol.

In another embodiment, the present invention provides a sample preparation composition as described herein, wherein the composition comprises RNase and DNase-free water.

### BRIEF DESCRIPTION OF DRAWINGS

FIG. 1. SARS-CoV-2 viability after contacting with Sample Preparation Reagents E and U. A) Sample Preparation Reagent E was mixed with infectious SARS-CoV-2 at either a 1 in 6 dilution (i.e. 20 μL Sample Preparation Reagent E added to 100 μL virus in cell culture media), or a 1 in 10 dilution (i.e., 10 μL Sample Preparation Reagent E added to 90 μL virus). Virus and Sample Preparation Reagent E mix were incubated for the indicated times and the virus titre determined by CCID₅₀ assays using Vero E6 cells. Average and standard deviation of virus titres (CCID₅₀/mL) is plotted for 1 in 10 dilution testing (squares); no virus titre was obtained for any time point at 1 in 6 dilution testing (circles). The limit of detection for this testing was 10² CCID₅₀/mL. B) (i) Premixing infectious SARS-CoV-2 with Sample Preparation Reagent U at a 1:1 ratio for 10 seconds prevented cell death of Vero E6 cells (no cytopathic effect was observable), whereas (ii) full cytopathic effect is observed in Vero E6 cells after SARS-CoV-2 infection without premixing with Sample Preparation Reagent.

FIG. 2. Sample Preparation Reagent E detected as little as ˜ 1 infectious particle in RT-qPCR tube. Cultured SARS-CoV-2 dilutions (20 μL) were incubated with Sample Preparation Reagent E (4 μL) for 10 minutes before RT-qPCR detection. Column purification (NucleoSpin RNA Virus Mini kit, Macherey-Nagel) of cultured SARS-CoV-2 dilutions (20 μL) was performed in parallel, and after multiple preparation and washing steps was eluted in 20 μL RNase-free water before subsequent RT-qPCR detection. RT-qPCR used the iTaq Universal Probes One-Step Kit (BioRad) with primers and probe amplifying the E gene (E-gene/BioRad RT-qPCR). Cultured virus was processed in duplicate, and then tested in duplicate using RT-qPCR, resulting in quadruplicate testing per virus concentration. Average and standard deviation Ct values are plotted for Sample Preparation Reagent E processed samples (circles) and column purified samples (squares).

FIG. 3. COVID-positive samples (n=38; 20 μL) were incubated for 10 minutes at room temperature with either 2 μL or 4 μL of Sample Preparation Reagent E, after which 5 μL mixtures were added into a RT-qPCR (final volume 20 μL). RT-qPCR used the SensiFAST Probe Lo-ROX kit (Bioline, Meridian Bioscience) with primers and probe amplifying the ORF1ab gene (ORF1ab-gene/Bioline RT-qPCR). Average Ct values from both 2 μL (circles) and 4 μL (squares) Sample Preparation Reagent E volumes trialled showed 97.73% correlation with Ct values obtained by Roche MagNA Pure 96 purification sample processing, when assessed by Pearson product moment correlation coefficient (Microsoft Excel). Not detected samples were assigned a Ct value at the cut-off (40) for this analysis.

FIG. 4. Samples from FIG. 3 were re-incubated with Sample Preparation Reagent E (4 μL) for 10 minutes (in duplicate), before ORF1ab-gene/Bioline RT-qPCR testing. Average Ct values obtained from both replicate 1 (squares) and replicate 2 (circles) showed 93.58% correlation with Ct values obtained by Roche MagNA Pure sample processing, when assessed by Pearson product moment correlation coefficient (Microsoft Excel). Not detected samples were assigned a Ct value at the cut-off (40) for this analysis.

FIG. 5. Ten COVID-positive samples (20 μL) were mixed with Sample Preparation Reagent E (4 μL) in duplicate (5:1 ratio of sample:Sample Preparation Reagent E), and incubated for 10 minutes at room temperature, before adding 5 μL processed sample into a RT-qPCR (final volume 20 μL). RT-qPCR used the SensiFAST Probe Lo-ROX One-Step Kit (Bioline, Meridian Bioscience) with primers and probe amplifying the ORF1ab gene (ORF1ab-gene/Bioline RT-qPCR). Average Ct values (circles) showed 96.17% correlation with Ct values obtained by Roche MagNA Pure sample processing, when assessed by Pearson product moment correlation coefficient (Microsoft Excel). The ten COVID-positive samples were also tested “neat” (without processing) by directly adding 5 μL of the samples into the ORF1ab-gene/Bioline RT-qPCR. Average Ct values (squares) showed 92.03% correlation with Ct values obtained by Roche MagNA Pure sample processing, when assessed by Pearson product moment correlation coefficient (Microsoft Excel). Not detected samples were assigned a Ct value at the cut-off (40) for this analysis.

FIG. 6. The ten COVID-positive samples from FIG. 5tested “neat” (without processing) or by processing with Sample Preparation Reagent E (1:5 ratio) and subsequently tested by ORF1ab-gene/Bioline RT-qPCR were directly compared by Pearson product moment correlation coefficient (Microsoft Excel). The cross-comparison of Ct-values (circles) showed 95.48% correlation between Ct values. Not detected samples were assigned a Ct value at the cut-off (40) for this analysis.

FIG. 7. Phase separation determination of Sample Preparation Reagent formulations. The phase separation of 408 formulations with varying concentrations of NaOH (0-1000 mM), betaine (0-2.5 M) and isopropanol (0-80%) was determined. 176 of the 408 formulations were in a single phase and this is shown in the Figure, where the concentrations of each ingredient (log 2 scaled axes) are displayed. Graph depicts the maximum relative percentage of isopropanol that allows for incorporation of betaine and NaOH at varying concentrations, yet remains monophasic: concentrations of betaine and NaOH that remain soluble in the presence of 80% isopropanol (triangles); additional betaine and NaOH concentrations that become soluble in 60% isopropanol (open circles), 40% isopropanol (diamonds), 20% isopropanol (squares), and no isopropanol (closed circle). Specific formulations indicated are Sample Preparation Reagent U (cross, x), Sample Preparation Reagent E (plus, +) and the maximum concentrations that all ingredients can be combined before phase separation (asterisk, *).

FIG. 8. Detection of SARS-CoV-2 down to 1000 copies/mL. Testing utilized ddPCR-quantitated SARS-CoV-2 RNA as template. RT-qPCR used the SensiFAST Probe Lo-ROX One-Step Kit (Bioline, Meridian Bioscience) with primers and probe amplifying the ORF1ab gene (n=2; squares), the E gene (n=4; diamonds) and the N gene using primers designated as N1 (n=2; circles) or N2 (n=5; triangles). Dotted line shows log 10 trendline of the average of all primer Ct-values combined.

FIG. 9. Detection of a UV-inactivated SARS-CoV-2 down to 10⁻⁶ dilution. Testing utilized UV-inactivated SARS-CoV-2 as template, which was mixed with Sample Preparation Reagent E for 10 min at room temperature at a ratio of 5:1 sample to Sample Preparation Reagent E before adding 5 μL processed sample into a RT-qPCR (final volume 20 μL) to detect SARS-CoV-2. Ct-values obtained from each dilution of UV-inactivated SARS-CoV-2 processed and tested using RT-qPCR using the SensiFAST Probe Lo-ROX One-Step Kit (Bioline, Meridian Bioscience) with primers and probe combinations amplifying (A) the N gene using combinations designated N1 (n=2; squares), N2 (n=5; triangles) and N3 (n=2; diamonds) and (B) the ORF1ab gene (n=2; squares) and the E gene (n=4; triangles).

FIG. 10. Schematic of process used for bacterial enumeration. For this experiment, 0.5 McFarland standard solutions were prepared from cultured Escherichia coli isolates in either 0.9% sterile saline or sterile ultra-pure molecular grade water. Serial dilutions were prepared with either 0.9% sterile saline or sterile ultra-pure molecular grade water and 10 μL drops of each dilution placed in triplicate on pre-warmed Columbia horse blood agar (HBA) plates. Experiment was repeated three times.

FIG. 11. Schematic of bacterial inactivation testing. For this, six different samples (prepared 0.5 McFarland bacterial solutions) and Sample Preparation Reagent E mixtures were prepared (Table 13), along with a positive control (0.5 McFarland bacterial solution mixed with either 0.9% sterile saline or sterile ultra-pure molecular grade water). The mixtures were incubated for 10 min at room temperature, then 10 μL drops of each mixture and the positive control placed onto triplicate pre-warmed Columbia horse blood agar (HBA) plates, with the experiment repeated three times.

FIG. 12. Bacterial inactivation testing images. (A) Schematic of culture plate showing placement of different inactivation mixtures. (B) Results of the first experimental repeat (Replicate 1, Table 14) using 0.5 McFarland solutions prepared in either sterile ultra-pure molecular grade water (water) or 0.9% sterile saline (saline). Grey circles were added to the NDM73 saline pictures to highlight the location of E. coli growth after overnight incubation at 37° C. White spots seen at the same location on all plates are reflections from the camera when the pictures were taken.

FIG. 13. Analytical sensitivity testing of a bla_{KPC} RPA-LFS test using Sample Preparation Reagent E to prepare samples. 0.5 McFarland E. coli bacterial solutions, prepared in either sterile A) 0.9% saline or B) ultra-pure molecular grade water, were mixed at different ratios with the Sample Preparation Reagent E, incubated for 10 min at room temperature and then diluted, either 1 in 2, or 1 in 5, with sterile ultra-pure molecular grade water. Serial dilutions (using sterile 0.9% saline or ultra-pure molecular grade water) prepared from those mixtures were trialled using a bla_{KPC} RPA-LFS test, with replicates in triplicate or quadruplicate as indicated. Each experiment was split into two runs, with each run containing one positive control and one no template control (negative). Normalised black pixel values were determined using the ImageJ software, with the cut-off for each experiment determined to be three times the standard deviation of the two negative samples, with * denoting positive samples (above the cut-off). Lateral flow pictures and figure of normalised black pixel values shown are the result of one of the replicate experiments. Values shown under “+ results/#runs” and “correct results (%)” are a summary of the replicate experiments.

FIG. 14. Analytical sensitivity determination of dengue virus (DENV) 1-4 recombinase polymerase amplification lateral flow detection (RPA-LFD) tests using plasmid DNA. Sensitivity testing used NS5 gene fragment RNA transcripts diluted 10-fold in nuclease-free water for DENV-1 RPA-LFD (A), DENV-2 RPA-LFD (B), DENV-3 RPA-LFD (C) and DENV-4 RPA-LFD (D). Photograph of lateral flow strips with control bands (all samples) and test bands (positive samples) compared to copy number of serially diluted plasmid DNA (copies/μL) and no template control (NTC) (left). Normalised pixel density (normalised black values) from the lateral flow test strip displayed (middle). Positive results compared to number of runs at that dilution was used to calculate the percentage of positive tests performed at that dilution (right).

FIG. 15. Analytical sensitivity determination of dengue virus (DENV) 1-4 reverse-transcription recombinase polymerase amplification lateral flow detection (RT-RPA-LFD) tests using reverse transcribed RNA. Sensitivity testing used NS5 gene fragment RNA transcripts diluted 10-fold in nuclease-free water for DENV-1 RT-RPA-LFD (A), DENV-2 RT-RPA-LFD (B), DENV-3 RT-RPA-LFD (C) and DENV-4 RT-RPA-LFD (D). Photograph of lateral flow strips with control bands (all samples) and test bands (positive samples) compared to copy number of serially diluted template RNA (copies/μL) and no template control (NTC) (left). Normalised pixel density (normalised black values) from the lateral flow test strip displayed (middle). Positive results compared to number of runs at that dilution was used to calculate the percentage of positive tests performed at that dilution (right).

FIG. 16. Analytical sensitivity of dengue virus (DENV) reverse-transcription recombinase polymerase amplification lateral flow detection (RT-RPA-LFD) tests using DENV isolate RNA. Sensitivity testing used purified RNA (NucleoSpin RNA Virus Mini kit, Macherey-Nagel) of DENV-1 (A), DENV-2 (B), DENV-3 (C) and DENV-4 (D) isolates. Copy number of RNA isolates was determined by universal dengue RT-qPCR. RT-qPCR used transcribed RNA as standard, which originated from a plasmid containing the Capsid peptide and NS5 region of DENV-1. Photograph of lateral flow strips with control bands (all samples) and test bands (positive samples) compared to copy number of serially diluted kit-extracted template RNA (copies/μL) and no template control (NTC) (left). Normalised pixel density (normalised black values) from the lateral flow test strip displayed (middle). Positive results compared to number of runs at that dilution was used to calculate the percentage of positive tests performed at that dilution (right).

FIG. 17. Analytical serotype-specificity of dengue virus (DENV) reverse-transcription recombinase polymerase amplification lateral flow detection (RT-RPA-LFD) tests using kit-extracted dengue virus isolates. For specificity testing, purified RNA (NucleoSpin RNA Virus Mini kit, Macherey-Nagel) of DENV-1 (ET243), DENV-2 (New Guinea C), DENV-3 (ET209) and DENV-4 (ET288) isolates at 10⁵ copies/μL was used in DENV-1 RT-RPA-LFD (A), DENV-2 RT-RPA-LFD (B), DENV-3 RT-RPA-LFD (C) and DENV-4 RT-RPA-LFD (D) tests. Copy number (copies/μL) of 10-fold serially diluted purified viral RNA was determined by RT-qPCR using transcribed RNA as standard, which originated from a plasmid containing the Capsid peptide and NS5 region of DENV-1. Photograph of lateral flow strips with control bands (all samples) and test bands (positive samples), nuclease-free water was used as no template control (NTC) (left). Normalised pixel density (black values) from the test displayed (middle). Positive results compared to number of runs tested displayed as percentage of correct results from all runs (right).

FIG. 18. Analytical sensitivity of dengue virus 1 (DENV-1) reverse-transcription recombinase polymerase amplification lateral flow detection (RT-RPA-LFD) tests in presence or absence of Sample Preparation Reagent E. Sensitivity testing used rapidly processed (Sample Preparation Reagent E) (A, C, E) and non-processed (B, D, F) DENV-1 (ET243) isolate samples in cell culture supernatant. Processing incubation temperatures included room temperature (A, B), 30° C. (C, D) and on ice (E, F). Photograph of lateral flow strips with control bands (all samples) and test bands (positive samples), nuclease-free water was used as no template control (NTC) (left). Normalised pixel density (black values) from the test displayed (middle). Positive results compared to number of runs tested displayed as percentage of correct results from all runs (right). TCID₅₀/mL were determined by TCID₅₀ assays using C6/36 cells and calculated as described by Reed and Muench (1938).

FIG. 19. Analytical sensitivity of dengue virus (DENV) reverse-transcription recombinase polymerase amplification lateral flow detection (RT-RPA-LFD) tests assessed isolate cell culture supernatants rapidly processed with Sample Preparation Reagent E. Sensitivity testing used rapidly processed RNA (Sample Preparation Reagent E) of DENV-1 (ET243) (A), DENV-1 (TC861HA) (B), DENV-2 (New Guinea C) (C), DENV-3 (ET209) (D) and DENV-4 (ET288) (E) isolates with respective DENV RT-RPA-LFD. Photograph of lateral flow strips with control bands (all samples) and test bands (positive samples), nuclease-free water was used as no template control (NTC) (left). Normalised pixel density (black values) from the test displayed (middle). Positive results compared to number of runs tested displayed as percentage of correct results from all runs (right). TCID₅₀/mL were determined by TCID₅₀ assays using C6/36 cells and calculated as described by Reed and Muench.

FIG. 20. Analytical serotype-specificity of dengue virus (DENV) reverse-transcription recombinase polymerase amplification lateral flow detection (RT-RPA-LFD) tests using synthetic RNA. Specificity was tested with NS5 gene fragment RNA transcripts of DENV-1 (A), DENV-2 (B), DENV-3 (C) and DENV-4 (D) at 10⁵ copies/μL. Photograph of lateral flow strips with control bands (all samples) and test bands (positive samples) compared to each DENV RNA sample and no template control (NTC) (left). Normalised pixel density (black values) from the test displayed (middle). Positive results compared to number of runs tested using different DENV serotypes displayed as percentage of correct results from all runs (right).

FIG. 21. Analytical specificity of dengue virus (DENV) reverse-transcription recombinase polymerase amplification lateral flow detection (RT-RPA-LFD) tests testing flaviviruses. For this, RNA was isolated with TRIzol reagent from harvested cell culture supernatants infected with Japanese encephalitis Nakayama virus (JEV), West Nile virus subtype Kunjin NSW 2011 (KUNV), Murray Valley encephalitis virus 151 (MVEV), Yellow Fever virus 17D (YFV) and Zika virus MR766 (ZIKV), and tested along with transcribed RNA (denoted with #) of DENV-1, -2, -3, and -4 as positive controls using DENV-1 RT-RPA-LFD (A), DENV-2 RT-RPA-LFD (B), DENV-3 RT-RPA-LFD (C) and DENV-4 RT-RPA-LFD (D) tests.

FIG. 22. Sample Preparation Reagent E inactivates DENV-1. DENV-1 (ET243) was incubated with Sample Preparation Reagent E at a 5:1 ratio (i.e. 20 μL Sample-Preparation Reagent E added to 100 μL virus in cell culture media containing 2% foetal bovine serum, 2 mM glutamine and 1× Antibiotic-Antimycotic) for the indicated times. Virus titre was determined by TCID₅₀ assays using C6/36 cells and calculated as described by Reed and Muench. This experiment was performed three times, with one sample taken per time point.

FIG. 23. Optimising the amount of Sample Preparation Reagent E required to efficiently process mock dengue-infected blood samples. Testing used DENV-4 (ET288) isolate spiked into K3 EDTA whole blood, rapidly processed with Sample Preparation Reagent E, followed by Dengue RT-RPA-LFD testing. TCID₅₀/mL was determined by TCID₅₀ assays using C6/36 cells and calculated as described by Reed and Muench. Sample: Sample preparation Reagent E ratios and dilution ratios (left). Photograph of lateral flow strips with control bands (all samples) and test bands (positive samples), nuclease-free water was used as no template control (NTC) and transcribed DENV-4 RNA as positive control (PTC, at 104 copies/μL) (middle left). Normalised pixel density (black values) from the test displayed (middle right). Positive results compared to number of runs tested displayed as percentage of correct results from all runs (right). “Extraction” as used in the Figures (e.g. FIGS. 23-28) refers to a reaction sample prepared by contacting a Sample with the Sample Preparation Reagent at the recited ratios.

FIG. 24. Optimising the amount of Sample Preparation Reagent E required to efficiently process mock dengue-infected plasma and serum samples. Testing used DENV-3 (ET209) isolate spiked into plasma or serum, rapidly processed with rapidly processed with Sample Preparation Reagent E, followed by Dengue RT-RPA-LFD testing. TCID₅₀/mL was determined by TCID₅₀ assays using C6/36 cells and calculated as described by Reed and Muench (1938). Sample Preparation and dilution ratios (left). Photograph of lateral flow strips with control bands (all samples) and test bands (positive samples), nuclease-free water was used as no template control (NTC) and transcribed DENV-3 RNA as positive control (PTC, at 103 copies/μL) (middle left). Normalised pixel density (black values) from the test displayed (middle right). Positive results compared to number of runs tested displayed as percentage of correct results from all runs (right).

FIG. 25. Dengue virus 1 (DENV-1) reverse-transcription recombinase polymerase amplification lateral flow detection (RT-RPA-LFD) tests using human mock samples rapidly processed with Sample Preparation Reagent E. A DENV-1 (ET243) isolate with known viral titre (TCID₅₀/mL) was spiked into human K3 EDTA whole blood, plasma or serum. Mock-samples were processed using sample:Sample Preparation Reagent E ratios of 1:1 (blood), 2:1 (Plasma), and 5:1 (serum). Mixtures were incubated for 10 min at Room temperature followed by 1:1 dilution with water, from which 1 μL was used for DENV-1 RT-RPA-LFD testing. Photographs of example lateral flow strips show control bands (all samples) and test bands (positive samples); nuclease-free water was used as no template control (NTC) and transcribed DENV-1 RNA as positive control (PTC, at 10⁶ copies/μL) (middle left). Normalised pixel density (black values) from the test displayed (middle right). Positive results compared to number of runs tested displayed as percentage of correct results from all runs, followed by Ct values and copy number from comparative testing with extracted RNA (NucleoSpin RNA Virus Mini kit, Macherey-Nagel) using a universal dengue RT-qPCR (right). RT-qPCR used transcribed RNA as standard, which originated from a plasmid containing the Capsid and NS5 region of DENV-1.

FIG. 26. Dengue virus 2 (DENV-2) reverse-transcription recombinase polymerase amplification lateral flow detection (RT-RPA-LFD) tests using human mock samples rapidly processed with Sample Preparation Reagent E. A DENV-2 (New Guinea C) isolate with known viral titre (TCID₅₀/mL) was spiked into human K3 EDTA whole blood, plasma or serum. Mock-samples were processed using sample:Sample Preparation Reagent E ratios of 1:1 (blood), 2:1 (Plasma), and 5:1 (serum). Mixtures were incubated for 10 min at Room temperature followed by 1:1 dilution with water, from which 1 L was used for DENV-2 RT-RPA-LFD testing. Photographs of example lateral flow strips show control bands (all samples) and test bands (positive samples); nuclease-free water was used as no template control (NTC) and transcribed DENV-1 RNA as positive control (PTC, at 10⁶ copies/μL) (middle left). Normalised pixel density (black values) from the test displayed (middle right). Positive results compared to number of runs tested is displayed (second from right) and was used to calculate the percentage of correct results from all runs (far right column).

FIG. 27. Dengue virus 3 (DENV-3) reverse-transcription recombinase polymerase amplification lateral flow detection (RT-RPA-LFD) tests using human mock samples rapidly processed with Sample Preparation Reagent E. A DENV-3 (ET209) isolate with known viral titre (TCID₅₀/mL) was spiked into human K3 EDTA whole blood, plasma or serum. Mock-samples were processed using sample:Sample Preparation Reagent E ratios of 1:1 (blood), 2:1 (Plasma), and 5:1 (serum). Mixtures were incubated for 10 min at Room temperature followed by 1:1 dilution with water, from which 1 OL was used for DENV-3 RT-RPA-LFD testing. Photographs of example lateral flow strips show control bands (all samples) and test bands (positive samples); nuclease-free water was used as no template control (NTC) and transcribed DENV-1 RNA as positive control (PTC, at 104 copies/μL) (middle left). Normalised pixel density (black values) from the test displayed (middle right). Positive results compared to number of runs tested is displayed (second from right) and was used to calculate percentage of correct results from all runs (far right column).

FIG. 28. Dengue virus 4 (DENV-4) reverse-transcription recombinase polymerase amplification lateral flow detection (RT-RPA-LFD) tests using human mock samples rapidly processed with Sample Preparation Reagent E. A DENV-4 (ET288) isolate with known viral titre (TCID₅₀/mL) was spiked into human K3 EDTA whole blood, plasma or serum. Mock-samples were processed using sample:Sample Preparation Reagent E ratios of 1:1 (blood), 2:1 (Plasma), and 5:1 (serum). Mixtures were incubated for 10 min at Room temperature followed by 1:1 dilution with water, from which 1 μL was used for DENV-4 RT-RPA-LFD testing. Photographs of example lateral flow strips show control bands (all samples) and test bands (positive samples); nuclease-free water was used as no template control (NTC) and transcribed DENV-1 RNA as positive control (PTC, at 10⁶ copies/μL) (middle left). Normalised pixel density (black values) from the test displayed (middle right). Positive results compared to number of runs tested is displayed (second from right), and was used to calculate the percentage of correct results from all runs (far right column).

FIG. 29. Rapid dengue virus (DENV) 1-4 serotyping tests of individual infected or uninfected mosquitoes and comparative universal dengue RT-qPCR testing. Testing used Sample Preparation Reagent U to rapidly process mosquito bodies, followed by DENV-1 RT-RPA-LFD (A), DENV-2 RT-RPA-LFD (B), DENV-3 RT-RPA-LFD (C) and DENV-4 RT-RPA-LFD (D). Comparative RT-qPCR was performed by column-purifying RNA from mosquito heads followed by testing using a universal dengue RT-qPCR targeting the capsid gene. (i) Sample description, (ii) serotype-specific mosquito sample number, (iii) Ct values obtained with universal dengue RT-qPCR from kit-extracted mosquito heads, (iv) calculated copy number of universal dengue RT-qPCR from kit-extracted mosquito heads, (v) photograph of lateral flow strips with control bands (all samples) and test bands (positive samples) of rapidly extracted mosquito bodies using the respective DENV RT-RPA-LFD tests, (vi) blank normalised pixel density (black values) from the corresponding lateral flow strip of rapidly extracted mosquito bodies, (vii) RT-RPA-LFD test result of mosquito bodies displayed as positive (+) or negative (−) test result, (viii) number of mosquito bodies that tested positive compared to total number of mosquito bodies tested with respective DENV RT-RPA-LFD, (ix) percentage (%) of infected mosquitoes detected and (x) sensitivity of the DENV RT-RPA-LFD test with 95% confidence interval (CI).

FIG. 30. Rapid dengue virus (DENV) 1-4 serotyping tests of pools of 5 mosquitoes, and comparative testing using a universal dengue RT-qPCR. Testing used Sample Preparation Reagent U to rapidly process pools of 5 mosquito bodies, followed by DENV-1 RT-RPA-LFD (A), DENV-2 RT-RPA-LFD (B), DENV-3 RT-RPA-LFD (C) and DENV-4 RT-RPA-LFD (D) tests. Comparative RT-qPCR analysis was performed by column-purifying RNA from pools of 5 mosquito heads, followed by universal dengue RT-qPCR targeting the capsid gene. (i) sample description, (ii) serotype-specific mosquito sample number, (iii) Ct values obtained with universal dengue RT-qPCR from kit-extracted mosquito heads, (iv) calculated copy number of universal dengue RT-qPCR from kit-extracted mosquito heads, (v) photograph of lateral flow strips with control bands (all samples) and test bands (positive samples) of rapidly extracted mosquito bodies using the respective DENV RT-RPA-LFD tests, (vi) blank normalised pixel density (black values) from the corresponding lateral flow strip of rapidly extracted mosquito bodies, (vii) RT-RPA-LFD test result of mosquito bodies displayed as positive (+) or negative (−) test result, (viii) number of mosquito bodies that tested positive compared to total number of mosquito bodies tested with respective DENV RT-RPA-LFD, (ix) percentage (%) of infected mosquitoes detected and (x) sensitivity of the DENV RT-RPA-LFD test with 95% confidence interval (CI).

FIG. 31. Dengue virus (DENV) reverse-transcription recombinase polymerase amplification lateral flow detection (RT-RPA-LFD) protocol for testing DENV-infected and uninfected mosquitoes. Easy result interpretation with lateral flow detection technology. A single control line depicts absence of DENV in an uninfected mosquito. The appearance of a control and test line confirm that the mosquito was infected with DENV.

FIG. 32. Analytical sensitivity of the Wolbachia RPA-LFD test using plasmid containing the wsp gene: (A) Lateral flow detection (LFD) images show control bands (all samples) and test bands (positive samples) with corresponding copy numbers of ten-fold serially diluted plasmid DNA (copies/μL). (B) Graph represents the normalized pixel densities (black values) of individual test bands from corresponding dilution. (C) Positive results compared to the number of times the test was conducted. (D) % correct result demonstrates the analytical sensitivity as a percentage from all runs, which consistently shows a test line for 20 copies/μL.

FIG. 33. Optimization of sample preparation conditions using individual mosquitoes processed in either NaOH/SDS or Sample Preparation Reagent U followed by dilution from 1/5 to 1/100 before Wol RPA-LFD testing. (A) Lateral flow detection (LFD) images show an example uninfected and infected mosquito processed with either NaOH/SDS or Sample Preparation Reagent U followed by Wol RPA-LFD testing. (B) Graph represents the average and standard deviation (n=5) LFD pixel densities (black values) of individual mosquitoes processed either with Sample Preparation Reagent U or NaOH/SDS and then diluted either 1 in 2, 1 in 5, 1 in 10, 1 in 50, or 1 in 100, followed by Wol RPA-LFD testing. Statistical analysis using two-tailed T-Test to compare the blank normalized pixel densities of test bands from NaOH/SDS and Sample Preparation Reagent U resulted in p-value of 0.016, which shows significant improvement in LFD pixel densities (black values) obtained when processing mosquitoes with Sample Preparation Reagent U.

FIG. 34. Diagnostic testing of the rapid Wolbachia (Wol) test using laboratory infected and uninfected mosquitoes. (A) Lateral flow detection (LFD) images show control bands (all samples) and test bands (positive samples) of individual mosquitoes (n=20) and pools of 5 mosquitoes (n=6) processed with rapid Wol test. (B) Graph represents the normalized pixel densities (black values) of individual test bands from corresponding samples. (C) Positive results compared to the number of times the test was conducted. (D) % correct result demonstrates the analytical sensitivity as a percentage from all runs, which shows 100% accuracy in testing both individual and pooled mosquitoes.

FIG. 35. Timing of individual mosquito Wolbachia infection detection using the rapid Wolbachia (Wol) test. Laboratory-infected and uninfected mosquitoes were tested fresh, or after being left in a humid chamber for one or two weeks. (A) Lateral flow detection (LFD) images show control bands (all samples) and test bands (positive samples) of mosquitoes left in traps for 0, one, or two weeks and processed with the rapid Wol test. (B) Graph represents the normalized pixel densities (black values) of individual test bands from corresponding samples. (C) Positive results compared to the number of mosquitoes tested. (D) % correct result demonstrates the analytical sensitivity as a percentage from all runs, which shows 100% sensitivity and specificity for mosquitoes left in traps for one week or less (n=20), or 90% sensitivity and 100% specificity for mosquitoes left in humid traps for two weeks (n=13).

FIG. 36. Timing of pooled mosquito Wolbachia infection detection using the rapid Wolbachia (Wol) test. Infected mosquito pools consisting of one laboratory-infected and four uninfected mosquitoes, or uninfected mosquito pools consisting of five uninfected mosquitoes were tested using the rapid Wol test after being left in a humid chamber for 0, 1 or 2 weeks. (A) Lateral flow detection (LFD) images show control bands (all samples) and test bands (positive samples) of pooled mosquitoes left in traps for 0, 1, or 2 weeks and processed with the rapid Wol test. (B) Graph represents the normalized pixel densities (black values) of individual test bands from corresponding samples. (C) Positive results compared to the number of mosquitoes tested. (D) % correct result demonstrates the analytical sensitivity as a percentage from all runs, which shows 100% sensitivity and specificity in for mosquitoes left in traps for one week or less and then pooled for testing (n=12), or 33% sensitivity and 100% specificity for testing mosquitoes left in humid traps for two weeks (n=6).

FIG. 37. Analytical sensitivity and yield analysis of Plasmodium falciparum infected blood diagnostic assays. (A) P. falciparum infected blood culture with known parasite concentration was diluted 10-fold in blood before testing. (B) Standard DNA extraction followed by qPCR determined cycle threshold (Ct) value, and calculated concentration (parasites/□□L) using quantified qPCR standards. The % yield from DNA extraction and qPCR detection was then determined by comparing to the known parasite concentration added at each dilution. (C) Rapid P. falciparum blood test results and effective parasite concentration/% yield used for subsequent testing after serial dilution in K3 EDTA blood. Representative photographs of resultant lateral flow strips (i) show position of control and test lines from which standardized black value (ii) was determined by ImageJ analysis of test line. The number of times a positive result was obtained per total number of replicate experiments (iii) was used to determine percentage positivity (iv).

FIG. 38. Clinical analysis of the rapid P. falciparum (Pf) blood test over an infection time-course. Stored K3 EDTA blood samples (n=13) from a single individual. The dashed line indicates the administration of antimalarial rescue medication at Day 8. (A) Geometric mean of parasites/500 μL was determined using ultra-sensitive qPCR. Results are also shown in parasites/IL of blood to assist with comparison. (B) Parallel standard qPCR testing performed, including cycle threshold values (Ct) and calculated parasites/IL determined from quantified qPCR standards. (C) Infection timepoint samples were collected during study. (D) Rapid Pf test results from the study, including (i) representative photographs of resultant lateral flow strips show position of control and test lines alongside the collection times (days) of blood (parasites/μL) from which standardized black value (ii) was determined by ImageJ analysis of test line. The number of times a positive result was obtained per total number of replicate experiments (iii) was used to determine percentage positivity (iv).

FIG. 39. Analytical sensitivity of the P. falciparum (Pf) RPA-LFD test was 4 copies/μL. Ten-fold dilutions of plasmid containing a portion of the P. falciparum 18Sr DNA target gene, or a water no template control (NTC) were tested with the Pf RPA-LFD test. Representative photographs of resultant lateral flow strips (A) show position of control and test lines alongside plasmid concentration (copies/μL) from which standardized black value (B) was determined by ImageJ analysis of test line. The number of times a positive result was obtained per total number of replicate experiments (C) was used to determine percentage positivity (D).

FIG. 40. Rapid P. falciparum test applied to detection of infected or uninfected Anopheles stephensi mosquitoes. Fresh or frozen infected mosquitoes were tested in batches as indicated, alongside uninfected mosquitoes (a representative negative uninfected mosquito shown per testing batch). Photographs of resultant lateral flow strips (A) show position of control and test lines alongside mosquito sample groups from which standardized black value (B) was determined by ImageJ analysis of test line. The number of times a positive result was obtained per total number of samples tested (C) was used to determine percentage positivity (D).

FIG. 41. Rapid P. falciparum test applied to detection of infected Anopheles stephensi mosquitoes in pools (top), or when left in traps for up to 8 days (bottom). Top panel shows test results for infected pools that each contained one A. stephensi mosquito fed from P. falciparum infected blood cultures, mixed with 19 known uninfected mosquitoes (or 20 known uninfected mosquitoes; Uninfected pools). Bottom panel shows results from testing individual A. stephensi mosquitoes fed with P. falciparum infected blood cultures, that were subsequently left in environmental chambers simulating a tropical environment for 8 days, prior to testing. Photographs of resultant lateral flow strips (A) show position of control and test lines alongside mosquito sample groups from which standardized black value (B) was determined by ImageJ analysis of test line. The number of times a positive result was obtained per total number of samples tested (C) was used to determine percentage positivity (D).

FIG. 42. Analytical sensitivity of Hepatopancreatic parvoviruses (HPV) RPA lateral-flow strip assay. (A) Photograph of lateral-flow strips with control bands (all samples) and test bands (positive samples) compared to copy number of serial diluted synthetic template DNA shown as plasmid (copies/μL) and water as a negative control. (B) Normalised pixel density (black values) from the assay displayed in (A) which was used to calculate positives (labelled by *) and negatives. (C) Positive results compared to number of individual runs. (D) Analytical sensitivity displayed as percentage of correct results. No., number; Pos., positive.

FIG. 43. Diagnostic sensitivity and specificity of Hepatopancreatic parvoviruses (HPV) RPA lateral-flow strip test using samples processed with Sample Preparation Reagent U. (A) Photograph of lateral-flow strips with control and test bands compared to real-time qPCR results, water or positive control #(2.42×10⁶ copies/μL synthetic template DNA). (B) Normalised pixel density from the assay displayed in (A) showing positive samples or positive control (both labelled by *) and negative water controls. (C) Positive results compared to number of individual runs. (D) Sensitivity and specificity displayed as percentage of correct results. n.d., not detected; No., number; Pos., positive.

FIG. 44. Detection of a Hepatopancreatic parvovirus (HPV)-positive sample spiked into HPV-negative wildtype banana shrimp samples with the HPV RPA lateral-flow strip test after processing with Sample Preparation Reagent U. (A) Photograph of lateral-flow strips with control and test bands compared to qPCR results, water or positive control #(2.42×106 copies/μL synthetic template DNA). (B) Normalised pixel density from the assay displayed in (A) showing positive samples or positive control (both labelled by *) and negative water controls. (C) Positive results compared to number of individual runs. (D) Sensitivity and specificity displayed as percentage of correct results. No., number; Pos., positive.

FIG. 45. Serial dilutions of an Hepatopancreatic parvoviruses (HPV)-positive banana shrimp sample assessed with the HPV RPA lateral-flow strip test after processing with Sample Preparation Reagent U. (A) Photograph of lateral-flow strips with control and test bands compared to qPCR results, water or positive control #(2.42×10⁶ copies/μL synthetic template DNA). (B) Normalised pixel density from the assay displayed in (A) showing positive samples or positive control (both labelled by *) and negative water controls. (C) Positive results compared to number of individual runs. (D) Sensitivity and specificity displayed as percentage of correct results. No., number; Pos., positive.

FIG. 46. Analytical sensitivities of three Nipah virus recombinase-based isothermal amplification lateral flow detection assays with RNA transcripts. Sensitivity testing used Nucleoprotein (N) gene fragment RNA transcripts diluted 10-fold in water for RT-nfoRPA-LFD (A), RT-exoRPA-LFD (B), and RT-RAA-LFD (C). Images of lateral flow strips with two bands (control and test band) indicates the sample is positive for NiV synthetic RNA transcript, and single control band indicates a valid reaction with negative sample. Photograph of lateral flow strips with control bands (all samples) and test bands (positive samples) compared to copy number of serially diluted NiV RNA (copies/μL) and no template control (NTC) (left). Normalised pixel density (normalised black values) from the lateral flow test strip displayed (middle). Positive samples compared to number of samples tested at that dilution was used to calculate the percentage of positive tests performed at that dilution (right).

FIG. 47. Analytical specificities of three Nipah virus recombinase-based isothermal amplification lateral flow detection tests. Specificity testing used synthetic RNA of NiV and HeV, and viral RNA extracts from alphavirus CHIKV, and flaviviruses DENV-1, DENV-2, DENV-3, DENV-4, JEV, MVEV, WNVKUNJ, YFV and ZIKV for RT-nfoRPA-LFD (A), RT-exoRPA-LFD (B), and RT-RAA-LFD (C). Images of lateral flow strips with two bands (control and test band) indicates the sample is positive for respective viral RNA extract, and single control band indicates a valid reaction with negative sample. Nuclease-free water was tested as the no template control (NTC; left). Normalised pixel density (normalised black values) from the test displayed (middle). Positive samples compared to number of samples tested using different viral RNA transcripts and extracts were used to calculate percentage of positive samples (right).

FIG. 48. Sensitivity and specificity of three rapid NiV tests. Sensitivity testing used rapidly processed NiVB and NiVM strain isolates for RT-nfoRPA-LFD (A), RT-exoRPA-LFD (B), and RT-RAA-LFD (C) assays compared to TCID₅₀/mL determined by immunolabeling assays and copies/reaction determined by comparative Taqman PCR. Images of lateral flow strips with two bands (control and test band) indicates the sample is positive for NiV, and single control band indicates a valid reaction with negative sample. Photograph of lateral flow strips with control bands (all samples) and test bands (positive samples) compared to titre of rapidly processed serially diluted NiV isolate (TCID₅₀/mL) and no template control (NTC) (left). Normalised pixel density (normalised black values) from the lateral flow test strip displayed (middle). Positive samples compared to number of samples tested at that dilution was used to calculate the percentage of positive tests performed at that dilution (right).

FIG. 49. Analytical sensitivity of HeV RT-RPA-LFD assay testing synthetic HeV RNA. Sensitivity testing used 10-fold serially diluted synthetic RNA from HeV. Images of lateral flow strips with two lines (control and test line) indicated the sample is positive detecting HeV-specific RNA, and single control lines indicated a valid reaction with negative sample, such as nuclease-free water as no template control (NTC) (left). Normalised pixel densities (normalised black values) from the lateral flow strips displayed (middle). Positive (Pos.) samples compared to number (No.) of samples tested at that dilution was used to calculate the percentage of positive tests performed at that dilution (right).

FIG. 50. Analytical specificity of HeV RT-RPA-LFD assay testing purified RNA of other viruses. Specificity testing used kit-purified RNA of Nipah virus Bangladesh (NiV_{B}) and Malaysia (NiV_{M}) strains, dengue virus (DENV-1, -2, -3, and -4), and TRIzol-purified RNA of chikungunya virus (CHIKV), Japanese encephalitis virus (JEV), Murray Valley encephalitis virus (MVEV), yellow fever virus (YFV), West Nile virus (subtype Kunjin; WNVKUNJ), and Zika virus (ZIKV), as well as, synthetic HeV RNA (10⁶ copies/μL) as positive template control (PTC). Images of lateral flow strips with two lines (control and test line) indicated the sample is positive detecting HeV-specific RNA, and single control lines indicated a valid reaction with negative sample, such as nuclease-free water as no template control (NTC) (left). Normalised pixel densities (normalised black values) from the lateral flow strips displayed (middle). Positive (Pos.) samples compared to number (No.) of samples tested of that purified viral RNA sample was used to calculate the percentage of positive tests performed with that purified viral RNA sample (right).

FIG. 51. Detection of HeV isolate in viral transport medium with the rapid Hendra test. Viral transport medium containing gamma-irradiated HeV isolate was diluted in viral transport medium and analysed with the rapid Hendra test (left) or viral RNA was isolated using a kit and then assessed by Taqman PCR (right). Rapid Hendra test: Images of lateral flow strips with control lines (all samples including viral transport medium as no template control (NTC)) and test lines (positive samples); Normalised pixel densities (normalised black values) from the lateral flow strips displayed; Positive (Pos.) samples compared to number (No.) of samples tested at that dilution was used to calculate the percentage of positive tests performed at that dilution. Virus isolation and Taqman PCR: Comparative PCR testing cycle threshold (Ct) and calculated copy number (Copies/reaction).

FIG. 52. Sample Preparation Reagent E inactivates Nipah virus. Nipah virus (Bangladesh isolate) was incubated with Sample Preparation Reagent E in a 1:1 (i.e. 250 μL Sample Preparation Reagent E to 250 UL virus in cell culture media MEM containing 10% foetal bovine serum, 100U/mL penicillin, 100 μg/mL streptomycin and 0.25 μg/mL Amphotericin B added), 1:5 and 1:9 ratio for the indicated times at room temperature and the virus titre determined by TCID₅₀ assays using Vero cells. Mixtures were titrated by 10-fold serial dilutions on Vero cells in 96-well plates. After 3-4 days, titre was determined by scoring individual wells as either positive or negative for the presence of cytopathic effect (CPE) typical of Nipah virus (syncytia). TCID₅₀/mL was calculated as described by Reed and Muench This experiment was performed one time, with three samples taken per time point. Dotted line indicates limit of detection of assays based on a starting 1/10 dilution of samples. Phosphate buffered saline (PBS) was used as a negative control.

FIG. 53. PCR testing of Nipah virus RNA. This experiment was repeated twice and each graph (top and bottom) is from a different experiment. Sample Preparation Reagent E was mixed with Nipah virus RNA and tested neat, or diluted 1 in 2 in water by adding 2 μL to the RT-PCR master mix.

FIG. 54. 1% agarose gel of Sample Preparation Reagent E prepared canine swabs. DNA (20 μL) was mixed with 4 μL DNA electrophoresis sample loading dye (6×, BioRad) and electrophoresed at 100V for 50 minutes. The Gene Ruler DNA ladder mix (100-10,000 bp range, Thermofisher) was also included in one of the lanes to assess the size of the extracted DNA. Z1-Z3 are Dog swabs exposed to Sample Preparation Reagent E diluted in water, whereas Z4 & Z5 are Dog swabs exposed to Sample Preparation Reagent E diluted in AE buffer.

FIG. 55. Agarose gel visualisation of genomic prawn DNA prepared using Sample Preparation Reagent E. DNA samples (20 μL) were mixed with 4 μL DNA electrophoresis sample loading dye (6×, BioRad) and electrophoresed at 100V for 50 minutes. The Gene Ruler DNA ladder mix (100-10,000 bp range, Thermofisher) was also included in one of the lanes to assess the size of the extracted DNA.

Error! Reference source not found. Persistence of low, medium and high concentrations of DNA spiked into Sample Preparation Reagent E or molecular grade water. Aliquots of the spiked Sample Preparation Reagent E were prepared and stored at 4° C. until use. At the respective timepoints, aliquots of the mixtures were diluted 1:1 in molecular grade water and tested with the bla_{OXA-48} in-house TaqMan assay. Negative controls (NTC and water mixed with Sample Preparation Reagent E) gave consistent negative results and are not shown in the graph. Cycle threshold was set at 0.05. TCE stands for Sample Preparation Reagent E.

### DETAILED DESCRIPTION

### I. Terms

Unless otherwise noted, technical terms are used according to conventional usage.

Definitions of common terms in molecular biology may be found in Lewin's Genes X, ed. Krebs et al., Jones and Bartlett Publishers, 2009 (ISBN 0763766321); Kendrew et al. (eds.), The Encyclopedia of Molecular Biology, published by Blackwell Publishers, 1994 (ISBN 0632021829); Robert A. Meyers (ed.), Molecular Biology and Biotechnology: a Comprehensive Desk Reference, published by Wiley, John & Sons, Inc., 1995 (ISBN 0471186341); and George P. Redei, Encyclopedic Dictionary of Genetics, Genomics, Proteomics and Informatics, 3rd Edition, Springer, 2008 (ISBN: 1402067534).

The following explanations of terms and methods are provided to better describe the present disclosure and to guide those of ordinary skill in the art to practice the present disclosure. The singular forms “a,” “an,” and “the” refer to one or more than one, unless the context clearly dictates otherwise. For example, the term “comprising a nucleic acid molecule” includes single or plural nucleic acid molecules and is considered equivalent to the phrase “comprising at least one nucleic acid molecule.” As used herein, “comprises” means “includes.” Thus, “comprising A or B,” means “including A, B, or A and B,” without excluding additional elements.

All publications, patent applications, patents, and other references mentioned herein are incorporated by reference in their entirety for all purposes.

In order to facilitate review of the various embodiments of this disclosure, the following explanations of specific terms are provided:

Amplification: Increasing the number of copies of a nucleic acid molecule, such as a gene or fragment of a gene, for example at least a portion of a nucleic acid molecule (e.g. a viral nucleic acid, a prokaryotic nucleic acid, a eukaryotic nucleic acid, synthetic nucleic acid). The products of an amplification reaction are called amplification products. An example of in vitro amplification is the polymerase chain reaction (PCR), in which a sample (such as a biological sample from a subject) is contacted with a pair of oligonucleotide primers, under conditions that allow for hybridization of the primers to a nucleic acid molecule in the sample. The primers are extended under suitable conditions, dissociated from the template, and then re-annealed, extended, and dissociated to amplify copies of the nucleic acid molecule.

Other examples of in vitro amplification techniques include real-time PCR, real-time quantitative PCR (qPCR), reverse transcription real-time PCR, reverse transcription real-time quantitative PCR (RT-qPCR), droplet digital PCR (ddPCR), ligase chain reaction (LCR), Recombinase-Polymerase Amplification (RPA), loop-mediated isothermal amplification (LAMP; see Notomi et al., Nucl. Acids Res. 28: e63, 2000), reverse-transcription LAMP (RT-LAMP), strand displacement amplification (see U.S. Pat. No. 5,744,311), transcription-mediated amplification (see U.S. Pat. No. 5,399,491), transcription-free isothermal amplification (see U.S. Pat. No. 6,033,881), repair chain reaction amplification (see WO 90/01069); ligase chain reaction amplification (see EP-A-320 308), gap filling ligase chain reaction amplification (see U.S. Pat. No. 5,427,930), coupled ligase detection and PCR (see U.S. Pat. No. 6,027,889), and NASBA™ RNA transcription-free amplification (see U.S. Pat. No. 6,025,134).

Conditions sufficient for: Any environment that permits the desired activity, for example, in the context of sample preparation, that permits inactivation of any infectious agent in the sample and/or release of nucleic acids from the sample, or in the context of amplification, that permits specific binding or hybridization between two nucleic acid molecules or that permits reverse transcription and/or amplification of a nucleic acid. Such an environment may include, but is not limited to, particular incubation conditions (such as time and or temperature) or presence and/or concentration of particular factors, for example in a solution (such as buffer(s), salt(s), metal ion(s), detergent(s), nucleotide(s), enzyme(s), and so on).

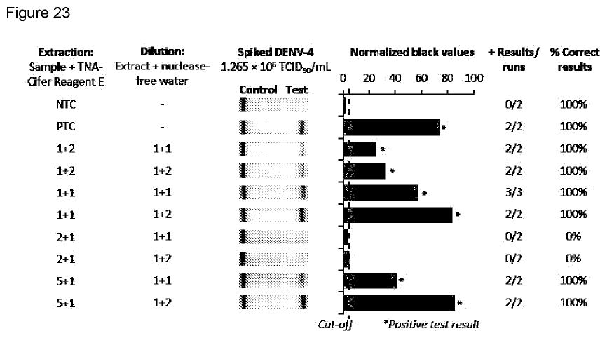

Contact; and Contacting Placement in direct physical association; for example, in solid and/or liquid form. For example, in the context of the sample preparation methods described herein, contacting includes mixing a sample with a Sample Preparation Reagent, in solution, and can include mixing and incubating a sample and Sample Preparation Reagent. “Extraction” as used in the Figures (e.g. FIGS. 23 to 28) refers to a reaction sample prepared by contacting a Sample with the Sample Preparation Reagent at the recited ratios.

Detectable label: A compound or composition that is conjugated directly or indirectly to another molecule (such as a nucleic acid molecule) to facilitate detection of that molecule. Specific non-limiting examples of labels include fluorescent and fluorogenic moieties (e.g., fluorophores), chromogenic moieties, haptens (such as biotin, digoxigenin, and fluorescein), affinity tags, and radioactive isotopes (such as 32P, 33P, 35S, and 1251). The label can be directly detectable (e.g., optically detectable) or indirectly detectable (for example, via interaction with one or more additional molecules that are in turn detectable). Methods for labelling nucleic acids, and guidance in the choice of labels useful for various purposes, are discussed, e.g., in Sambrook and Russell, in Molecular Cloning: A Laboratory Manual, 3rd Ed., Cold Spring Harbor Laboratory Press (2001) and Ausubel et al, in Current Protocols in Molecular Biology, Greene Publishing Associates and Wiley-Intersciences (1987, and including updates).

In one embodiment, the detectable label is a binding pair of moieties that have a specific binding affinity for each other. In one embodiment, the binding pair is biotin/avidin (or biotin/streptavidin). In another embodiment, nucleic acids are labelled with biotin. For example, the present invention describes herein probes labelled with biotin for use in the methods described herein.

Fluorophore: A chemical compound, which when excited by exposure to a particular stimulus, such as a defined wavelength of light, emits light (fluoresces), for example, at a different wavelength (such as a longer wavelength of light).

Fluorophores are part of the larger class of luminescent compounds. Luminescent compounds include chemiluminescent molecules, which do not require a particular wavelength of light to luminesce, but rather use a chemical source of energy. Therefore, the use of chemiluminescent molecules (such as aequorin) eliminates the need for an external source of electromagnetic radiation, such as a laser.

In particular examples, a fluorophore is used as a donor fluorophore or as an acceptor fluorophore. “Acceptor fluorophores” are fluorophores which absorb energy from a donor fluorophore, for example in the range of about 400 to 900 nm (such as in the range of about 500 to 800 nm). Acceptor fluorophores generally absorb light at a wavelength which is usually at least 10 nm higher (such as at least 20 nm higher) than the maximum absorbance wavelength of the donor fluorophore, and have a fluorescence emission maximum at a wavelength ranging from about 400 to 900 nm. Acceptor fluorophores have an excitation spectrum that overlaps with the emission of the donor fluorophore, such that energy emitted by the donor can excite the acceptor. Ideally, an acceptor fluorophore is capable of being attached to a nucleic acid molecule.

In a particular example, an acceptor fluorophore is a dark quencher, such as Dabcyl, QSY7 (Molecular Probes), QSY33 (Molecular Probes), BLACK HOLE QUENCHERS™ (Biosearch Technologies; such as BHQ0, BHQ1, BHQ2, and BHQ3), ECLIPSE™ Dark Quencher (Epoch Biosciences), or IOWA BLACK™ (Integrated DNA Technologies). A quencher can reduce or quench the emission of a donor fluorophore.

“Donor Fluorophores” are fluorophores or luminescent molecules capable of transferring energy to an acceptor fluorophore, in some examples generating a detectable fluorescent signal from the acceptor. Donor fluorophores are generally compounds that absorb in the range of about 300 to 900 nm, for example about 350 to 800 nm. Donor fluorophores have a strong molar absorbance coefficient at the desired excitation wavelength.

Isolated: An “isolated” biological component (such as a nucleic acid) has been substantially separated or purified away from other biological components in which the component naturally occurs, such as other chromosomal and extrachromosomal DNA, RNA, and proteins. Nucleic acids that have been “isolated” include nucleic acids purified by standard purification methods. Isolated does not require absolute purity, and can include protein, peptide, or nucleic acid molecules that are at least 50% isolated, such as at least 75%, 80%, 90%, 95%, 98%, 99%, or even 99.9% isolated.

The present invention provides methods of preparing nucleic acids from a sample. In one embodiment, the nucleic acid is from a microbe, such as bacteria, fungi, protozoa, parasites, and viruses. In one embodiment, the nucleic acid is from an infectious agent.

Accordingly, in one embodiment, the infectious agent is a DNA virus or an RNA virus. In one embodiment the virus is selected from the group consisting of dsDNA viruses, ssDNA viruses, dsRNA viruses, (+) ssRNA viruses, (−) ssRNA viruses, ssRNA-RT viruses and dsDNA-RT viruses.

In another embodiment the virus is selected from the group consisting of Dengue, Herpes Simplex virus (e.g. HSV-1 and HSV-2), a norovirus, an arbovirus (e.g. Barmah Forest virus (BFV), Ross River virus (RRV), West Nile virus (WNV), yellow fever virus (YFV), Murray Valley encephalitis virus (MVEV), Japanese encephalitis virus (JEV), Human Immunodeficiency virus (HIV), dengue viruses, Zika virus, Measles, Mumps, Hendra virus, Nipah virus (e.g., humans and animals), Hepatitis (e.g. Hepatitis B, Hepatitis C), Severe acute respiratory syndrome coronavirus (e.g. SARS-CoV or SARS-CoV-2). In a preferred embodiment, the virus is Hendra virus, Nipah virus, Cedar virus, Dengue virus, Ross River Virus, Barmah Forest virus, Chikungunya virus, prawn Hepatopancreatic parvoviruses, and rabies virus.

In another embodiment, the infectious agent is a bacteria selected from the group consisting of Bordetella, Mycoplasma pneumoniae, Clostridioides difficile (formerly Clostridium difficile), Mycoplasma genitalium, Q-fever (Coxiella burnetii), Legionella spp, Helicobacter, Chlamydia (e.g., Chlamydia trachomatis, C. pneumoniae, C. psittaci, C. pecorum), Mycobacterium tuberculosis and non-TB mycobacteria, Streptococcus pyogenes (Group A strep), Streptococcus pneumoniae, Corynebacterium diphtheriae (diphtheria), Enterococcus faecium, Enterococcus faecalis, Clostridium tetani (tetanus), Syphilis (Treponema pallidum), Neisseria gonorrhoeae, Neisseria meningitidis, Staphylococcus aureus, Klebsiella pneumoniae, Acinetobacter baumannii, Pseudomonas aeruginosa, Enterobacter cloacae, Enterobacter spp, Wolbachia spp, Salmonella spp, Staphylococcus spp., and Escherichia coli (E. coli).

In another embodiment, the infectious agent is a parasite selected from the group consisting of Plasmodium spp, Toxoplasma spp, Giardia spp, Trypanosoma spp, Leishmania spp, Strongyloides spp., Schistosoma spp. Cryptosporidium spp, and Trichinella spp.

In another embodiment, the infectious agent is a human pathogenic fungus, for example, Cryptococcus, Aspergillus and Candida spp.

In another embodiment, the infectious agent is a gut or lung microbiome-derived organism.

In one embodiment, the virus is Severe acute respiratory syndrome coronavirus 2 (SARS-CoV-2): SARS-CoV-2 is the virus strain that causes coronavirus disease 2019 (COVID-19), a respiratory illness. It is colloquially known as the coronavirus, and was previously referred to by its provisional name 2019 novel coronavirus (2019-nCoV). SARS-CoV-2 is a positive-sense single-stranded RNA virus. It is contagious in humans, and the World Health Organization (WHO) has designated the COVID-19 pandemic as a Public Health Emergency of International Concern.

Taxonomically, SARS-CoV-2 is a strain of Severe acute respiratory syndrome-related coronavirus (SARS-CoV). It is believed to have zoonotic origins and has close genetic similarity to bat coronaviruses, suggesting it emerged from a bat-borne virus. An intermediate animal reservoir such as a pangolin is also thought to be involved in its introduction to humans. The virus shows little genetic diversity, indicating that the spillover event introducing SARS-CoV-2 to humans is likely to have occurred in late 2019.

Epidemiological studies estimate each infection results in 1.4 to 3.9 new ones when no members of the community are immune and no preventive measures taken, and further mutations are occurring that are leading to greater infectivity, with mutant strains labelled alpha, beta, gamma, delta, etc. The virus is primarily spread between people through close contact and via respiratory droplets produced from coughs or sneezes. It mainly enters human cells by binding to the receptor angiotensin converting enzyme 2 (ACE2).

Each SARS-CoV-2 virion is approximately 50-200 nanometres in diameter. Like other coronaviruses, SARS-CoV-2 has four structural proteins, known as the S (spike), E (envelope), M (membrane), and N (nucleocapsid) proteins; the N protein holds the RNA genome, and the S, E, and M proteins together create the viral envelope. The spike protein, which has been imaged at the atomic level using cryogenic electron microscopy, is the protein responsible for allowing the virus to attach to and fuse with the membrane of a host cell.

Malaria is a mosquito-borne pathology caused by Plasmodium parasites. The parasites are spread to people through the bites of infected female Anopheles mosquitoes. Five Plasmodium species cause malaria in humans: Plasmodium falciparum, Plasmodium vivax, Plasmodium ovale, Plasmodium malariae, and Plasmodium knowlesi. Among them, according to the World Health Organization (WHO), Plasmodium falciparum and Plasmodium vivax are responsible for the greatest threat. P. falciparum is the most prevalent malaria parasite on the African continent and is responsible for most malaria-related deaths globally. P. vivax is the dominant malaria parasite in most countries outside of sub-Saharan Africa.

Dengue virus (DENV), a member of the genus Flavivirus within the family Flaviviridae with four serotypes, is one of the most significant mosquito-borne viruses with an estimated 3 billion people living in affected areas. Dengue causes fevers and flu-like symptoms. A subsequent infection with a different serotype can lead to potentially fatal severe dengue, including dengue haemorrhagic fever, which can cause serious bleeding, blood pressure decreases and sometimes death.

NASBA: nucleic acid sequence-based amplification NASBA, which is initiated with reverse transcription of target RNA by a sequence-specific reverse primer to create an RNA/DNA duplex. RNase H is then used to degrade the RNA template, allowing a forward primer containing a promoter, such as the T7 promoter, to bind and initiate elongation of the complementary strand, generating a double-stranded DNA product. The RNA polymerase promoter-mediated transcription of the DNA template then creates copies of the target RNA sequence. Importantly, each of the new target RNAs can be detected by the guide RNAs thus further enhancing the sensitivity of the assay. The NASBA reaction has the additional advantage of being able to proceed under moderate isothermal conditions, for example at approximately 41° C., making it suitable for systems and devices deployed for early and direct detection in the field and far from clinical laboratories.

RPA: recombinase polymerase amplification (RPA) reaction may be used to amplify the target nucleic acids. RPA reactions employ recombinases which are capable of pairing sequence-specific primers with homologous sequence in duplex DNA. If target DNA is present, DNA amplification is initiated and no other sample manipulation such as thermal cycling or chemical melting is required. The entire RPA amplification system is stable as a dried formulation and can be transported safely without refrigeration. RPA reactions may also be carried out at isothermal temperatures with an optimum reaction temperature of 37-42° C. The sequence specific primers are designed to amplify a sequence comprising the target nucleic acid sequence to be detected. In certain example embodiments, an RNA polymerase promoter, such as a T7 promoter, is added to one of the primers. This results in an amplified double-stranded DNA product comprising the target sequence and an RNA polymerase promoter. After, or during, the RPA reaction, an RNA polymerase is added that will produce RNA from the double-stranded DNA templates. RPA reactions can also be used to amplify target RNA. The target RNA is first converted to cDNA using a reverse transcriptase, followed by second strand DNA synthesis, at which point the RPA reaction proceeds as outlined above.

PCR: As used herein, polymerase chain reaction (PCR) includes real-time/quantitative PCR (qPCR), reverse transcription quantitative PCR (RT-qPCR), digital droplet PCR (ddPCR) and the like.

Loop-mediated isothermal amplification (LAMP): A method for amplifying DNA. The method is a single-step amplification reaction utilizing a DNA polymerase with strand displacement activity (e.g., Notomi et al., Nucl. Acids. Res. 28: E63, 2000; Nagamine et al., Mol. Cell. Probes 16:223-229, 2002; Mori et al., J. Biochem. Biophys. Methods 59:145-157, 2004). At least four primers, which are specific for six regions within a target nucleic acid sequence, are typically used for LAMP. The primers include a forward outer primer (F3), a backward outer primer (B3), a forward inner primer (FIP), and a backward inner primer (BIP). A forward loop primer (Loop F), and a backward loop primer (Loop B) can also be included in some embodiments. The amplification reaction produces a stem-loop DNA with inverted repeats of the target nucleic acid sequence. Reverse transcriptase can be added to the reaction for amplification of RNA target sequences. This variation is referred to as RT-LAMP.

Primer: Primers are short nucleic acids, generally DNA oligonucleotides 10 nucleotides or more in length (such as 10-60, 15-50, 20-45, or 20-40 nucleotides in length). Primers may be annealed to a complementary target DNA strand by nucleic acid hybridization to form a hybrid between the primer and the target DNA strand, and then extended along the target DNA strand by a DNA polymerase enzyme. Primer pairs can be used for amplification of a nucleic acid sequence, e.g., by the polymerase chain reaction (PCR), LAMP, RT-LAMP, or other nucleic acid amplification methods known in the art.

Probe: A probe typically comprises an isolated nucleic acid (for example, at least 10 or more nucleotides in length) with an attached detectable label or reporter molecule. Typical labels include haptens, radioactive isotopes, ligands, chemiluminescent agents, fluorophores, and enzymes.

Methods for labelling oligonucleotides and guidance in the choice of labels appropriate for various purposes are discussed, e.g., in Sambrook et al. (2001) and Ausubel et al. (1987).

Sample (or biological sample): A biological specimen containing DNA (for example, genomic DNA or cDNA), RNA (including mRNA), protein, or combinations thereof. Examples include, but are not limited to isolated nucleic acids, cells, cell lysates, tissues, autopsy samples, bone marrow aspirates, blood, serum, plasma, urine, cerebrospinal fluid, middle ear fluids, breast milk, bronchoalveolar lavage, tracheal aspirates, sputum, oral fluids, nasopharyngeal aspirates, oropharyngeal aspirates, saliva, oral swabs, eye swabs, cervical swabs, vaginal swabs, rectal swabs, stool, stool suspensions, wastewater, soil, whole animals (e.g. mosquitoes) and plant material.

Reaction sample: As used herein, a reaction sample is formed by contacting a sample comprising nucleic acids with a composition comprising a quaternary ammonium compound, a denaturing agent and a precipitating agent.

Subject: Any multi-cellular vertebrate organism, such as human and non-human mammals (including non-human primates). In one example, a subject is known to be or is suspected of being infected with an infectious agent.

### II. Methods of Preparing Nucleic Acids

Disclosed herein are methods of preparing nucleic acids for nucleic acid amplification from a sample, said method comprising: contacting a sample comprising nucleic acids with a composition comprising a quaternary ammonium compound, a denaturing agent and a precipitating agent to form a reaction sample. The disclosed methods can be used to detect nucleic acid of infectious agents in a sample (such as in a sample from a subject infected with or suspected to be infected with an infectious agent). The disclosed methods can be used to detect a specific nucleic acid in a sample for genomic testing, for example for, diagnosis of a disease or condition.

For example, in Examples 2, 3, 4, 5, 6, 8, 9, 10, 11, 12, 13, 14 and 15 the present inventors have demonstrated that a composition comprising a quaternary ammonium compound, a denaturing agent and a precipitating agent can be contacted with a sample comprising nucleic acids to form a reaction sample, and the nucleic acids in the reaction sample can be amplified.

In Example 21, the present inventors have demonstrated that a composition comprising a quaternary ammonium compound, a denaturing agent and a precipitating agent can be contacted with a sample comprising nucleic acids to form a reaction sample, and the nucleic acids in the reaction sample can be detected using a detection system, such as a bead-based microarray.

In one embodiment the quaternary ammonium compound is betaine or betaine (mono)hydrate (not betaine HCl).

The present inventors have demonstrated in Example 7 that up to 2.5 M betaine can be included in the Sample Preparation Reagent. Accordingly, in one embodiment betaine is included in the Sample Preparation Reagent at a level of at least 0.1, 0.2, 0.3, 0.4, 0.5, 0.6, 0.7, 0.8, 0.9, 1, 1.1, 1.2, 2, 2.1, 2.2, 2.3, 2.4, or 2.5 M betaine.

The present inventors have demonstrated in Example 8 that 0.078125 M betaine can be included in the Sample Preparation Reagent and allow nucleic acid amplification. Accordingly, in one embodiment betaine is included in the Sample Preparation Reagent at a level of at least 0.078125, 0.3125, 0.625, 1.25 M or 2.5 M betaine.

In a preferred embodiment, betaine is included in the Sample Preparation Reagent at a level of at least 0.8, 0.9, 1, 1.1, 1.2, 1.33, 2, 2.1, 2.2, 2.3, 2.4, or 2.5 M betaine.

In a preferred embodiment the composition comprises from about 0.078 M to about 2.5 M betaine.

In another preferred embodiment the composition comprises from about 0.08 M to about 1.33 M betaine.

Sample preparation for nucleic acid amplification involving precipitating agents such as isopropanol, ethanol, and methanol involve removal of the precipitating agent since residual amounts of isopropanol, ethanol, and/or methanol have inhibitory effects on enzyme-dependent downstream applications such as PCR or RPA and next generation sequencing. In contrast, the present inventors have demonstrated that the sample preparation compositions described herein can be used for downstream applications without removal of precipitating agents expected to interfere with such downstream applications.

In another embodiment, the denaturing agent is selected from the group consisting of NaOH and KOH.

The present inventors have demonstrated in Example 7 that up to 1000 mM NaOH can be included in the Sample Preparation Reagent. Accordingly, in one embodiment NaOH is included in the Sample Preparation Reagent at a level of at least 100, 200, 300, 400, 500, 600, 700, 800, 900 or 1000 mM NaOH.

The present inventors have demonstrated in Example 8 that up to 1000 mM NaOH can be included in the Sample Preparation Reagent and allow nucleic acid amplification. Accordingly, in one embodiment NaOH is included in the Sample Preparation Reagent at a level of at least 15, 100, 200, 300, 400, 500, 600, 700, 800, 900 or 1000 mM NaOH.

In a preferred embodiment the composition comprises from about 15 mM to about 1000 mM denaturing agent.

In a preferred embodiment, NaOH is included in the Sample Preparation Reagent at a level of at least 125, 200, 300, 400, 500, 600, 700, 800, 900 or 1000 mM NaOH.

In a preferred embodiment the composition comprises from about 0.125 M to about 0.210 M NaOH.

In another embodiment, the precipitating agent is selected from the group consisting of isopropanol, ethanol, and methanol.

The present inventors have demonstrated in Example 7 that up to 80% isopropanol can be included in the Sample Preparation Reagent. Accordingly, in one embodiment isopropanol is included in the Sample Preparation Reagent at a level of at least 10, 20, 30, 40, 50, 60, 70, 80, 90 or 100% isopropanol.

The present inventors have demonstrated in Example 8 that up to 80% isopropanol can be included in the Sample Preparation Reagent and allow nucleic acid amplification. Accordingly, in one embodiment isopropanol is included in the Sample Preparation Reagent at a level of at least 10, 20, 30, 40, 50, 60, 70, or 80% isopropanol.

In a preferred embodiment, isopropanol is included in the Sample Preparation Reagent at a level of at least 31%, 40, 50, 60, 70, or 80% isopropanol.

In a preferred embodiment the composition comprises from about 10% to about 80% precipitating agent.

In another preferred embodiment the composition comprises from about 31% to about 80% precipitating agent.

In one embodiment the present invention provides a method as described herein, wherein the composition comprises up to 80% isopropanol, up to 125 mM NaOH, and up to 0.16 M betaine.

In one embodiment the present invention provides a method as described herein, wherein the composition comprises up to 80% isopropanol, up to 31 mM NaOH, and up to 0.31 M betaine.

In one embodiment the present invention provides a method as described herein, wherein the composition comprises up to 1000 mM NaOH, up to 0.31 M betaine, and up to 60% isopropanol.

In one embodiment the present invention provides a method as described herein, wherein the composition comprises up 1000 to mM NaOH, up to 0.625 M betaine, and up to 40% isopropanol.

In one embodiment the present invention provides a method as described herein, wherein the composition comprises up to 2.5 M Betaine, up to 500 mM NaOH, and up to 20% isopropanol.

In one embodiment the present invention provides a method as described herein, wherein the composition comprises 1.25 M Betaine, 200 mM NaOH, and 40% isopropanol. This Sample Preparation Reagent is referred to herein as the Sample Preparation Reagent U.

In one embodiment the present invention provides a method as described herein, wherein the composition comprises 1.125 M Betaine, 180 mM NaOH, and 36% isopropanol. This Sample Preparation Reagent is referred to herein as the Sample Preparation Reagent E.

The present inventors have demonstrated in the Examples that detergents can be omitted from the Sample Preparation Reagents described herein, while still allowing release of nucleic acids from a sample into the reaction sample. Accordingly, in one embodiment, the present invention provides a method as described herein, wherein the composition does not comprise a detergent.

In one embodiment, the detergent to be avoided is sodium dodecyl sulfate (SDS).

The present inventors have also demonstrated in the Examples that chelating agents such as ethylenediaminetetraacetic acid (EDTA) can be omitted from the Sample Preparation Reagents described herein, while still allowing release of nucleic acids from a sample into the reaction sample, and avoiding degradation of the nucleic acids. Accordingly, in one embodiment, the present invention provides a method as described herein, wherein the composition does not comprise EDTA or a chelating agent.

The present inventors have also demonstrated in the Examples that agents that function to inhibit secondary structures in nucleic acids, such as dimethylsulfoxide (DMSO), can be omitted from the Sample Preparation Reagents described herein, while still allowing release of nucleic acids from a sample into the reaction sample, and subsequent amplification. Accordingly, in one embodiment, the present invention describes a method as described herein, wherein the composition does not comprise DMSO.

In one embodiment, the present invention provides a method as described herein wherein the composition further comprises RNase- and DNase-free water.

The present inventors have also demonstrated in the Examples that the Sample Preparation Reagents described herein can be used without purification of nucleic acids from the reaction sample following contacting the sample with the Sample Preparation Reagent.

Accordingly, in one embodiment, the present invention provides a method as described herein wherein the nucleic acids are not separated from the reaction sample prior to nucleic acid amplification.

The present inventors have also demonstrated in the Examples that the Sample Preparation Reagents described herein can be mixed with the sample at different ratios and allow amplification of nucleic acids in the sample. The present inventors have also demonstrated that the reaction sample can be diluted (e.g., in water) following contacting the sample with the Sample Preparation Reagents described herein and allow amplification of nucleic acids in the sample.

As used herein, a ratio of X+Y (for example in the Figures) denotes a ratio of X: Y. For example, a ratio of 1+5 is used to denote a ratio of 1:5, and is used interchangeably with a ratio of 1:5 etc.

Accordingly, in one embodiment, the reaction sample is diluted prior to nucleic acid amplification.

Table 1 sets out exemplary ratios and/or dilutions for use in the methods described herein.

TABLE 1
Suggested ratios and volumes of sample:Sample Preparation
Reagent E with or without a post-incubation dilution step.
Conditions for liquid samples

| | | Dilution in |
|---|---|---|
| Sample/Reagent E | | water |
| Ratio | Suggested volumes | after incubation |
| 1:1 | 10 μL Sample + 10 μL Reagent E | No dilution |
| 1:2 | 7.5 μL Sample + 15 μL Reagent E | No dilution |
| 1:5 | 4 μL Sample + 20 μL Reagent E | No dilution |
| 1:10 | 2 μL Sample + 20 μL Reagent E | No dilution |
| 2:1 | 15 μL Sample + 7.5 μL Reagent E | No dilution |
| 5:1 | 20 μL Sample + 4 μL Reagent E | No dilution |
| 10:1 | 20 μL Sample + 2 μL Reagent E | No dilution |
| 1:1 | 10 μL Sample + 10 μL Reagent E | 1 in 2 |
| | | (5 μL into 5 μL |
| | | water) |
| 1:2 | 7.5 μL Sample + 15 μL Reagent E | 1 in 2 |
| 1:5 | 4 μL Sample + 20 μL Reagent E | 1 in 2 |
| 1:10 | 2 μL Sample + 20 μL Reagent E | 1 in 2 |
| 2:1 | 15 μL Sample + 7.5 μL Reagent E | 1 in 2 |
| 5:1 | 20 μL Sample + 4 μL Reagent E | 1 in 2 |
| 10:1 | 20 μL Sample + 2 μL Reagent E | 1 in 2 |
| 1:1 | 10 μL Sample + 10 μL Reagent E | 1 in 5 (5 μL into |
| | | 20 μL water) |
| 1:2 | 7.5 μL Sample + 15 μL Reagent E | 1 in 5 |
| 1:5 | 4 μL Sample + 20 μL Reagent E | 1 in 5 |
| 1:10 | 2 μL Sample + 20 μL Reagent E | 1 in 5 |
| 2:1 | 15 μL Sample + 7.5 μL Reagent E | 1 in 5 |
| 5:1 | 20 μL Sample + 4 μL Reagent E | 1 in 5 |
| 10:1 | 20 μL Sample + 2 μL Reagent E | 1 in 5 |

Conditions for solid samples

| | | | | Dilution in water | |
|---|---|---|---|---|---|
| Sample/Reagent | Sample | TNA-Cifer Reagent E | | after 10 min | Results |
| E Ratio | volume (μg) | volume (μL) | | incubation | (e.g. PCR Ct) |
| 5:1 | 50 μg | 10 | μL | No dilution | |
| 2:1 | (or one “unit”) | 25 | μL | No dilution | |
| 1:1 | convenient to | 50 | μL | No dilution | |
| 1:2 | your trial, | 100 | μL | No dilution | |
| 1:5 | (e.g. a nasal | 250 | μL | No dilution | |
| 1:10 | swab*) | 500 | μL | No dilution | |
| 5:1 | 50 μg | 10 | μL | 1 in 2 | |
| | (or one “unit”) | | | (5 μL into 5 μL | |
| | convenient to | | | water) | |
| 2:1 | your trial* | 25 | μL | 1 in 2 | |
| 1:1 | (e.g. a nasal | 50 | μL | 1 in 2 | |
| 1:2 | swab*) | 100 | μL | 1 in 2 | |
| 1:5 | | 250 | μL | 1 in 2 | |
| 1:10 | | 500 | μL | 1 in 2 | |
| 5:1 | 50 μg | 10 | μL | 1 in 5 | |
| | (or one “unit”) | | | (5 μL into 20 μL | |
| | convenient to | | | water) | |
| 2:1 | your trial* | 25 | μL | 1 in 5 | |
| 1:1 | (e.g. a nasal | 50 | μL | 1 in 5 | |
| 1:2 | swab*) | 100 | μL | 1 in 5 | |
| 1:5 | | 250 | μL | 1 in 5 | |
| 1:10 | | 500 | μL | 1 in 5 | |

*For consistency of results, we adjust Reagent E to the number of ug or units in the sample. For example, for 5:1 trials with 55 μg sample, add 11 μL TNA-Cifer Reagent E. For larger samples (e.g. a nasal swab) larger volume may be required (e.g. beginning at 500 μL and moving up to 1 mL, etc.)

Accordingly, in one embodiment, the present invention provides a method as described herein, wherein the contacting is performed at a sample to composition (e.g. a Sample Preparation Reagent described herein) ratio of 1:1 or greater. For example, a ratio of 1:1, 2:1, 3:1, 4:1, 5:1, 6:1, 7:1, 8:1, 9:1 or 10:1 or greater.

In a preferred embodiment, the present invention provides a method of preparing nucleic acids for nucleic acid amplification from a sample, said method comprising contacting a sample comprising nucleic acids with a composition comprising a quaternary ammonium compound, a denaturing agent and a precipitating agent to form a reaction sample, wherein the contacting is performed at a sample:composition ratio of between 10:1 and 1:10, wherein the sample is culture media, and wherein the reaction sample is diluted 1 in 2 prior to nucleic acid amplification.

In a preferred embodiment, the present invention provides a method of preparing nucleic acids for nucleic acid amplification from a sample, said method comprising contacting a sample comprising nucleic acids with a composition comprising a quaternary ammonium compound, a denaturing agent and a precipitating agent to form a reaction sample, wherein the contacting is performed at a sample:composition ratio of 1:1, wherein the sample is blood, and wherein the reaction sample is diluted 1 in 2 prior to nucleic acid amplification.

In a preferred embodiment, the present invention provides a method of preparing nucleic acids for nucleic acid amplification from a sample, said method comprising contacting a sample comprising nucleic acids with a composition comprising a quaternary ammonium compound, a denaturing agent and a precipitating agent to form a reaction sample, wherein the contacting is performed at a sample:composition ratio of 2:1, wherein the sample is plasma, and wherein the reaction sample is diluted 1 in 2 prior to nucleic acid amplification.

In a preferred embodiment, the present invention provides a method of preparing nucleic acids for nucleic acid amplification from a sample, said method comprising contacting a sample comprising nucleic acids with a composition comprising a quaternary ammonium compound, a denaturing agent and a precipitating agent to form a reaction sample, wherein the contacting is performed at a sample:composition ratio of 5:1, wherein the sample is serum, and wherein the reaction sample is diluted 1 in 2 prior to nucleic acid amplification.

Accordingly, in one embodiment, the present invention provides a method as described herein, wherein the contacting is performed at a sample to composition ratio of 1:1 or less. For example, a ratio of 1:1, 1:2, 1;3, 1:4, 1:5, 1:6, 1:7, 1:8, 1:9, or 1:10 or less.

In a preferred embodiment, the present invention provides a method of preparing nucleic acids for nucleic acid amplification from a sample, said method comprising contacting a sample comprising nucleic acids with a composition comprising a quaternary ammonium compound, a denaturing agent and a precipitating agent to form a reaction sample, wherein the contacting is performed at a sample:composition ratio of 1:5, wherein the sample is culture media.

In a preferred embodiment, the present invention provides a method of preparing nucleic acids for nucleic acid amplification from a sample, said method comprising contacting a sample comprising nucleic acids with a composition comprising a quaternary ammonium compound, a denaturing agent and a precipitating agent to form a reaction sample, wherein the contacting is performed at a sample:composition ratio of 1:5, wherein the sample is a nasal swab.

In one embodiment, the contacting of a sample with a Sample Preparation Reagent described herein is performed for at least 10, 20, 30, 40, 50, or 60 seconds, or at least 2, 3, 4, 5, 6, 7, 8, 9, 10, 15, 20, 25, 30, 40, 45, 50, 55, or 60 minutes.

In one embodiment, the contacting is performed for less than 15 minutes.

In another embodiment, the contacting of a sample with a Sample Preparation Reagent described herein is performed at room temperature.

In another embodiment, the contacting of a sample with a Sample Preparation Reagent described herein is performed on ice.

The present inventors have demonstrated in the Examples that a Sample Preparation Reagent described herein can be used to prepare reaction samples comprising nucleic acids released from serum, plasma, tissue, bacterial culture, whole blood, culture supernatants, mosquitoes, and liquids. Accordingly, the methods the present invention can be used with a number of sample types.

In one embodiment, the present invention provides a method as described herein, wherein the sample comprises nucleic acids. Appropriate samples for use in the methods disclosed herein include any conventional biological sample obtained from an organism or a part thereof, such as a plant, animal, bacteria, etc.

In particular embodiments, the biological sample is obtained from an animal subject, such as a human subject. A biological sample is any solid or fluid sample obtained from, excreted by or secreted by any living organism, including, without limitation, single celled organisms, such as bacteria, yeast, protozoans, and amoebas among others, as well as multicellular organisms (such as plants or animals, including samples from a healthy or apparently healthy human subject or a human patient affected by a condition or disease to be diagnosed or investigated, such as an infection with a pathogenic microorganism (e.g. a pathogenic bacterium or virus). For example, a biological sample can be a biological fluid obtained from, for example, blood, plasma, serum, urine, stool, sputum, mucous, lymph fluid, synovial fluid, bile, ascites, pleural effusion, seroma, saliva, cerebrospinal fluid, aqueous or vitreous humour, or any bodily secretion, a transudate, an exudate (for example, fluid obtained from an abscess or any other site of infection or inflammation), or fluid obtained from a joint (for example, a normal joint or a joint affected by disease, such as rheumatoid arthritis, osteoarthritis, gout or septic arthritis), or a swab of skin or mucosal membrane surface.

A sample can also be a sample obtained from any organ or tissue (including a biopsy or autopsy specimen, such as a tumour biopsy) or can include a cell (whether a primary cell or cultured cell) or medium conditioned by any cell, tissue or organ.

Accordingly, in one embodiment, the present invention provides a method as described herein, wherein the sample is selected from the group consisting of cells, tissues, autopsy samples, bone marrow aspirates, blood, serum, plasma, urine, cerebrospinal fluid, middle ear fluids, breast milk, bronchoalveolar lavage, tracheal aspirates, sputum, oral fluids, nasopharyngeal aspirates, oropharyngeal aspirates, saliva, oral swabs, eye swabs, cervical swabs, vaginal swabs, rectal swabs, stool, stool suspensions, wastewater, soil, and plant material.

In other embodiments, a sample may be an environmental sample, such as water, soil, or a surface such as industrial or medical surface.

In other embodiments, the systems, devices, and methods, disclosed herein are directed to detecting the presence of one or more microbial agents in a sample, such as a biological sample obtained from a subject, wastewater, soil and plant material.

In one embodiment, the microbe may be a bacterium, a fungus, a yeast, a protozoan, a parasite, or a virus. Accordingly, the methods disclosed herein can be adapted for use in other methods (or in combination) with other methods that require quick identification of microbe species, monitoring the presence of microbial proteins (antigens), antibodies, antibody genes, detection of certain phenotypes (e.g. bacterial resistance), monitoring of disease progression and/or outbreak, and antibiotic screening. Because of the rapid and sensitive diagnostic capabilities of the embodiments disclosed herein, the embodiments disclosed herein may be used as guide therapeutic regimens, such as a selection of the appropriate antibiotic or antiviral.

The embodiments disclosed herein may also be used to screen environmental samples (air, water, surfaces, food etc.) for the presence of microbial contamination.

In one embodiment, the present invention provides a method to identify the microbial species, such as bacterial, viral, fungal, yeast, or parasitic species, or the like. Particular embodiments disclosed herein describe methods and systems that will identify and distinguish microbial species within a single sample, or across multiple samples, allowing for recognition of many different microbes. The present methods allow the detection of pathogens and distinguishing between two or more species of one or more organisms, e.g., bacteria, viruses, yeast, protozoa, and fungi or a combination thereof, in a biological or environmental sample, by detecting the presence of a target nucleic acid sequence in the sample. A positive signal obtained from the sample indicates the presence of the microbe. Multiple microbes can be identified simultaneously using the methods of the invention.

Multiplex analysis of samples enables large-scale detection of samples, reducing the time and cost of analyses. The present invention provides herein methods for distinguishing between two or more species of one or more organisms in a sample. The methods are also amenable to detecting one or more species of one or more organisms in a sample.

In some embodiments, a method for detecting microbes in samples is provided comprising distributing a sample or set of samples into one or more individual discrete volumes, the individual discrete volumes comprising a Sample Preparation Reagent as described herein; incubating the sample or set of samples under conditions to allow nucleic acids to be made available within the sample for nucleic acid amplification. The one or more target molecules may be mRNA, DNA (e.g. gDNA, coding or non-coding), tRNA, rRNA or RNA comprising a target nucleotide sequence that may be used to distinguish two or more microbial species/strains from one another.

The present inventors have demonstrated in the Examples that samples comprising infectious agents including viruses, mosquitoes, and bacteria can be contacted with a Sample Preparation Reagent as described herein, and nucleic acids from the sample amplified.

Accordingly, in one embodiment, the present invention provides a method as described herein, wherein the sample is from a subject infected with or at risk of infection with an infectious agent.

In one embodiment, the present invention provides a method as described herein, wherein the infectious agent is selected from the group consisting of a bacterium, a fungus, a yeast, a protozoan, a parasite, or a virus.

The present inventors have demonstrated in Examples 1, 10 and 11 that contacting a sample comprising infectious agents renders the reaction sample non-infectious. Accordingly, in one embodiment, the present invention provides a method as described herein, wherein contacting the sample with the composition renders the infectious agent non-infectious.

In one embodiment, the present invention provides a method as described herein, further comprising performing nucleic acid amplification on the reaction sample.

In one embodiment the present invention provides a method as described herein wherein the nucleic acid amplification is selected from the group consisting of nucleic-acid sequenced-based amplification (NASBA), recombinase polymerase amplification (RPA), recombinase assisted amplification/recombinase aided amplification (RAA), loop-mediated isothermal amplification (LAMP), strand displacement amplification (SDA), helicase-dependent amplification (HDA), nicking enzyme amplification reaction (NEAR), polymerase chain reaction (PCR), multiple displacement amplification (MDA), rolling circle amplification (RCA), ligase chain reaction (LCR), and ramification amplification method (RAM). Any suitable RNA or DNA amplification technique may be used.

In another embodiment, the nucleic acid detection is the MassArray System (Agena Bioscience) which couples mass spectrometry with end-point PCR, enabling highly multiplexed reactions under universal cycling conditions to provide accurate, sensitive and rapid genetic analysis.

In another embodiment, the present invention provides a method as described herein, further comprising performing nucleic acid detection on the reaction sample.

In one embodiment, nucleic acid detection is performed using a method of detection as described herein. Nucleic acid detection methods are known in the art, and can include Bead-based Microarray Technology (Infinium Array, Illumina) on which probes bind to a complementary sequence in the sample DNA allowing allele identification, visualisation by gel electrophoresis and staining of DNA with intercalating dyes, or other methods of nucleic acid detection known in the art. Any suitable RNA or DNA detection technique may be used.

In one embodiment, the present invention provides a reaction sample prepared by a method as described herein.

In one embodiment the present invention provides a method of detecting nucleic acids in a sample comprising: contacting a sample comprising nucleic acids with a composition comprising a quaternary ammonium compound, a denaturing agent and a precipitating agent to form a reaction sample; optionally diluting the reaction sample; and performing nucleic acid amplification on the sample.

Different components or reagents useful for amplification of nucleic acids are described herein. For example, an amplification reagent as described herein may include a buffer, such as a Tris buffer. A Tris buffer may be used at any concentration appropriate for the desired application or use, for example including, but not limited to, a concentration of 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 25 mM, 50 mM, 75 mM, 1 M, or the like. One of skill in the art will be able to determine an appropriate concentration of a buffer such as Tris for use with the present invention.

A salt, such as magnesium chloride (MgCl₂), potassium chloride (KCl), or sodium chloride (NaCl), may be included in an amplification reaction, such as PCR, in order to improve the amplification of nucleic acid fragments. Although the salt concentration will depend on the particular reaction and application, in some embodiments, nucleic acid fragments of a particular size may produce optimum results at particular salt concentrations. Larger products may require altered salt concentrations, typically lower salt, in order to produce desired results, while amplification of smaller products may produce better results at higher salt concentrations. One of skill in the art will understand that the presence and/or concentration of a salt, along with alteration of salt concentrations, may alter the stringency of a biological or chemical reaction, and therefore any salt may be used that provides the appropriate conditions for a reaction of the present invention and as described herein.

Amplification reactions may include dNTPs and nucleic acid primers used at any concentration appropriate for the invention, such as including, but not limited to, a concentration of 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, 950 nM, 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, 150 mM, 200 mM, 250 mM, 300 mM, 350 mM, 400 mM, 450 mM, 500 mM, or the like. Likewise, a polymerase useful in accordance with the invention may be any specific or general polymerase known in the art and useful for the invention, including Taq polymerase, Q5 polymerase, or the like.

In some embodiments, amplification reagents as described herein may be appropriate for use in hot-start amplification. Hot start amplification may be beneficial in some embodiments to reduce or eliminate dimerization of adaptor molecules or oligos, or to otherwise prevent unwanted amplification products or artefacts and obtain optimum amplification of the desired product. Many components described herein for use in amplification may also be used in hot-start amplification. In some embodiments, reagents or components appropriate for use with hot-start amplification may be used in place of one or more of the composition components as appropriate. For example, a polymerase or other reagent may be used that exhibits a desired activity at a particular temperature or other reaction condition. In some embodiments, reagents may be used that are designed or optimized for use in hot-start amplification, for example, a polymerase may be activated after transposition or after reaching a particular temperature. Such polymerases may be antibody-based or aptamer-based. Polymerases as described herein are known in the art. Examples of such reagents may include, but are not limited to, hot-start polymerases, hot-start dNTPs, and photo-caged dNTPs. Such reagents are known and available in the art. One of skill in the art will be able to determine the optimal temperatures as appropriate for individual reagents.

Amplification of nucleic acids may be performed using specific thermal cycle machinery or equipment, and may be performed in single reactions or in bulk, such that any desired number of reactions may be performed simultaneously. In some embodiments, amplification may be performed using microfluidic or robotic devices, or may be performed using manual alteration in temperatures to achieve the desired amplification. In some embodiments, optimization may be performed to obtain the optimum reaction conditions for the particular application or materials. One of skill in the art will understand and be able to optimize reaction conditions to obtain sufficient amplification.

The present invention also provides a sample preparation composition as described herein, wherein the composition comprises a quaternary ammonium compound, a denaturing agent and a precipitating agent.

In one embodiment, the Sample Preparation Reagent is contacted with a sample to form a reaction sample.

In one embodiment, the quaternary ammonium compound is betaine.

In another embodiment, the denaturing agent is selected from the group consisting of NaOH, and KOH.

In another embodiment the present invention provides Sample Preparation Reagent composition as described herein, wherein the precipitating agent is selected from the group consisting of isopropanol, ethanol, and methanol.

In another embodiment the present invention provides a Sample Preparation Reagent composition as described herein, wherein the composition does not comprise a detergent.

In one embodiment, the detergent to be avoided is SDS.

In another embodiment the present invention provides a Sample Preparation Reagent composition as described herein, wherein the composition does not comprise EDTA.

In another embodiment the present invention provides a Sample Preparation Reagent composition as described herein, wherein the composition does not comprise DMSO.

In another embodiment the present invention provides a Sample Preparation Reagent composition as described herein, wherein the composition comprises up to 2.5 M betaine.

In another embodiment the present invention provides a Sample Preparation Reagent composition as described herein, wherein the composition comprises up to 1000 mM denaturing agent.

In another embodiment the present invention provides a Sample Preparation Reagent composition as described herein, wherein the composition comprises up to 80% precipitating agent.

In another embodiment the present invention provides a Sample Preparation Reagent composition as described herein, wherein the composition comprises up to 80% isopropanol, up to 125 mM NaOH, and up to 0.16 M betaine

In another embodiment the present invention provides a Sample Preparation Reagent composition as described herein, wherein the composition comprises up to 80% isopropanol, up to 31 mM NaOH, and up to 0.31 M betaine

In another embodiment the present invention provides a Sample Preparation Reagent composition as described herein, wherein the composition comprises up to 1000 mM NaOH, up to 0.31 M betaine, and up to 60% isopropanol

In another embodiment the present invention provides a Sample Preparation Reagent composition as described herein, wherein the composition comprises up to 1000 mM NaOH, up to 0.625 M betaine, and up to 40% isopropanol.

In another embodiment the present invention provides a Sample Preparation Reagent composition as described herein, wherein the composition comprises up to 2.5 M betaine, up to 500 mM NaOH, and up to 20% isopropanol.

In another embodiment the present invention provides a Sample Preparation Reagent composition as described herein, wherein the composition comprises 1.25 M betaine, 200 mM NaOH, and 40% isopropanol.

In another embodiment the present invention provides a Sample Preparation Reagent composition as described herein, wherein the composition comprises 1.125 M betaine, 180 mM NaOH, and 36% isopropanol.

In another embodiment the present invention provides a Sample Preparation Reagent composition as described herein, wherein the composition comprises RNase and DNase-free water.

The methods described herein may be used for any purpose for which detection of nucleic acids, is desirable, including diagnostic and prognostic applications, such as in laboratory and clinical settings.

The disclosed methods are highly sensitive and/or specific for detection of nucleic acids. In some examples, the disclosed methods can detect presence of 1 copy of virus nucleic acids (for example, at least 101, 102, 103, 104, 105, 106, 107, or more copies of nucleic acids) in a sample or a particular reaction volume (such as per mL reaction). In particular, non-limiting examples, the disclosed methods have a limit of detection of about 200 CCID₅₀/mL for SARS-CoV-2 RNA. However, one of ordinary skill in the art will recognize that the limit of detection of an assay depends on many factors (such as reaction conditions, amount and quality of starting material, and so on) and the limit of detection using particular amplification methods (e.g. RPA, qPCR, using primer sets), such as those disclosed herein, may be even less with modifications to the assay conditions.

One advantage of the disclosed methods is that they can detect presence of nucleic acid in a sample (for example, to diagnose SARS-CoV-2 infection) at an earlier time point in the course of infection than many currently available testing methods.

In some examples, the disclosed methods can predict with a sensitivity of at least 90% and a specificity of at least 90% for presence of nucleic acid (e.g. SARS-CoV-2 nucleic acid), such as a sensitivity of at least 91%, 92%, 93%, 94%, 95%, 96%, 97%, 98%, 99%, or even 100% and a specificity of at least of at least 91%, 92%, 93%, 94%, 95%, 96%, 97%, 98%, 99%, or even 100%.

Disclosed herein are methods for detecting nucleic acids in a sample utilizing isothermal amplification methods that can produce amplified nucleic acids in a short period of time. The isothermal nature of RPA, RT-RPA, LAMP and RT-LAMP allows for assay flexibility because it can be used with simple and inexpensive heating devices, which can facilitate nucleic acid detection in settings other than centralized clinical laboratories, including at the point-of-care (POC). POC testing is particularly important for diagnosis of infections with infectious agents, as it has the potential to reduce people who do not have follow up appointments, increase the number of individuals that become aware of their infection status (for example, at the time of their visit), and prevent community transmission.

In some embodiments, the methods include contacting a sample (such as a sample including or suspected to include SARS-CoV-2 nucleic acids) with at least one set of primers specific for a SARS-CoV-2 nucleic acid under conditions sufficient for amplification of the SARS-CoV-2 nucleic acid, producing an amplification product. In some examples, the primers amplify a SARS-CoV-2 nucleic acid having at least 80% sequence identify (such as at least 85%, 90%, 95%, 98%, or more sequence identity) to a reference sequence, or a portion thereof. In some examples, the methods further include reverse transcription of SARS-CoV-2 RNA in the sample, for example by contacting the sample with a reverse transcriptase. Contacting the sample with reverse transcriptase may be prior to contacting the sample with the one or more sets of primers or may be simultaneous with contacting the sample with the one or more sets of primers (for example in the same reaction mix with the primers). The amplification product is detected by any suitable method, such as with a colorimetric assay.

In some embodiments, the methods include contacting a sample (such as a sample including or suspected to include Plasmodial nucleic acids) with at least one set of primers specific for a Plasmodium nucleic acid under conditions sufficient for amplification of the Plasmodium nucleic acid, producing an amplification product. In some examples, the primers amplify a Plasmodium nucleic acid having at least 80% sequence identify (such as at least 85%, 90%, 95%, 98%, or more sequence identity) to a reference sequence, or a portion thereof. The amplification product is detected by any suitable method, such as with a colorimetric assay, such as with an intercalating dye.

### III. Primers and Kits

Primers (such as isolated nucleic acid primers) suitable for use in the disclosed methods are described herein. In some examples, the primers are suitable for detection of nucleic acids using PCR or RPA assays described herein.

In some embodiments, the primers are between 10 and 60 nucleotides in length, such as 10, 11, 12, 13, 14, 15, 16, 17, 18, 19, 20, 21, 22, 23, 24, 25, 26, 27, 28 29, 30, 31, 32, 32, 34, 35, 36, 37, 38, 39, 40, 41, 42, 43, 44, 45, 46, 47, 48, 49, 50, 51, 52, 53, 54, 55, 56, 57, 58, 59, or 60 nucleotides in length and are capable of hybridizing to, and in some examples, amplifying the disclosed nucleic acid molecules. In some examples, the primers are at least 10, 15, 20, 25, 30, 35, 40, 45, 50, 55, or 60 nucleotides in length. In other examples, the primers may be no more than 10, 15, 20, 25, 30, 35, 40, 45, 50, 55, or 60 nucleotides in length.

In some embodiments, the probes are between 46 and 52 nucleotides in length, such as 46, 47, 48, 49, 50, 51, or 52 nucleotides in length and are capable of hybridizing to the disclosed nucleic acid molecules.

In one embodiment, the primers include primers for amplification of the nucleocapsid (N) gene of SARS-CoV-2.

In one embodiment, the primers include primers for amplification of the E gene of SARS-CoV-2.

In one embodiment, the primers include primers for amplification of the ORF1ab gene of SARS-CoV-2.

In another embodiment, the primers include primers for amplification of the structural capsid protein gene of a hepatopancreatic parvovirus (HPV).

In another embodiment, the primers include primers for amplification of the NS5 gene of a Dengue virus.

In another embodiment, the primers include primers for amplification of a beta-lactamase gene of E. coli.

In another embodiment, the primers include primers for amplification of a wsp gene of Wolbachia.

In another embodiment, the primers include primers for amplification of an 18S rRNA gene of Plasmodium.

In some examples, at least one of the primers includes a detectable label, such as a fluorophore.

Although exemplary primer sequences are provided herein, the primer sequences can be varied slightly by moving the primer a few nucleotides upstream or downstream from the nucleotide positions that they hybridize to on the target nucleic molecule acid, provided that the primer is still specific for the target nucleic acid sequence. For example, variations of the primers disclosed can be made by “sliding” the probes or primers a few nucleotides 5′ or 3′ from their positions, and such variations will still be specific for the respective target nucleic acid sequence.

Also provided by the present disclosure are primers that include variations to the nucleotide sequences described herein, as long as such variations permit detection of the target nucleic acid molecule. For example, a primer can have at least 86% sequence identity such as at least 86%, 90% 91%, 92%, 93%, 94%, 95%, 96%, 97%, 98%, or 99% identity to a nucleic acid described herein. In such examples, the number of nucleotides does not change, but the nucleic acid sequence can vary at a few nucleotides, such as changes at 1, 2, 3, 4, 5, or 6 nucleotides.

The present application also provides primers that are slightly longer or shorter than the nucleotide sequences described herein as long as such deletions or additions permit amplification and/or detection of the desired target nucleic acid molecule. For example, a primer can include a few nucleotide deletions or additions at the 5′- or 3′-end of the primers described herein such as addition or deletion of 1, 2, 3, 4, 5, or 6 nucleotides from the 5′-or 3′-end, or combinations thereof (such as a deletion from one end and an addition to the other end). In such examples, the number of nucleotides changes.

Also provided are primers that are degenerate at one or more positions (such as 1, 2, 3, 4, 5, or more positions), for example, a primer that includes a mixture of nucleotides (such as 2, 3, or 4 nucleotides) at a specified position in the primer. In other examples, the primers disclosed herein include one or more synthetic bases or alternative bases (such as inosine). In other examples, the primers disclosed herein include one or more modified nucleotides or nucleic acid analogues, such as one or more locked nucleic acids (see, e.g., U.S. Pat. No. 6,794,499) or one or more superbases (Nanogen, Inc., Bothell, WA). In other examples, the primers disclosed herein include a minor groove binder conjugated to the 5′ or 3′ end of the oligonucleotide (see, e.g., U.S. Pat. No. 6,486,308).

The nucleic acid primers disclosed herein can be supplied in the form of a kit for use in the detection or amplification of one or more nucleic acids. In such a kit, an appropriate amount of one or more of the nucleic acid primers described herein are provided in one or more containers or in one or more individual wells or channels of a multi-well plate or card or a microfluidic device. A nucleic acid primer(s) may be provided suspended in an aqueous solution or as a freeze-dried or lyophilized powder, for instance. The container(s) in which the nucleic acid(s) are supplied can be any conventional container that is capable of holding the supplied form, for instance, microfuge tubes, multi-well plates, ampoules, or bottles. The kits can include either labelled or unlabelled nucleic acid primers (for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or more primers) for use in amplification and/or detection of SARS-CoV-2 nucleic acids.

One or more positive and/or negative control primers and/or nucleic acids also may be supplied in the kit. In the case of SARS-CoV-2, exemplary negative controls include non-SARS-CoV-2 nucleic acids or non-viral nucleic acids (such as human nucleic acids). Exemplary positive controls include primers and nucleic acids for amplification of human target nucleic acids (such as human β-actin or RNaseP) or primers and nucleic acids for other SARS-CoV-2 target nucleic acids (for example other regions of the SARS-CoV-2 gene or other SARS-CoV-2 genes).

One of ordinary skill in the art can select suitable positive and negative controls for the methods disclosed herein.

In some examples, one or more primers (such as one or more sets of primers), are provided in pre-measured single use amounts in individual, typically disposable, tubes, wells, microfluidic devices, or equivalent containers. In this example, the sample to be tested for the presence of the target nucleic acids can be added to the individual tube(s) or well(s) and amplification and/or detection can be carried out directly. The kit may also include additional reagents for the detection and/or amplification of nucleic acids, such as buffer(s), nucleotides (such as dNTPs), enzymes (such as DNA polymerase and/or reverse transcriptase), or other suitable reagents. The additional reagents may be in separate container(s) from the one or more primers or may be included in the same container as the primer(s).

The present disclosure is illustrated by the following non-limiting Examples.

### Examples

### EXAMPLE 1: Sample Preparation Reagent E Inactivates SARS-CoV-2 in Less than 30 Seconds

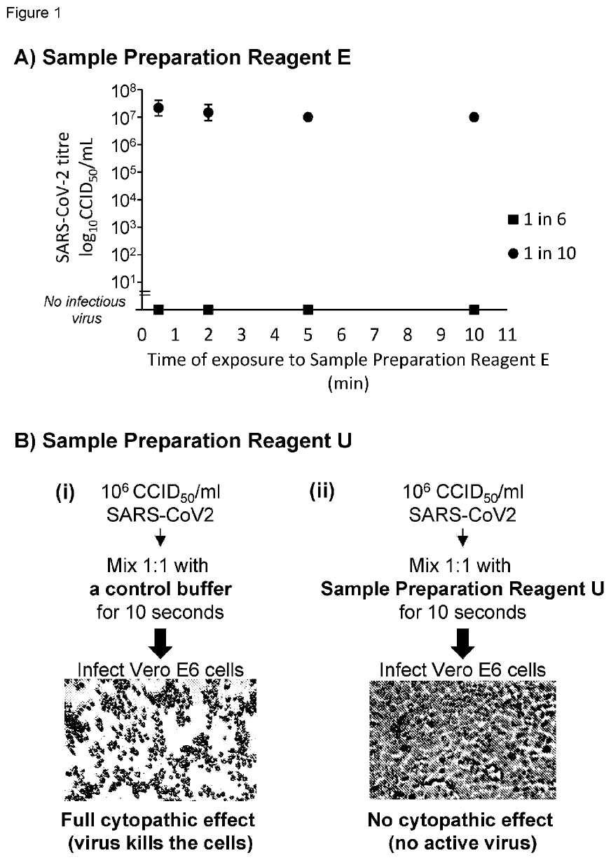

To determine if small amounts of Sample Preparation Reagent E could inactivate high concentrations of cultured SARS-CoV-2, the Sample Preparation Reagent E was mixed with cultured virus (107 CCID₅₀/mL) at a 1 in 6 dilution (i.e. 20 μL Sample Preparation Reagent E added to 100 UL virus in cell culture media containing glutamine and penicillin). Mixtures were incubated for zero time (no exposure to Sample Preparation Reagent E), 30 seconds, or 2, 5 and 10 minutes. Each time point had three replicates. Mixtures were then titrated by ten-fold serial dilutions and placed onto Vero E6 cells in 48-well plates. After 4 days cells were stained with crystal violet and the optical density (OD) observed by spectroscopy; virus induced cytopathic effects result in low OD, indicating the presence of virus (positive well). The virus titres were calculated using the Cell Culture Infectivity Dose 50 (CCID₅₀). The initial virus titre (time=0) was 7.3 log 10CCID₅₀/mL. Complete loss of detectable infectious virus was observed after incubation with the buffer for 30 seconds or longer (FIG. 1A). A similar experiment tested with Sample Preparation Reagent E diluted 1 in 10 (i.e. 10 μL Sample Preparation Reagent E added to 90 μL virus in cell culture media) did not inactivate the virus (FIG. 1A).

A third experiment demonstrated Sample Preparation Reagent U mixed 1:1 with virus in cell culture medium was able to completely inactivate the virus within 10 seconds. In brief, Sample Preparation Reagent U was mixed 1:1 with 10⁶ CCID₅₀/mL SARS-CoV-2 for 10 seconds, and the ability of SARS-CoV-2 to infect Vero E6 cells and induce a cytopathic effect was examined. Results (FIG. 1B) indicated that Sample Preparation Reagent E when contacted with 10⁶ CCID₅₀/mL SARS-CoV-2 prevented Vero E6 cell death.

The present inventors have demonstrated that Sample Preparation Reagent E mixed 1:1 with Nipah and Hendra virus inactivated the virus in 10 minutes.

This data demonstrates Sample Preparation Reagent E renders the infectious agent in the sample non-infectious within 30 seconds at a ratio of Sample: Sample Preparation Reagent E of 1:5. Sample Preparation Reagent U renders the infectious agent in the sample non-infectious within 10 seconds at a ratio of Sample Preparation Reagent U: sample of 1:1.

### EXAMPLE 2: Single Step Processing of Sample in Sample Preparation Reagent E Allows Analytical Sensitivity of Detection Down to ˜1 Infectious Particle Per RT-qPCR

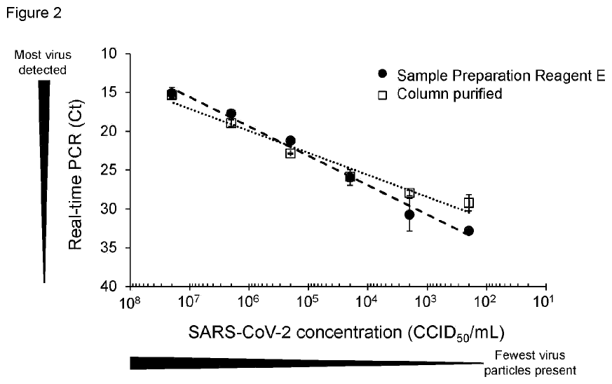

To determine if Sample Preparation Reagent E could process a range of cultured SARS-CoV-2 concentrations for subsequent reverse transcription quantitative PCR (RT-qPCR) detection, 20 μL cultured SARS-CoV-2 (10-fold dilutions of viral supernatant; concentrations ranging from 2×10⁷ to 2×10² CCID₅₀/mL) was mixed with 4 μL Sample Preparation Reagent E. After exactly 10 minutes incubation at room temperature, 5 μL of prepared samples were tested for RT-qPCR detection using 15 μL iTaq Universal Probes One-Step Kit (BioRad; Hercules, California, United States) with primers and probes to detect the E gene (E-gene/BioRad Rt-qPCR). As a comparison, the same viral concentrations were also tested for RT-qPCR detection after RNA was extracted from 20 μL culture supernatant using a column purification method (NucleoSpin RNA virus kit; Macherey-Nagel, Dueren, Germany). At the end of the purification RNA was eluted in 20 μL RNAse-free H₂O and 5 μL was used for RT-qPCR detection. Results demonstrated both sample preparation methods enabled detection of all tested virus concentrations, indicating Sample Preparation Reagent E could successfully process samples containing as little as 2×10² CCID₅₀/mL virus (which equates to ˜1 infectious particle in the PCR tube), as shown in FIG. 2. Back calculation using an RNA standard determined the concentration detected was approximately 9×10³ copies of RNA per reaction, and the RT-qPCR test detected 5×10³ copies per reaction, indicating that this level of detection is close to the limit of detection of the RT-qPCR system itself. Sample Preparation Reagent E trended slightly improved (lower) Ct counts when virus concentration was high (average ˜1 Ct count difference), and slightly poorer (higher) Ct counts when virus concentration was low, when compared to column-purified samples (average Ct count difference ˜2). With only four replicates per data point, these differences could not be considered significant. Quadruplicate detection of approximately a single infectious particle indicates RNA was present in higher concentrations than a single RNA copy in these samples, which is to be expected in a cell culture system where lysed replicating cells might release replicating RNA into the culture supernatant as well as “defective” or non-infectious viral RNA. RT-qPCR is typically more sensitive than viral titration assays, as has been demonstrated in the ability to detect RNA in patients in the absence of replicating infectious virus.

This data demonstrates that Sample Preparation Reagent E could successfully process samples containing as little as 2×10² CCID₅₀/mL virus (which equates to ˜1 infectious particle in the PCR tube).

### EXAMPLE 3: Samples can be Contacted with Sample Preparation Reagent E at Room Temperature

Sample processing trials in Examples 4 to 6 were performed using both, stored positive and negative COVID-19 sample banks:
A characterized sample bank of 41 stored COVID-19 positive patient samples (Table 2). These samples were collected in Virocult® transport medium and represented a range of Ct values ranging from 15 to 44. For comparative analysis, samples were re-processed using the Roche MagNA Pure 96 (Roche) before testing with two RT-qPCR tests: a test using primers and probe to detect the ORF1ab gene using the SensiFAST Probe Lo-ROX One-Step Kit reaction mix (Bioline, Meridian Sciences) (ORF1ab gene/Bioline RT-qPCR), and a test using primers and probe to detect the E-gene using the Qiagen One-Step RT-qPCR Kit (E gene/Qiagen RT-qPCR). Three COVID-19-positive samples in the bank repeatedly tested negative with both these comparative tests, suggesting degradation of samples; these samples were excluded from subsequent analysis.
COVID-19 negative samples (n=20) were also tested to confirm clinical specificity.

TABLE 2
Ct values of samples either extracted with the MagNa Pure 96 system or processed with Sample Preparation Reagent
E for 10 minutes at room temperature and assessed with either the ORF1ab -gene/Bioline or E-gene/Qiagen RT-qPCR.

| | | Stored COVID-19 | | | | | | | | | |
|---|---|---|---|---|---|---|---|---|---|---|---|
| | | positive patient | | ORF1ab - | | | | Sample | | | |
| | | samples collected in | | gene/Bioline | | E gene/Qiagen | | Preparation | | Repeat Sample | |
| | | Virocult ® transport | | RT-qPCR | | RT-qPCR | | Reagent E | | Preparation | |
| | | medium¹ | | (Roche MagNA | | (Roche MagNA | | volume trial³ | | Reagent E | |
| | | | Orig. | Pure XT) | | Pure XT) | | 4 μL | 2 μL | (4 μL; 5:1)^{2, 3} | |
| # | specimen type | Test & Ct | result | Rpt 1 | Rpt 2 | Rpt 1 | Rpt 2 | (5:1)² | (10:1) ² | Rpt 1 | Rpt 2 |
| 1 | Swab | ORF 33 | POS | ND | ND | ND | ND | ND | ND | ND | ND |
| 2 | Nasopharyngeal | ORF 31 | POS | 30.93 | 31.24 | 31.12 | 31.26 | 33.17 | 35.14 | 35.13 | 35.37 |
| 3 | Nasopharyngeal | ORF 31 | POS | 29.69 | 29.14 | 29.93 | 29.49 | 32.72 | 33.33 | 34.21 | 33.34 |
| 4 | Nasopharyngeal | ORF 25 | POS | 23.95 | 23.81 | 23.73 | 23.17 | 26.26 | 25.53 | 26.95 | 26.86 |
| 5 | Nasopharyngeal | ORF 28 | POS | 26.86 | 26.89 | 26.87 | 27.01 | 29.91 | 29.64 | 36.29 | 40.67 |
| | Oropharyngeal | | | | | | | | | | |
| 6 | Nasopharyngeal | ORF 18 | POS | 18.15 | 17.93 | 17.81 | 17.54 | 22.53 | 22.14 | 25.34 | 25.39 |
| 7 | Swab | ORF 16 | POS | 18.52 | 18.64 | 19.01 | 18.74 | 21.56 | 22.77 | 25.21 | 22.83 |
| 8 | Swab | ORF 34 | POS | 32.62 | 32.06 | 32.64 | 32.24 | 33.81 | 33.79 | ND | ND |
| 9 | Nasopharyngeal | ORF 22 | POS | 22.37 | 22.36 | 22.8 | 22.58 | 26.87 | 26.16 | 32.15 | 32.34 |
| 10 | Swab | ORF 24 | POS | 21.85 | 21.78 | 21.5 | 21.55 | 23.85 | 23.41 | 26.18 | 26.8 |
| 11 | Swab | ORF 21 | POS | 21.14 | 21.46 | 21.02 | 20.85 | 23.65 | 23.51 | 25.81 | 26.19 |
| 12 | Nasopharyngeal | ORF 19 | POS | 18.82 | 19.24 | 18.85 | 18.71 | 22.45 | 21.72 | 23.11 | 23.81 |
| 13 | Swab | ORF 15 | POS | 15.78 | 15.93 | 15.78 | 15.6 | 21.61 | 19.5 | 23.08 | 23.54 |
| 14 | Swab | ORF 29 | POS | 29.59 | 29.72 | 29.69 | 29.68 | 33.39 | 31.95 | 34.73 | 36.41 |
| 15 | Swab | ORF 24 | POS | 22.75 | 23.12 | 22.83 | 22.93 | 25.74 | 25.55 | 28.37 | 28.36 |
| 16 | Throat | ORF 18 | POS | 18.94 | 19.32 | 18.89 | 18.93 | 21.84 | 20.39 | 23.35 | 23.9 |
| 17 | Nasopharyngeal | ORF 16 | POS | 14.93 | 15.18 | 14.75 | 14.78 | 18.9 | 18.95 | 21.96 | 21.89 |
| 18 | Swab | ORF 26 | POS | 27.85 | 27.58 | 27.25 | 27.15 | 29.74 | 28.79 | 31.61 | 32.43 |
| 19 | Swab | E 23 | POS | 21.2 | 22.01 | 21.32 | 21.58 | 24.11 | 23.61 | 26.12 | 25.76 |
| 20 | Nasopharyngeal | E 19 | POS | 17.19 | 17.71 | 16.92 | 17.44 | 20.62 | 19.59 | 23.73 | 23.65 |
| 21 | Swab No Site Specified | E 23 | POS | 22.51 | 22.44 | 22.31 | 22.2 | 25.63 | 25.1 | 26.79 | 26.4 |
| 22 | Swab | E 28 | POS | 25.86 | 26.49 | 25.31 | 26.14 | 28.85 | 28.41 | 31.45 | 31.15 |
| 23 | Nasopharyngeal | E 30 | POS | 27.36 | 28.39 | 27.31 | 28.47 | 31.08 | 30.02 | 33.7 | 35.43 |
| 24 | Swab | E 25 | POS | 22.52 | 22.8 | 22.21 | 22.46 | 24.8 | 27.04 | 29.44 | 30.04 |
| 25 | Nasopharyngeal | E 27 | POS | 24.03 | 24.27 | 24.23 | 24.19 | 28.01 | 28.9 | 32.24 | 32.65 |
| | Oropharyngeal | | | | | | | | | | |
| 26 | Nasopharynx | E 44.8 | POS | ND | ND | ND | ND | ND | ND | ND | ND |
| 27 | Nasopharyngeal | E 22 | POS | 19.45 | 19.62 | 19.48 | 19.28 | 23.38 | 22.69 | 27.65 | 25.66 |
| 28 | Swab | E 30 | POS | 28.86 | 28.46 | 28.86 | 27.99 | 32.4 | 36.01 | 33.35 | 33.23 |
| 29 | Swab | E 20 | POS | 17.87 | 18.17 | 17.75 | 17.93 | 21.47 | 21 | 23.96 | 22.83 |
| 30 | Swab | E 28 | POS | 28.88 | 28 | 27.54 | 28.27 | 31.04 | 31.23 | 34.79 | 33.33 |
| 31 | Nasopharyngeal | E 21 | POS | 19.01 | 19.76 | 18.91 | 19.32 | 22.7 | 22.48 | 24.95 | 24.87 |
| 32 | Nasopharyngeal | E 29 | POS | 25.76 | 26.86 | 25.5 | 26.29 | 27.98 | 28.13 | 30.21 | 30.54 |
| 33 | Nasopharyngeal | E 32 | POS | 29.56 | 29.88 | 29.33 | 30.72 | 32.71 | 32.9 | 34.39 | 33.32 |
| | Oropharyngeal | | | | | | | | | | |
| 34 | Swab | E 20 | POS | 18.06 | 18.42 | 17.97 | 18.1 | 22.89 | 22.04 | 24.51 | 23.48 |
| 35 | Swab No Site Specified | E 34 | POS | 32.41 | 33.22 | 33.74 | 33.5 | ND | 37.57 | 36.63 | 36.48 |
| 36 | Swab | E 24 | POS | 20.91 | 21.53 | 20.76 | 21.19 | 23.89 | 25.01 | 26.42 | 25.17 |
| 37 | Nasopharyngeal | E 21 | POS | 18.87 | 19.95 | 18.8 | 19.51 | 22.12 | 20.33 | 23.64 | 23.99 |
| 38 | Naospharyngeal | E 25 | POS | 23.07 | 23.62 | 22.98 | 23.5 | 26.55 | 26.56 | 29.79 | 29.29 |
| 39 | Swab No Site Specified | E 19 | POS | 16.96 | 17.19 | 16.99 | 17.08 | 22.77 | 19.41 | 24.46 | 23.6 |
| 40 | Swab | E 29 | POS | 26.95 | 27.73 | 27.07 | 27.46 | 30.04 | 28.7 | 30.77 | 30.88 |
| 41 | Nasopharyngeal | E 41 | POS | ND | IND | ND | ND | ND | ND | ND | ND |
| | Oropharyngeal | | | | | | | | | | |

¹First column gives information about the original Ct values and tests used for comparison.
²Sample:Sample preparation reagent E ratio: 5:1 = 20 μL sample was mixed with 4 μL Sample Preparation Reagent E; 10:1 = 20 μL sample was mixed with 2 μL Sample Preparation Reagent E.
³For Sample Preparation Reagent E trials, after sample processing the Orf1ab-gene/Bioline RT-qPCR was performed to generate Ct values.
Legend: POS = positive, ORF = ORF1ab gene real-time RT-qPCR assay, E = E-gene real-time RT-qPCR assay; Rpt = trial repeat number, ND = Not detected.

Two stored COVID-19 positive patient samples (samples 15 and 16, Table 2, Table 3) collected in Virocult® transport medium (Cycle threshold values of 24 and 18) were incubated for 10 minutes with Sample Preparation Reagent E at different ratios at either 4° C. or at room temperature (Room Temp.), after which real-time RT-qPCR testing was performed using the ORF1ab-gene/Bioline RT-qPCR (5 μL of prepared samples were added into the RT-qPCR reaction, for a 20 μL final volume RT-qPCR reaction).

The Ct value was not significantly affected when incubation was performed at either 4° C. or room temperature (Table 3), and room temperature incubation was performed for all subsequent trials. Cycle threshold values were noted to alter when larger volumes of Sample Preparation Reagent E were included, and this was further tested in the subsequent experiments.

TABLE 3
Comparison of Ct values when samples were incubated with Sample
Preparation Reagent E (Reagent E) for 10 minutes at different
temperatures and tested with the ORF1ab-gene/Bioline real-time
RT-qPCR. Sample Number (Ct) links to information in Table 2.

| | Sample volume + | Incubation temperature | |
|---|---|---|---|
| Sample | Sample Preparation Reagent E | | Room |
| Number (Ct) | volume | Ice (4° C.) | Temp. |
| 15 (Ct 24) | 15 μL sample + 7.5 μL Reagent E | 26.86 | 27.56 |
| | 20 μL sample + 4 μL Reagent E | 25.48 | 25.83 |
| | 10 μL sample + 2 μL Reagent E | 25.73 | 25.64 |
| 16 (Ct 18) | 15 μL sample + 7.5 μL Reagent E | 21.38 | 22.55 |
| | 20 μL sample + 4 μL Reagent E | 20.41 | 21.5 |
| | 10 μL sample + 2 μL Reagent E | 20.17 | 22.8 |

Ct: Cycle threshold; Reagent E: Sample Preparation Reagent E; Room Temp.: Room temperature/25° C.

This data demonstrates no significant difference between reactions performed at room temperature compared to incubation at 4° C., indicating samples can be contacted with Sample Preparation Reagent E at room temperature.

### EXAMPLE 4: Sample Preparation Reagent E is Compatible with ORF1Ab Gene Primers and Probe Using the Bioline RT-qPCR Mix

Two stored COVID-19 positive patient samples collected in Virocult® transport medium (Ct values of 24 and 18 cycles; 20 μL sample) were incubated for 10 minutes at room temperature with either 4 μL or 2 μL of Sample Preparation Reagent E (Table 4), followed by RT-qPCR testing (5 μL prepared samples included in 20 μL final volume). Testing both samples indicated the Ct value was not significantly affected when using the ORF1ab gene primers and probe in a Bioline RT-qPCR mix (ORF1ab-gene/Bioline RT-qPCR). Testing also indicated (Table 4) the optimal sample/Sample Preparation Reagent E was a 20 μL sample mixed with either 4 μL or 2 μL Sample Preparation Reagent E, followed by direct testing in a RT-qPCR (5 μL processed sample mixture in a final RT-qPCR volume of 20 μL), as those samples had the least deviation from Ct count compared to the original samples.

TABLE 4
Sample Preparation Reagent E compatibility
with two RT-qPCR tests

| Sample volume + | | ORF1ab - | |
|---|---|---|---|
| Sample Preparation | Original | gene/Bioline | E-gene/Qiagen |
| Reagent-E volumes | Ct | Ct | Ct |
| 20 uL sample + 4 μL Reagent-E | 18 | 20.41 | 33.34 |
| 20 uL sample + 2 μL Reagent E | 18 | 20.17 | 32 |
| 20 uL sample + 4 μL Reagent E | 24 | 25.48 | ND |
| 20 uL sample + 2 μL Reagent E | 24 | 25.73 | ND |

Ct: cycle threshold; Reagent E: Sample Preparation Reagent E; ND: Not detected.

These data demonstrate that the addition of Sample Preparation Reagent E was observed to enable effective RT-qPCR testing using Orf1ab-gene primers and probe in a Bioline RT-qPCR mix.

### EXAMPLE 5: COVID-19 Positive Samples Prepared Using Sample Preparation Reagent E Retain >97% Sensitivity (Confidence Interval: 86.19% to 99.93%) During Repeated RT-qPCR Testing

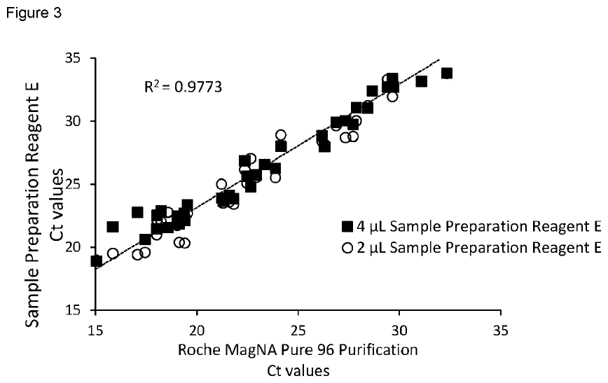

All 41 COVID-positive samples (20 μL; Table 2) were incubated for 10 minutes at room temperature with either 2 μL or 4 μL of Sample Preparation Reagent E, after which 5 μL mixtures were added into the ORF1ab-gene/Bioline RT-qPCR (final volume 20 μL). Use of 2 μL Sample Preparation Reagent E demonstrated identical sensitivity compared to the Roche MagNA pure processed control testing, whereas use of 4 μL Sample Preparation Reagent E missed detection of one sample, resulting in >97% sensitivity (confidence interval: 86.19% to 99.93%; Table 5, Table 2). Pearson analysis of Ct values for both Sample Preparation Reagent E results indicated a correlation of 97% (FIG. 3), with a median increase of three cycle threshold (Ct) counts.

TABLE 5
Clinical sensitivity of the Sample Preparation Reagent E
for processing nasal swabs followed by RT-qPCR detection

| Sample processing | Reagent E | Roche MagNA pure | | Sensitivity |
|---|---|---|---|---|
| system | results | Positive | Negative | (CI) |
| Reagent E (2 μL) | Positive | 38 | 0 | 100% |
| | Negative | 0 | 0 | (90.75%-100%) |
| Reagent E (4 μL) | Positive | 37 | 0 | 97.37% |
| | Negative | 1 | 0 | (86.19%-99.93%) |

Reagent E: Sample Preparation Reagent E; CI: Confidence Interval.

These data demonstrate that samples prepared using Sample Preparation Reagent E retain >97% sensitivity.

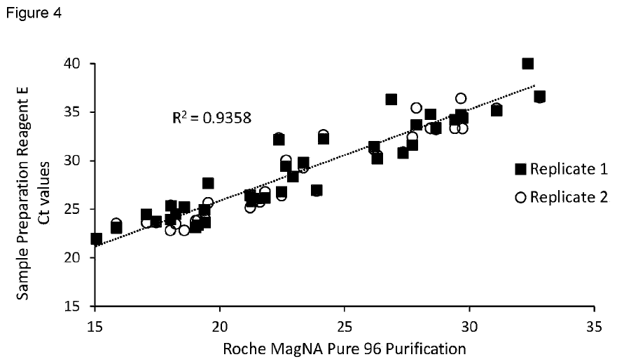

To confirm sensitivity when mixing 4 μL Sample Preparation Reagent E, all 38 samples (20 μL) were re-incubated with 4 μL Sample Preparation Reagent E for 10 minutes at room temperature, followed by placing 5 μL into the ORF1ab-gene/Bioline RT-qPCR (final volume 20 μL). One sample was not detected in this testing trial, confirming >97% sensitivity. Pearson correlation analysis indicated the Roche MagNA pure and Sample Preparation Reagent E had 93% correlation of Ct values, with the 4 μL Sample Preparation Reagent E leading to a median increase of 5 cycle threshold counts (FIG. 4).

These data indicate that following duplicate testing of the COVID-19 positive sample bank, mixing 4 μL Sample Preparation Reagent E with 20 L sample, confirmed >97% sensitivity.

Twenty samples that were negative for SARS-CoV-2 (20 μL) were also mixed with 4 μL Sample Preparation Reagent E followed by testing using the ORF1ab-gene/Bioline RT-qPCR. All provided negative results in the ORF1ab-gene/Bioline RT-qPCR, demonstrating the Sample Preparation Reagent E does not affect specificity of testing.

### EXAMPLE 6: Sample Preparation Reagent E does not Interfere with Testing of Unprocessed Samples

Some RT-qPCR kits enable samples to be tested without processing. In these experiments we determined if addition of Sample Preparation Reagent E would interfere with testing of unprocessed samples.

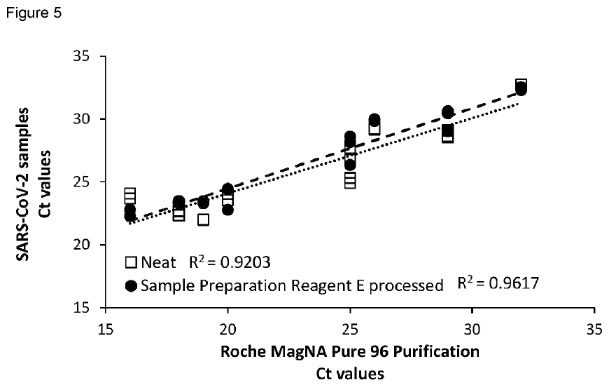

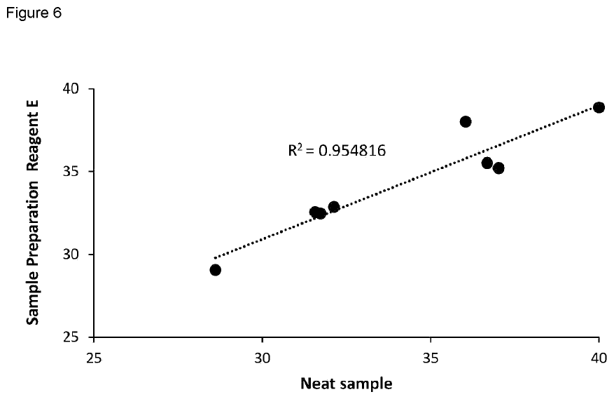

Ten COVID-19 positive samples were either tested “neat” by directly adding 5 μL into the ORF1ab-gene/Bioline RT-qPCR (20 μL final volume) or were first mixed with Sample Preparation Reagent E (20 μL sample mixed with 4 μL Sample Preparation Reagent E) and incubated for 10 minutes at room temperature before adding 5 μL of the processed sample in the ORF1ab-gene/Bioline RT-qPCR (20 μL final volume) (Table 6). All samples were tested in duplicate. Pearson analysis (FIG. 5) indicated Ct values from samples processed with Sample Preparation Reagent E correlated more closely (96%) than neat samples (92%) when compared to the Ct values obtained by RT-qPCR testing after purification using the Roche Magna Pure (Table 2). Furthermore, one sample (22, Ct count 28) was diluted 10-fold in Virocult® transport medium before being either tested both neat or after treatment with 4 μL Sample Preparation Reagent E as described above. There was no difference in Ct count or dilution at which testing became negative between the two testing methods (Table 7), confirmed by direct Pearson correlation of Ct counts of 95% (FIG. 6).

TABLE 6
Sample processing comparisons.

Average Ct count

| Sample | Roche Magna Pure | Neat samples | Reagent E |
|---|---|---|---|
| 4 | 23.88 | 25.11 | 26.355 |
| 6 | 18.04 | 22.515 | 23.39 |
| 7 | 18.58 | 23.88 | 22.545 |
| 18 | 27.715 | 29.22 | 29.925 |
| 29 | 18.02 | 23.8 | 23.61 |
| 32 | 26.31 | 28.6 | 29.04 |
| 33 | 29.72 | 32.75 | 32.425 |
| 38 | 23.345 | 27.235 | 28.39 |
| 39 | 17.075 | 22 | 23.385 |
| 40 | 27.34 | 29.09 | 30.54 |

Reagent E = Sample Preparation Reagent E.

TABLE 7
Sample processing dilution comparison.

| | | | | Sample Preparation | | |
|---|---|---|---|---|---|---|
| | Neat sample | | | Reagent E | | |
| | Rep1 | Rep2 | Rep3 | Rep1 | Rep2 | Rep3 |
| neat | 28.61 | NT | NT | 29.06 | NT | NT |
| 10-1 | 31.57 | 31.73 | 32.13 | 32.56 | 32.47 | 32.87 |
| 10-2 | 36.04 | 36.68 | 37.02 | 38.01 | 35.52 | 35.21 |
| 10-3 | Neg | 40.03 | Neg | 38.87 | 39.07 | Neg |
| 10-4 | Neg | Neg | Neg | Neg | Neg | Neg |

NT: Not tested.

This data indicates that Sample Preparation Reagent E does not interfere with testing of unprocessed samples using the Bioline RT-PCR, or sensitivity of detection. This data also demonstrates that inclusion of the Sample Preparation Reagent E improved the correlation of Ct counts when compared to Roche MagNA pure processed samples.

### EXAMPLE 7: Sample Preparation Reagent Solubility Testing

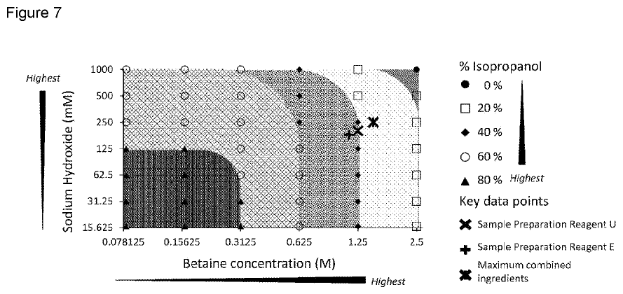

The range of concentrations of components of Sample Preparation Reagent were examined. In brief, the Sample Preparation Reagent ingredients were combined at different concentrations in a series of tests performed over four days, resulting in a total of 408 Sample Preparation Reagent formulations. Of the 408 Sample Preparation Reagent formulations prepared, 232 formulations showed an observable phase separation and were excluded from further testing. The remaining 176 Sample preparation reagent formulations stayed monophasic. The maximum concentration of each ingredient, along with the maximum concentrations of other ingredients to achieve this concentration, was identified (FIG. 7). This data demonstrates that up to 80% isopropanol could be incorporated, with a maximum of 125 mM NaOH if the betaine concentration was 0.16 M or less (alternatively, 80% isopropanol could be achieved with a maximum of 0.31 M betaine, if the NaOH concentration was 31 mM or less). Up to 1000 mM NaOH could be incorporated, with a maximum of 0.31 M Betaine if the concentration of isopropanol was 60% or less (alternatively, 1000 mM NaOH could be achieved with 0.625 M betaine if the isopropanol concentration was dropped to 40%). Up to 2.5 M Betaine could be incorporated, with up to 500 mM NaOH, but only at 20% isopropanol concentrations or less.

### EXAMPLE 8: Sample Preparation Reagent E Formulation Testing

The ability of Sample Preparation Reagent E formulations (Table 8) to process SARS-CoV-2 (for subsequent RT-qPCR testing) were trialled.

TABLE 8
Sample Preparation Reagent formulations
tested for processing of SARS-CoV-2

| | | Betaine | Isopropanol | NaOH |
|---|---|---|---|---|
| | | (M) | (%) | (mM) |
| 1 | Starting from this recipe | 1.25 | 0.4 | 250 |
| 2 | Add less sodium | 1.25 | 0.4 | 125 |
| | hydroxide (125) | | | |
| 3 | Add less sodium | 1.25 | 0.4 | 62.5 |
| | hydroxide (62.5) | | | |
| 4 | Add less sodium | 1.25 | 0.4 | 31.25 |
| | hydroxide (31.25) | | | |
| 5 | Add less sodium | 1.25 | 0.4 | 15.625 |
| | hydroxide (15.625) | | | |
| 6 | Add no sodium | 1.25 | 0.4 | 0 |
| | hydroxide (0) | | | |
| 7 | Add less betaine (0.625) | 0.625 | 0.4 | 250 |
| 8 | Add less betaine (0.3) | 0.3125 | 0.4 | 250 |
| 9 | Add less betaine (0.15) | 0.15625 | 0.4 | 250 |
| 10 | Add less betaine (0.078125) | 0.078125 | 0.4 | 250 |
| 11 | Add less isopropanol (20) | 1.25 | 20 | 250 |
| 12 | Add less isopropanol (0) | 1.25 | 0 | 250 |
| 13 | Add more betaine (2.5) | 2.5 | 0.2 | 250 |
| 14 | Add more isopropanol (60%) | 0.625 | 0.6 | 125 |
| 15 | Add more isopropanol (80%) | 0.3125 | 0.8 | 31.25 |
| 16 | Add more sodium | 1.25 | 0.2 | 500 |
| | hydroxide (500) | | | |
| 17 | Add more sodium | 1.25 | 0.2 | 1000 |
| | hydroxide (1000) | | | |
| 18 | Maximise (no isopropanol) | 2.5 | 0 | 1000 |
| 19 | Sample Preparation | 1.25 | 0.4 | 200 |
| | Reagent U | | | |
| 20 | Sample Preparation | 1.125 | 0.36 | 180 |
| | Reagent E | | | |
| 24 | H₂O only | 0 | 0 | 0 |

A UV-inactivated SARS-CoV-2 standard had previously been analysed and determined that the lowest dilution that could consistently be detected by RT-qPCR (limit of detection) was 1/1,000,000 (10⁻⁶) (see Example 9). This study tested virus diluted at this limit of detection concentration (10⁻⁶) as well as 10× higher concentration (10⁻⁵) for comparison.

In brief, 4 μL of each Sample Preparation Reagent formulation was added to 20 μL of either 10⁻⁵ or 10⁻⁶ dilutions of UV-inactivated COVID before incubation for 10 minutes at Room Temperature. 5 μL of these reactions were then dispensed into 15 μL of two RT-qPCR mixes: the SensiFAST Probe Lo-ROX One-Step Kit (Bioline), and the iTaq Universal Probe (BioRad). Both mixes contained primers and probe for amplification of the N2 gene developed by the USA Center for Disease Control (CDC) synthesized by Integrated DNA Technologies and prepared as a primer/probe mix at concentrations advised by the CDC (1.5 μL of the mixture was incorporated within the 15 μL of the RT-qPCR master mixes). Reaction mixes were placed into a Quant Studio 5 real-time PCR thermocycler (Thermo Fisher) beginning with 5 min at 50° C., and 2 min at 95° C., followed by 50 cycles of 3 sec at 95° C. and 30 sec at 60° C. For positive results, the cycle at which fluorescence appeared over baseline was recorded (Cylce threshold, Ct).

The cycle threshold for each sample tested is provided in Table 9. The smaller a cycle threshold number, the more effective the Sample preparation reagent was at preparing the sample for detection by RT-qPCR, and reducing inhibition during detection. If the virus was not able to be detected, the cycle threshold signal was returned as “undetermined” (listed as ND or not detected in the table). Cycle threshold values obtained that were improved compared to a water-only formulation control are shown in Table 10.

TABLE 9
Effectiveness of detection of different Sample Preparation Reagents,
as determined by decreased RT-qPCR cycle threshold (Ct) values.

| | Formulation trialled | | | Cycle threshold values¹ | | | |
|---|---|---|---|---|---|---|---|
| | Betaine | Isopropanol | NaOH | BioRad RT-qPCR | | Bioline RT-qPCR | |
| Formulation notes | (M) | (%) | (mM) | 10⁻⁶ | 10⁻⁵ | 10⁻⁶ | 10⁻⁵ |
| Starting from this | 1.25 | 40% | 250 | 33.268 | 30.493 | 35.018 | 31.860 |
| recipe | | | | | | | |
| Add less sodium | 1.25 | 40% | 125 | 32.317 | 29.177 | 34.261 | 31.358 |
| hydroxide (125) | | | | | | | |
| Add less sodium | 1.25 | 40% | 62.5 | 32.689 | 30.070 | 34.881 | 31.713 |
| hydroxide (62.5) | | | | | | | |
| Add less sodium | 1.25 | 40% | 31.25 | 35.269 | 31.685 | 35.443 | 32.682 |
| hydroxide (31.25) | | | | | | | |
| Add less sodium | 1.25 | 40% | 15.625 | 35.708 | 32.956 | 34.521 | 33.660 |
| hydroxide (15.625) | | | | | | | |
| Add no sodium | 1.25 | 40% | 0 | 35.679 | ND | 36.265 | ND |
| hydroxide (0) | | | | | | | |
| Add less | 0.625 | 40% | 250 | 33.099 | 29.569 | 35.233 | 31.358 |
| betaine (0.625) | | | | | | | |
| Add less | 0.3125 | 40% | 250 | 33.357 | 30.088 | 34.122 | 31.368 |
| betaine (0.3) | | | | | | | |
| Add less | 0.15625 | 40% | 250 | 33.926 | 29.717 | 34.495 | 31.593 |
| betaine (0.15) | | | | | | | |
| Add less | 0.078125 | 40% | 250 | 32.686 | 29.272 | 34.216 | 31.171 |
| betaine (0.078125) | | | | | | | |
| Add less | 1.25 | 20 | 250 | 34.257 | 29.889 | 35.885 | 32.408 |
| isopropanol (20) | | | | | | | |
| Add less | 1.25 | 0 | 250 | 33.438 | 31.559 | 35.021 | 32.547 |
| isopropanol (0) | | | | | | | |
| Add more | 2.5 | 20% | 250 | 32.958 | 29.864 | 35.291 | 32.525 |
| betaine (2.5) | | | | | | | |
| Add more | 0.625 | 60% | 125 | 32.863 | 29.009 | 35.098 | 30.928 |
| isopropanol (60%) | | | | | | | |
| Add more | 0.3125 | 80% | 31.25 | 33.764 | 29.529 | 34.964 | 31.253 |
| isopropanol (80%) | | | | | | | |
| Add more sodium | 1.25 | 20% | 500 | 33.308 | 30.553 | ND | 32.565 |
| hydroxide (500) | | | | | | | |
| Add more sodium | 1.25 | 20% | 1000 | ND | ND | 37.537 | ND |
| hydroxide (1000) | | | | | | | |
| Maximise (no | 2.5 | 0% | 1000 | ND | ND | 35.867 | 33.074 |
| isopropanol) | | | | | | | |
| Sample Preparation | 1.25 | 40% | 200 | 33.184 | 29.759 | 34.586 | 31.438 |
| Reagent U | | | | | | | |
| Sample Preparation | 1.125 | 36% | 180 | 34.093 | 29.903 | 35.753 | 31.482 |
| Reagent E | | | | | | | |
| H20 only | 0 | 0% | 0 | 34.476 | | 37.782 | |
| NTC | N/A | | | ND | ND | ND | ND |

¹Cycle threshold values obtained for dilution of inactivated SARS-CoV-2 trialled (10⁻⁵ or 10⁻⁶; 20 μL volume), after mixing with the respective Sample Preparation Reagent formulation and incubation for 10 minutes, followed by RT-qPCR testing with primers and probe amplifying the N gene (N2 primers) using either the SensiFAST Probe Lo-ROX One-Step Kit (Bioline; Bioline RT-qPCR) or the iTaq Universal Probe kit (BioRad; BioRad RT-qPCR).
NTC: No template control; ND: No amplification was detected (undetermined).

TABLE 10
Sample Preparation Reagent Formulations with Ct values
improved compared to a water-only formulation.

| | Formulation trialled | | | Ct value improved c.f. water¹ | | | |
|---|---|---|---|---|---|---|---|
| | Betaine | Isopropanol | NaOH | BioRad RT-qPCR | | Bioline RT-qPCR | |
| Formulation notes | (M) | (%) | (mM) | 10⁻⁶ | 10⁻⁵ | 10⁻⁶ | 10⁻⁵ |
| Starting from | 1.25 | 40% | 250 | + | + | + | + |
| this recipe | | | | | | | |
| Add less sodium | 1.25 | 40% | 125 | + | + | + | + |
| hydroxide (125) | | | | | | | |
| Add less sodium | 1.25 | 40% | 62.5 | + | + | + | + |
| hydroxide (62.5) | | | | | | | |
| Add less sodium | 1.25 | 40% | 31.25 | − | + | + | + |
| hydroxide (31.25) | | | | | | | |
| Add less sodium | 1.25 | 40% | 15.625 | − | + | + | + |
| hydroxide (15.625) | | | | | | | |
| Add no sodium | 1.25 | 40% | 0 | − | − | + | − |
| hydroxide (0) | | | | | | | |
| Add less | 0.625 | 40% | 250 | + | + | + | + |
| betaine (0.625) | | | | | | | |
| Add less | 0.3125 | 40% | 250 | + | + | + | + |
| betaine (0.3) | | | | | | | |
| Add less | 0.15625 | 40% | 250 | + | + | + | + |
| betaine (0.15) | | | | | | | |
| Add less | 0.078125 | 40% | 250 | + | + | + | + |
| betaine (0.078125) | | | | | | | |
| Add less | 1.25 | 20 | 250 | + | + | + | + |
| isopropanol (20) | | | | | | | |
| Add less | 1.25 | 0 | 250 | + | + | + | + |
| isopropanol (0) | | | | | | | |
| Add more | 2.5 | 20% | 250 | + | + | + | + |
| betaine (2.5) | | | | | | | |
| Add more | 0.625 | 60% | 125 | + | + | + | + |
| isopropanol (60%) | | | | | | | |
| Add more | 0.3125 | 80% | 31.25 | + | + | + | + |
| isopropanol (80%) | | | | | | | |
| Add more sodium | 1.25 | 20% | 500 | + | + | − | + |
| hydroxide (500) | | | | | | | |
| Add more sodium | 1.25 | 20% | 1000 | − | − | + | − |
| hydroxide (1000) | | | | | | | |
| Maximise (no | 2.5 | 0% | 1000 | − | − | + | + |
| isopropanol) | | | | | | | |
| Sample Preparation | 1.25 | 40% | 200 | + | + | + | + |
| Reagent U | | | | | | | |
| Sample Preparation | 1.125 | 36% | 180 | + | + | + | + |
| Reagent E | | | | | | | |
| H20 only | 0 | 0% | 0 | − | − | − | − |

¹Further analysis of the experiment shown in Table 9: dilution of inactivated SARS-CoV-2 trialled (10⁻⁵ or 10⁻⁶) mixed with formulation shown and subsequently tested for SARS-CoV-2 using either the SensiFAST Probe Lo-ROX One-Step Kit (Bioline; Bioline RT-qPCR) or the iTaq Universal Probe kit (BioRad; BioRad RT-qPCR). Cycle threshold values were subtracted from the water-only formulation cycle threshold values. A positive result (+) indicates the number was lower than the water only trial (i.e. an improved cycle threshold value compared to water only was obtained).

Sample Preparation Reagent U comprising 1.25 M Betaine, 0.4% isopropanol and 200 mM NaOH performed similarly to Sample Preparation Reagent E comprising 1.125 M Betaine, 0.36% isopropanol and 180 mM NaOH.

The present inventors have demonstrated that while iTaq assays are more affected by 1000 mM NaOH than Bioline assays, Sample Preparation Reagent E formulations can be used successfully with different RT-qPCR mixes.

Results were analysed by comparing Ct count improvement over the Reagent U and Reagent E formulations, by looking at the difference in Ct count from an average of the Reagent U and Reagent E formulation Ct count (Table 11):

Sodium hydroxide:
125 mM NaOH provided a 0.7 Ct improvement over Sample Preparation Reagent E and U formulations
Concentrations of 250 mM or more were associated with higher Ct counts
Concentrations of 31.25 mM or less were associated with higher Ct counts
1000 mM interferes with detection

### Isopropanol:

Higher isopropanol (60% and 80%) was associated with decreased Ct counts
Less isopropanol (0/20%) was associated with significantly higher Ct counts across the board

Betaine:
Between 0.078125 and 2.5 mM provides suitable detection.

TABLE 11
Analysis of formulations with improved cycle-threshold values compared to Reagent E and Reagent U formulations.

| | | | | | Significant | | | | Ct difference for | | | | | | | | |
|---|---|---|---|---|---|---|---|---|---|---|---|---|---|---|---|---|---|
| | | Formulation trialled | | | Improvement² | | | | Significant imprv.³ | | | | Ct deviation⁴ | | | | |
| Formulation | | Betaine | Isopropanol | NaOH | iTaq | | Bioline | | iTaq | | Bioline | | iTaq | | Bioline | | Av. |
| notes¹ | | (M) | (%) | (mM) | 10⁻⁶ | 10⁻⁵ | 10⁻⁶ | 10⁻⁵ | 10⁻⁶ | 10⁻⁵ | 10⁻⁶ | 10⁻⁵ | 10⁻⁶ | 10⁻⁵ | 10⁻⁶ | 10⁻⁵ | dev.⁵ |
| 1 | Starting from | 1.25 | 0.4 | 250 | − | − | − | − | − | − | − | − | 0.37 | −0.66 | 0.15 | −0.40 | −0.13 |
| | this recipe | | | | | | | | | | | | | | | | |
| 2 | Add less | 1.25 | 0.4 | 125 | + | + | − | + | 1.32 | 0.65 | − | 0.10 | 1.32 | 0.65 | 0.91 | 0.10 | 0.75 |
| | sodium | | | | | | | | | | | | | | | | |
| | hydroxide | | | | | | | | | | | | | | | | |
| | (125) | | | | | | | | | | | | | | | | |
| 3 | Add less | 1.25 | 0.4 | 62.5 | − | − | − | − | − | − | − | − | 0.95 | −0.24 | 0.29 | −0.25 | 0.19 |
| | sodium | | | | | | | | | | | | | | | | |
| | hydroxide | | | | | | | | | | | | | | | | |
| | (62.5) | | | | | | | | | | | | | | | | |
| 4 | Add less | 1.25 | 0.4 | 31.25 | − | − | − | − | − | − | − | − | −1.63 | −1.85 | −0.27 | −1.22 | −1.25 |
| | sodium | | | | | | | | | | | | | | | | |
| | hydroxide | | | | | | | | | | | | | | | | |
| | (31.25) | | | | | | | | | | | | | | | | |
| 5 | Add less | 1.25 | 0.4 | 15.625 | − | − | − | − | − | − | − | − | −2.07 | −3.12 | 0.65 | −2.20 | −1.69 |
| | sodium | | | | | | | | | | | | | | | | |
| | hydroxide | | | | | | | | | | | | | | | | |
| | (15.625) | | | | | | | | | | | | | | | | |
| 6 | Add no | 1.25 | 0.4 | 0 | − | | − | | − | | − | | −2.04 | | −1.10 | | −1.57 |
| | sodium | | | | | | | | | | | | | | | | |
| | hydroxide | | | | | | | | | | | | | | | | |
| | (0) | | | | | | | | | | | | | | | | |
| 7 | Add less | 0.625 | 0.4 | 250 | − | + | − | + | − | 0.26 | − | 0.10 | 0.54 | 0.26 | −0.06 | 0.10 | 0.21 |
| | betaine | | | | | | | | | | | | | | | | |
| | (0.625) | | | | | | | | | | | | | | | | |
| 8 | Add less | 0.3125 | 0.4 | 250 | − | − | − | + | − | − | − | 0.09 | 0.28 | −0.26 | 1.05 | 0.09 | 0.29 |
| | betaine | | | | | | | | | | | | | | | | |
| | (0.3) | | | | | | | | | | | | | | | | |
| 9 | Add less | 0.15625 | 0.4 | 250 | − | − | − | − | − | − | − | − | −0.29 | 0.11 | 0.67 | −0.13 | 0.09 |
| | betaine | | | | | | | | | | | | | | | | |
| | (0.15) | | | | | | | | | | | | | | | | |
| 10 | Add less | 0.078125 | 0.4 | 250 | − | + | − | + | − | 0.56 | − | 0.29 | 0.95 | 0.56 | 0.95 | 0.29 | 0.69 |
| | betaine | | | | | | | | | | | | | | | | |
| | (0.078125) | | | | | | | | | | | | | | | | |
| 11 | Add less | 1.25 | 20 | 250 | − | − | − | − | − | − | − | − | −0.62 | −0.06 | −0.72 | −0.95 | −0.59 |
| | isopropanol | | | | | | | | | | | | | | | | |
| | (20) | | | | | | | | | | | | | | | | |
| 12 | Add less | 1.25 | 0 | 250 | − | − | − | − | − | − | − | − | 0.20 | −1.73 | 0.15 | −1.09 | −0.62 |
| | isopropanol | | | | | | | | | | | | | | | | |
| | (0) | | | | | | | | | | | | | | | | |
| 13 | Add more | 2.5 | 0.2 | 250 | − | − | − | − | − | − | − | − | 0.68 | −0.03 | −0.12 | −1.07 | −0.13 |
| | betaine (2.5) | | | | | | | | | | | | | | | | |
| 14 | Add more | 0.625 | 0.6 | 125 | − | + | − | + | − | 0.82 | − | 0.53 | 0.78 | 0.82 | 0.07 | 0.53 | 0.55 |
| | isoproanol | | | | | | | | | | | | | | | | |
| | (60%) | | | | | | | | | | | | | | | | |
| 15 | Add more | 0.3125 | 0.8 | 31.25 | − | + | − | + | − | 0.30 | − | 0.21 | −0.13 | 0.30 | 0.21 | 0.21 | 0.15 |
| | isoproanol | | | | | | | | | | | | | | | | |
| | (80%) | | | | | | | | | | | | | | | | |
| 16 | Add more | 1.25 | 0.2 | 500 | − | − | | − | − | − | | − | 0.33 | −0.72 | | −1.10 | −0.50 |
| | sodium | | | | | | | | | | | | | | | | |
| | hydroxide | | | | | | | | | | | | | | | | |
| | (500) | | | | | | | | | | | | | | | | |
| 17 | Add more | 1.25 | 0.2 | 1000 | | | − | | | | − | | | | −2.37 | | −2.37 |
| | sodium | | | | | | | | | | | | | | | | |
| | hydroxide | | | | | | | | | | | | | | | | |
| | (1000) | | | | | | | | | | | | | | | | |
| 18 | Maximise | 2.5 | 0 | 1000 | | | − | − | | | − | − | | | −0.70 | −1.61 | −1.16 |
| | (no | | | | | | | | | | | | | | | | |
| | isopropanol) | | | | | | | | | | | | | | | | |
| 19 | Sample | 1.25 | 0.4 | 200 | − | − | − | − | − | − | − | − | 0.45 | 0.07 | 0.58 | 0.02 | 0.28 |
| | Preparation | | | | | | | | | | | | | | | | |
| | Reagent U | | | | | | | | | | | | | | | | |
| 20 | Sample | 1.125 | 0.36 | 180 | − | − | − | − | − | − | − | − | −0.45 | −0.07 | −0.58 | −0.02 | −0.28 |
| | Preparation | | | | | | | | | | | | | | | | |
| | Reagent E | | | | | | | | | | | | | | | | |
| 24 | H20 1e10\hat{ }5 | 0 | 0 | 0 | − | NT | − | NT | − | NT | − | NT | −0.84 | NT | −2.61 | NT | −1.73 |

¹Further analysis of the experiment shown in Table 9: dilution of inactivated SARS-CoV-2 trialled (10⁻⁵ or 10⁻⁶) mixed with formulation shown, and subsequently tested for SARS-CoV-2 using either the SensiFAST Probe Lo-ROX One-Step Kit (Bioline; Bioline RT-qPCR) or the iTaq Universal Probe kit (BioRad; BioRad RT-qPCR).
²Significant improvement: Each formulation was analysed to determine if the cycle threshold (Ct) value was significantly improved compared to the Reagent E and Reagent U formulations, as defined by a Ct value that was two standard deviations below the average Reagent E and Reagent U formulations average Ct-value (for each RT-qPCR kit, and at each dilution of inactivated SARS-CoV-2). “+” symbol and grey shading indicate any sample with significantly improved Ct-value.
³Ct difference for significantly improved formulations: For the significantly improved formulations (see note 2), the Ct-value difference from the average Sample Preparation Reagent E and U formulations is shown, for each RT-qPCR kit trialled, and at each dilution of inactivated SARS-CoV-2.
⁴Ct deviation: The Ct-value difference compared to the average Reagent E and U formulations for all formulations, for each RT-qPCR kit trialled, and at each dilution of inactivated SARS-CoV-2. The darker the shading, the more improved a Ct value was obtained. White indicates inferior or no Ct-values obtained.
⁵Av dev: The average Ct-value difference across all trials (both inactivated SARS-CoV-2 dilutions tested, and both RT-qPCR mixes tests) is shown. The darker the shading, the more improved an average Ct value was obtained. White indicates inferior or no average Ct-values obtained.
NT = Not tested.

Additional formulations of Sample Preparation Reagent were trialled to determine the robustness of formulation and final pH of the reagent.

Results indicated the functional performance of Sample Preparation Reagent for inactivation and qPCR or RT-qPCR detection was extremely robust—the reagent could be 15% more or less concentrated and still pass all functional testing (Table 12). Not until a single ingredient was removed (Formulation 6) or a different ingredient reduced by 83% (Formulation pH1), did functional testing failures begin to be apparent.

In regard to pH testing, the pH of all the undiluted formulations was very high, and only one undiluted formulation had a significant pH change (as defined by >+2 standard deviations, or 90% confidence). However, if the formulations were diluted 1/6 in PBS, we were able to observe significant pH deviations from the base formulation trialled.

TABLE 12
Alternative Sample Preparation Reagent formulation testing

| | | Testing performed | | | | | | |
|---|---|---|---|---|---|---|---|---|
| | | | pH | SARS- | Dengue | Klebseilla | K. | S. |
| Formulation | | | (diluted 1/6 | CoV-2 | virus | pneumoniae | pneumoniae | abony |
| ID | Change | pH | with PBS) | detect¹ | inactiv.² | inactiv.³ | detect.⁴ | inactiv.⁵ |
| Sample | Standard Reagent E | 14.47 (±0.07) | 10.55 (±0.03) | PASS | PASS | PASS | PASS | PASS |
| Preparation | formulation: 1.16M | | | | | | | |
| Reagent E | Betaine, 37% | | | | | | | |
| | Isopropanol, 0.183M | | | | | | | |
| | Sodium hydroxide | | | | | | | |
| Formulation | 7.5% more | 14.52 | 10.76 | PASS | PASS | PASS | PASS | PASS |
| 1: (Sample | concentrated than | | | | | | | |
| Preparation | Reagent E: 1.25M | | | | | | | |
| Reagent U) | Betaine, 40% | | | | | | | |
| | Isopropanol, 0.2M | | | | | | | |
| | Sodium hydroxide | | | | | | | |
| Formulation | 15% more | 14.50 | 10.98 | PASS | PASS | PASS | PASS | PASS |
| 2 | concentrated than | | | | | | | |
| | Reagent E: 1.33M | | | | | | | |
| | Betain, 43% | | | | | | | |
| | Isopropanol, 0.210M | | | | | | | |
| | Sodium Hydroxide | | | | | | | |
| Formulation | 7.5% less | 14.50 | 8.85 | PASS | PASS | PASS | PASS | PASS |
| 3 | concentrated than | | | | | | | |
| | Reagent E: 1.07M | | | | | | | |
| | Betain, 34% | | | | | | | |
| | Isopropanol, 0.169M | | | | | | | |
| | Sodium Hydroxide | | | | | | | |
| Formulation | 15% less concentrated | 14.45 | 9.02 | PASS | PASS | PASS | PASS | PASS |
| 4 | than Reagent E: 0.99M | | | | | | | |
| | Betaine, 31% | | | | | | | |
| | Isopropanol, 0.156M | | | | | | | |
| | Sodium hydroxide | | | | | | | |
| Formulation | 93% less Betaine, | 14.23 | 8.47 | PASS | PASS | PASS | PASS | PASS |
| 5 | 62% more Isopropanol | | | | | | | |
| | 32% less Sodium | | | | | | | |
| | hydroxide: | | | | | | | |
| | 0.08M Betaine, 60% | | | | | | | |
| | Isopropanol, 0.125M | | | | | | | |
| | Sodium hydroxide | | | | | | | |
| Formulation | No betaine | 14.5 | 8.39 | FAIL | PASS | PASS | PASS | PASS |
| 6 | 62% more Isopropanol | | | (only 1/3 | | | | |
| | 32% less Sodium | | | replicates | | | | |
| | hydroxide: | | | at LOD) | | | | |
| | 60% Isopropanol, | | | | | | | |
| | 0.125M Sodium | | | | | | | |
| | hydroxide | | | | | | | |
| Formulation | 83% less Sodium | 14.45 | 7.73 | PASS | FAIL | PASS | PASS | PASS |
| pH 1 | hydroxide: | | | | (4.86 | | | |
| | 1.16M Betaine, 37% | | | | TCID₅₀/mL | | | |
| | Isopropanol, 0.03M | | | | c.f. 5.99 | | | |
| | Sodium hydroxide | | | | for no | | | |
| | | | | | Reagent) | | | |

Legend: Inactiv.—Inactivation; Detect.—detection.
¹For SARS-CoV_2 detection, a pass indicates that after sample processing with 5 parts sample and 1 part Sample Preparation Reagent E, the virus was detected by RT-qPCR down to the limit of detection (3/3 replicates at 32 CCID₅₀/mL or 64 RNA copies/reaction) and Ct values were consistent for the test and reference sample (<±1.65 Ct value standard deviation).
²For Dengue virus inactivation, a pass indicates that after sample processing with 5 parts sample (DENV-1, ET00.243, 2 × 10⁶ TCID₅₀/mL in cell culture media) mixed with 1 part Sample Preparation Reagent E, the virus was inactivated with no virus detected by TCID₅₀ ELISA testing.
³For K. Pneumoniae inactivation, a pass indicates that after sample processing with 1 part sample (0.5 McFarland's standard, 9.33 × 10⁷ cfu/mL bacterial culture in saline) mixed with 1 part Sample Preparation Reagent E, the bacteria was inactivated, with no bacterial growth observed by bacterial culture testing.
⁴For K. Pneumoniae detection, a pass indicates that after sample processing with 1 part sample (0.5 McFarland's standard, 9.33 × 10⁷ of bacterial culture in saline) mixed with 1 part Sample Preparation Reagent E, the bacteria was detected by real-time PCR down to the limit of detection (a 10⁻⁴ dilution, 2/2 replicates) and Ct values were consistent for the test and reference sample (<±1.65 Ct value standard deviation).
⁵For S. abony inactivation, a pass indicates that after sample processing with 1 part bacterial culture sample (5 × 10⁸ Cfu/mL) mixed with 1 part Sample Preparation Reagent E, the bacteria was inactivated, with no bacterial growth observed by bacterial culture testing.

These results suggest that while Sample Preparation Reagent E formulation is extremely robust (inactivation and detection activity tolerates+/−15% ingredient concentrations), the pH of accurately formulated Sample Preparation Reagent E can be monitored by diluting 1/6 in PBS

### EXAMPLE 9: Sample Preparation Reagent E Allows Detection of Inactivated SARS-CoV-2 Down to 10⁻⁶ Dilution

The ability of Sample Preparation Reagent E to allow detection of a UV-inactivated SARS-CoV-2 was examined.

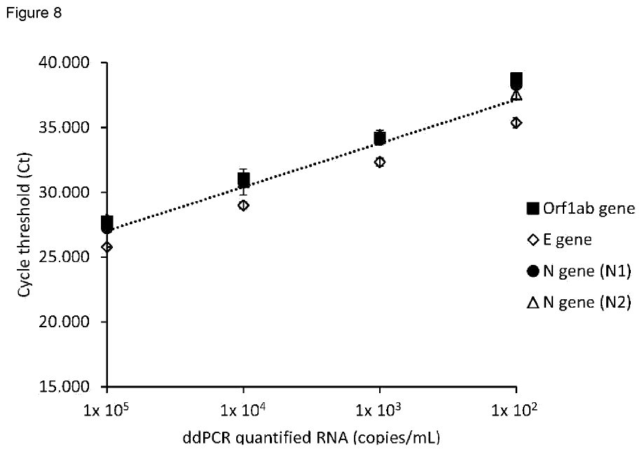

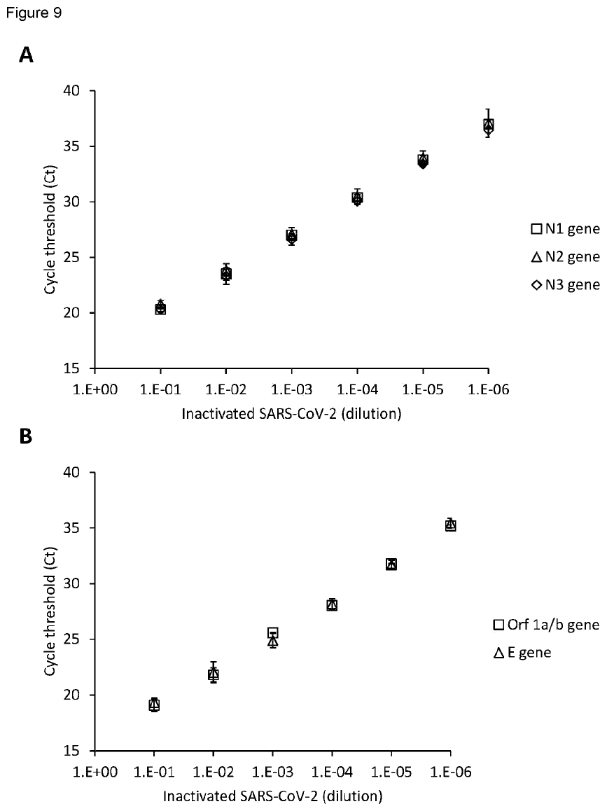

In brief, first ddPCR quantified RNA template was used in a Rox Sensifast (Bioline/Meridian) RT-qPCR using primers and a probe that amplify either the ORF1ab gene, the N gene (designated N2 primers and probe), or the E gene. As shown in FIG. 8, all primer/probe combinations provided detection down to 1000 copies/mL. A UV-inactivated SARS-CoV-2 was then mixed with Sample Preparation Reagent E for 10 minutes prior to use in a Rox Sensifast (Bioline/Meridian) RT-qPCR using primers and a probe that amplify either the ORF1ab gene, three different primers that amplify parts of the N gene (N1, N2, and N3) or the E gene, as shown in FIG. 9, all primer/probe combinations provided detection down to 10⁻⁶ dilution.

### EXAMPLE 10: Sample Preparation Reagent E Inactivates Samples Containing Bacterial Infectious Agents and can be Used for Downstream RPA and Real-Time PCR Testing

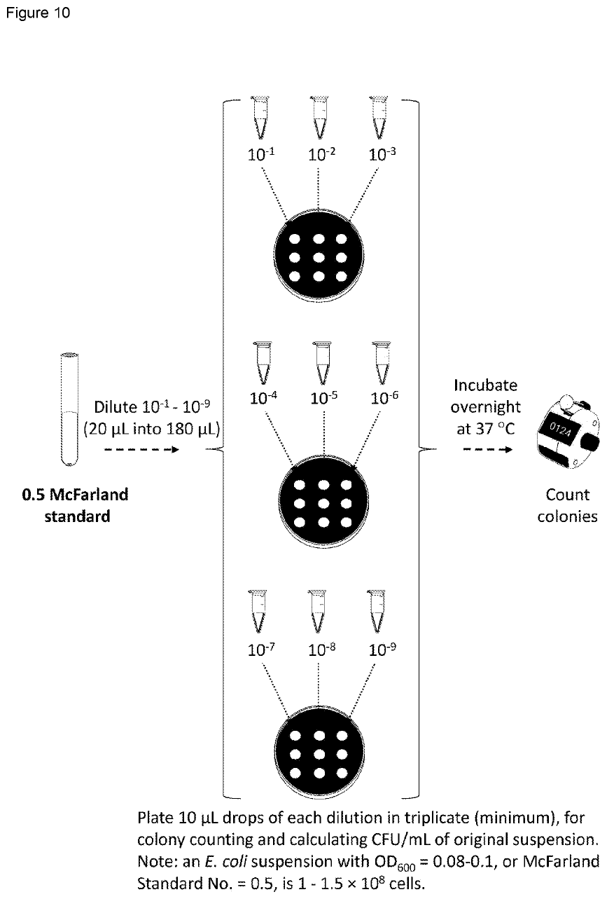

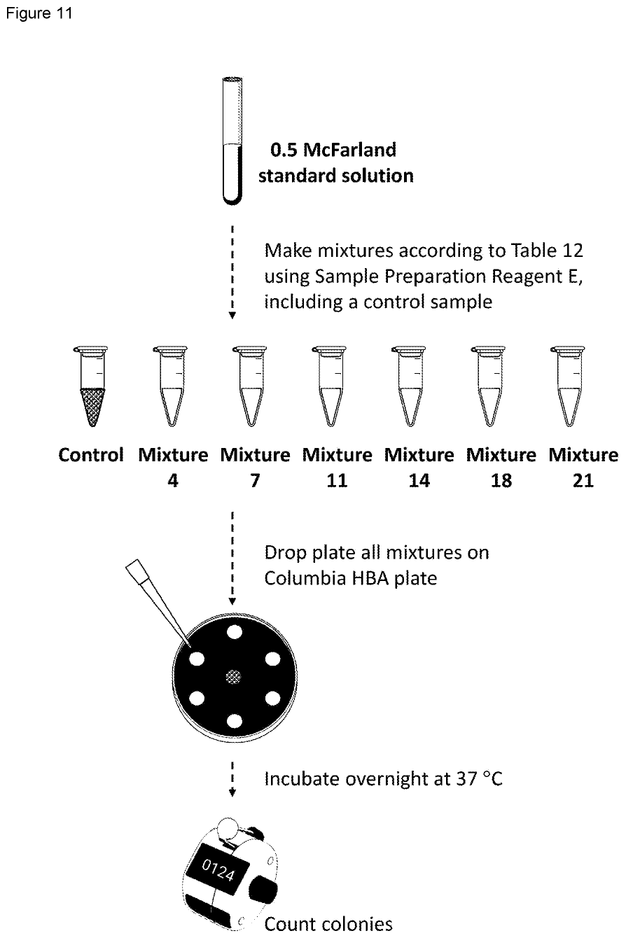

The ability of Sample Preparation Reagent E to inactivate infectious samples was examined. In brief, bla_{KPC} positive and bla_{KPC} negative E. coli strains were streaked on pre-warmed horse blood agar (HBA) plates, and individual colonies picked and resuspended in 2 mL of either 0.9% sterile saline or sterile ultra-pure molecular grade water following an overnight incubation at 37° C. The bacteria solutions were adjusted to a 0.5 McFarland standard using either 0.9% sterile saline or sterile ultra-pure molecular grade water, after which the bacteria were enumerated (see schematic FIG. 10), and the ability of the Sample Preparation Reagent E to inactivate the bacteria tested as per schematic FIG. 11 and Table 13.

TABLE 13
Bacterial inactivation trial protocol. Ratios and dilution information
of the sample:Sample Preparation Reagent E mixtures.

| | | | Dilution | | |
|---|---|---|---|---|---|
| Mixture | Sample:Reagent E | | following | Sample:solution | |
| no. | ratio | Suggested volumes | incubation | final ratio | Sample % |
| 1 | 1:1 | 10 μl Sample + 10 μl Reagent E | No dilution | 1:1 | 50.00% |
| 2 | 1:2 | 7.5 μl Sample + 15 μl Reagent E | No dilution | 1:2 | 33.33% |
| 3 | 1:5 | 4 μl Sample + 20 μl Reagent E | No dilution | 1:5 | 16.66% |
| 4 | 1:10 | 2 μl Sample + 20 μl Reagent E | No dilution | 1:10 | 9.09% |
| 5 | 2:1 | 15 μl Sample + 7.5 μl Reagent E | No dilution | 2:1 | 66.00% |
| 6 | 5:1 | 20 μl Sample + 4 μl Reagent E | No dilution | 5:1 | 83.33% |
| 7 | 10:1 | 20 μl Sample + 2 μl Reagent E | No dilution | 10:1 | 90.90% |
| 8 | 1:1 | 10 μl Sample + 10 μl Reagent E | 1 in 2 | 1:3 | 25.00% |
| 9 | 1:2 | 7.5 μl Sample + 15 μl Reagent E | 1 in 2 | 1:5 | 16.67% |
| 10 | 1:5 | 4 μl Sample + 20 μl Reagent E | 1 in 2 | 1:11 | 8.33% |
| 11 | 1:10 | 2 μl Sample + 20 μl Reagent E | 1 in 2 | 1:21 | 4.55% |
| 12 | 2:1 | 15 μl Sample + 7.5 μl Reagent E | 1 in 2 | 1:2 | 33.00% |
| 13 | 5:1 | 20 μl Sample + 4 μl Reagent E | 1 in 2 | 5:7 | 41.67% |
| 14 | 10:1 | 20 μl Sample + 2 μl Reagent E | 1 in 2 | 5:6 | 45.45% |
| 15 | 1:1 | 10 μl Sample + 10 μl Reagent E | 1 in 5 | 1:9 | 10.00% |
| 16 | 1:2 | 7.5 μl Sample + 15 μl Reagent E | 1 in 5 | 1:14 | 6.67% |
| 17 | 1:5 | 4 μl Sample + 20 μl Reagent E | 1 in 5 | 1:29 | 3.33% |
| 18 | 1:10 | 2 μl Sample + 20 μl Reagent E | 1 in 5 | 1:54 | 1.82% |
| 19 | 2:1 | 15 μl Sample + 7.5 μl Reagent E | 1 in 5 | 2:13 | 13.20% |
| 20 | 5:1 | 20 μl Sample + 4 μl Reagent E | 1 in 5 | 1:5 | 16.67% |
| 21 | 10:1 | 20 μl Sample + 2 μl Reagent E | 1 in 5 | 2:9 | 18.18% |

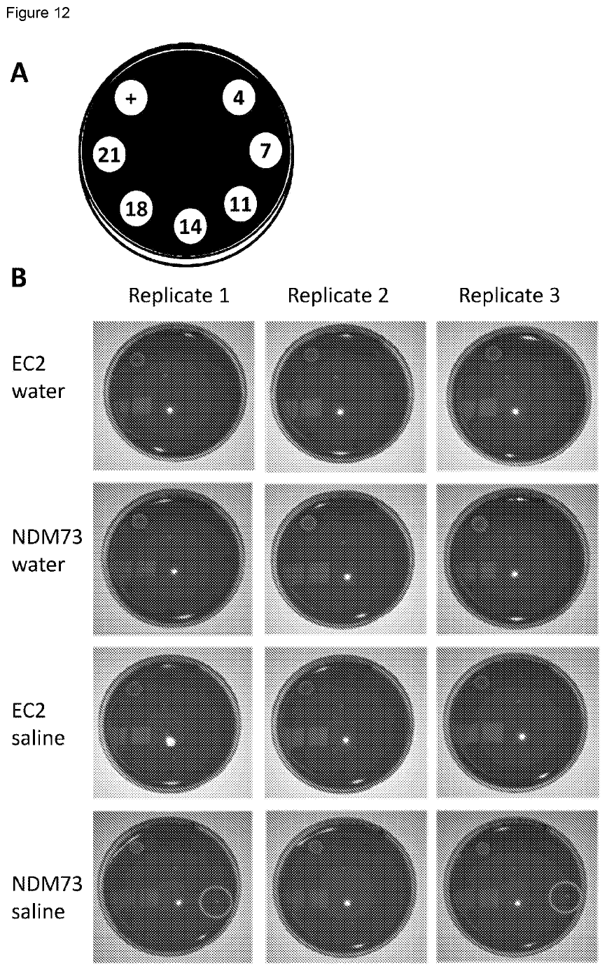

Legend: Grey highlight indicates mixtures tested in initial inactivation assays (FIG. 12). Post-incubation dilutions (1 in 2 or 1 in 5) were done with sterile ultra-pure molecular grade water. Reagent E = Sample Preparation Reagent E.

Mixtures in grey were selected for testing (Table 13, FIG. 12) for initial trials. Of those six mixtures, mixtures 4, 11, 14, 18 and 21 showed no growth in the three replicates that were performed in triplicate (Table 14), indicating these mixtures inactivated the tested E. coli strains (EC2 or NDM73, in 0.9% sterile saline or sterile ultra-pure molecular grade water). Mixture 7 showed no E. coli colonies for EC2 when the 0.5 McFarland bacteria solution was prepared with sterile ultra-pure molecular grade water (Table 14), indicating Sample Preparation Reagent E inactivated E. coli EC2 at higher Sample: Sample Preparation Reagent Ratios (1:10).

TABLE 14
Bacterial inactivation experimental results showing growth
after mixing E. coli bla_{KPC} positive sample (EC2) and E. coli
bla_{KPC} negative sample (NDM73) with Sample Preparation
Reagent E at different initial ratios.

| Mixture¹ | | | | | |
|---|---|---|---|---|---|
| Sample²:Reagent³ | | | | | |
| ratio + | | EC2 in | NDM73 in | EC2 in | NDM73 in |
| Dilution | Replicate⁵ | water | water | saline | saline |
| Mixture 4 | 1 | 0/3 | 0/3 | 0/3 | 0/3 |
| 1:10 | 2 | 0/3 | 0/3 | 0/3 | 0/3 |
| Neat⁴ | 3 | 0/3 | 0/3 | 0/3 | 0/3 |
| Mixture 7 | 1 | 0/3 | 0/3 | 0/3 | 2/3⁶ |
| 10:1 | 2 | 0/3 | 0/3 | 1/3⁶ | 0/3 |
| neat | 3 | 0/3 | 1/3⁶ | 0/3 | 0/3 |
| Mixture 11 | 1 | 0/3 | 0/3 | 0/3 | 0/3 |
| 1:10 + | 2 | 0/3 | 0/3 | 0/3 | 0/3 |
| 1 in 2 | 3 | 0/3 | 0/3 | 0/3 | 0/3 |
| Mixture 14 | 1 | 0/3 | 0/3 | 0/3 | 0/3 |
| 10:1 + | 2 | 0/3 | 0/3 | 0/3 | 0/3 |
| 1 in 2 | 3 | 0/3 | 0/3 | 0/3 | 0/3 |
| Mixture 18 | 1 | 0/3 | 0/3 | 0/3 | 0/3 |
| 1:10 + | 2 | 0/3 | 0/3 | 0/3 | 0/3 |
| 1 in 5 | 3 | 0/3 | 0/3 | 0/3 | 0/3 |
| Mixture 21 | 1 | 0/3 | 0/3 | 0/3 | 0/3 |
| 10:1 + | 2 | 0/3 | 0/3 | 0/3 | 0/3 |
| 1 in 5 | 3 | 0/3 | 0/3 | 0/3 | 0/3 |

¹A selection of mixtures from Table 13 were prepared and incubated for 10 min at room temperature followed by dilution (if required) with sterile ultra-pure molecular grade water.
²Reagent: Sample Preparation Reagent E.
³Sample: 0.5 McFarland prepared either in sterile ultra-pure molecular grade water or 0.9% sterile saline as shown.
⁴Neat: no dilution was carried out on the mixtures.
⁵Each experiment was performed in triplicate, and repeated three times over three consecutive weeks
⁶Mixtures showing growth have been highlighted in grey for ease of identification.

Further experiments were carried out with the E. coli EC2 and NDM73 isolates, following the previously described method (see FIG. 10 and FIG. 11), to determine the most effective ratios of sample to Sample Preparation Reagent E that led to sample inactivation without a post-incubation dilution step. The amount of sample that was mixed with Sample Preparation Reagent E trialled in this second inactivation experiment can be found in Table 15. Results showed that E. coli EC2 and NDM73 isolates could be inactivated when between 40% and 80% of Sample Preparation Reagent E were added to the sample, without carrying out a post-incubation dilution step (Table 16).

TABLE 15
Different mixtures of sample (prepared as 0.5 McFarland
standards either in sterile 0.9% saline or sterile ultra-
pure molecular grade water) with Sample Preparation Reagent
E trialled in the second inactivation experiment.

| | 0.5 | 0.5 | Sample | Sample |
|---|---|---|---|---|
| | McFarland | McFarland | Preparation | Preparation |
| | standard | standard | Reagent | Reagent E |
| Mixture no. | (%) | (μL) | E (%) | (μL) |
| 1 | 90 | 45 | 10 | 5 |
| 2 | 80 | 40 | 20 | 10 |
| 3 | 70 | 35 | 30 | 15 |
| 4 | 60 | 30 | 40 | 20 |
| 5 | 50 | 25 | 50 | 25 |
| 6 | 40 | 20 | 60 | 30 |
| 7 | 30 | 15 | 70 | 35 |
| 8 | 20 | 10 | 80 | 40 |
| 9 | 10 | 5 | 90 | 45 |

TABLE 16
Results from second bacterial inactivation trial showing growth after mixing E. coli bla_{KPC} positive sample
(EC2) and E. coli bla_{KPC} negative sample (NDM73) with Sample Preparation Reagent E at different ratios.

| | EC2 water | | | NDM73 water | | | EC2 saline | | | NDM73 saline | | |
|---|---|---|---|---|---|---|---|---|---|---|---|---|
| Reagent E | Exp. 1 | Exp. 2 | Exp. 3 | Exp. 1 | Exp. 2 | Exp. 3 | Exp. 1 | Exp. 2 | Exp. 3 | Exp. 1 | Exp. 2 | Exp. 3 |
| Positive Control | TMTC | TMTC | TMTC | TMTC | TMTC | TMTC | TMTC | TMTC | TMTC | TMTC | TMTC | TMTC |
| | TMTC | TMTC | TMTC | TMTC | TMTC | TMTC | TMTC | TMTC | TMTC | TMTC | TMTC | TMTC |
| | TMTC | TMTC | TMTC | TMTC | TMTC | TMTC | TMTC | TMTC | TMTC | TMTC | TMTC | TMTC |
| Mixture 1 | 0 | 0 | 0 | 0 | 0 | 0 | 1 | 3 | 0 | 1 | 0 | 1 |
| (10% Reagent E) | 0 | 0 | 0 | 0 | 0 | 0 | 0 | 2 | 1 | 0 | 0 | 0 |
| | 0 | 0 | 0 | 0 | 0 | 0 | 0 | 0 | 0 | 0 | 0 | 0 |
| Mixture 2 | 0 | 0 | 0 | 0 | 0 | 0 | 0 | 1 | 0 | 0 | 0 | 0 |
| (20% Reagent E) | 0 | 0 | 0 | 0 | 0 | 0 | 0 | 1 | 0 | 0 | 0 | 0 |
| | 0 | 0 | 0 | 0 | 0 | 0 | 0 | 0 | 0 | 0 | 0 | 0 |
| Mixture 3 | 0 | 0 | 0 | 0 | 0 | 0 | 0 | 1 | 0 | 0 | 0 | 0 |
| (30% Reagent E) | 0 | 0 | 0 | 0 | 0 | 0 | 0 | 0 | 0 | 0 | 0 | 0 |
| | 0 | 0 | 0 | 0 | 0 | 0 | 0 | 0 | 0 | 0 | 0 | 0 |
| Mixture 4 | 0 | 0 | 0 | 0 | 0 | 0 | 0 | 0 | 0 | 0 | 0 | 0 |
| (40% Reagent E) | 0 | 0 | 0 | 0 | 0 | 0 | 0 | 0 | 0 | 0 | 0 | 0 |
| | 0 | 0 | 0 | 0 | 0 | 0 | 0 | 0 | 0 | 0 | 0 | 0 |
| Mixture 5 | 0 | 0 | 0 | 0 | 0 | 0 | 0 | 0 | 0 | 0 | 0 | 0 |
| (50% Reagent E) | 0 | 0 | 0 | 0 | 0 | 0 | 0 | 0 | 0 | 0 | 0 | 0 |
| | 0 | 0 | 0 | 0 | 0 | 0 | 0 | 0 | 0 | 0 | 0 | 0 |
| Mixture 6 | 0 | 0 | 0 | 0 | 0 | 0 | 0 | 0 | 0 | 0 | 0 | 0 |
| (60% Reagent E) | 0 | 0 | 0 | 0 | 0 | 0 | 0 | 0 | 0 | 0 | 0 | 0 |
| | 0 | 0 | 0 | 0 | 0 | 0 | 0 | 0 | 0 | 0 | 0 | 0 |
| Mixture 7 | 0 | 0 | 0 | 0 | 0 | 0 | 0 | 0 | 0 | 0 | 0 | 0 |
| (70% Reagent E) | 0 | 0 | 0 | 0 | 0 | 0 | 0 | 0 | 0 | 0 | 0 | 0 |
| | 0 | 0 | 0 | 0 | 0 | 0 | 0 | 0 | 0 | 0 | 0 | 0 |
| Mixture 8 | 0 | 0 | 0 | 0 | 0 | 0 | 0 | 0 | 0 | 0 | 0 | 0 |
| (80% Reagent E) | 0 | 0 | 0 | 0 | 0 | 0 | 0 | 0 | 0 | 0 | 0 | 0 |
| | 0 | 0 | 0 | 0 | 0 | 0 | 0 | 0 | 0 | 0 | 0 | 0 |
| Mixture 9 | 0 | 0 | 0 | 1 | 0 | 0 | 0 | 0 | 0 | 0 | 0 | 0 |
| (90% Reagent E) | 0 | 0 | 0 | 0 | 0 | 0 | 0 | 0 | 0 | 0 | 0 | 0 |
| | 0 | 0 | 0 | 0 | 0 | 0 | 0 | 0 | 0 | 0 | 0 | 0 |

Legend: Mixtures from Table 15 were prepared and incubated for 10 min at room temperature, then plated and incubated as per Schematic FIG. 11. Percentage of Sample Preparation Reagent E in each mixture is shown. Samples tested were 0.5 McFarland bacterial solutions prepared either in sterile ultra-pure molecular grade water or 0.9% sterile saline as shown. Experiment has been carried out three times, with mixtures that showed growth highlighted in grey for ease of identification. No post-incubation dilutions were carried out in this experiment. TMTC: too many colonies to count.

In addition to the ability to inactivate E. coli isolates, the Sample Preparation Reagent E was also trialled for compatibility with processing samples, followed by detection of the bla_{KPC} gene with an RPA-LFD test. To do this, a bla_{KPC} test was developed by identifying consensus regions within an alignment of bla_{KPC} variant sequences, and creating a set of oligonucleotide primers (forward and reverse) and probes that were optimised for amplification and detection by RPA-LFD (Table 17). Similar tests were developed for detection of bla_{IMP} and bla_{OXA-48}, with the selection of primers and probes for those tests also shown in Table 17.

TABLE 17
Primers and probes for the development of RPA-LFD tests
for bla_{KPC}, bla_{IMP}, and bla_{OXA-48}.-*

| Gene | Primer | | | Labelled/ |
|---|---|---|---|---|
| target | type | ID | Sequence (5′ → 3′) | unlabelled |
| KPC | Forward | K_F1 | CACTGTGCAGCTCATTCAAGGGCTTTC | unlabelled |
| | | | TTG (SEQ ID NO: 9) | |
| KPC | Forward | K_F9 | CGCTTCCCACTGTGCAGCTCATTCAAG | unlabelled |
| | | | GGC (SEQ ID NO: 10) | |
| KPC | Forward | K_F10 | CTTCCCACTGTGCAGCTCATTCAAGGG | unlabelled |
| | | | CTTTC (SEQ ID NO: 11) | |
| KPC | Forward | K_F11 | CCACTGTGCAGCTCATTCAAGGGCTTT | unlabelled |
| | | | CTTG (SEQ ID NO: 12) | |
| KPC | Forward | K_F12 | CACTGTGCAGCTCATTCAAGGGCTTTC | unlabelled |
| | | | TTGC (SEQ ID NO: 15) | |
| KPC | Probe | K_P3 | /56-FAM/CCGGCTTGCTGGACACACC | labelled, FAM |
| | | | CATCCGTTACGGC/idSp/AAAATGCG | |
| | | | CTGGTTC/3SpC3/ (SEQ ID NO: | |
| | | | 16) | |
| KPC | Probe | K_P8 | /56-FAM/CGGCTTGCTGGACACACCC | labelled, FAM |
| | | | ATCCGTTACGGC/idSp/AAAATGCGC | |
| | | | TGGTTC/3SpC3/ (SEQ ID NO: | |
| | | | 17) | |
| KPC | Reverse | K_R3 | /5BiosG/CATGCCTGTTGTCAGATAT | labelled, biotin |
| | | | TTTTCCGAGATG (SEQ ID NO: | |
| | | | 18) | |
| KPC | Reverse | K_R6 | /5BiosG/CAAATTGGCGGCGGCGTTA | labelled, biotin |
| | | | TCACTGTATTG (SEQ ID NO: 19) | |
| KPC | Reverse | K_R7 | /5BiosG/CTTCAGCAACAAATTGGCG | labelled, biotin |
| | | | GCGGCGTTATC (SEQ ID NO: 20) | |
| IMP | Forward | I2_F3 | CAGCACGGGCGGAATAGAGTGGCTTAA | unlabelled |
| | | | TTCTC (SEQ ID NO: 21) | |
| IMP | Forward | I2_F5 | CATAGCGACAGCACGGGCGGAATAGAG | unlabelled |
| | | | TGG (SEQ ID NO: 22) | |
| IMP | Forward | I2_F6 | CATAGCGACAGCACGGGCGGAATAGAG | unlabelled |
| | | | TGGC (SEQ ID NO: 23) | |
| IMP | Forward | I2_F7 | CTCAATCTATCCCCACGTATGCATCTG | unlabelled |
| | | | AATTAAC (SEQ ID NO: 24) | |
| IMP | Forward | I2_F8 | ATAGAGTGGCTTAATTCTCAATCTATC | unlabelled |
| | | | CCCACGTATG (SEQ ID NO: 26) | |
| IMP | Forward | I2_F9 | CATTTTCATAGCGACAGCACGGGCGGA | unlabelled |
| | | | ATAG (SEQ ID NO: 27) | |
| IMP | Probe | I2_P1 | /56-FAM/CGGTAAGGTTCAAGCTAAA | labelled, FAM |
| | | | AATTCATTTAGCG/idSp/AGTTAGCT | |
| | | | ATTGGCTAG/3SpC3/ (SEQ ID | |
| | | | NO: 28) | |
| IMP | Probe | I2_P2 | /56-FAM/CAAGCTAAAAATTCATTTA | labelled, FAM |
| | | | GCGGAGTTAGCTATTG/idSp/CTAGT | |
| | | | TAAAAATAAAATTG/3SpC3/ (SEQ | |
| | | | ID NO: 29) | |
| IMP | Probe | I2_P3 | /56-FAM/CAAGCTAAAAATTCATTTA | labelled, FAM |
| | | | GCGGAGTTAGCTATTGG/idSp/TAGT | |
| | | | TAAAAATAAAATTG/3SpC3/ (SEQ | |
| | | | ID NO: 32) | |
| IMP | Reverse | I2_R3 | /5BiosG/CCAAACCACTACGTTATCT | labelled, biotin |
| | | | GGAGTGTGCCC (SEQ ID NO: 33) | |
| IMP | Reverse | I2_R7 | /5BiosG/AGGCAACCAAACCACTACG | labelled, biotin |
| | | | TTATCTGGAG (SEQ ID NO: 34) | |
| IMP | Reverse | I2_R8 | /5BiosG/CAGGCAACCAAACCACTAC | labelled, biotin |
| | | | GTTATCTGGAG (SEQ ID NO: 35) | |
| OXA-48 | Forward | O_F1 | ATGCGTGTATTAGCCTTATCGGCTGTG | unlabelled |
| | | | TTTTTGGTG (SEQ ID NO: 36) | |
| OXA-48 | Forward | O_F2 | CGTGTATTAGCCTTATCGGCTGTGTTT | unlabelled |
| | | | TTGGTG (SEQ ID NO: 37) | |
| OXA-48 | Forward | O_F3 | AGCCTTATCGGCTGTGTTTTTGGTGGC | unlabelled |
| | | | ATCG (SEQ ID NO: 38) | |
| OXA-48 | Forward | O_F4 | CGATTATCGGAATGCCTGCGGTAGCAA | unlabelled |
| | | | AGGAATGG (SEQ ID NO: 39) | |
| OXA-48 | Probe | O_P1 | /56-FAM/CGATTATCGGAATGCCTGC | labelled, FAM |
| | | | GGTAGCAAAGGAATGG/idSp/AAGAA | |
| | | | AACAAAAGTTGG/3SpC3/ (SEQ ID | |
| | | | NO: 40) | |
| OXA-48 | Probe | O_P2 | /56-FAM/ATGCCTGCGGTAGCAAAGG | labelled, FAM |
| | | | AATGGCAAGAAAACAAAAG/idSp/TG | |
| | | | GAATGCTCACTTTACTG/3SpC3/ | |
| | | | (SEQ ID NO: 41) | |
| OXA-48 | Probe | O_P5 | /56-FAM/CGGTAGCAAAGGAATGGCA | labelled, FAM |
| | | | AGAAAACAAAAGTTGG/idSp/ATGCT | |
| | | | CACTTTACTG/3SpC3/ (SEQ ID | |
| | | | NO: 42) | |
| OXA-48 | Reverse | O_R4 | /5BiosG/CCGTTTAAGATTATTGGTA | labelled, biotin |
| | | | AATCCTTGCTG (SEQ ID NO: 43) | |
| OXA-48 | Reverse | O_R5 | /5BiosG/CTTGGTTCGCCCGTTTAAG | labelled, biotin |
| | | | ATTATTGGTAAATCC (SEQ ID NO: | |
| | | | 44) | |
| OXA-48 | Reverse | O_R7 | /5BiosG/ATGCGGGTAAAAATGCTTG | labelled, biotin |
| | | | GTTCGCCCGTTTAAG (SEQ ID NO: | |
| | | | 45) | |
| OXA-48 | Reverse | O_R8 | /5BiosG/CCAGAGCACAACTACGCCC | labelled, biotin |
| | | | TGTGATTTATG (SEQ ID NO: 46) | |

*After optimisation of the protocol, DNA amplification was carried out using the TwistAmp ™ nfo kit (TwistDX, USA) by mixing 29.5 μL rehydration buffer, 2.1 μL forward primer (10 μM), 2.1 μL biotin-labelled reverse primer (10 μM), and 0.6 μL 56-FAM labelled probe containing an internal THF residue and a 3′ C3 spacer (10 μM), with nuclease-free water to a final volume of 40 μL and then adding this mixture to the RPA pellet. The reaction mix was then split across 5 tubes (8 μL per tube) and 1 μL of template DNA (or water in case of the no template controls) and 1 μL of 140 mM magnesium acetate added to each tube. After incubating the mixture for 10-15 minutes at 39° C., 2 μL of the amplified product was then added to HybriDetect lateral flow strips (Milenia biotec, Germany) pre-activated with 8 μL of 0.4% casein in PBS-T (137 mM NaCl, 2.7 mM KCl, 10 mM Na₂HPO₄, 1.8 mM KH₂PO₄ and 0.1% Tween 20, pH 9.0). Lateral flow strips were then placed into 100 μL of borate running buffer (100 mM H₃BO₃, 100 mM Na₂B₄O₇, 1% BSA, 0.05% Tween 20, pH 8.8) for 5 minutes before analysing the strips visually, as well as taking a photo of the strips with the Vilber Lourmat (France) gel documentation imaging system and analysing the picture using Image J (National Institutes of Health, Bethesda, USA). Singleplex and multiplex testing identified different sets of primers to give high detection sensitivity, indicating that each
primer and probe could be advantageous depending on the environment of the reaction mixes.

After development of the bla_{KPC} RPA-LFD test compatibility of the Sample Preparation Reagent E with the bla_{KPC} RPA-LF test was trialled. For this, Sample Preparation Reagent E was combined with E. coli EC2 (bla_{KPC} positive sample) or NDM73 (bla_{KPC} negative sample) using mixture conditions 11, 14, 18 or 21 (Table 13) and incubated for 10 minutes at room temperature then diluted as required for the mixture. Mixtures (1 L) were then used for bla_{KPC} detection with the bla_{KPC} RPA-LFD test. Results demonstrated bla_{KPC} could be correctly detected in all samples that contained bla_{KPC} positive E. coli EC2, and was correctly not detected in the bla_{KPC} negative E. coli NDM73 (results not shown).

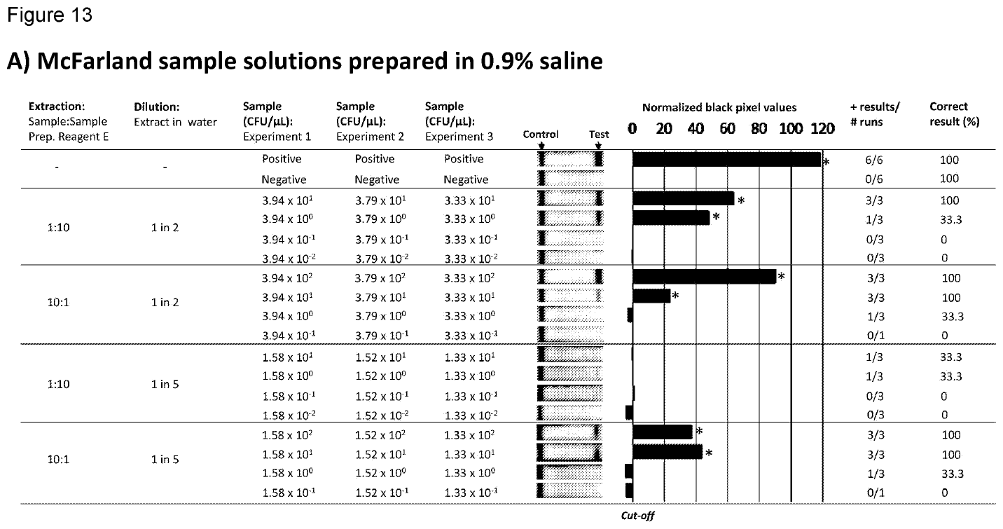

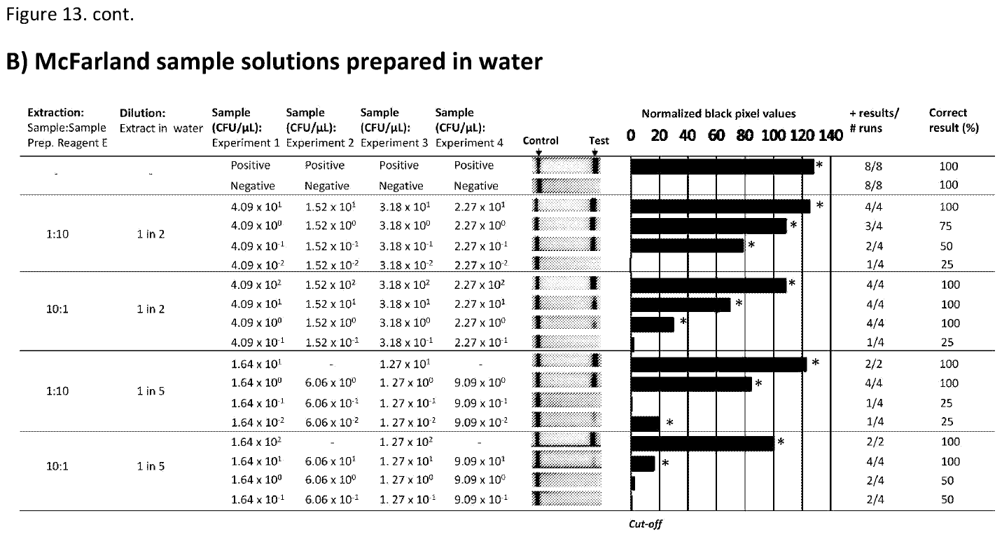

Further experiments aimed to determine the number of colony forming units (CFU/μL) that could be detected with the bla_{KPC} RPA-LFD test using the same McFarland E. coli culture mixtures (11, 14, 18 and 21, Table 13). Briefly, E. coli EC2 (0.5 McFarland standard prepared in either sterile 0.9% saline or sterile ultra-pure molecular grade water) was mixed with the Sample Preparation Reagent E at either a 1:10 or 10:1 ratio. The mixtures were then incubated for 10 min at room temperature, followed by post-incubation dilution (either 1 in 2, or 1 in 5 in sterile water). Processed samples were then tested with the bla_{KPC} RPA-LFD test (FIG. 13). Results showed that when McFarland standards were prepared with water, all combinations of Sample Preparation Reagent E mixtures enabled bla_{KPC} RPA-LFD detection down to between 101 and 10° CFU/μL (FIG. 13B). Similarly, McFarland standards prepared with saline (FIG. 13A) and tested the same way were also detected at a consistent 101 CFU/μL when the sample:Sample Preparation Reagent E ratio was 10:1, with the 1:10 ratio consistently detected at 101 CFU/μL for the 1 in 2 dilution only.

Sample Preparation Reagent E was also mixed 1:1 with 0.5 McFarland bacterial solutions of antimicrobial resistant Klebsiella pneumonia and Enterobacter hormaechei (prepared in either sterile 0.9% saline or sterile ultra-pure molecular grade water). Following a 10 minute incubation at room temperature, the mixtures were then drop-plated onto Columbia horse blood agar to determine Sample Preparation Regent E's ability to inactivate the tested McFarland bacterial solutions. The mixtures were also serially diluted (1 in 10) and 1 μL of each dilution added to either a RPA-LFD or real-time PCR (QuantiTect Probe mastermix; Qiagen) reaction. Results showed that both bacterial strains were inactivated (no growth observed) by the Sample Preparation Reagent E (Table 18), and detected with good analytical sensitivity using both real-time PCR and RPA-LFD-based bla_{KPC}, bla_{OXA-48} and bla_{IMP} tests respectively (Table 28). This data demonstrates that the Sample Preparation Reagent E is able to inactivate K. pneumoniae and E. hormaechei bacterial cells and can be used to process bacterial samples for downstream nucleic acid detection testing (e.g., PCR or RPA-LFD test).

TABLE 18
Summary of the Sample Preparation Reagent E's inactivation
testing with K. pneumoniae & E. hormaechei.¹

| | | # | # | Total | Overall |
|---|---|---|---|---|---|
| Bacterium | #Passes | Watches | Fails | criteria | acceptance |
| Klebsiella | 3 | 0 | 0 | 1 | Pass |
| pneumoniae | | | | | |
| Enterobacter | 3 | 0 | 0 | 1 | Pass |
| hormaechei | | | | | |

¹Inactivation testing was repeated three times and “Pass” given to each experiment that showed no bacterial growth after a 0.5 McFarland standard of the respective bacteria was incubated 1:1 with the Sample Preparation Reagent E for 10 min at room temperature. Overall acceptance rating of “Pass” is given to inactivation tests with ≤2 “Watches” and 0 “Fails”

TABLE 19
Summary of the Sample Preparation Reagent E's performance
testing (compatibility with RPA and real-time PCR).¹

| | | # | # | Total | Overall |
|---|---|---|---|---|---|
| Bacterium | #Passes | Watches | Fails | criteria | acceptance |
| Klebsiella | 12 | 0 | 0 | 4 | Pass |
| pneumoniae | | | | | |
| Enterobacter | 12 | 0 | 0 | 4 | Pass |
| hormaechei | | | | | |

¹Compatibility was tested by serially diluting inactivated 0.5 McFarland standards of the respective bacteria and testing it with RPA-LFD and real-time PCR. Experiment was repeated three times for both, using a) sterile saline or b) sterile water as diluent, and “Pass” given if the test could detect down to at least a 10¹ (RPA-LFD) or 10⁰ CFU/μL value (PCR). Overall acceptance rating of “Pass” is given to inactivation tests with ≤2 “Watches” and 0 “Fails”.

Further inactivation testing was performed using two solid (Columbia horse blood agar or Trypticase soy agar plates) and two liquid (Muller Hinton broth or Tryptic soy broth) media to further test Sample Preparation Reagent E's ability to inactivate bacteria. For this, 0.5 McFarland standards were prepared for Klebsiella pneumoniae, Escherichia coli and Enterobacter hormaechei in either 0.9% sterile saline or sterile ultra-pure molecular grade water, then the standards mixed with 0-90% of Sample Preparation Reagent E (in 10% increments) and incubated for 10 min at room temperature. After incubation, the mixtures were added to either a) Muller Hinton broth or b) Tryptic soy broth, or drop-plated onto c) Columbia horse blood agar or d) Trypticase soy agar plates. Results overall matched previous inactivation testing, with the 1:1 ratio of sample: Reagent E confirmed as an effective ratio for inactivating the above Gram-negative bacteria (Table 20 & Table 21).

TABLE 20
Summary of inactivation testing using two solid and two liquid growth
media. 0.5 McFarland standards prepared in sterile saline were used.¹

| % SPR in | K. pneumoniae in saline | | | | E. coli in saline | | | | E. hormaechei in saline | | | |
|---|---|---|---|---|---|---|---|---|---|---|---|---|
| mixture | TSB | MHB | TSA | HBA | TSB | MHB | TSA | HBA | TSB | MHB | TSA | HBA |
| 100 | Pass | Pass | Pass | Pass | Pass | Pass | Pass | Pass | Pass | Pass | Pass | Pass |
| 90 | Fail | Pass | Pass | Pass | Pass | Fail | Pass | Pass | Pass | Pass | Fail | Fail |
| 80 | Pass | Pass | Fail | Pass | Pass | Pass | Pass | Pass | Pass | Pass | Pass | Pass |
| 70 | Pass | Pass | Pass | Pass | Pass | Pass | Pass | Pass | Pass | Pass | Pass | Pass |
| 60 | Pass | Pass | Pass | Pass | Pass | Pass | Pass | Pass | Pass | Pass | Pass | Pass |
| 50 | Pass | Pass | Pass | Pass | Pass | Pass | Pass | Pass | Pass | Pass | Pass | Pass |
| 40 | Pass | Pass | Pass | Pass | Pass | Pass | Pass | Pass | Pass | Pass | Pass | Pass |
| 30 | Pass | Pass | Pass | Pass | Pass | Pass | Pass | Pass | Pass | Pass | Pass | Pass |
| 20 | Pass | Fail | Pass | Pass | Pass | Pass | Pass | Pass | Fail | Fail | Fail | Fail |
| 10 | Fail | Fail | Fail | Fail | Pass | Fail | Fail | Fail | Fail | Fail | Fail | Fail |
| 0 | Fail | Fail | Fail | Fail | Fail | Fail | Fail | Fail | Fail | Fail | Fail | Fail |

¹Experiment was carried out in triplicate and repeated three times; TSB: Tryptic soy broth, MHB: Muller Hinton broth, TSA: Trypticase soy agar, HBA: Columbia horse blood agar, SPR: Sample Preparation Reagent E; Pass: no growth observed in any of the 9 replicates; Fail: growth observed in one or more of the 9 replicates

TABLE 21
Summary of inactivation testing using two solid and two liquid growth
media. 0.5 McFarland standards prepared in sterile water were used.1

| % SPR in | K. pneumoniae in water | | | | E. coli in water | | | | E. hormaechei in water | | | |
|---|---|---|---|---|---|---|---|---|---|---|---|---|
| mixture | TSB | MHB | TSA | HBA | TSB | MHB | TSA | HBA | TSB | MHB | TSA | HBA |
| 100 | Pass | Pass | Pass | Pass | Pass | Pass | Pass | Pass | Pass | Pass | Pass | Pass |
| 90 | Pass | Fail | Pass | Pass | Pass | Pass | Pass | Pass | Pass | Pass | Pass | Pass |
| 80 | Pass | Pass | Pass | Pass | Pass | Pass | Pass | Pass | Pass | Pass | Pass | Pass |
| 70 | Pass | Pass | Pass | Pass | Pass | Pass | Pass | Pass | Pass | Pass | Pass | Pass |
| 60 | Pass | Pass | Pass | Pass | Pass | Pass | Pass | Pass | Pass | Pass | Pass | Pass |
| 50 | Pass | Pass | Pass | Pass | Pass | Pass | Pass | Pass | Pass | Pass | Pass | Pass |
| 40 | Pass | Pass | Pass | Pass | Pass | Pass | Pass | Pass | Pass | Pass | Pass | Pass |
| 30 | Pass | Pass | Pass | Pass | Pass | Pass | Pass | Pass | Pass | Pass | Pass | Pass |
| 20 | Pass | Pass | Pass | Pass | Pass | Pass | Pass | Pass | Pass | Fail | Pass | Fail |
| 10 | Fail | Fail | Fail | Pass | Pass | Pass | Pass | Fail | Fail | Fail | Fail | Fail |
| 0 | Fail | Fail | Fail | Fail | Fail | Fail | Fail | Fail | Fail | Fail | Fail | Fail |

¹Experiment was carried out in triplicate and repeated three times; TSB: Tryptic soy broth, MHB: Muller Hinton broth, TSA: Trypticase soy agar, HBA: Columbia horse blood agar, SPR: Sample Preparation Reagent E; Pass: no growth observed in any of the 9 replicates; Fail: growth observed in one or more of the 9 replicates.

In addition to testing additional growth media, a shortening of the incubation time was also tested. For this, 0.5 McFarland standards were prepared for Klebsiella pneumoniae, Escherichia coli and Enterobacter hormaechei in either 0.9% sterile saline or sterile ultra-pure molecular grade water, then the standards mixed 1:1 with Sample Preparation Reagent E and incubated for either 10 min, 5 min or 2 min at room temperature. After incubation, the mixtures were added to either a) Muller Hinton broth or b) Tryptic soy broth, or drop-plated onto c) Columbia horse blood agar or d) Trypticase soy agar plates. Overall results showed that incubation time for the above Gram-negative bacteria could be shortened to 2 min (Table 22).

TABLE 22
Summary of different incubation times on Sample Preparation Reagent
E's ability to inactivate bacteria when mixed 1:1 with 0.5 McFarland
standards prepared in either sterile saline or water.¹

| Incubation | | E. coli | | | | K. pneumoniae | | | | E. hormaechei | | | |
|---|---|---|---|---|---|---|---|---|---|---|---|---|---|
| time | | TSB | MHB | TSA | HBA | TSB | MHB | TSA | HBA | TSB | MHB | TSA | HBA |
| 2 | min | Pass | Pass | Pass | Pass | Pass | Pass | Pass | Pass | Pass | Pass | Pass | Pass |
| 5 | min | Pass | Pass | Pass | Pass | Pass | Pass | Pass | Pass | Pass | Pass | Pass | Pass |
| 10 | min | Pass | Pass | Pass | Pass | Pass | Pass | Pass | Pass | Pass | Pass | Pass | Pass |

¹Experiment was carried out in triplicate for both, McFarland standards prepared in sterile saline or sterile water. Controls (not shown; 1:1 of 0.5 McFarland standards:sterile saline) showed growth for each of the triplicates. TSB: Tryptic soy broth, MHB: Muller Hinton broth, TSA: Trypticase soy agar, HBA: Columbia horse blood agar; Pass: no growth observed in any of the 3 replicates irrespective of how McFarland standards were prepared; Fail: growth observed in one or more of the 3 replicates irrespective of how McFarland standards were prepared.

In order to determine the effect of increasing amounts of Sample Preparation Reagent E prepared samples when added to a real-time PCR reaction, a 0.5 McFarland standard of K. pneumoniae was prepared in 0.9% sterile saline, and the standard diluted with 0.9% sterile saline. The different dilutions were then mixed 1:1 with Sample Preparation Reagent E and incubated for 10 min at room temperature. After incubation, different amounts of the mixture were tested with the bla_{OXA-48} in-house real-time PCR assay. Results showed that increased amounts of Sample Preparation Reagent E negatively affected the success of the real-time PCR reaction (Table 23). These results demonstrate that successful detection using Sample Preparation Reagent E can be judiciously achieved by putting lower sample volumes into the real-time PCR mix rather than higher sample volumes. This counter-intuitive approach can lead to very high sensitivity of detection.

TABLE 23
Summary of the effect of increasing Sample Preparation Reagent E
in a real-time PCR mastermix on the success of the PCR reaction.¹

| | Volume of | Volume of | | |
|---|---|---|---|---|
| McFarland | mixture | Reagent E | Relative Template | |
| standard | loaded (μL) | loaded (μL) | concentration | Pass/Fail |
| 0.5 | 1 | 0.5 | 1x | Pass |
| 0.25 | 2 | 1 | 1x | Pass |
| 0.1 | 5 | 2.5 | 1x | Fail |
| 0.25 | 1 | 0.5 | 0.5x | Pass |
| 0.125 | 2 | 1 | 0.5x | Pass |
| 0.1 | 1 | 0.5 | 0.2x | Pass |
| 0.02 | 5 | 2.5 | 0.2x | Fail |

1. a 0.05 Cycle Threshold was Chosen and Samples with Ct Values Above the Threshold were Given a “Pass”.

Sample preparation Reagent E was also mixed 1:1 with the Gram-positive organism Staphylococcus epidermidis (a range of dilutions prepared using PBS as the diluent). The bacteria was found to be either completely inactivated, or lead to >4 logs (10,000×) reduction in bacterial concentrations (Cfu/mL) in a number of different incubation conditions including occasional vortexing, constant vortexing and constant vortexing with beads (to simulate bead beating or homogenization) (Table 24). Agents that can reduce bacterial concentration by greater than 4 logs (10,000×) are considered effective disinfectants in some disinfection circumstances. These results demonstrate that Sample Preparation Reagent E can also inactivate or disinfect Gram-positive bacteria.

TABLE 24
Sample Preparation Reagent E inactivation of Gram-
positive bacteria Staphylococcus epidermidis

| | | Experiment 1 | | Experiment 2 | |
|---|---|---|---|---|---|
| | Incubation | | Log | | Log10 |
| Sample | conditions | Cfu/mL | reduction¹ | Cfu/mL | reduction¹ |
| No Reagent | No vortexing | 3 × 10⁸ | NA | 2.67 × 10⁸ | NA |
| E control | | | | | |
| Bacteria:Reagent | Occasional | 5.3 × 10³ | 4.8 | 0 | 8.4 |
| E | vortexing | | | | |
| (1:1) | Constant vortexing | 0 | 8.5 | 0 | 8.4 |
| | Constant vortexing | 6 × 10³ | 4.7 | 0 | 8.4 |
| | with | | | | |
| | homogenization | | | | |
| | beads | | | | |

¹Log reduction: Log₁₀ reduction in bacterial Cfu/mL compared to the no-TNA-Cifer control.

Sample Preparation Reagent E was also tested for detection of Bacillus thuringiensis (BT) spores. In the first experiment, BT spores were diluted in TNA-Cifer reagent to concentrations of 2045, 20 and 0.2 IU/μL, and incubated at room temperature for 10 minutes with constant vortexing. Sample mixtures were then either tested directly, or diluted 1/2, 1/5 or 1/10 in water prior to PCR testing for the 16s rRNA bacterial gene using the iTaq Universal Sybr-Green Supermix (BioRad;Table 25). This experiment was repeated and then tested using the iTaq Universial Probe Supermix (BioRad; Table 26). Results demonstrated that BT spores could be detected after incubation with Sample Preparation Reagent E, and were detected most effectively when tested more diluted—that is, with a 1/5 or 1/10 post-incubation dilution. These results demonstrate that TNA-Cifer Reagent E can process spore samples for subsequent PCR detection.

TABLE 25
Sample preparation Reagent E can process BT spores
for subsequent SYBR-Green PCR detection

| | Post-incubation dilution | | | | | | | | | |
|---|---|---|---|---|---|---|---|---|---|---|
| BT spore | 1x | | 1 in 2 | | 1 in 5 | | 1 in 10 | | Purified BT | |
| concentration | | Ct | | Ct | | Ct | | Ct | | Ct |
| IU/μL | Ct Av | Stdev | Ct Av | Stdev | Ct Av | Stdev | Ct Av | Stdev | Ct Av | Stdev |
| 2045 | Undet | NA | Undet | NA | Undet | NA | Undet | NA | 15.3 | 0.10 |
| 20.45 | Undet | NA | Undet | NA | Undet | NA | 20.8 | 0.2 | 18.4 | 0.45 |
| 0.2 | Undet | NA | 29.3 | NA | 24.4 | 1.0 | 25.2 | 0.2 | 22.0 | 0.39 |

Samples (5 μL) were added to a 15 μL PCR master mix for a 20 μL final volume reaction)Ct Av: average cycle threshold values for all replicates; Ct Stdev: standard deviation cycle threshold values for all replicates; Undet: undetected; NA: Not applicable.

TABLE 26
Sample preparation Reagent E can process BT spores
for subsequent Probe based PCR detection

| | Post-incubation dilution | | | | | | | | | |
|---|---|---|---|---|---|---|---|---|---|---|
| | 1x | | 1 in 2 | | 1 in 5 | | 1 in 10 | | Purified BT | |
| BT spore | | Ct | | Ct | | Ct | | Ct | | Ct |
| concentration | Ct Av | Stdev | Ct Av | Stdev | Ct Av | Stdev | Ct Av | Stdev | Ct Av | Stdev |
| 382 | Undet | NA | Undet | NA | 20.3 | 0.17 | 21.2 | 0.11 | 18.0 | 0.02 |
| 3.8 | 18.4 | 0.58 | 25.4 | 0.03 | 26.9 | 0.07 | 28.3 | 0.09 | 21.5 | 0.10 |
| 0.038 | 27.6 | 0.18 | 32.2 | 0.07 | 33.3 | 0.17 | 34.2 | 0.27 | 24.8 | 0.04 |

Samples (5 μL) were added to a 15 μL PCR master mix for a 20 μL final volume reaction)Ct Av: average cycle threshold values for all replicates; Ct Stdev: standard deviation cycle threshold values for all replicates; Undet: undetected; NA: Not applicable.

### EXAMPLE 11: Sample Preparation Reagent Formulations can be Used to Process Samples for RT-RPA-LFD of Dengue Virus Serotypes (DENV 1-4) and can Inactivate Dengue Virus

The ability of Sample Preparation Reagent E to process and inactivate samples comprising Dengue virus for subsequent reverse transcription recombinase polymerase amplification lateral flow detection (RT-RPA-LFD) was examined.

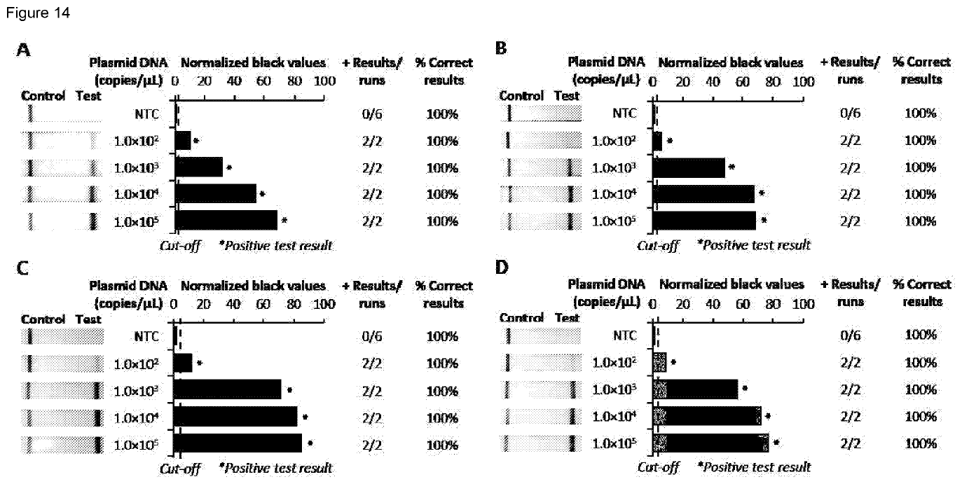

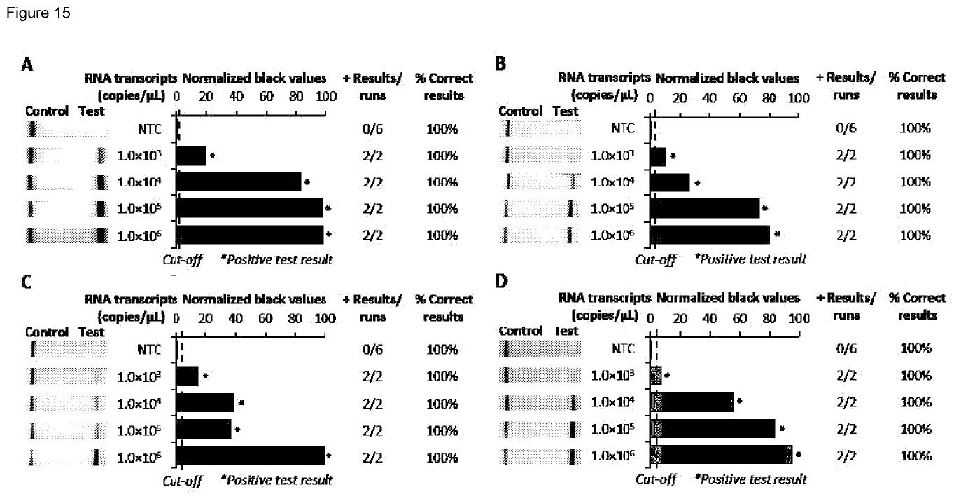

To develop rapid, low-resource, and serotype-specific DENV tests, for each DENV serotype, we designed a recombinase polymerase amplification (RPA) test that targeted a conserved region within the Dengue NS5 gene. Primers and probes were designed to enable subsequent lateral flow detection (LFD), such that appearance of a test line signified positive detection of a DENV serotype (Table 27). RPA primer and probe optimisation were performed first using plasmid DNA, and were subsequently confirmed to be suitable for reverse transcription RPA (RT-RPA) using RNA transcripts. The analytical sensitivity of the final optimised tests was determined using 10-fold dilutions of both plasmid (FIG. 14) and RNA-transcripts (FIG. 15). Test lines were observed to appear for very low concentrations of both plasmid DNA and RNA transcripts. Image J analysis of test band intensity revealed the DENV 1-4 RPA-LFD and RT-RPA-LFD tests were able to consistently detect down to 102 copies/μL of plasmid DNA and 103 copies/μL of RNA transcripts.

TABLE 27
Primers and probes for development of RPA-LFD tests for
the four Dengue virus serotypes.

| Gene | Primer | | | Labelled/ |
|---|---|---|---|---|
| target | type | ID | Sequence (5′ → 3′) | unlabelled |
| DENV-1 | Forward | D1-F3 | CATGGATCATATGAGGTCAARCCATCAGGA | unlabelled |
| | | | TCAGC (SEQ ID NO: 47) | |
| DENV-1 | Forward | D1-F5 | CATCAGGATCAGCCTCATCHATGGTSAATG | unlabelled |
| | | | GHGTG (SEQ ID NO: 48) | |
| DENV-1 | Forward | D1-F7 | GGHGTGGTGAGAYTGCTMACCAAACCATGG | unlabelled |
| | | | GATG (SEQ ID NO: 49) | |
| DENV-1 | Reverse | D1-R2 | [5′ Biotin] | labelled, biotin |
| | | | CATRATTTGTGCTGTGCCTCGTTTYGCTYT | |
| | | | TGGTG (SEQ ID NO: 50) | |
| DENV-1 | Reverse | D1-R3 | [5′ Biotin] | labelled, biotin |
| | | | GTCACCTCCATRATTTGTGCTGTGCCTCGT | |
| | | | TTYGC (SEQ ID NO: 51) | |
| DENV-1 | Reverse | D1-R4 | [5′ Biotin] | labelled, biotin |
| | | | GCTGTCACCTCCATRATTTGTGCTGTGCCT | |
| | | | CGTT (SEQ ID NO: 52) | |
| DENV-1 | Probe | D1-P1 | 5′ FAM] | labelled, FAM |
| | | | CATYCCCATGGTCACACAAATAGCYATGAC | |
| | | | [Internal dS Spacer]GAYACHACAC | |
| | | | CCTTT[3′ C3 spacer] (SEQ ID | |
| | | | NO: 53) | |
| DENV-1 | Probe | D1-P2 | [5′ FAM] | labelled, FAM |
| | | | CATGGTCACACAAATAGCYATGACTGAYAC | |
| | | | [Internal dS Spacer] ACACCCTTY | |
| | | | GGACAAC [3′ C3 spacer] (SEQ ID | |
| | | | NO: 54) | |
| DENV-2 | Forward | D2-F1 | HTAHGAHCAAGACCACCCATACAAAACNTG | unlabelled |
| | | | GGCNTAC (SEQ ID NO: 55) | |
| DENV-2 | Forward | D2-F2 | AAAACNTGGGCNTACCANGGNAGCTANGAA | unlabelled |
| | | | AC (SEQ ID NO: 56) | |
| DENV-2 | Forward | D2-F3 | GGNAGCTANGAAACAAAACANACTGGATCA | unlabelled |
| | | | GCA (SEQ ID NO: 57) | |
| DENV-2 | Forward | D2-F4 | CAYGGYAGYTAYGAAACAAAACARACNGGA | unlabelled |
| | | | TCAGC (SEQ ID NO: 58) | |
| DENV-2 | Forward | D2-F5 | CRTGGGCHTACCAYGGYAGYTAYGAAACAA | unlabelled |
| | | | AACARACNGG (SEQ ID NO: 59) | |
| DENV-2 | Reverse | D2-R1 | [5′ FAM] | labelled, FAM |
| | | | GTRTCHACTTTYTCTTTRAAVACRCGYTGY | |
| | | | TGTCC (SEQ ID NO: 60) | |
| DENV-2 | Reverse | D2-R2 | [5′ FAM] | labelled, FAM |
| | | | GGGTTCTCGTGTCCACTTTYTCTTTRAAAA | |
| | | | CRCGC (SEQ ID NO: 61) | |
| DENV-2 | Probe | D2-P1 | [5′ Biotin] | labelled, biotin |
| | | | GRYTGCTRACMAAACCTHGGGAYGTYVTYC | |
| | | | C [Internal dS spacer] AYGGTRA | |
| | | | CACARATGG [3′ C3 spacer] (SEQ | |
| | | | ID NO: 62) | |
| DENV-3 | Forward | D3-F1 | AACATGGCACTATGATGAHGAAAAYCCYTA | unlabelled |
| | | | YAAAACG (SEQ ID NO: 63) | |
| DENV-3 | Forward | D3-F2 | AAAAYCCYTAYAAAACGTGGGCTTACCATG | unlabelled |
| | | | GATC (SEQ ID NO: 64) | |
| DENV-3 | Forward | D3-F3 | TGAHGAAAAYCCYTAYAAAACGTGGGCTTA | unlabelled |
| | | | C (SEQ ID NO: 65) | |
| DENV-3 | Forward | D3-F4 | GARAAYCCYTACAAAACGTGGGCTTACCAY | unlabelled |
| | | | GGATCYTATG (SEQ ID NO: 66) | |
| DENV-3 | Forward | D3-F5 | CTTACCAYGGATCBTATGAAGTHAARGCCA | unlabelled |
| | | | CAGGC (SEQ ID NO: 67) | |
| DENV-3 | Reverse | D3-R1 | [5′ FAM] | labelled, FAM |
| | | | GTGTCCACTTTCTCTTTRAARACYCTYTGC | |
| | | | TGSCC (SEQ ID NO: 68) | |
| DENV-3 | Probe | D3-P1 | [5′ Biotin] | labelled, biotin |
| | | | GATAAAYGGAGTYGTGAAACTYCTCACNAA | |
| | | | [Internal dS spacer] CCRTGGGAT | |
| | | | GTGGTKCC [3′ C3 spacer] (SEQ | |
| | | | ID NO: 69) | |
| DENV-3 | Probe | D3-P2 | [5′Biotin]GATAAAYGGAGTYGTGAAAC | labelled, biotin |
| | | | TYCTCACNAA[Internal dS spacer] | |
| | | | CCRTGGGATGTGGTKCC[3′ C3 | |
| | | | spacer] (SEQ ID NO: 70) | |
| DENV-4 | Forward | D4-F1 | AGAAACHTGGCAYTATGAYCADGAAAAYCC | unlabelled |
| | | | ATACAG (SEQ ID NO: 71) | |
| DENV-4 | Forward | D4-F2 | GAAGCTATGAAGCYCCYTCGACAGGCTCDG | unlabelled |
| | | | CNTCYTC (SEQ ID NO: 72) | |
| DENV-4 | Forward | D4-F3 | TCGACAGGCTCTGCATCCTCCATGGTGAAC | unlabelled |
| | | | GGGGTVG (SEQ ID NO: 73) | |
| DENV-4 | Forward | D4-F4 | TTCGACAGGCTCWGCRTCCTCCATGGTGAA | unlabelled |
| | | | CGGGG (SEQ ID NO: 74) | |
| DENV-4 | Forward | D4-F5 | CCTTCGACAGGCTCWGCRTCCTCCATGGTG | unlabelled |
| | | | AACGGGGTRG (SEQ ID NO: 75) | |
| DENV-4 | Forward | D4-F6 | CATGGRAGCTATGAAGCYCCTTCGACAGGC | unlabelled |
| | | | TCWGC (SEQ ID NO: 76) | |
| DENV-4 | Forward | D4-F7 | AGCTATGAAGCTCCTTCGACAGGCTCWGCR | unlabelled |
| | | | TCCTC (SEQ ID NO: 77) | |
| DENV-4 | Reverse | D4-R1 | [5′ FAM] | labelled, FAM |
| | | | GTRTCNACCTTCTCYTTRAACACTCTYTGT | |
| | | | TGCCC (SEQ ID NO: 78) | |
| DENV-4 | Probe | D4-P1 | [5′ Biotin] | labelled, biotin |
| | | | GGGAYGTRRTTCCRATGGTGACYCAGTTRG | |
| | | | C [Internal dS spacer] ATGACAG | |
| | | | AYACAACCC [3′ C3 spacer] (SEQ | |
| | | | ID NO: 79) | |
| DENV-4 | Probe | D4-P2 | [5′Biotin] | labelled, biotin |
| | | | AAAACCYTGGGAYGTRATTCCRATGGTGAC | |
| | | | [Internal dS spacer] CAGTTRGCC | |
| | | | ATGACAGA [3′ C3 spacer] | |
| | | | (SEQ ID NO: 80) | |

*Each respective DENV 1-4 test was prepared separately using the TwistAmp ™ nfo kit (TwistDX, Cambridge, United Kingdom), with final reaction conditions of 1x rehydration buffer and ⅕ rehydrated lyophilized pellet, forward primer (420 nM), reverse primer (420 nM), and probe (120 nM). For homogenized mosquito testing, Ribolock (10 U) and Moloney Murine Leukemia virus reverse transcriptase (mMLV, 40 U) were included with the individual DENV 1-4 RPA tests, along with 1 μL extracted RNA and magnesium acetate (14 mM), to a final reaction volume of 10 μL. For testing RNA-transcripts, betaine (120 mM) was also included with the DENV 1-4 RT-RPA tests. Reactions were incubated at 39° C. for 20 minutes before lateral flow detection. For plasmid DNA control testing, Ribolock, mMLV, and betaine were excluded. 1 μL DNA sample was added before activation by magnesium acetate (14 mM), to a final reaction volume of 10 μL. Reactions were incubated for 15 minutes at 39° C. before lateral flow detection. After the RPA (DNA plasmid control) or RT-RPA (RNA transcripts and mosquitoes) incubation, 2 μL of the amplified RPA reaction mix was added to the sample pad of the lateral flow strip (Milenia Biotec, Giessen, Germany), which had been pre-activated by the addition of 8 μL 0.4% casein in PBST to the sample pad. Strips were then placed in 100 μL running buffer (100 mM H₃BO₃, 100 mM Na₂B₄O₇, 1% bovine serum albumin, 0.05% Tween 20, pH 8.8) for five minutes at room temperature, analyzed visually and photographed using a digital camera (MultiDoc-ItTM Digital Imaging System: Upland, CA, USA) or scanned using the Epson Perfection V39 Flatbed Scanner (Epson, New South Wales, Australia). On visual analysis, a single control line depicted the absence of DENV and the appearance of two lines i.e., a test line along with the control line indicated the presence of DENV. Grey-scale converted images were analyzed using ImageJ software (National Institute of Health, USA) by measuring the mean grey value and subtracting it from the maximum grey value for a given area. The average of two neighbouring white spaces is subtracted from the band intensity to normalize the results for each test band. A sample was classified as positive when the normalized band intensity was three times higher than the standard deviation of the two neighbouring white space values. During multiplex testing and testing with other reaction mixes, such as RAA (Qitian, China) we identified that different primers could be optimal in different circumstances, depending on the final reaction mix constituents.

FIG. 14 shows the diagnostic sensitivities using NS5 gene plasmid DNA diluted 10-fold in water for the four serotype-specific DENV RPA-LFD tests.

FIG. 15 shows the diagnostic sensitivities using NS5 gene fragment RNA transcripts diluted 10-fold in water for the four serotype-specific DENV RT-RPA-LFD tests.

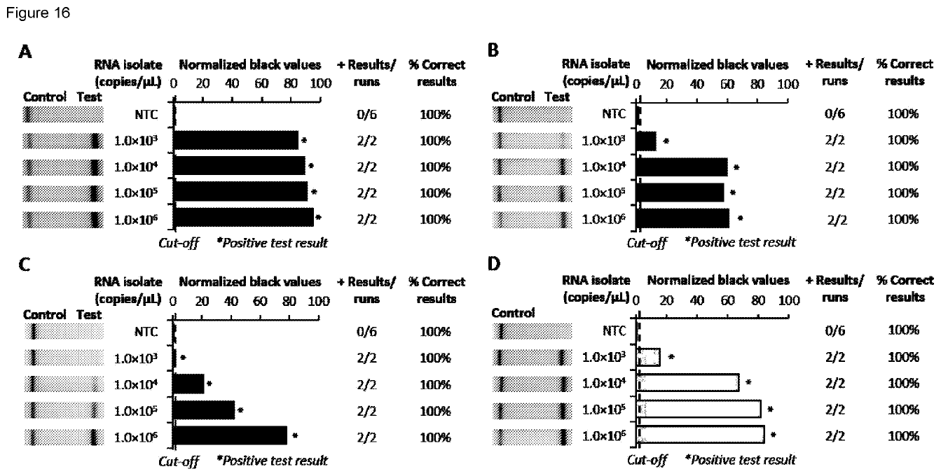

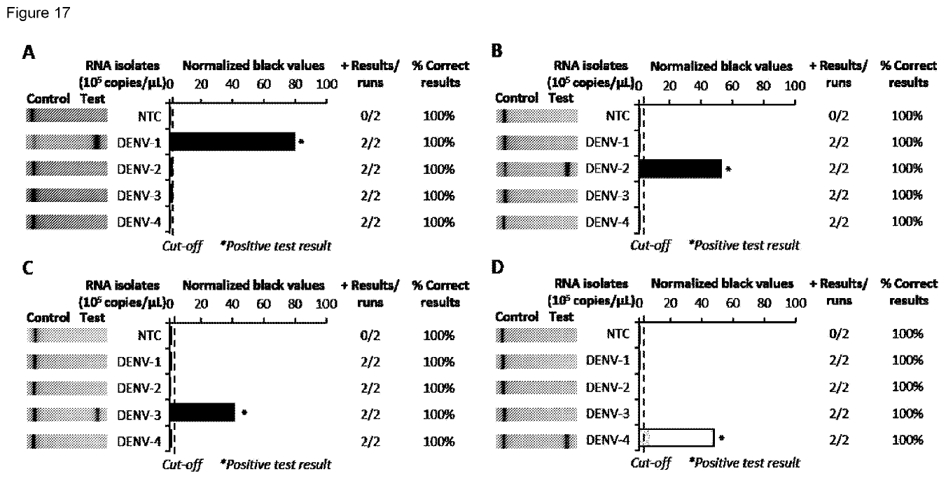

A universal dengue RT-qPCR was performed [as described in Pyke, Alyssa T., Wendy Gunn, Carmel Taylor, Ian M. Mackay, Jamie McMahon, Lauren Jelley, Ben Waite, and Fiona May. 2018. “On the Home Front: Specialised Reference Testing for Dengue in the Australasian Region” Tropical Medicine and Infectious Disease 3, no. 3:75 and Pyke, Alyssa T., Wendy Gunn, Carmel Taylor, Ian M. Mackay, Jamie McMahon, Lauren Jelley, Ben Waite, and Fiona May. 2019. “Erratum: Pyke, A. T. et al. On the Home Front: Specialized Reference Testing for Dengue in the Australasian Region. Trop. Med. Infect. Dis. 2018, 3, 75” Tropical Medicine and Infectious Disease 4, no. 4:129]. Table 28 shows the analytical sensitivities of a universal dengue RT-qPCR (2.89-33.10 TCID₅₀/mL) using RNA that was kit-extracted from viral culture supernatant, and FIG. 16 shows the analytical sensitivities of serotype-specific dengue tests (detection down to 1000 copies/μL) using kit-extracted RNA from viral culture supernatant of dengue virus 1 to 4 (DENV-1-4). FIG. 17 shows the analytical specificities of serotype-specific dengue tests using kit-extracted RNA from viral culture supernatant of dengue virus 1 to 4 (DENV-1-4) at 10⁵ copies/μL.

TABLE 28
Universal dengue RT-qPCR, targeting the capsid gene of
the four dengue serotypes, using 10-fold serial-diluted
RNA that was kit-extracted (NucleoSpin RNA Virus Mini
kit, Macherey-Nagel) from viral culture supernatants.

| | | | Calculated |
|---|---|---|---|
| DENV type (isolate) & | Titre | Cycle Threshold | concentration |
| Serial dilution | (TCID₅₀/ml) | value (Ct) | (copies/μL) |
| DENV-1 (ET243) | | | |
| Undiluted | 1.35 × 10⁶ | 17.17 | 1.61 × 10⁶ |
| 10⁻¹ | 1.35 × 10⁵ | 20.06 | 2.74 × 10⁵ |
| 10⁻² | 1.35 × 10⁴ | 21.76 | 1.03 × 10⁵ |
| 10⁻³ | 1.35 × 10³ | 28.99 | 1.67 × 10³ |
| 10⁻⁴ | 1.35 × 10² | 31.95 | 3.66 × 10² |
| 10⁻⁵ | 1.35 × 10¹ | 37.32 | 1.59 × 10¹ |
| 10⁻⁶ | 1.35 × 10⁰ | n.d. | — |
| DENV-1 (TC861HA) | | | |
| Undiluted | 2.17 × 10⁶ | 17.94 | 5.10 × 10⁵ |
| 10⁻¹ | 3.16 × 10⁵ | 19.26 | 2.50 × 10⁵ |
| 10⁻² | 3.16 × 10⁴ | 23.36 | 2.03 × 10⁴ |
| 10⁻³ | 3.16 × 10³ | 27.27 | 1.99 × 10³ |
| 10⁻⁴ | 3.16 × 10² | 31.75 | 1.59 × 10² |
| 10⁻⁵ | 3.16 × 10² | 35.83 | 1.45 × 10¹ |
| 10⁻⁶ | 3.16 × 10⁰ | 39.11 | 1.55 × 10⁰ |
| DENV-2 (NGC) | | | |
| Undiluted | 1.81 × 10⁵ | 17.99 | 6.10 × 10⁶ |
| 10⁻¹ | 1.81 × 10⁴ | 21.50 | 3.81 × 10⁵ |
| 10⁻² | 1.81 × 10³ | 25.73 | 3.24 × 10⁴ |
| 10⁻³ | 1.81 × 10² | 30.79 | 7.50 × 10³ |
| 10⁻⁴ | 1.81 × 10¹ | 33.96 | 2.18 × 10² |
| 10⁻⁵ | 1.81 × 10⁰ | 38.34 | 4.61 × 10⁰ |
| 10⁻⁶ | 1.81 × 10⁻¹ | n.d. | — |
| DENV-3 (ET209) | | | |
| Undiluted | 2.89 × 10⁵ | 19.32 | 1.37 × 10⁶ |
| 10⁻¹ | 2.89 × 10⁴ | 23.23 | 9.49 × 10⁴ |
| 10⁻² | 2.89 × 10³ | 25.48 | 2.18 × 10⁴ |
| 10⁻³ | 2.89 × 10² | 30.18 | 1.02 × 10³ |
| 10⁻⁴ | 2.89 × 10¹ | 33.54 | 1.13 × 10² |
| 10⁻⁵ | 2.89 × 10⁰ | 37.44 | 8.86 × 10⁰ |
| 10⁻⁶ | 2.89 × 10⁻¹ | n.d. | — |
| DENV-4 (ET288) | | | |
| Undiluted | 3.31 × 10⁶ | 18.31 | 1.20 × 10⁶ |
| 10⁻¹ | 3.31 × 10⁵ | 20.71 | 2.94 × 10⁵ |
| 10⁻² | 3.31 × 10⁴ | 24.97 | 2.22 × 10⁴ |
| 10⁻³ | 3.31 × 10³ | 28.38 | 1.66 × 10³ |
| 10⁻⁴ | 3.31 × 10² | 32.65 | 1.01 × 10² |
| 10⁻⁵ | 3.31 × 10¹ | 37.55 | 8.76 × 10⁰ |
| 10⁻⁶ | 3.31 × 10⁰ | n.d. | — |

n.d.: not detected.

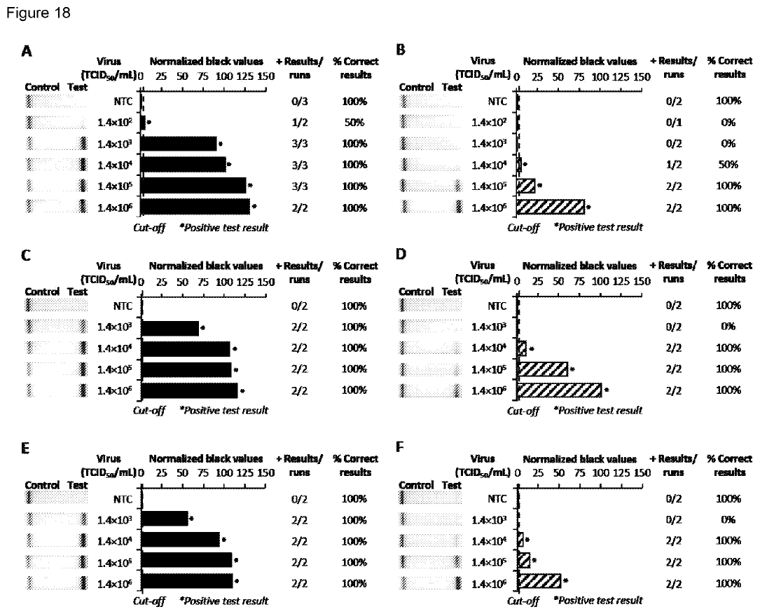

FIG. 18shows that Sample Preparation Reagent E could rapidly process DENV-1 virus in cell culture supernatant when contacted with sample for 10 minutes at a ratio of 5:1 sample:Sample Preparation Reagent E (20 μL sample+4 μL Sample Preparation Reagent E) at room temperature (FIG. 18A), 30° C. (FIG. 18C) and on ice (FIG. 18E) compared to controls.

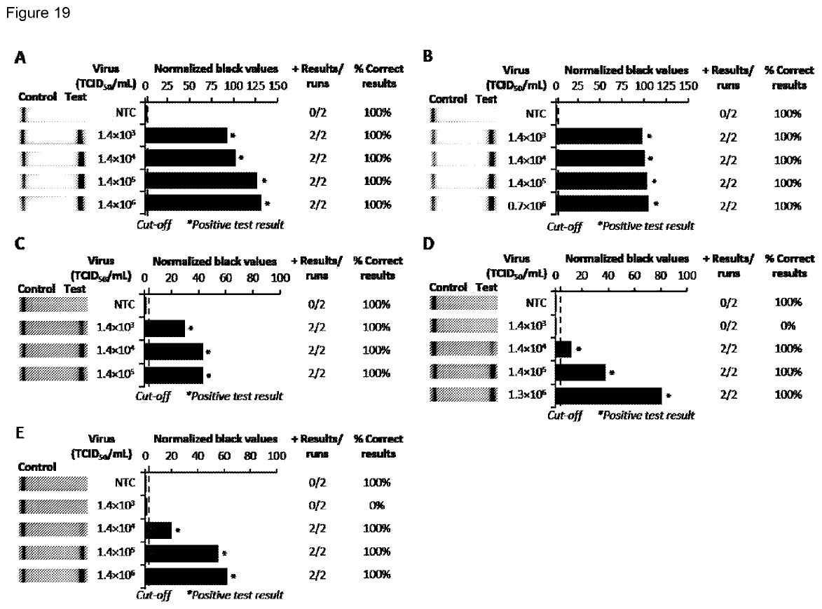

FIG. 19 shows analytical sensitivities of serotype-specific dengue tests obtained from viral culture supernatant containing five different dengue virus isolates, processed using Sample Preparation Reagent E for RT-RPA-LFD testing, and demonstrates detection down to 1,400-14,000 TCID₅₀/mL.

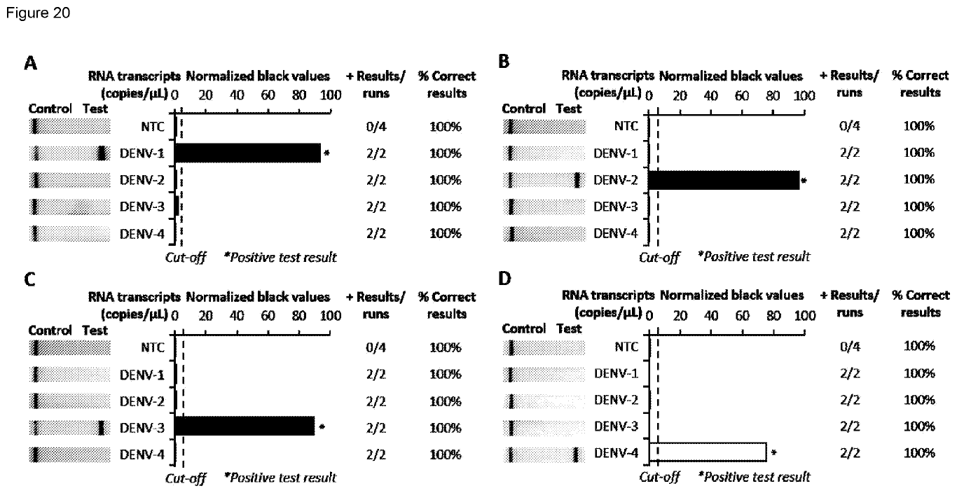

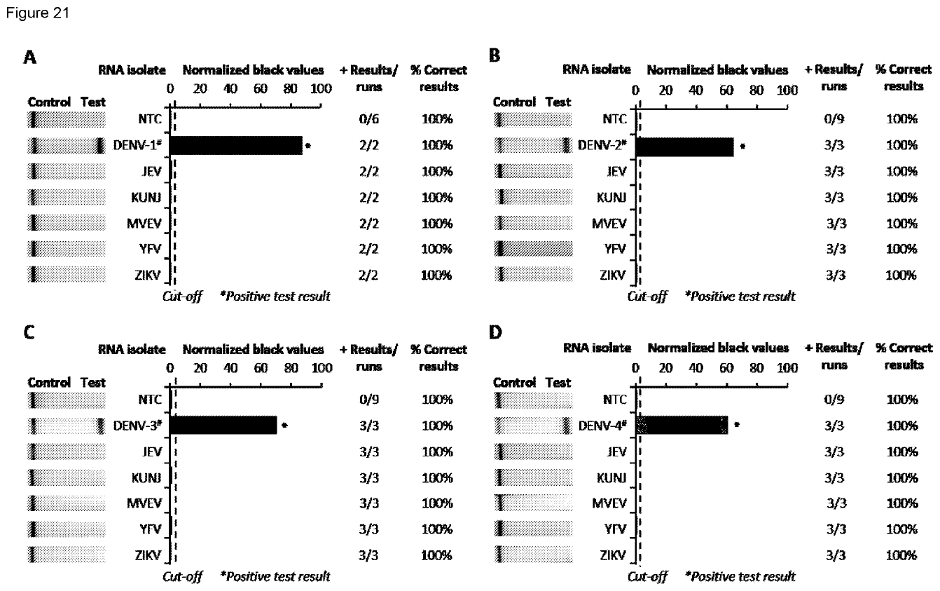

To confirm each DENV 1-4 RPA-LFD test was specific only for the respective serotyping, we determined if each serotype-specific test could detect high concentrations (105 copies/μL) of RNA transcripts from each DENV serotype. For each test, a strong test band indicated that the tests only detected their own serotype and did not detect any other DENV serotype, indicating each test was specific to its serotype specific target (FIG. 20). We also confirmed the DENV-specific tests could not detect other flaviviruses by trialling detection of RNA that was extracted by traditional liquid phase separation method from ZIKV, JEV, MVEV, YFV and KUNV cell culture cells or supernatants in TRIzol™ Reagent (Invitrogen by Thermo Fisher Scientific Australia Pty Ltd) (FIG. 21). No test lines appeared when any of the respective flaviviral RNA-extracts were applied to any of the DENV 1-4 RT-RPA-LFD tests, indicating that the assays were specific to their respective DENV 1-4 target.

To determine if small amounts of Sample Preparation Reagent E could inactivate high concentrations of DENV-1, cultured virus (106 TCID₅₀/mL) was mixed with Sample Preparation Reagent E at a ratio of 1:1, 2:1 or 5:1 (sample:Sample Preparation Reagent E; Table 29) in cell culture media containing 2% foetal bovine serum, 2 mM glutamine and 1× Antibiotic-Antimycotic). Mixtures were incubated for 10 minutes at room temperature and titrated by ten-fold serial dilutions on C6/36 cells in 96 well plates. After 7 days the virus titres were calculated using the Tissue Culture Infectivity Dose 50 (TCID₅₀). The viral titre without exposure to Sample Preparation Reagent E was also determined. Complete loss of detectable infectious virus was observed at all Sample Preparation Reagent E ratios trialled (Table 29). The virus remained infectious when not exposed to Sample Preparation reagent E (Table 29).

TABLE 29
Sample Preparation Reagent E inactivates DENV-1 (ET243)
at 10 min room temperature incubation at 1:1, 2:1 and
5:1 ratio (sample:Sample Preparation Reagent or cell
culture medium). Average ± standard deviation (n = 3).

| | DENV-1 titre, log₁₀ TCID₅₀/mL | | |
|---|---|---|---|
| Ratio | 1:1 | 2:1 | 5:1 |
| No Sample Preparation | 6.27 ± 0.40 | 6.74 ± 0.38 | 6.18 ± 0.66 |
| Reagent E | | | |
| With Sample Preparation | 0 ± 0 | 0 ± 0 | 0 ± 0 |
| Reagent E | | | |

All four DENV serotypes were completely inactivated by mixing Sample Preparation Reagent E with the respective DENV at a 1:5 ratio and 10 minute room temperature incubation (Table 30).

TABLE 30
Sample Preparation Reagent E inactivates all four DENV
(DENV-1 ET243, DENV-2 NGC, DENV-3 ET209, DENV-4 ET288)
at 10 min room temperature incubation at a 5:1 ratio
(sample:Sample Preparation Reagent E or cell culture
medium). Average ± standard deviation (n = 3).

| | No Sample Preparation | With Sample Preparation |
|---|---|---|
| | Reagent E | Reagent E |
| Ratio | 5:1 | 5:1 |
| DENV-1 titre, log₁₀ | 6.18 ± 0.66 | 0 ± 0 |
| TCID₅₀/mL | | |
| DENV-2 titre, log₁₀ | 6.65 ± 0.13 | 0 ± 0 |
| TCID₅₀/mL | | |
| DENV-3 titre, log₁₀ | 5.73 ± 0.07 | 0 ± 0 |
| TCID₅₀/mL | | |
| DENV-4 titre, log₁₀ | 6.09 ± 0.41 | 0 ± 0 |
| TCID₅₀/mL | | |

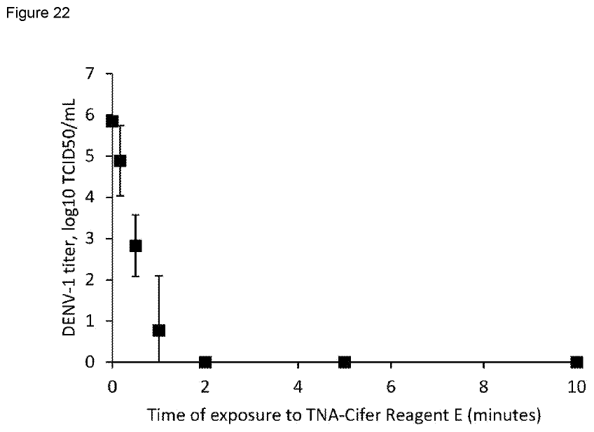

To determine how quickly Sample Preparation Reagent E could inactivate high concentrations of DENV-1, cultured virus (106 TCID₅₀/mL) was mixed with Sample Preparation Reagent E at a 5:1 ratio (i.e. 20 μL Sample Preparation Reagent E added to 100 μL virus in cell culture media containing 2% foetal bovine serum, 2 mM glutamine and 1× Antibiotic-Antimycotic). Mixtures were incubated for zero time (no exposure to Sample Preparation Reagent E), 10 and 30 seconds, or 1, 2, 5, and 10 minutes. Mixtures were then titrated by ten-fold serial dilutions on C6/36 cells in 96 well plates. After 7 days the virus titres were calculated using the Tissue Culture Infectivity Dose 50 (TCID₅₀). The initial virus titre (time=0) was 5.8 log 10 TCID₅₀/mL. After 1 minute exposure, the titre dropped to 0.7 log 10 TCID₅₀/mL. Complete loss of detectable infectious virus was observed after incubation with the reagent for 2 minutes or longer (FIG. 22). This data demonstrates Sample Preparation Reagent E renders DENV-1 in cell culture medium non-infectious at a sample:Sample Preparation Reagent E ratio of 5:1 or less (e.g., 2:1 and 1:1) and complete inactivation occurs within 2 minutes.

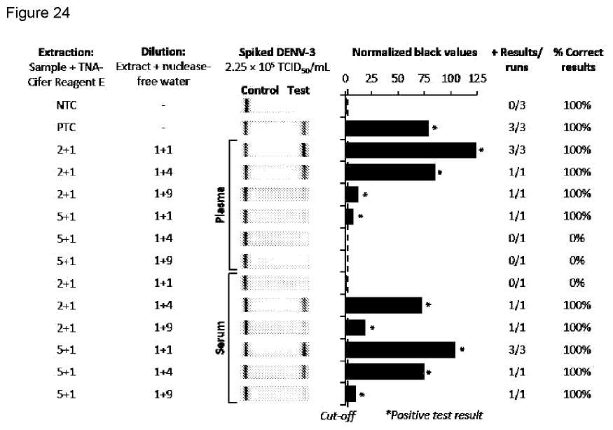

Sample Preparation Reagent E was used to rapidly process samples including human whole blood, plasma and serum for subsequent DENV RT-RPA-LFD testing. Mock samples were generated by spiking high titres of virus into clinical matrices. FIG. 23 and FIG. 24 show initial optimization experiments to identify extraction and dilution ratios with DENV-4 mock blood, and DENV-3 mock plasma and serum, respectively. This method development determined different sample:Sample Preparation Reagent E ratios were required for blood (1:1), 2:1 (plasma) and 5:1 (serum), followed by a 10-minute incubation, and subsequent dilution (1:1) with nuclease-free water prior to DENV RT-RPA-LFD testing.

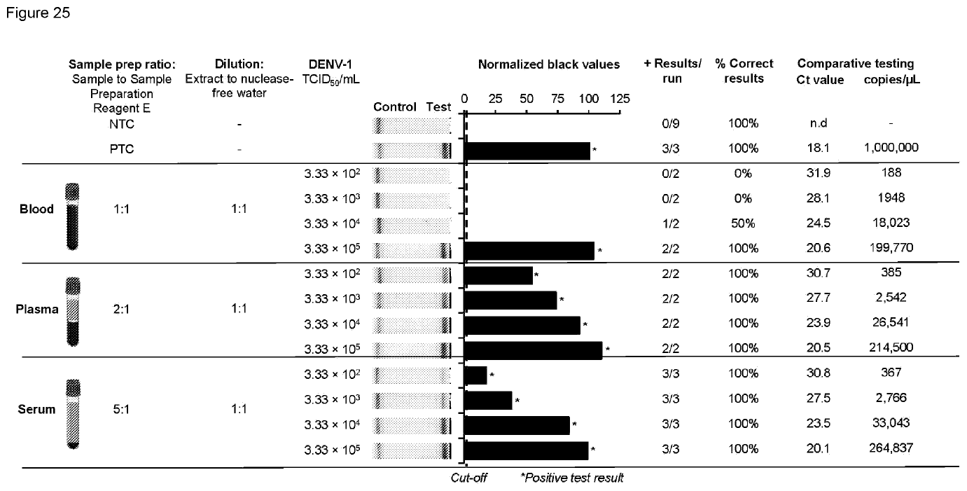

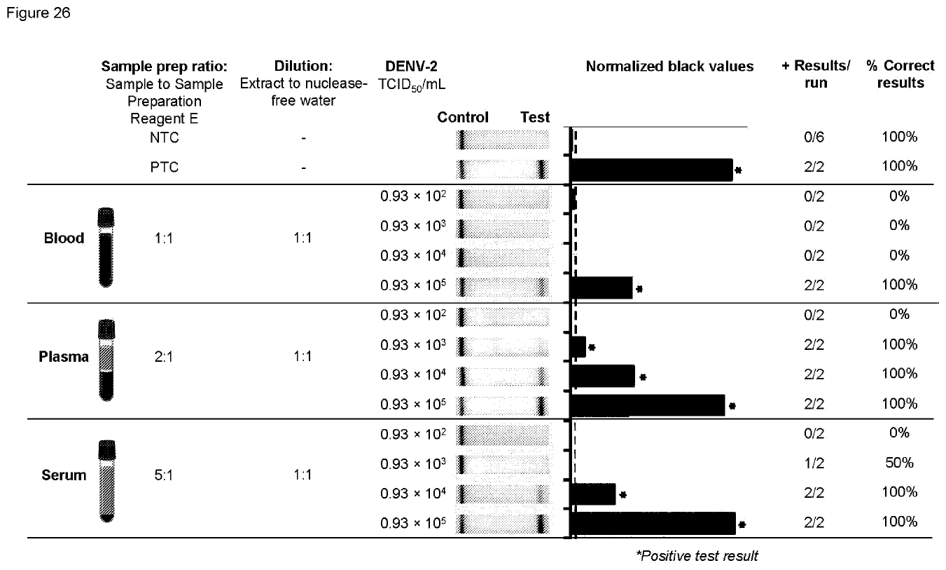

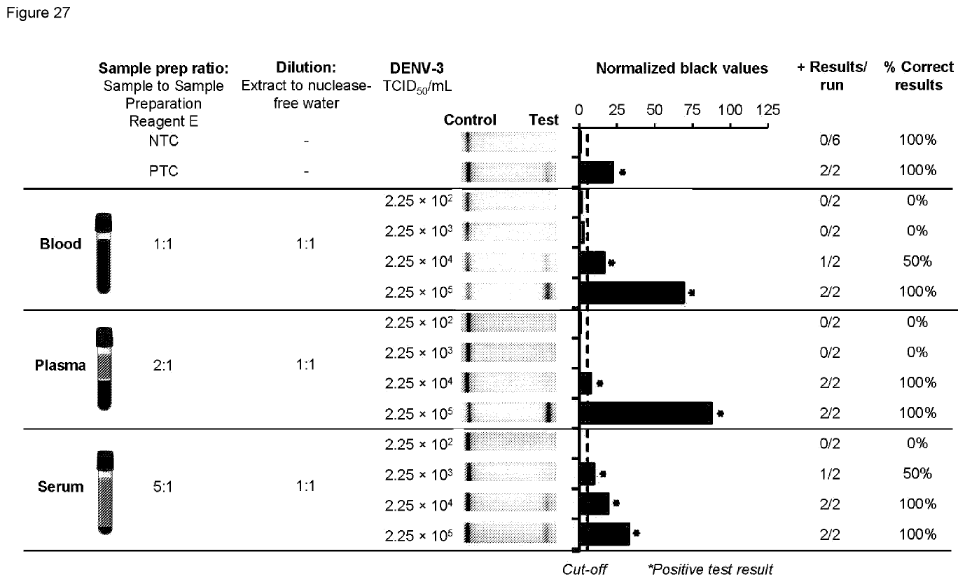

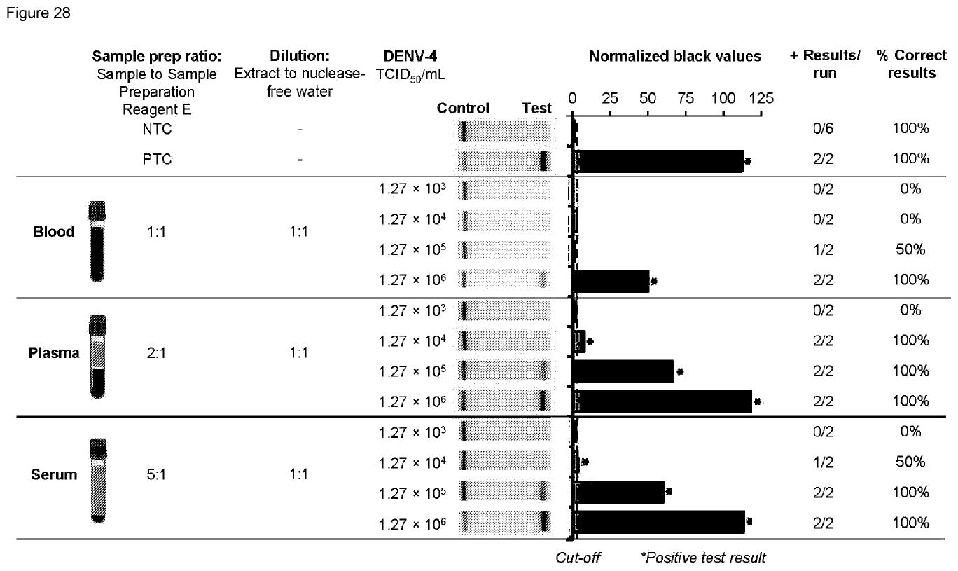

FIG. 25 shows DENV-1 mock blood, plasma and serum samples, and sample:Sample Preparation Reagent E ratios, as well as comparative testing using kit-extracted RNA of the same mock samples analysed by universal dengue RT-qPCR. FIG. 26, FIG. 27, and FIG. 28, show extraction and dilution ratios for Sample Preparation Reagent E used with DENV-2,-3 and -4 mock samples.

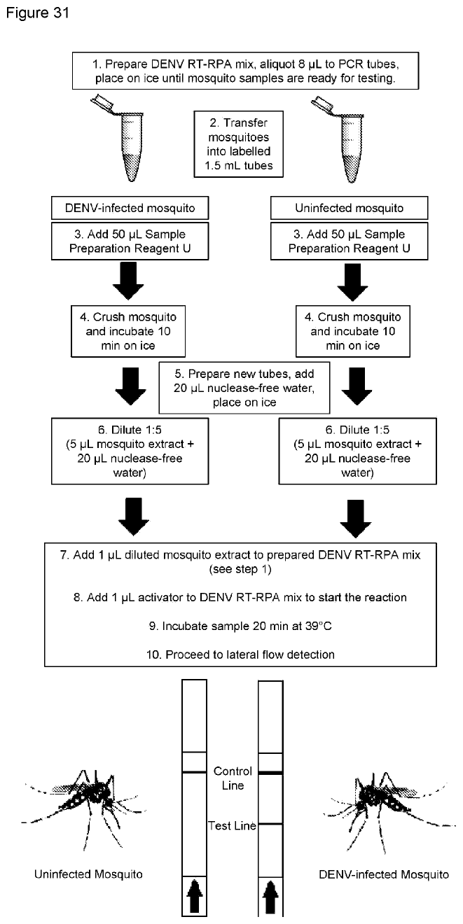

To apply the DENV 1-4 RT-RPA-LFD test for low-resource mosquito surveillance, a method to quickly process mosquitoes for subsequent molecular testing in a format that did not require laboratory-based instruments was developed (FIG. 31). The method used Sample Preparation Reagent U, as well as a pestle and tube for homogenizing the mosquitoes. In brief, individual mosquito bodies were homogenized with a pestle in 50 μL of Sample Preparation Reagent U and incubated at 4° C. for 10 minutes. Extracts were then immediately diluted 1:5 in RNase-free water, and 1 μL of this diluted extract was used as a template for subsequent RT-RPA reaction. For testing mosquito pools containing 5 mosquitoes, 125 μL Sample Preparation Reagent U was used, followed by dilution at 1:10 in RNase-free water, to reduce the amount of potential inhibitory by-products in the mosquito homogenate.

After 10 minutes incubation on ice, the homogenate was then diluted before 1 DL was applied for downstream testing using one of the four DENV 1-4 RT-RPA-LFD tests. The combined steps of sample preparation, dilution, RT-RPA, and LFD formed the final version of the rapid DENV-1-4 serotyping tests.

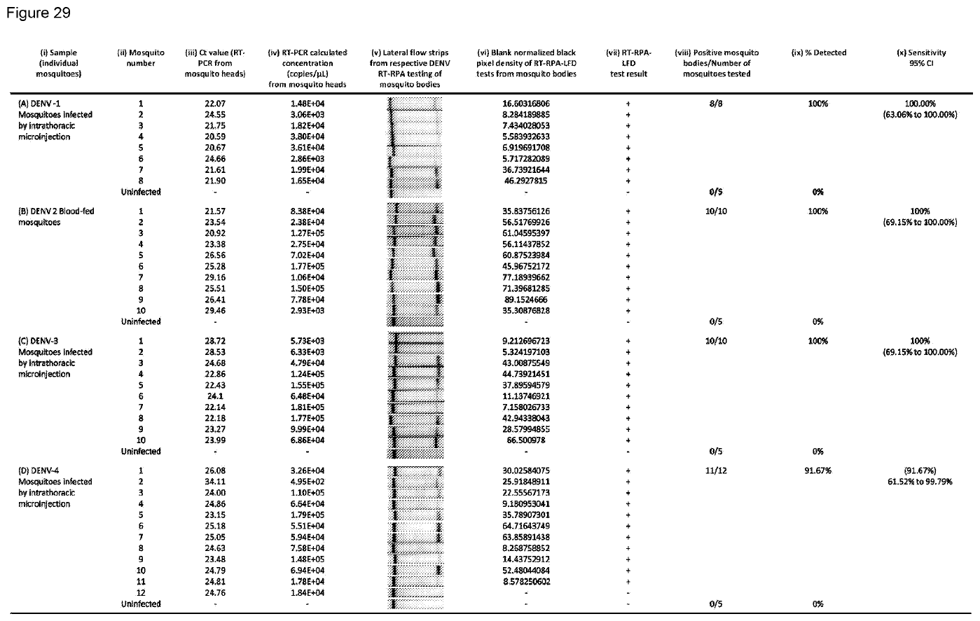

FIG. 29 shows the results of these rapid tests for the detection of individual DENV-infected and uninfected Aedes aegypti mosquitoes (bodies), and data obtained by collecting the mosquito heads for parallel universal DENV RT-qPCR testing.

For the rapid DENV-1 serotyping test, intrathoracically DENV-1 injected (n=8) or uninfected (n=5) Aedes aegypti mosquitoes were tested. Both RT-qPCR and the rapid DENV-1 serotyping test detected virus in all DENV-1 infected mosquito heads and bodies respectively, and not in the uninfected mosquitoes. These results confirmed a 100% diagnostic sensitivity for the rapid DENV-1 serotyping test compared to RT-qPCR (95% Confidence interval, CI: 63.06% to 100%), as well as 100% diagnostic specificity (CI: 48% to 100%). The rapid DENV-2 serotyping test was similarly trialled, this time using blood-fed DENV-2 infected mosquitoes (n=10) or uninfected mosquitoes (n=5). Again, both RT-qPCR and the rapid test detected virus in all DENV-2 infected blood-fed mosquitoes, and not in the uninfected mosquitoes, confirming 100% diagnostic sensitivity and specificity for the rapid DENV-2 serotyping test. Similar results were observed when testing the rapid DENV-3 serotyping test, using uninfected (n=5) mosquitoes, or mosquitoes infected with DENV-3 infected by intrathoracic microinjection (n=10). Again, both the RT-qPCR and the rapid test detected virus in all infected mosquitoes and not the uninfected mosquitoes, confirming 100% diagnostic sensitivity and specificity for our rapid DENV-3 serotyping test. The rapid DENV-4 test was similarly trialled using uninfected mosquitoes (n=5) or mosquitoes infected by intrathoracic DENV-4 microinjection (n=12). In this trial, all 12 RT-qPCR tests confirmed presence of the DENV-4 infection, however, the rapid DENV-4 test only detected 11/12 of those infections. These results indicated a diagnostic specificity of our rapid DENV-4 test of 92% sensitivity (62% to 100%), with 100% diagnostic specificity (CI: 48%-100%).

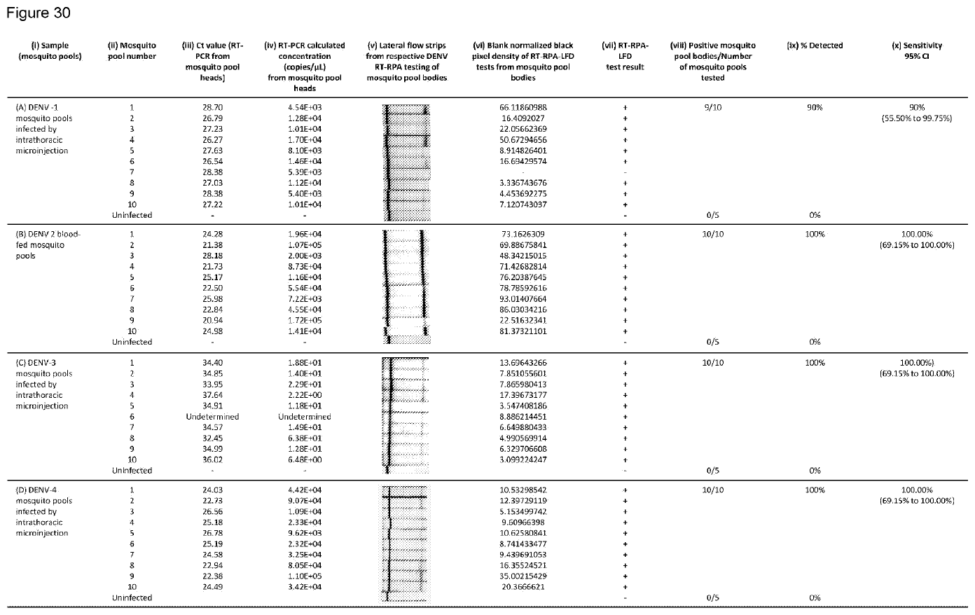

Following the testing of individual mosquitoes, the detection of DENV 1-4 in pools of mosquitoes containing 1 DENV-infected and 4 uninfected Aedes aegypti mosquitoes was examined. We used the respective serotype-specific RT-RPA-LFD tests for testing the pooled mosquito bodies and the DENV 1-4 universal RT-qPCR for testing the pooled mosquito heads as described above (FIG. 30).

Pooled DENV-1 (n=10) infected mosquito bodies tested with the rapid DENV-1 serotyping tests showed test bands in 9 mosquito pools, and no test bands appearing with the uninfected mosquito pools (n=5) (FIG. 30A). This result demonstrated 90% sensitivity (95% confidence interval, CI: 56%-100%) compared to the universal RT-qPCR. Tests bands were observed for all DENV-2 infected mosquito body pools (n=10), and no test bands were observed in pooled uninfected mosquito bodies (n=5), indicating detection of DENV-2 infected mosquitoes with 100% sensitivity (95% CI=69.15% to 100.00%) (FIG. 30B). Similar to the RT-qPCR from pooled mosquito heads, 10 out of 10 DENV-2 PCR positive infected mosquitoes tested positive with RT-RPA-LFD. These results were in concordance with the RT-qPCR results from the mosquito heads (n=10), where 10 out of 10 infected mosquito pools tested positive with RT-RPA-LFD. Similar results were observed with DENV-3 (n=10) and DENV-4 (n=10) infected pooled mosquito bodies, with test bands appearing with all the mosquito pools tested with respective RT-RPA-LFD tests, and no test bands appearing with any of the uninfected mosquito pools (n=5 each assay) (FIG. 30C&D). These results are in 100% accordance with the RT-qPCR results of the tested pooled mosquito heads.

FIG. 31 provides a flowchart and schematic for testing mosquitos.

### EXAMPLE 12: RPA-LFD Assay for the Detection Wolbachia Following Sample Preparation with Sample Preparation Reagent U

This example describes the development and validation of a rapid diagnostic test based on recombinase polymerase amplification (RPA) and lateral flow technology for the detection of Wolbachia, which is relatively simple, cost-effective and efficient, provides the same accuracy and sensitivity as conventional PCR assays, and is suitable for field experimentation for assessing the presence of Wolbachia as it requires only a 39° C. thermal block for implementation.

### Mosquitoes

The wAlbB2 Ae. aegypti strain, transinfected with the Wolbachia wAlbB strain isolated from Aedes albopictus, was obtained from Professor Zhiyong Xi at Michigan State University. The strain was originally created by microinjection of embryo cytoplasm from Aedes albopictus (USA Houston strain) into eggs from a colony of Ae. aegypti established from collections at Waco, Texas, USA. The strain was subsequently maintained within the QIMR Berghofer insectary (28° C., 70% relative humidity and 12:12 hr dark: night light cycling with 30-minute dawn/dusk periods). Eggs for these experiments were generated by blood feeding adult mosquitoes on a human volunteer according to QIMR Berghofer Human Research Ethics protocol P361. Eggs were also obtained from a Wolbachia-free colony of Ae. aegypti established in the QIMR Berghofer Insectary from collections in Cairns, Australia, in 2014.

Eggs were hatched and larvae were allocated into 3 L of rainwater in plastic trays (48×40×7 cm) at a density of 300 larvae per tray. Larvae were fed ground TetraMin Tropical Flakes fish food (Tetra, Melle, Germany) ad libitum. Pupae were transferred to an open container of rainwater within a 30×30×30 cm mesh-sided cage (BugDorm, MegaView Science Education Services Co., Taichung, Taiwan) for adult emergence. Adult mosquitoes were provided with cotton wool pledgets soaked with 10% sugar solution.

In order to simulate scenarios where mosquitoes are caught by mosquito traps in the field and spend periods of time in the catch compartment of the trap before collection and diagnostic analysis, a BG Sentinel mosquito trap (Biogents) within an environmental chamber (Panasonic) was assembled. The BG trap was left running and the chamber was set to provide natural ambient temperature fluctuations experienced within the field range of Ae. aegypti. Temperature and humidity set points were based on actual recordings made during summer in Cairns, north Queensland. Adult mosquitoes aged 3-5 days old were anaesthetized using CO₂, placed onto a petri dish on ice, and allocated into three treatment groups: (1) Fresh-mosquitoes were maintained in a cage under insectary conditions described above for two weeks; (2) Dried one week-mosquitoes were maintained in a cage in the insectary as described for one week, then enclosed in a gauze bag and placed into the catch bag of the BG trap for one week 2; and (3) Dried two weeks-mosquitoes were enclosed alive in a gauze bag which was placed in the catch bag of the BG trap for a two week period. Mosquitoes from the three treatment groups were collected and allocated into 1.5 ml microcentrifuge tubes for processing, either individually or in pools consisting of one WB2 female with four uninfected mosquitoes.

### DNA Standards and Oligonucleotides

A 391 bp region of the wsp gene derived from wAlbA isolate of Aedes albopictus (Accession number JX129187.1) cloned into the pBIC-A plasmid (2070 bp) was synthesized by Bioneer Pacific (Kew East, VIC, AU), and used as the DNA template for optimization and analytical sensitivity testing. The plasmid was resuspended in DNase-RNase free water and stored at −80° C., before determination of concentration (ng/μL) using the Qubit dsDNA HS Assay Kit (Invitrogen, Eugene, OR, USA) and subsequent calculation of copies/μL based on the plasmid template size (2461 bp). Primers and probes were also synthesized by Bioneer Pacific (Kew East, VIC, AU): unlabelled forward primers were Bio-RP purified (near-HPLC purification), whereas labelled reverse primers and probes were HPLC purified. Primer and probe stocks were resuspended to 100 μM concentration in DNase-RNase-free water before storage at −80C.

### Wol RPA-LFD

Single mosquitoes were homogenized in 50 μL of either Sample Preparation Reagent U or NaOH/SDS (0.05M NaOH, 0.25% SDS) in a 1.5 ml tube. Mosquito pools containing either 5 uninfected mosquitoes or 1 infected mosquito mixed with 4 uninfected mosquitoes were homogenized in 200 μL Sample Preparation Reagent U. Samples were incubated for 10 minutes at room temperature to leave sufficient time for DNA to be released from the disrupted cells, before immediate dilution, where 10 μL of sample was mixed with 40 μL of DNAse- and RNase-free water.

DNA in diluted samples, or plasmid DNA, or water in control experiments, was then amplified using the TwistAmp nfo Kit (TwistDx, Cambridge UK) by mixing (1 μL) with 8 μL of RPA reaction mix, which had been prepared as a 40 L mastermix (29.5 μL rehydration buffer, 3.9 μL molecular grade water and 6.6 L primer/probe mix). The final concentration of primers and probes in each assay tube was 420 nM Forward primer, 420 nM Reverse primer, and 480 nM Probe. Each 9 μL reaction was started by addition of 1 μL 140 mM magnesium acetate (total reaction volume per tube 10 μL). The reaction mix was incubated in a heating block for 25 minutes at 39° C. with brief mixing after 10⁻¹⁵ minutes of incubation and at the end of the 25 minutes incubation period.

After amplification, 2 μL of RPA amplicons were dropped onto the middle of the sample application area of a HybriDetect MGHD 1 strip (Milenia Biotec GmbH, Gieβen, Germany), which was blocked with 8 μL casein buffer (0.4% casein, 0.1% Tween in PBS, pH 9). Strips were then placed into tubes containing 100 μL of borate buffer (100 mM H₃BO₃, 100 mM Na₂B₄O₇, 1% BSA, 0.05% Tween 20, pH 8.8), incubated at room temperature for 5 minutes and imaged immediately. All experiments were performed at least three times if not specified otherwise.

### Statistical Analysis

Photographic images of lateral flow strips from the RPA-LFD assay were analysed digitally by ImageJ software (National Institute of Health) according to author's instructions. ImageJ analyses the faint bands which are not clearly distinguishable as positives by measuring the black pixel density of the bands. The mean grey value is subtracted from maximum grey value using a fixed area of measurement and to normalize each test band, average of two neighbouring white spaces if subtracted from the band intensity. A positive sample has 1.3 times higher pixel density than standard deviation of two neighbouring white spaces. The diagnostic assessment of the Wolbachia RPA-LFD assay was conducted using the online MedCalc's diagnostic test evaluation calculator to determine the sensitivity, specificity, accuracy, confidence intervals, positive and negative predictive values

### Results

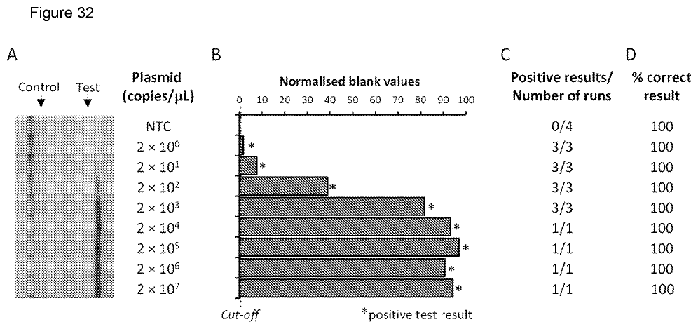

A rapid Wolbachia test was developed by targeting the universal gene coding for Wolbachia outer surface protein (wsp) using a novel sample extraction buffer followed by recombinase polymerase amplification (RPA) and lateral flow detection (LFD). Primers and probes for the Wol RPA-LFD test were designed by manually aligning 1509 published endosymbiont sequences of Wolbachia (the full range at the time of design). Three different pairs of forward and reverse primers, along with labelled probes were tested with the synthetic Wolbachia plasmid template for the RPA reaction followed by lateral flow assay detection (Table 31). The optimal conditions for amplification and detection of plasmid were determined to be incubation at 39° C. for 25 minutes using the primer and probe combinations Forward 2, Reverse 3, and Probe 1, followed by 5 minutes of incubation of the lateral flow strip in the running buffer at room temperature. Repeat testing of plasmid serial dilutions using this optimized combination indicated clear appearance of the test line when as little as 20 copies/μL of plasmid was present when viewing the results visually (FIG. 32A). In addition, subsequent pixel-density analysis of images indicated as little as 2 copies/μL plasmid could be reliably detected in all trials (FIG. 32B-D).

TABLE 31
RPA primers and nfo probes used for
detection of Wolbachia*

| Name | Sequence (5′-3′ ) |
|---|---|
| Forward Primer 1 | GNHGGNGGTGBHGCRTTTGGYTAYAAAATGG |
| (SEQ ID NO: 1) | |
| Forward Primer 2 | GYTAYAAAATGGAHGAHATYAGDGTTGAHDTTGAAGG |
| (SEQ ID NO: 2) | |
| Forward Primer 3 | GAHGAHATYAGDGTTGAHDTTGAAGGNVTHTAYTC |
| (SEQ ID NO: 3) | |
| Reverse Primer 1 | [5′ FAM]CRTADCTVACACCAGCYYTTDCTTGAYVAGC |
| (SEQ ID NO: 4) | |
| Reverse Primer 2 | [5′ FAM]GYTTGAYTTCNGGNGTTAYDTCRTADCTVACACC |
| (SEQ ID NO: 5) | |
| Reverse Primer 3 | [5′ FAM]GTTAYDTCRTADCTVACACCAGCYYTTDCTTG |
| (SEQ ID NO: 6) | |
| Probe 1 (SEQ ID | [5′ Biotin]TTGAAGATATGCCTATCACTCCATAYRTTG |
| NO: 7) | [Internal dS spacer] TGTTGGYGTTGGTGC [3′ |
| | C3 spacer] |
| Probe 2 (SEQ ID | [5′ Biotin]GTKAAYGTNTATTACGATDTAGCNATTGAAG |
| NO: 8) | [Internal dS spacer] TATGCCTATCACTCC [3′ |
| | C3 spacer] |

*DNA in diluted samples, or plasmid DNA or water in control experiments, was amplified using the TwistAmp nfo Kit (TwistDx, Cambridge UK) by mixing 1 μL of diluted sample with 8 μL of RPA reaction mix, which had been prepared as a 40 μL mastermix (29.5 μL rehydration buffer, 3.9 μL molecular grade water and 6.6 μL primer/probe mix). The final concentration of primers and probes in each assay tube was 420 nM Forward primer, 420 nM Reverse primer, and 480 nM Probe. Each 9 L reaction was started by addition of 1 μL 140 mM magnesium acetate (total reaction volume per tube 10 μL). The reaction mix was incubated in a heating block for 25 minutes at 39° C. with brief mixing after 10-15 minutes of incubation and at the end of the 25 minutes incubation period. After amplification, 2 μL of RPA amplicons were dropped onto the middle of the sample application area of a HybriDetect MGHD 1 strip (Milenia Biotec GmbH, Gießen, Germany), which was blocked with 8 μL casein buffer (0.4% casein, 0.1% Tween in PBS, pH 9). Strips were then placed into tubes containing 100 μL of borate buffer (100 mM H₃BO₃, 100 mM Na₂B₄O₇, 1% BSA, 0.05% Tween 20, pH 8.8), incubated at room temperature for 5 minutes and imaged immediately. All experiments were performed at least three times if not specified otherwise. Photographic images of lateral flow strips from the RPA-LFD assay were analyzed digitally by ImageJ software (National Institute
of Health, USA). ImageJ analyzes the faint bands which are not clearly distinguishable as positives by measuring the black pixel density of the bands. The mean grey value was subtracted from maximum grey value using a fixed area of measurement and to normalize each test band, average of two neighboring white spaces if subtracted from the band intensity. A positive sample had 1.3 times higher pixel density than standard deviation of two neighboring white spaces. The diagnostic assessment of the Wolbachia RPA-LFD assay was conducted using the online MedCalc's diagnostic test evaluation calculator to determine the sensitivity, specificity, accuracy, confidence intervals, positive and negative predictive values.

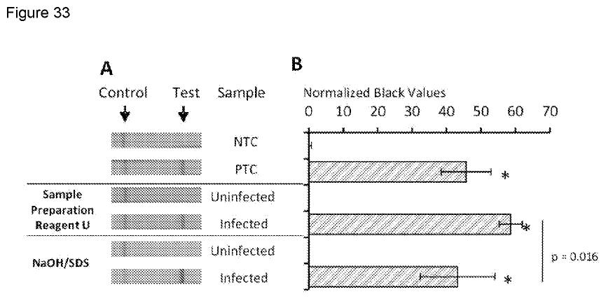

For mosquito testing, field-friendly sample processing techniques were compared using two novel Sample Preparation Reagents: NaOH/SDS (NS) and Sample Preparation Reagent U). Samples were homogenized in these reagents for 5 minutes and immediately diluted in water (either 1/2, 1/5, 1/10, 1/50 or 1/100) before testing diluted sample using the Wol RPA-LFD test. Sample Preparation Reagent U processed samples showed consistently solid test bands with darker pixel intensity for Wolbachia infected mosquitoes (FIG. 33A-B) regardless of the water dilution trialled, and no test band in uninfected mosquitoes. In contrast, NS extracts showed variable test band intensities, and an average decreased pixel intensity compared to Sample Preparation Reagent U processed samples, and this was determined significant using a two-tailed T-Test (p-value of 0.016; FIG. 33C). Sample Preparation Reagent U was thus chosen and combined with the Wol RPA-LFD test to create a rapid Wolbachia (Wol) test that was used for all subsequent mosquito testing experiments. The 1 in 5 dilution in water was nominally chosen for all subsequent sample processing.

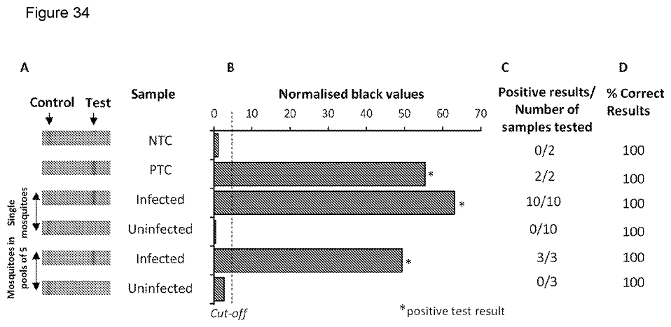

The utility of the rapid Wol test for mosquito screening was explored using laboratory infected or uninfected mosquitoes. The rapid Wol test detected Wolbachia infection with 100% accuracy in individual mosquitoes (n=20), as well as pools of 5 mosquitoes, where infected pools consisted of a single infected mosquito mixed with four uninfected mosquitoes (n=6) (FIG. 34).

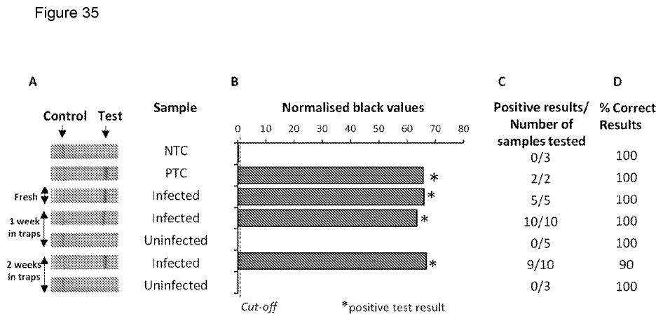

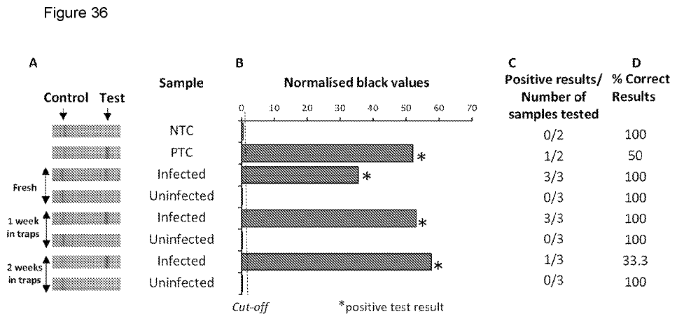

The ability of the rapid Wol test to detect laboratory-infected mosquitoes left in a humid environment simulating a trap for either 1 or 2 weeks was tested. Results demonstrated that the test could determine infection in a single mosquito either tested fresh, or left for 1 week in traps with 100% accuracy (n=15) (FIG. 35). However, after 2 weeks in traps the accuracy of individual mosquito testing dropped to 92.31% (n=13), as one of the known-infected mosquitoes failed to be detected using the Wol RPA-LFD test. The effectiveness of testing mosquitoes fresh or left in traps up to 1 week, but not two weeks, became more apparent when testing mosquito pools. In this experiment, a single infected mosquito was mixed with four uninfected mosquitoes, or five uninfected mosquitoes were tested. The test again showed 100% accuracy for detecting pools of mosquitoes tested fresh or left in traps for one week (n=12; FIG. 36) but showed only 33% sensitivity when testing for infection at 2 weeks (n=6), with only one of the three infected pools showing a positive test at that time point.

If all mosquito testing results are taken together, testing for Wolbachia infection both individually and in pools could detect infection with 96.1% accuracy (93.62% sensitivity, 100% specificity, 100% positive predictive value, 90.91% negative predictive value) if mosquitoes were left in simulated trap environments for 2 weeks or less (n=77). Importantly, the data demonstrates 100% accuracy when testing mosquitoes or pools left for 1 week or less (n=58; 100% sensitivity and specificity, 100% positive predictive value and negative predictive value).

### EXAMPLE 13: RPA-LFD Assay for the Detection of P. Falciparum in Blood Samples Following Sample Preparation with Sample Preparation Reagent U

A rapid test for Plasmodium falciparum in human blood (rapid Pf blood test), with potential for implementation in low resource settings, was developed. This rapid Pf blood test provides improved sensitivity compared to microscopy or RDTs. The test uses a manual workflow consisting of (1) Sample Preparation Reagent U, which is able to prepare samples for molecular genetic testing in 5 minutes that circumvents the need to centrifuge sample and extract DNA prior to testing; (2) a 10-minute isothermal amplification using Recombinase Polymerase Amplification (RPA; TwistDx, Cambridge, UK) that requires only a heat block at 39° C. for operation; followed by (3) a 5-minute lateral flow detection. By combining these three manual testing steps together, the rapid Pf blood test can detect samples in only 20 minutes, without requiring any sophisticated laboratory equipment: only a battery powered heating block and pipettes for liquid handling are required, making the test suitable for field applications. Similar work was performed to demonstrate detection of P. vivax (data not shown).

Seventeen frozen human blood samples infected with the P. falciparum strain 3D7-MBE008 were used for validation of the rapid Pf blood test. These samples had been sequentially collected in K3 EDTA tubes from a human volunteer who was experimentally infected with P. falciparum within a larger study testing the safety and infectivity of a new P. falciparum (3D7-MBE008) strain. Briefly, a blood sample was taken pre-infection and then at intervals over 10 days. Anti-malarial treatment was first administered at Day 8 and further blood samples were collected up to 72 hours afterwards, alongside ongoing rescue drug treatment. In the original study, following column-purification of the parasite DNA in these samples, purified DNA was tested in triplicate using an ultra-sensitive qPCR to determine the Geometric Mean of Parasites/500 UL of Packed RBCs. These results were supplied along with stored blood samples. To prevent testing bias, samples were blinded prior to testing with the rapid Pf blood test.

### Parasite Cultures

Malaria parasite cultures of the P. falciparum 3D7 strain were maintained, grown to between 3-7% parasitaemia, prepared for fluorescence-activated cell sorting (FACS) to verify the number of parasites present. One culture was used to create qPCR control standards and gave a final parasite concentration of 3,190,900 p/mL blood. DNA from this control standard (500 μL) was purified using a QIAmpDNA Mini Kit (QIAGEN, Australia) as per the manufacturer's instructions using a 100 μL elution volume (i.e., 5× concentration). A second culture was used to create a parasite sample. The culture was adjusted to 50% haematocrit and parasite concentration determined by both microscopy, and the number of red blood cells per mL calculated using a haemocytometer. The parasite sample was then diluted 10-fold using K3 EDTA blood, and used as a control sample for comparing efficacy of standard qPCR and the rapid Pf test.

### RPA Oligonucleotides

Primers (forward and reverse) and a probe for recombinase polymerase amplification were designed to target the 18S rRNA gene, using 57 aligned sequences from GenBank (Table 32). Primers and probes were resuspended using DNase and RNase-free water to 100 μM concentrations before storage at −80° C., and diluted to 10 UM for use as working stocks. The concentration (18S gene copies per μL) of the resuspended plasmid was determined using the dsDNA HS Assay Kit (Life Technologies, Singapore).

TABLE 32
RPA primers and nfo probes used for detection of
P. falciparum and P. vivax.*

| | Primer | | | Labelled/ |
|---|---|---|---|---|
| Gene target | type | ID | Sequence (5′ → 3′) | unlabelled |
| 18S rRNA | Forward | M. Falcip F1 | GTTAAGGGAGTGAAGACGATCAGATA | unlabelled |
| Gene | | | CCGTCG (SEQ ID NO: 81) | |
| (Plasmodium | | | | |
| general) | | | | |
| | Forward | M. Falcip F2 | GAGTGAAGACGATCAGATACCGTCGT | unlabelled |
| | | | AATCTTAAC (SEQ ID NO: 82) | |
| | Forward | M. Falcip F3 | GGAGTGAAGACGATCAGATACCGTCG | unlabelled |
| | | | TAATC (SEQ ID NO: 83) | |
| | Reverse | M. Falcip R1 | GACTTTGATTTCTCATAAGGTACTGA | unlabelled |
| | | | AGGAAGC (SEQ ID NO: 84) | |
| | Reverse | M. Falcip R2 | CTTTGATTTCTCATAAGGTACTGAAG | unlabelled |
| | | | GAAGCAATC (SEQ ID NO: 85) | |
| | Reverse | M. Falcip R3 | CCCAAAGACTTTGATTTCTCATAAGG | unlabelled |
| | | | TACTGAAGG (SEQ ID NO: 86) | |
| | Reverse | M. Falcip R3- | [5′ FAM]CCCAAAGACTTTGATTTC | labelled, |
| | | FITC | TCATAAGGTACTGAAGG (SEQ ID | FAM |
| | | | NO: 87) | |
| 18s rRNA | Probe | M. Falcip P2 | [5′ Biotin]GGTGTTGGATGAAAG | labelled, |
| gene P. | | | TGTTAAAAATAAAAGT [Internal | biotin |
| falciparum | | | dS spacer] ATCTTTCGAGGTGAC | |
| | | | [3′ C3 spacer] (SEQ ID NO: | |
| | | | 88) | |
| | Probe | M. Falcip P3 | [5′ Biotin]CTAGGTGTTGGATGA | labelled, |
| | | | AAGTGTTAAAAATAA [Internal | biotin |
| | | | dS spacer] AGTCATCTTTCGAGG | |
| | | | [3′ C3 spacer] (SEQ ID NO: | |
| | | | 89) | |
| 18s rRNA | Probe | P. vivax | [5′ Biotin]GACTAGGCTTTGGAT | labelled, |
| gene P. | | Probe 1 | GAAAGATTTTAAAATAAG | biotin |
| vivax | | | [Internal dS spacer]GTTTTC | |
| | | | TCTTCGGAG [3′ C3s pacer] | |
| | | | (SEQ ID NO: 90) | |
| | Probe | P. vivax | [5′ Biotin]CTATGCCGACTAGGC | labelled, |
| | | Probe 2 | TTTGGATGAAAGATT [Internal | biotin |
| | | | dS spacer] TAAAATAAGAGTTTT | |
| | | | C [3′ C3 spacer] (SEQ ID | |
| | | | NO: 91) | |

*The optimal primers for amplifying the respective Plasmodium species was dependent on the final embodiment of the reaction mix.

Standard Whole-Blood qPCR Test

Whole-blood samples (50 μL) were extracted using the QIAmp DNA Mini kit (QIAGEN) as per the manufacturer's instructions with a 50 μL elution volume. Sample were then testing by qPCR using oligonucleotides and cycling conditions used were those previously developed. Briefly, the reaction mix consisted of 12.5 μL Quantitect Probe PCR Mix (Qiagen, Australia), 10 pmol of each primer (PerFAL-Forward: CTT TTG AGA GGT TTT GTT ACT TTG AGT AA (SEQ ID NO: 13) and PerFAL-Reverse: TAT TCC ATG CTG TAG TAT TCA AAC ACA (SEQ ID NO: 14), 4 pmol of probe (PerFAL-probe: Fam-TGT TCA TAA CAG ACG GGT AGT CAT GAT TGA GTT CA-BHQ1 (SEQ ID NO: 25) and 5 μL of template DNA in a 25 μL final reaction volume. Amplification was performed in a Rotorgene 6000 (Qiagen, Australia) under the following conditions: 15 minutes incubation at 95° C., followed by 45 cycles of 95° C. for 15 seconds and 60° C. for 1 minute.

Rapid Plasmodium falciparum blood test (Rapid Pf blood test).

Sample preparation: Blood (10 μL) was mixed 1:1 with 10 μL of Sample Preparation Reagent U and incubated at RT for 5 minutes. From this mixture, 10 μL was then added to 10 μL of 140 mM Magnesium acetate, and 2 μL of this mixture was immediately used for isothermal amplification.

Isothermal amplification: Sample (2 μL) was mixed with 8 μL freshly prepared recombinase polymerase amplification (RPA) mixture, followed by incubation at 39° C. for 10 minutes. The final concentration of reactants in each tube was: 420 nM Forward and Reverse primers, 120 nM probe, 1× Rehydration buffer & pellet mixture.

Lateral flow detection: Immediately following incubation, 2 μL of the amplicons were transferred to the sample pad of a HybriDetect lateral flow strip (Milenia Biotec GmbH, Gieβen, Germany), which had been pre-prepared by the addition of 8 μL blocking buffer (0.4% casein, 0.1% Tween in PBS, pH 9) to the sample pads of the strips. Strips were subsequently placed into tubes with the sample pads immersed in 100 μL of borate buffer (100 mM H₃BO₃, 100 mM Na₂B₄O₇, 1% BSA, 0.05% Tween 20, pH 8.8), and left to wick along the strips for 5 minutes before appearance of test bands was observed by eye, and subsequently imaged.

Data analysis

Strips were scanned and analysed using ImageJ software (National Institutes of Health, MD, USA). Greyscale-converted images were used to determine band -intensity, by measuring the mean grey value (limit to threshold), using a fixed area measurement, and subtracting from the maximum threshold value. For each test band, an average of the neighbouring white space of all LF strips in that experiment was subtracted from the band intensity to normalize the results. To define a sample as positive the test band values were standardized by subtracting a cut-off value (three standard deviations above the average of the negative control test band intensities). A standardized value of greater than 0 was a positive result.

Comparative analysis of tests was performed using a diagnostic sensitivity calculator with exact Clopper-Pearson 95% confidence intervals.

### Results

Recombinase polymerase amplification followed by lateral flow detection (rapid Pf RPA-LFD), which enables sensitive and rapid amplification, but requires only a simple heating block for operation, was used. After optimisation of primers and probes, the optimised rapid Pf RPA-LFD was combined with a Sample Preparation Reagent U, to construct a rapid Pf blood test. In this test, whole blood is mixed with the Sample Preparation Reagent and incubated for 5 minutes at room temperature, followed by dilution in magnesium acetate. Diluted sample is then immediately used to detect P. falciparum using the Pf RPA-LFD test, which includes incubation for 10 minutes at 39° C., followed by lateral flow detection. The entire test, from sample to result, can be performed in 20 minutes.

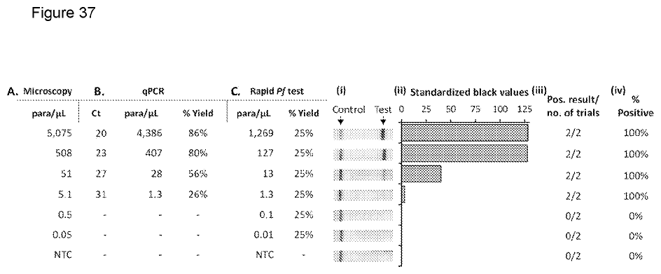

The rapid Pf blood test was tested using a quantified parasite sample diluted 10-fold in K3-EDTA collected blood and compared to a standard whole-blood qPCR. Results for the Rapid Pf blood test (FIG. 37) showed a decrease in lateral flow detection (LFD) test band intensity as the parasite concentration decreased, reaching a limit of detection of 5 parasites/□L from the original parasites sample. The standard qPCR test similarly detected decreasing amounts of parasite as the concentration was reduced, reaching the same limit of detection (5 parasites/μL). Interestingly, qPCR estimates using a second quantified standard determined an estimated standard concentration of the original sample. From these estimates, the % yield for DNA extraction and detection at each concentration was calculated. For the standard DNA extraction and qPCR, the yield was high at high parasite concentrations (86%), but reduced to only 26% at the lowest parasite concentration, despite an equivalent protocol eluting with the same volume as the incoming sample. These results indicate that the column purification performed in this study began to lose up to 75% of DNA at the lower concentrations of parasite tested. These results indicate the rapid Pf blood test, despite diluting sample 1/4 prior to testing, can detect to a similar sensitivity at low concentrations of parasite (˜5 parasite/μL) compared to a standard whole-blood DNA column purification followed by qPCR.

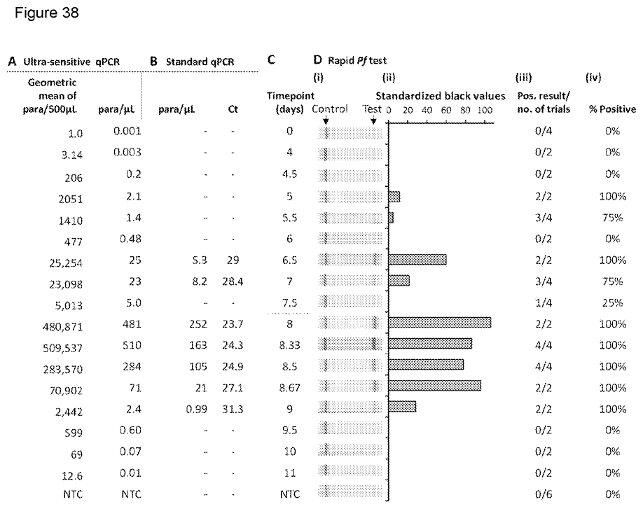

The rapid Pf blood test was clinically tested using 17 samples taken from a single patient, who was experimentally infected with P. falciparum during a clinical trial. The patient was infected on Day 1 and blood was sampled regularly until parasite loads increased, with treatment beginning on Day 8. The study treated the patient prior to symptoms appearing, and thus represents a test of the rapid Pf blood test to detect asymptomatic samples. Results demonstrated our rapid test began to detect infections around 5 days post-infection (FIG. 38), which was one day earlier than the whole-blood qPCR testing. However, with the cyclical nature of the P. falciparum infection changing the daily concentration levels, consistent detection using the rapid Pf blood test was observed from Day 8 onwards (the time point the patient was treated with an anti-malarial drug), and this was consistent with the whole-blood qPCR detection results. During replicate testing, some samples showed inconsistent results, and these were repeated to confirm sensitivity, along with a selection of strong positive and negative samples to confirm result consistency. The inconsistent test results were clarified upon repeat testing. Overall testing results indicate that our rapid Pf blood test was able to consistently detect 25 parasites/DL with 100% Sensitivity (CI: 77%-100%, n=14), and this result was equivalent to the whole-blood qPCR (100% sensitivity, CI: 88%-100%, n=28). However, when testing for detection of 1.5 parasites/μL or higher, our test was 83% sensitive (CI: 65%-94%, n=30), and this was superior to the whole-blood qPCR that was only 78% sensitive (CI: 64%-88%; n=54). These results suggest that the rapid Pf blood test can improve reliability of detection compared to whole-blood qPCR when smaller concentrations of parasite are present.

When compared to RDT detection, which reliably detect only >200 parasites/μL clinical sensitivities, the detection of >25 parasites/μL using the rapid Pf blood test suggests our rapid test could reliably provide an 8-fold improvement in sensitivity compared to RDTs. In addition, with reported 60% clinical sensitivities of some RDTs, our 80% sensitivity of detection down to >1.5 parasites/μL represents a potential 100× improvement in clinical sensitivity. Critically, this >25 parasites/μL sensitivity enables detection of asymptomatic carriers who can have small levels of parasite in the blood (<150 parasites/μL). The test provides a rapid format for improved whole-blood detection of P. falciparum in low-resource settings, providing results within 20 minutes. This would enable patients to be provided with treatment on the same day of testing, reducing the need to follow up on a subsequent day, to help reduce transmission chains in endemic countries.

The ability of the rapid Pf blood test to detect parasites in blood was examined using blood spiked with known concentrations of P. falciparum, mixed with Sample Preparation Reagent U at specified ratios to sample volume, incubated for 5 minutes at room temperature. These blood/sample prep mixes were then mixed with Magnesium acetate (140 mM) or water, and then used immediately in the rapid Pf blood test. This data (not shown) demonstrates that a ratio of blood: Sample Preparation Reagent of from 2:1 to 1:1, when subsequently diluted with Magnesium acetate (140 mM) at a ratio of mix: Magnesium acetate of from 1:1 to 1:4 provides optimal detection. For example, a mixture of equal volume blood: Sample Preparation Reagent, followed by addition of equal volume MgOAc (n=54) demonstrated >96% diagnostic sensitivity and 100% specificity (95% confidence interval, CI: 80-100%) at detecting >50 parasites/μL.

### EXAMPLE 14: RPA-LFD Assay for the Detection of P. Falciparum in Mosquitoes Following Sample Preparation with Sample Preparation Reagent U

This example describes application of the rapid and sensitive Plasmodium falciparum test for the detection of infection in mosquitoes, suitable for low-resource implementation. The test uses a novel 10-minute sample preparation method that requires only tube, pestle, and a liquid reagent. Sensitive 10-minute isothermal amplification of the Plasmodium 18S rRNA gene is performed using recombinase polymerase amplification (RPA followed by lateral flow detection), thus requiring only a single temperature heating block for operation.

### Mosquitoes

Anopheles stephensi (Sind-Kasur strain) were obtained from the University of Nijmegen, The Netherlands, and maintained in colony at the QIMR Berghofer Medical Research Institute insectary at 27° C., 70-80% relative humidity and 12:12 hour day: night light cycle. Adult mosquitoes were fed on 8% sucrose with para-aminobenzoic acid (PABA). Female mosquitoes were collected at 3-4 days old, starved for 15 hours, and 100 mosquitoes were placed into paper cups with gauze lids. A blood and parasite inoculum was prepared by mixing 650 μL of Plasmodium infected red blood cells obtained from a culture of Plasmodium falciparum (NF54 strain) with 600 L of AB blood serum. Mosquitoes were allowed to feed on the inoculum via an artificial membrane feeding apparatus for 30 minutes. After feeding, mosquitoes that were not engorged were discarded and remaining mosquitoes were incubated in an environmental chamber for 10 days before dissection.

### RPA Oligonucleotides

Primers (forward and reverse) and a probe for recombinase polymerase amplification were designed to target the 18S rRNA gene, using 57 aligned sequences from GenBank (Table 32). Primers and probes were resuspended using DNase and RNase-free water to 100 IM concentrations before storage at −80° C., and diluted to 10 M for use as working stocks. The concentration (18S gene copies per IL) of the resuspended plasmid was determined using the dsDNA HS Assay Kit (Life Technologies, Singapore).

### Microscopy.

Mosquitoes were dissected and their midguts were examined for the presence and quantity oocysts using mercurochrome stain and microscopy according to standard procedures.

### Quantitative PCR Assay

After staining and microscopy, DNA was extracted from dissected midguts by homogenizing samples in 260 μl of QIAGEN ATL buffer using a micropestle and storing samples at −20° C. Prior to DNA extraction; samples were thawed, 40 μL of Proteinase K was added to each tube and samples were incubated at 56° C. overnight. DNA was then extracted from samples using the QIAGEN DNeasy blood and tissue DNA extraction kit (QIAGEN, Hilden, Germany) according to the manufacturer's instructions. Quantitative PCR was performed using the QIAGEN QuantiNova Probe qPCR kit with an additional 1 mM MgCl₂. qPCR reactions consisted of 7.5 L of QIAGEN QuantiNova 2× qPCR master mix, 1.5 L of 1:1 dilution of DNA and QuantiNova yellow sample dilution buffer, 0.1 μM of each primer and 0.1 μM of a CY5-conjugated Taqman probe targeting a conserved region of the P. falciparum 18S gene (Rockett et al. 2011, Malar J. 2011; 10:48) in a total volume of 15 μL. The qPCR assay was performed on a Corbett Rotorgene 6000 thermocycler (Corbett Research, Sydney, Australia) under the following conditions: 95° C. for 2 min, followed by 40 cycles of 95° C. for 5 s and 60° C. for 30 s.

### Sample Preparation

P. falciparum fed mosquitoes were crushed in 50 μL of Sample Preparation Reagent U and incubated at RT for 10 minutes. Pooled mosquitoes were crushed in 200 μL Sample Preparation Reagent U. From this mixture, 10 μL was then added to 40 μL of RNase and DNase-free water and 1 L immediately used for isothermal amplification.

### Isothermal Amplification

Sample (1 μL) was mixed with 8 μL freshly-prepared recombinase polymerase amplification (RPA) mixture, followed by addition of 1 μL 140 mM Magnesium acetate to start the reactions, and incubation at 39° C. for 10 minutes. The final concentration of reactants in each tube was: 420 nM Forward and Reverse primers, 120 nM probe, 1× Rehydration buffer & pellet mixture, and 14 mM Magnesium acetate.

### Lateral Flow Detection

Immediately following incubation, 2 μL of the amplicons were transferred to the sample pad of a HybriDetect lateral flow strip (Milenia Biotec GmbH, Gieβen, Germany), which had been pre-prepared by the addition of 8 μL blocking buffer (0.4% casein, 0.1% Tween in PBS, pH 9) to the sample pads of the strips [16]. Strips were subsequently placed into tubes with the sample pads immersed in 100 μL of borate buffer (100 mM H3BO3, 100 mM Na2B4O7, 1% BSA, 0.05% Tween 20, pH 8.8), and left to wick along the strips for 5 minutes before appearance of test bands was observed by eye and imaged.

### Data Analysis

Strips were imaged using a digital imaging system and analysed using ImageJ software (National Institutes of Health, MD, USA). Greyscale-converted images were used to determine band -intensity, by measuring the mean grey value (limit to threshold), using a fixed area measurement, and subtracting from the maximum threshold value. For each test band, an average of the neighbouring white space of all LF strips in that experiment was subtracted from the band intensity to normalize the results. To define a sample as positive the test band values were standardized by subtracting a cutoff value (three standard deviations above the average of the negative control test band intensities). A standardized value of greater than 0 was a positive result.

Comparative analysis of tests was performed using a diagnostic sensitivity and specificity calculator with exact Clopper-Pearson 95% confidence intervals.

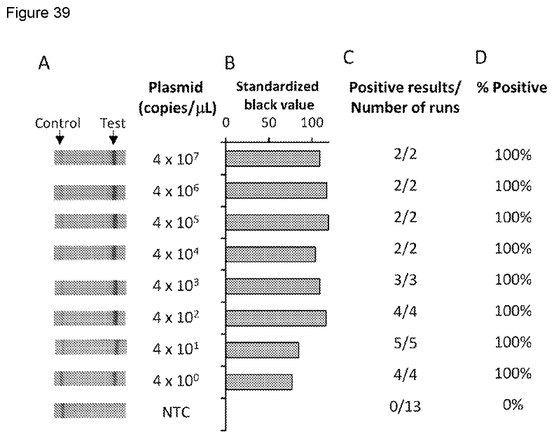

Analytical sensitivity of the optimal Pf RPA-LFD test (FIG. 39) using the quantified DNA standard indicated observed test band intensity was robust, with the strips producing clearly visible test lines at all concentrations tested (FIG. 39A). A reduction in the intensity of the control band was concurrently observed as the plasmid template concentration increased, as expected when increasing amounts of amplicon reduce availability of labelled conjugate binding to the control antibody. Analytical sensitivity analysis, as determined both by eye (FIG. 39A) and by image analysis of pixel density (FIG. 39B-D), indicated a limit of detection of 4 copies/μL.

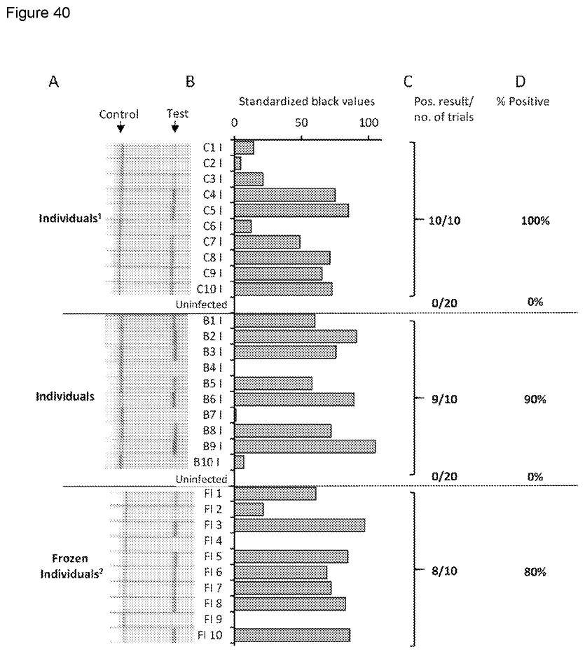

The rapid Pf RPA-LFD was combined with a Sample Preparation Reagent U, to construct a rapid Pf test for detection of infected mosquitoes. To trial the rapid Pf test, Anopheles stephensi mosquitoes were fed on P. falciparum infected blood cultures and harvested 10 days post meal. Whole mosquitoes were crushed in Sample Preparation Reagent U and incubated for 10 minutes, diluted in water (1 in 5), and then tested (1 μL) in the optimised Pf RPA-LFD test. Thirty mosquitoes fed on P. falciparum blood cultures were tested (20 tested fresh and 10 frozen and subsequently thawed for testing; FIG. 40). Of the infected mosquitoes, twenty-seven were positive using the rapid Pf test, indicating a 90% prevalence of Plasmodium falciparum in the blood-fed mosquitoes. However, not every line was a strong positive, suggesting the test was either weakly detecting low concentrations of parasite or giving non-specific amplification, either due to the Sample Preparation Reagent or the crushed blood-fed mosquito background. Analytical sensitivity testing of Sample Preparation Reagent U spiked into plasmid DNA did not indicate an increase in non-specific amplification (data not shown), although results showed an impact on sensitivity, resulting in a limit of detection to 40 copies/DL. However, applying the rapid Pf test to 40 uninfected mosquitoes (20 fed on uninfected blood cultures, and 20 not blood-fed) produced a clear negative result for all uninfected mosquitoes tested, demonstrating 100% diagnostic specificity (95% Confidence interval, CI: 91%-100%; n=40). These results suggested that the crushed blood-fed mosquito background did not affect test specificity, and that any observable band, regardless of intensity, was indicative of a mosquito that had been exposed to P. falciparum.

To compare the effectiveness of the rapid Pf test, microscopy was performed on an additional 39 mosquitoes from the same batch of A. stephensi fed on P. falciparum infected blood cultures. All mosquitoes were dissected, and their midguts examined by microscopy for the presence of oocysts. A total of 24 mosquitos were observed to have oocysts in their midgut (range 1-19, mean 4), indicating a microscopy positive prevalence rate of 62% (n=39; Table 33). While microscopy is considered the gold standard of detection of Plasmodium in mosquitoes, there is the possibility of failing to identify parasites, especially at low numbers. Therefore, DNA was extracted from the midguts of 19 mosquitoes already examined by microscopy and evaluated using qPCR, resulting in 14 mosquitoes testing positive by qPCR (Table 33) and indicating a qPCR-positive prevalence rate of 73% (n=19). However, two of the microscopy negative mosquitoes were detected as positive by qPCR, and one qPCR negative result contained the largest oocyte number by microscopy, indicating this sample was also positive. By combining results, we determined four mosquitoes were negative by both tests, suggesting an estimated “true” infection prevalence of 79% (15 positives out of 19 mosquitoes; Table 33 and Table 34).

TABLE 33
Estimation of Infection prevalence for Anopheles stephensi
fed on infected P. falciparum blood cultures, determined
by microscopy and qPCR on a subset of 19 mosquitoes.

| | | Microscopy | | |
|---|---|---|---|---|
| | | Positive | Negative | Total |
| qPCR | Positive | 12 | 2 | 14 |
| | Negative | 1 | 4 | 5 |
| | Total | 13 | 6 | 19 |

A comparative analysis of the diagnostic sensitivity of each test was determined using the combined microscopy and qPCR estimated prevalence as the “true prevalence” of infection, with results presented in Table 34. Both qPCR and microscopy failed to detect some of the “true positives,” with diagnostic sensitivities calculated to be 93% for qPCR (CI: 68%-100%, n=19), and 77% for microscopy (CI: 59%-90%, n=39). For RPA testing, the infected mosquito testing results (n=30) were combined with the known uninfected mosquito test results (n=40), reducing the calculated prevalence of infection in the combined mosquito pool to 34% (n=70). The rapid Pf test determined a higher infection prevalence compared to the “true prevalence” estimate, resulting in 3 positive results being relabelled as “false positive” results. Using these calculations, the rapid Pf test diagnostic sensitivity was 100% (CI: 86%-100%, n=70), superior to microscopy and equivalent to qPCR. From this data set, the calculated diagnostic specificity was 94% (CI: 82%-99%).

TABLE 34
Performance of Microscopy, qPCR and RPA compared to estimated true disease

| | | | | Disease | | Prevalence^{c} | |
|---|---|---|---|---|---|---|---|
| | Result^{a} | | | (estimated)^{b} | | (No. | Sensitivity |
| Test method | | I | U | Positive | Negative | mosquitoes) | (95% CI)^{d} |
| Microscopy | Positive | 24 | | 24 | 0 | 79% (n = 39) | 77% |
| | Negative | 15 | | 7 | 8 | | (CI: 59%-90%) |
| qPCR | Positive | 14 | | 14 | 0 | 79% (n = 19) | 93% |
| | Negative | 5 | | 1 | 4 | | (CI: 68%-100%) |
| Rapid P. | Positive | 27 | 0 | 24 | 3 | 34% (n = 70) | 100% |
| falciparum | Negative | 3 | 40 | 0 | 43 | | (CI: 86%-100%) |
| test | | | | | | | |

^{a}Test results obtained when testing Anopheles stephensi mosquitoes fed on P. falciparum-infected blood cultures (I) or know uninfected mosquitoes (U).
^{b}Results when compared to the Estimated true disease, calculated form the estimated prevalence
^{c}which was determined to be 79% for Anopheles stephensi mosquitoes fed on P. falciparum-infected blood cultures, based on combined microscopy and qPCR results. For RPA, which also tested 40 known negative uninfected mosquitoes, the combined estimated prevalence for all mosquito testing was calculated to be 34% (n = 70).
^{d}Clopper-Pearson confidence intervals

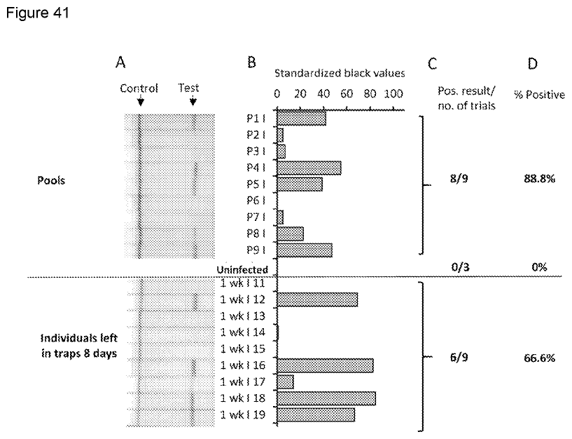

Parasite detection is usually performed in mosquito pools, due to the large number of mosquitoes caught for examination in the field. The ability of the rapid Pf test to detect parasites was examined when a single mosquito (from the same batch of A. stephensi fed on P. falciparum infected blood cultures) was pooled with 19 known uninfected mosquitoes. Because of the larger sample mass, pools were homogenized in 200 μL Sample Preparation Reagent U before incubation, dilution, and subsequent testing using the Pf RPA-LFD test. Eight of 9 pools were determined to be positive (FIG. 41, top panel), indicating an 89% positivity rate, which was consistent with the individual mosquito testing estimates. All three pools containing only uninfected mosquitoes were negative, confirming the specificity of the rapid Pf test. The results (n=69; Table 35) indicate 100% diagnostic sensitivity (CI: 59-100%) and 98.4% specificity (CI: 91-100%).

TABLE 35
Calculated sensitivity and specificity of the rapid P. falciparum
when applied to the different trials performed in this study.

| | | A. stephensi | | | | | Calc. expected | | |
|---|---|---|---|---|---|---|---|---|---|
| | | mosquito testing | | | Disease | | prevalence^{c} | Rapid Pf test | Rapid Pf test |
| | Mosquito | result^{a} | | | (estimated)^{b} | | (no. | Sensitivity | Specificity |
| | trial | | I | U | Positive | Negative | mosquitoes) | (95% CI)^{d} | (95% CI)^{d} |
| Rapid | Individual | Positive | 27 | 0 | 24 | 3 | 34% | 100% | 93% |
| Pf | | Negative | 3 | 40 | 0 | 43 | (n = 70) | (CI: 86%-100%) | (CI: 82%-99%) |
| test | Pools | Positive | 8 | 0 | 7 | 1 | 10.3% | 100% | 98% |
| | | Negative | 1 | 60 | 0 | 61 | (n = 69) | (CI: 59%-100%) | (CI: 91%-100%) |
| | 8 days old | Positive | 6 | 0 | 6 | 0 | 79% | N/A | N/A |
| | | Negative | 3 | 0 | 1 | 2 | (n = 9) | | |
| | Overall | Positive | 41 | 0 | 38 | 3 | 25.6% | 100% | 97% |
| | | Negative | 7 | 100 | 0 | 107 | (n = 148) | (CI: 91%-100%) | (CI: 92%-99%) |

^{a}Test results obtained when testing Anopheles stephensi mosquitoes fed on P. falciparum-infected blood cultures (I) or known uninfected mosquitos (U).
^{b}Results when compared to the Estimated true disease, calculated from the estimated expected prevalence
^{c}determined to be 79% for Anopheles stephensi mosquitoes fed on P. falciparum-infected blood cultures, based on combined microscopy and qPCR results, and then adjusted depending on the number of known uninfected mosquitoes also included in the trial.
^{d}Clopper-Pearson confidence intervals.

The ability of the rapid P. falciparum test could detect infected mosquitoes left in traps for up to a week, to simulate a weekly testing regime, was examined. Individual mosquitoes were left in traps for 8 days in a chamber that simulated a tropical environment. Six of 9 individual mosquitoes tested using the rapid P. falciparum test were positive after being held in these conditions (FIG. 41, bottom panel). This 67% positivity rate is lower than the positivity rate obtained from individual and pool testing, but within the standard error of testing, indicating the rapid P. falciparum test could be implemented for weekly trap testing regimes. When combining all testing performed in the study (Table 35) and comparing results to the estimated expected prevalence determined by microscopy and PCR testing (combined with known uninfected mosquitoes), the rapid P. falciparum test indicated 41 positive results, with a potential seven “false negatives” results, and correctly did not detect 100 uninfected mosquitoes. These results (n=148) indicating a diagnostics sensitivity of 100% (CI: 91-100%) and diagnostic specificity of 97% (CI: 92-99%), regardless of whether testing was performed on individual mosquitoes or pools, and whether mosquitoes were fresh, frozen, or left in traps for up to one week.

### EXAMPLE 15: RPA-LFD Assay for the Detection of Hepatopancreatic Parvoviruses in Prawns (Fenneropenaeus merguiensis) Following Sample Preparation with Sample Preparation Reagent U

Viral diseases are a major concern in aquaculture, especially in prawn rearing. The Hepatopancreatic parvoviruses (HPV) are common viruses of prawn and fish comprising Penaeus monodon densovirus (PmDNV), Penaeus merguiensis densovirus (PmeDNV), and Penaeus chinensis densovirus (PchDNV). These viruses reduce the survival of larvae in banana shrimp (Fenneropenaeus merguiensis) and can stunt growth at juvenile stages in grow-out ponds for both Japanese tiger prawn (Penaeus japonicas) and farmed black tiger shrimp (Penaeus monodon).

The ability to detect HPV in banana shrimp (Fenneropenaeus merguiensis) and detection of amplified products by lateral flow dipstick was examined.

### Animal Samples

All the specimens were identified as banana shrimp (Fenneropenaeus merguiensis) by morphological keys. Shrimp hepatopancreases were aseptically dissected, tissue kept in RNA-later solution and stored at −80° C. until used. Samples of farmed banana shrimp were collected at harvest time after 140 days of grow-out. Details on rearing conditions, breeding practices, genotyping, and pedigree construction were described previously. The genotypes, based on DNA microsatellites, were used to assign animals to full-subgroups thus establishing a pedigree. In the present study, samples were selected from 3 full-sib groups previously quantified to have low (102-103), medium (104-105), and high (106-107) HPV copy number. Wildtype banana shrimps were HPV-screened by qPCR and identified as HPV negative.

DNA Extraction and Real-Time qPCR

Genomic DNA was extracted using a DNeasy® Blood & Tissue Kit (Qiagen) according to the manufacturer's instructions. DNA concentration was measured using a NanoDrop™ 2000 spectrophotometer (Thermo Fisher Scientific) and normalized to a concentration of 800 ng/μL. Extracted DNA was stored at −80° C.

HPV titres were quantified by qPCR using the SensiMix HRM kit (Bioline) with a Rotor-Gene 6000 thermal cycler (Corbett Research). Validated primer sequences described by La Fauce et al. (2007) were used for qPCR: HPV140 forward, 5′-CTA CTC CAA TGG AAA CTT CTG AGC-3′ (SEQ ID NO: 30), and HPV140 reverse, 5′-GTG GCG TTG GAA GGC ACT TC-3′ (SEQ ID NO: 31).

### Primer and Probe Design for RPA

Primers targeting the structural capsid protein gene of HPV and probes were designed manually via a multiple sequence alignment using the GeneDoc software program (Table 36).

TABLE 36
Primers and Probes for the detection of prawn
hepatopancreas structural capsid protein gene.

| Primer | | | Labelled/ |
|---|---|---|---|
| type | ID | Sequence (5′ → 3′) | unlabelled |
| Forward | HPV-F1 | CCGCCGCVCCGCAVCAVMDAKCTGCDNCRG | unlabelled |
| | | (SEQ ID NO: 92) | |
| Forward | HPV-F2 | CGCCGCVCCGCAVCAVMDAKCTGCDNCRGG | unlabelled |
| | | (SEQ ID NO: 93) | |
| Forward | HPV-F3 | ACCGCCGCVCCGCAVCAVMDAKCTGCDNCR | unlabelled |
| | | GG (SEQ ID NO: 94) | |
| Forward | HPV-F4 | CCGCCGCVCCGCAVCAVMDAKCTGCDNCRG | unlabelled |
| | | G (SEQ ID NO: 95) | |
| Forward | HPV-F5 | ACCGCCGCVCCGCAVCAVMDAKCTGCDNCR | unlabelled |
| | | G (SEQ ID NO: 96) | |
| Forward | HPV-F6 | [5′ Biotin] | labelled, |
| | | GGAAACYTCTGARYCMGGRVYYACCGCCGC | biotin |
| | | RCCSC (SEQ ID NO: 97) | |
| Reverse | HPV-R1 | AGRTCRAYDGGYTKRTTGCGGKGGCGYTGG | unlabelled |
| | | (SEQ ID NO: 98) | |
| Reverse | HPV-R2 | GTTKBAGRTCRAYDGGYTKRTTGCGGKGGC | unlabelled |
| | | G (SEQ ID NO: 99) | |
| Reverse | HPV-R3 | CCRATGTGTTKBAGRTCRAYDGGYTKRTTG | unlabelled |
| | | CGG (SEQ ID NO: 100) | |
| Reverse | HPV-R4 | GTGTTKBAGRTCRAYDGGYTKRTTGCGGKG | unlabelled |
| | | GC (SEQ ID NO: 101) | |
| Reverse | HPV-R5 | GTGTTKBAGRTCRAYDGGYTKRTTGCGG | unlabelled |
| | | (SEQ ID NO: 102) | |
| Reverse | HPV-R6 | CCRATGTGTTKBAGRTCRAYDGGYTKRTTG | unlabelled |
| | | C (SEQ ID NO: 103) | |
| Probe | HPV-P2 | [5′ FAM] | labelled, |
| | | GGCGYTGGAAKGMAYYKYYDGDRTTTYTHC | FAM |
| | | C [Internal dS Spacer] TACCCTG | |
| | | CDGWYTCDCC [3′ C3 spacer] (SEQ | |
| | | ID NO: 104) | |
| Probe | HPV-P5 | [5′ FAM] | labelled, |
| | | CGYTGGAAKGMAYYKYYDGDRTTTYTHCCR | FAM |
| | | TA [Internal dS Spacer] CCTGCD | |
| | | GWYTCDCCTCC [3′ C3 spacer] | |
| | | (SEQ ID NO: 105) | |

*The optimal amplifying primers was dependent on the final embodiment of the reaction mix.

### Tissue Sample Preparation for RPA

Tissue samples were prepared combining 40 μg hepatopancreas with 50 μl of Sample Preparation Reagent U). Field-suitable crude lysate DNA extraction was performed using a homogenizing pestle in a 1.5 ml tube. Samples were diluted 1/5 in water to have a final concentration of 160 ng/μL.

Isothermal DNA Amplification with RPA and Lateral-Flow Strip Detection

Lateral-flow strip-RPA assays were performed using a TwistAmp nfo Kit (TwistDx, Cambridge UK) as described by the manufacturer with minor adaptions. Briefly, 29.5 L rehydration buffer, 5.9 μL molecular grade water and 4.6 μL primer/probe mix (forward primer 400 nM, reverse primer 400 nM, probe 120 nM) were added to one pellet and mixed by pipetting up and down. Then 8 μL of the master reaction mix were distributed to tubes and 1 μL of DNA template, tissue extract or control was added. Finally, the reaction was started by adding 1 μL 140 mM magnesium acetate (total reaction volume per tube 10 μL). The reaction mix was incubated in a heating block for 25 minutes at 39° C. with brief mixing after 10⁻¹⁵ minutes of incubation and at the end of the 25 minutes incubation period. After amplification, 2 μL of RPA amplicons were dropped onto the middle of the sample application area of a HybriDetect MGHD 1 strip (Milenia Biotec GmbH, Gieβen, Germany), which was blocked with 8 μL casein buffer (0.4% casein, 0.1% Tween in PBS, pH 9). Strips were then placed into tubes containing 100 L of borate buffer (100 mM H3BO3, 100 mM Na2B4O7, 1% BSA, 0.05% Tween 20, pH 8.8), incubated at room temperature for 5 minutes and imaged immediately. All experiments were performed at least three times if not specified otherwise.

### Analytical Sensitivity of HPV RPA Lateral-Flow Strip Assay

A DNA template corresponding to position 3928-4404 of Penaeus chinensis hepandensovirus (Accession number: AY008257) was synthesized as a double-stranded DNA gBlock® from Integrated DNA technologies (Coralville, IA, USA) and quantitated using the Qubit™ dsDNA HS Assay Kit (Thermo Fisher Scientific, Mulgrave, VIC, AU). To evaluate the sensitivity of the assay, 10-fold serial dilutions of the HPV template DNA were tested. Additionally, the HPV DNA template was used as a positive control (2.42E+06 copies/μL) in all experiments.

### Diagnostic Sensitivity and Specificity of HPV RPA Lateral-Flow Strip Assay

To validate clinical sensitivity and specificity, 160 ng (1 μL) hepatopancreas crude lysate DNA dilutions were assayed per reaction and compared to HPV titre results obtained by qPCR. In total, six HPV negative (not detected), six low (102-103), twelve medium (104-105), and six high (106-107) HPV copy number samples were tested.

### Imaging and Statistical Data Analyses

Strips were imaged using the MultiDoc-It Digital Imaging System (Upland, CA, USA) and analysed using ImageJ software (National Institutes of Health, MD, USA). Greyscale-converted images were used to determine band -intensity, by measuring the mean grey value (limit to threshold), using a fixed area measurement, and subtracting from the maximum mean grey value (255). For each test band, the average of two neighbouring relative white spaces was subtracted from the band intensity to normalize the results. A sample was defined as positive if the normalised band intensity was 3 times higher than the standard deviation of the two neighbouring white space values.

### Design of RPA Primer and Probe Sets

To develop a rapid assay for detection of HPV in prawns, we targeted the structural capsid protein gene of HPV, as this region has previously been used for development of qPCR assays [Knibb W, Quinn J, Kuballa A, Powell D, Remilton C, Nguyen NH. Yearly, pond, lineage and family variation of hepatopancreatic parvo-like virus (HPV) copy number in banana shrimp Fenneropenaeus merguiensis. J Invertebr Pathol. 2015; 128:73-9; and Phuthaworn C, Nguyen NH, Quinn J, Knibb W. Moderate heritability of hepatopancreatic parvovirus titre suggests a new option for selection against viral diseases in banana shrimp (Fenneropenaeus merguiensis) and other aquaculture species. Genet Sel Evol. 2016; 48 (1): 64.]. By analysing multiple sequence alignments of hepandensovirus sequences from a variety of Penaeus and Fenneropenaeus sp., highly conserved regions were chosen for primer and probe design suitable for use in an RPA reaction. The developed test uses the recombinase enzyme to insert biotin and FITC-labelled primers and probes into the double stranded template at a single temperature of 39° C. for 25 min, followed by lateral flow strip detection of the biotin-FITC dual-labelled amplicon.

### Analytical Sensitivity of HPV RPA Lateral-Flow Strip Assay

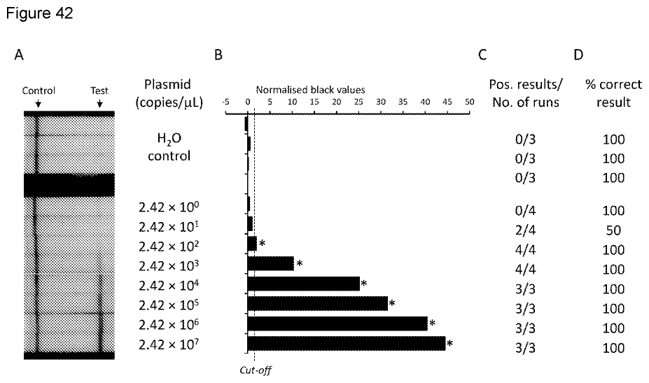

To test the analytical sensitivity (detection limit) of the Prawn HPV RPA test, serial dilutions of a synthetic template DNA with known copy number were assessed. Strong bands were observed by eye down to 104 copies of DNA, with a fainter positive band visible at 103 copies (FIG. 42). With digital analysis the HPV RPA lateral-flow assay robustly detected down to <242 genome equivalent copies per reaction. Further, in 2 out of 4 separate runs, the assay showed a positive result with 24 copies per reaction. Concerning analytical specificity, we observed a faint band in the negative controls when running time exceeded 5 minutes, thus potentially generating false-positive results.

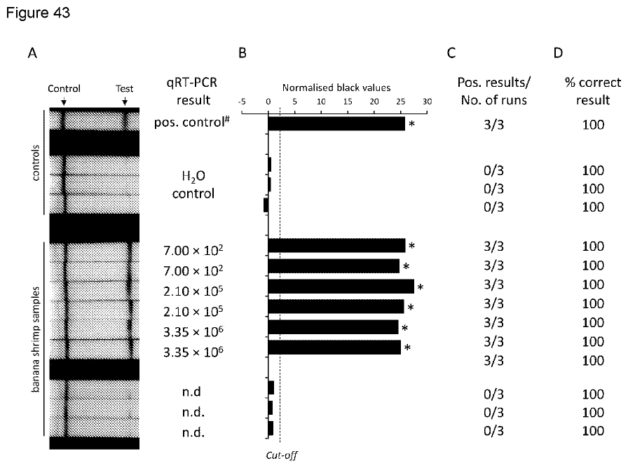

To validate the diagnostic accuracy of the rapid HPV assay for use in aquaculture facilities, 30 samples with known HPV titre determined by qPCR to contain high (106-107 copies, n=6), medium (104-10⁵ copies, n=12), low (102-103 copies, n=6) or non-detectable (negative, n=6) amounts of HPV were tested. The rapid HPV RPA lateral-flow strip test consistently detected all HPV-positive samples (n=24) as HPV-positive, regardless of high, medium or low HPV copy number. Further, all samples containing non-detectable amounts of HPV previously determined by qPCR were identified by HPV RPA test as negative (n=9). Hence, the test did not produce any false-positive results in the HPV-negative samples (FIG. 43). Taken together, these results (n=33) suggest that the rapid HPV assay detects HPV with high diagnostic accuracy, with 100% diagnostic sensitivity (95% confidence interval, CI: 86-100%) and 100% specificity (CI: 66-100%)

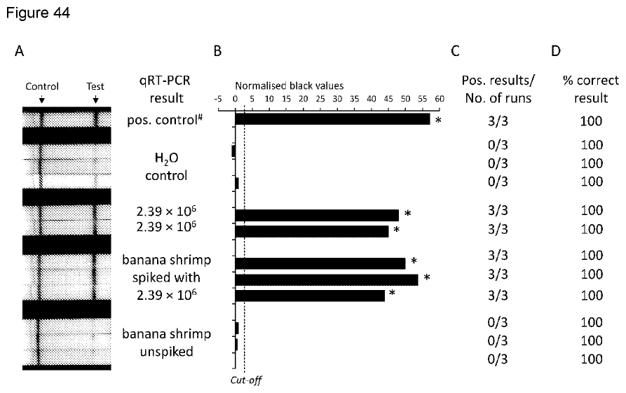

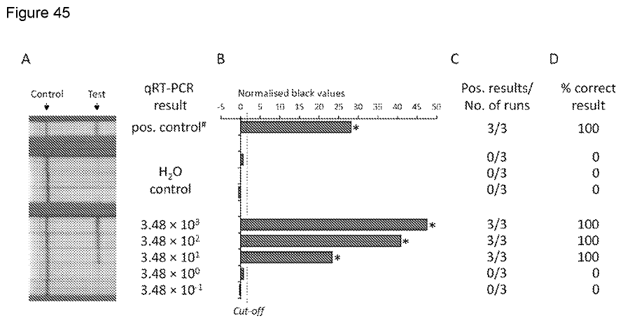

The diagnostic sensitivity of the RPA lateral-flow strip assay was determined by spiking negative-tested wildtype banana shrimp samples with an HPV-positive sample (high copy number, ratio 1:1). The assay successfully detected HPV in the spiked samples, but not in the unspiked controls (FIG. 44). To further trial the sensitivity of the assay we choose to serially dilute an HPV-positive sample (low HPV titre determined by qPCR) in a banana shrimp sample matrix. Comparable with the results obtained by serial diluting a synthetic DNA template (FIG. 45), we efficiently detected the serial diluted HPV-positive sample with the lowest detectable HPV titre being 3.48×101 suggesting a detection limit of <35 genome equivalent copies per reaction (FIG. 45). These results further confirm that the rapid HPV RPA lateral-flow strip assay has high diagnostic accuracy.

These data demonstrate that in the analytical sensitivity test, the HPV RPA lateral-flow strip assay detected <242 copies per reaction in 100% of the cases, respectively (FIG. 42), and therefore has an analytical detection limit of <242 genome equivalent copies per reaction. In an analytical sensitivity test in banana shrimp matrix, the rapid HPV lateral-flow strip assay showed a detection limit of <35 HPV copies per reaction (FIG. 45). The level of sensitivity was sufficient to detect HPV in samples with high (106-107), medium (104-105) and low (102-103) copy number of the virus (FIG. 43). Samples, which had a not detectable HPV titre by qPCR, were correctly identified as being HPV-negative by the assay, indicating high diagnostic specificity in addition to high diagnostic sensitivity.

### EXAMPLE 16: Sample Preparation Reagent E can Inactivate Henipaviruses and Enable Sensitive Detection Using RT-RPA

To develop rapid, low-resource Nipah virus (NiV) assays, two reverse transcription recombinase polymerase amplification (RT-RPA) tests and one reverse transcription recombinase-aided amplification (RT-RAA) test were designed that targeted a conserved region within the Nucleocapsid protein gene of NiV. Primers and probes were designed to enable subsequent lateral flow detection (LFD), such that appearance of a test line signified positive detection of a NiV (Table 37). Different combinations of forward and reverse primers, and probes were tested using reverse transcribed RNA as template to identify an optimized combination used for all testing.

TABLE 37
Primers and Probes for the detection of Nipah virus
Nucleocapsid protein gene.

| Primer | | | Labelled/ |
|---|---|---|---|
| type | ID | Sequence (5′ → 3′) | unlabelled |
| Forward | NIV F5 | AGGATTCTTCGCAACCATCAGATTYGGGTTG | unlabelled |
| | | (SEQ ID NO: 106) | |
| Forward | NIV F6 | ATTCTTCGCAACCATCAGATTYGGGTTGGAG | unlabelled |
| | | (SEQ ID NO: 107) | |
| Forward | NIV F7 | GCAACCATCAGATTYGGGTTGGAGACAAGG | unlabelled |
| | | (SEQ ID NO: 108) | |
| Forward | NIV F8 | ATTCTTCGCAACCATCAGATTYGGGTTGGAGA | unlabelled |
| | | C (SEQ ID NO: 109) | |
| Forward | NIV F9 | TTCTTCGCAACCATCAGATTYGGGTTGGAGAC | unlabelled |
| | | AAG (SEQ ID NO: 110) | |
| Forward | NIV F10 | GGATTCTTCGCAACCATCAGATTYGGGTTGGA | unlabelled |
| | | GAC (SEQ ID NO: 111) | |
| Forward | NIV F11 | CAGGATTCTTCGCAACCATCAGATTYGGGTTG | unlabelled |
| | | GAG (SEQ ID NO: 112) | |
| Reverse | NIV R1 | [5′ FAM] GAATTGATTCTTCAAGAAGCACC | labelled, |
| | | ATATAAGGG (SEQ ID NO: 113) | FAM |
| Reverse | NIV R5 | [5′ FAM] TCAAGAAGCACCATATAAGGGGC | labelled, |
| | | TCTTGGG (SEQ ID NO: 114) | FAM |
| Reverse | NIV R6 | [5′ FAM] TTAGTCTGAATTGATTCTTCAAG | labelled, |
| | | AAGCACC (SEQ ID NO: 115) | FAM |
| Probe | NIV P1 | [5′ Biotin] GCACTCAAYGAATTCCAGAG | labelled, |
| | | TGACCTCAAC [Internal dS spacer] | biotin |
| | | CCATCAARAGCTTGATG [3′ C3 | |
| | | spacer] (SEQ ID NO: 116) | |
| Probe | NIV P2 | [5′ Biotin] ATTCCAGAGTGACCTCAACA | labelled, |
| | | CCATCAARAGC [Internal dS spacer] | biotin |
| | | TGATGCTACTCTACAG [3′ C3 spacer] | |
| | | (SEQ ID NO: 117) | |

### Nipah Virus RPA and RAA Test Results

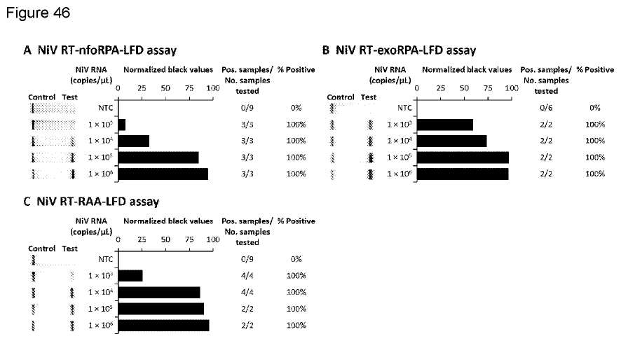

NiV isothermal assay analytical sensitivity. To test the analytical sensitivity of three NiV assays (RT-nfoRPA-LFD, RT-exoRPA-LFD and RT-RAA-LFD), serial dilutions of a synthetic template RNA with known copy number were assessed. The analytical sensitivity ranged from the highest concentration tested (1×10⁶ copies/μL) to as little as 1000 copies/μL for all three tests (FIG. 46A-C).

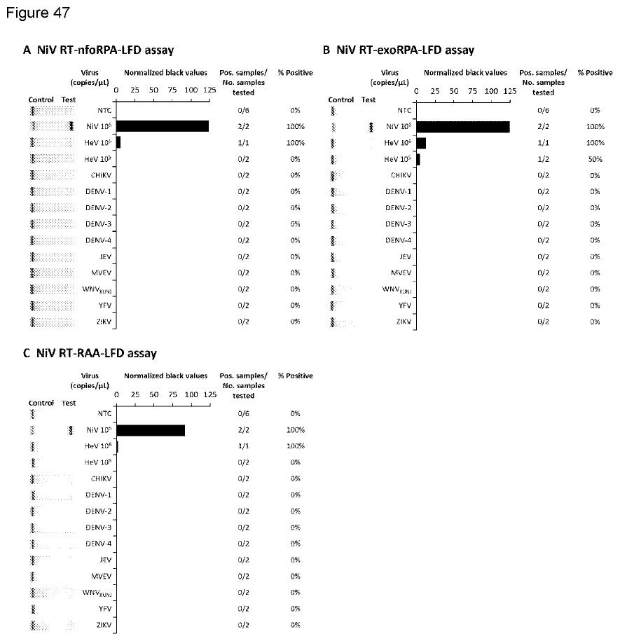

NiV isothermal assay specificity. Since symptoms of NiV infection are similar to other febrile diseases, specific diagnosis is critical for containment of an outbreak and to facilitate appropriate patient care. To confirm our NiV assays were specific for NiV, we next trialled our three tests against synthetic HeV RNA transcripts and RNA extracts from Chikungunya virus (CHIKV), Dengue virus serotypes 1˜4 (DENV 1-4), Japanese encephalitis virus (JEV), Murray Valley encephalitis virus (MVEV), West Nile virus Kunjin strain (WNVKUNJ), Yellow fever virus (YFV), and Zika virus (ZIKV). Our Nipah tests did not detect CHIKV or any flaviviruses, however HeV was detected at very high concentrations (10⁶ copies/μL) showing faint positive test bands (FIG. 47A-C). Testing HeV at lower concentration (10⁵ copies/μL) revealed no positive test result with the RT-nfoRPA-LFD and RT-RAA-LFD. The RT-exoRPA-LFD resulted in a positive test result in one out of two replicates, suggesting more pronounced non-specific detection of synthetic HeV RNA compared to the other two test formats.

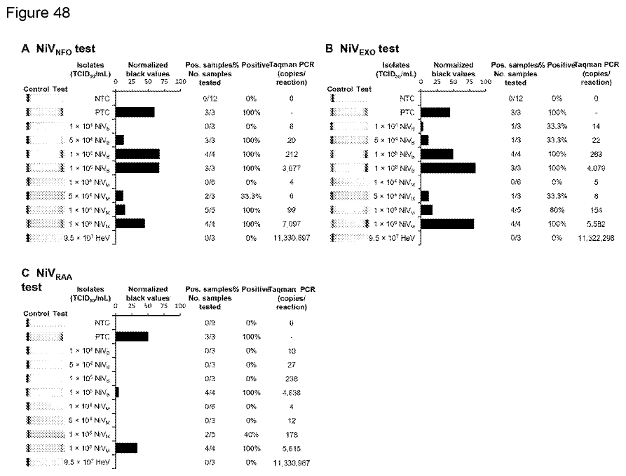

Sensitivity and specificity of rapid NiV tests using rapidly processed gamma-irradiated henipavirus isolate samples. By combining the rapid sample preparation method with our three isothermal NiV assays, we were able to develop three rapid NiV tests able to provide results in 30 minutes from sample to results. The rapid NiV_{NFO}, rapid NiV_{EXO} and rapid NiV_{RAA} tests combined rapid sample preparation followed by RT-nfoRPA-LFD, RT-exoRPA-LFD, or RT-RAA-LFD, respectively. To assess strain-specific sensitivities, two distinct strains of gamma-irradiated NiV isolates, Bangladesh (NiV_{B}) and Malaysia (NiV_{M}), were serially diluted in viral transport medium and tested with each rapid NiV test. Using this approach, we detected both NiVB and NiVM with two of the three rapid NiV tests (NIVNFO & NiV_{EXO}) in the range of 50,000-100,000 TCID₅₀/mL, the equivalent of approximately 50-100 infectious particles per microliter (FIG. 48A,B). However, the rapid NiV_{RAA} test was shown to detect virus at only 1,000,000 TCID₅₀/mL (FIG. 48C). All three NiV tests did not detect HeV at very high concentrations (95,000,000 TCID₅₀/mL).

Combining all gamma-irradiated virus testing results from FIG. 48 together, we assessed the diagnostic sensitivity and specificity of the test, for detection of henipavirus isolate samples at 100,000 TCID₅₀/mL or higher (n=25). The best performing test, the rapid NiV_{NFO} test, had 100.0% diagnostic sensitivity (95% CI: 79.4%-100.0%) and 100.0% diagnostic specificity (95% CI: 29.24%-100.0%). The other two Rapid NiV tests (NiV_{EXO} & NiV_{RAA}) demonstrated diagnostic sensitivities of 93.8% (95% CI: 69.8%-99.8%) and 62.5% (95% CI: 35.4%-84.8%) respectively, with both showing 100% diagnostic specificity (95% Cl: 29.2%-100.0%) (n=25).

### Hendra Virus RPA Oligonucleotides

To develop a rapid, low-resource Hendra virus (HeV) assay, a reverse transcription recombinase polymerase amplification (RT-RPA) test was designed that targeted a conserved region within the Nucleocapsid protein gene of HeV. Primers and probes were designed were manually to enable subsequent lateral flow detection (LFD), such that appearance of a test line signified positive detection of a HeV (Table 38). Different combinations of forward and reverse primers, and probes were tested using reverse transcribed RNA as template to identify an optimized combination used for all testing.

TABLE 38
Primers and Probes for the detection of Hendra virus
Nucleocapsid protein gene.

| Primer | | | Labelled/ |
|---|---|---|---|
| type | ID | Sequence (5′ → 3′) | unlabelled |
| Forward | HeV F5 | CCGGCTTCTTTGCGACTATCAGATTCGGTC | unlabelled |
| | | (SEQ ID NO: 118) | |
| Forward | HeV F6 | GAGGAAACAGGAATGGCCGGCTTCTTTGCG | unlabelled |
| | | (SEQ ID NO: 119) | |
| Forward | HeV F7 | AATTATGTCGAGGAAACAGGAATGGCCGGC | unlabelled |
| | | (SEQ ID NO: 120) | |
| Reverse | HeV R3 | [5′ Biotin]CCTGGTGCAAACTTTGTCT | labelled, |
| | | GAATRGATTCC (SEQ ID NO: 121) | biotin |
| Reverse | HeV R4 | [5′ Biotin]TGGATAACCACCTGGTGCA | labelled, |
| | | AACTTTGTCTG (SEQ ID NO: 122) | biotin |
| Reverse | HeV R6 | [5′ Biotin]AAACTTTGTCTGAATRGAT | labelled, |
| | | TCCTCAAGGAG (SEQ ID NO: 123) | biotin |
| Probe | HeV P1 | [5′ FAM]AGTTCCAGAGYGATCTCAATAC | labelled, |
| | | CATCAAAG[Internal dS spacer]GC | FAM |
| | | TGATGCTGCTCTAC[3′ C3 spacer] | |
| | | (SEQ ID NO: 124) | |
| Probe | HeV P2 | [5′ FAM]GATCTCAATACCATCAAAGGGC | labelled, |
| | | TGATGCTG[Internal dS spacer]TC | FAM |
| | | TACAGAGAAATAG [3′ C3 spacer] | |
| | | (SEQ ID NO: 125) | |

### Hendra Virus RPA and RAA Test Results

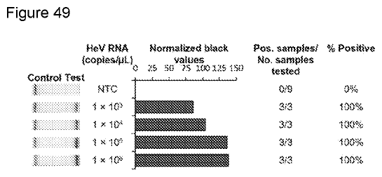

Analytical sensitivity of the HeV RT-RPA-LFD assay using synthetic RNA. The sensitivity of the HeV RT-RPA-LFD assay was first assessed using synthetic RNA. Under these conditions the HeV RT-RPA-LFD assay was reproducibly able to detect 1000 copies/μL of synthetic HeV RNA (FIG. 49).

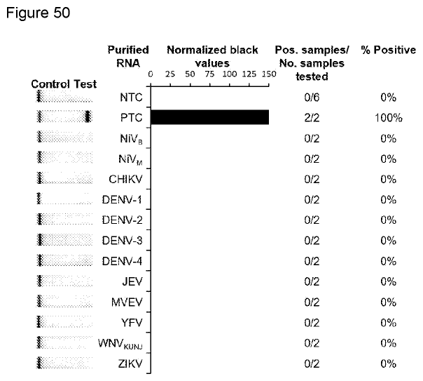

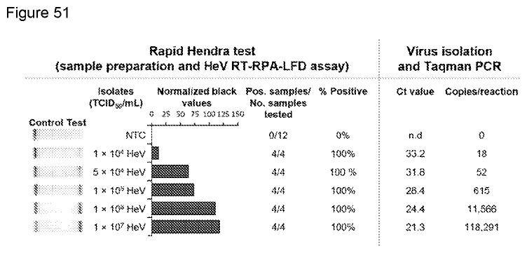

Analytical specificity of the HeV RT-RPA-LFD assay using purified virus RNA. The analytical specificity of the optimized HeV RT-RPA-LFD assay was assessed using purified RNA from Nipah virus, chikungunya virus and a range of other flaviviruses (FIG. 50). Apart from HeV no other viruses were detected by the assay.

Detection of HeV in viral transport medium. A rapid HeV test protocol was designed by combining sample processing with the HeV RT-RPA-LFD assay. Optimisation testing indicated that sample preparation by mixing Sample Preparation Reagent E to virus at a 1:1 ratio, followed by a 1/6 dilution in water, enabled samples to be subsequently detected by the HeV RT-RPA-LFD (data not shown). This optimised rapid HeV test protocol was trialled using gamma-irradiated HeV spiked into viral transport medium (Minimal Essential Medium containing 0.1% bovine serum albumin, 500 U/mL Penicillin, 500 μg/mL Streptomycin and 2500 μg/mL Fungizone) that is routinely used to collect veterinary samples. The rapid Hendra test detected a minimum of 10,000 TCID₅₀/mL of HeV (Error! Reference source not found.).

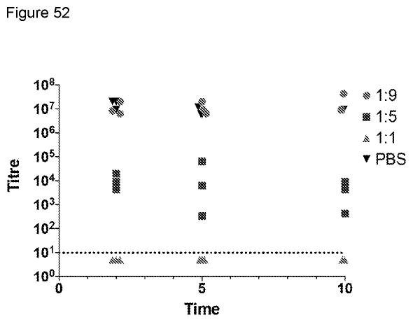

Sample Preparation Reagent E inactivation conditions for henipaviruses. Sample Preparation Reagent E inactivated Nipah virus (NiV). Initial testing was performed by mixing Sample Preparation Reagent E at different ratios with the virus (1:1, 5:1, 9:1; virus to Sample Preparation Reagent E), and incubating for 2, 5 and 10 minutes. Inactivation was observed by Median Tissue Culture Infectious Dose (TCID₅₀) assays and virus recovery studies. The virus titre after no exposure to Sample Preparation Reagent E was ˜5×10⁷ TCID₅₀/mL. NiV exposure at 9:1 (virus to Sample Preparation Reagent E) had no effect on Nipah virus infectivity levels. Exposure at 5:1, resulted in diminished NiV titres (102-105 TCID₅₀/mL). Complete loss of detectable infectious virus was only observed when NiV was exposed to Sample Preparation Reagent E at 1:1 ratio, regardless of incubation time (2, 5 and 10 minutes) (FIG. 52).

In a virus recovery study, where Nipah virus was exposed to Vero cells, wells showing no evidence of cytopathic effect were passaged and re-incubated with fresh cells, for up to three passages (Table 39). Virus exposed to mixtures containing Sample Preparation Reagent E at ratios of 1:9 (Sample Preparation Reagent E to virus) and 1:5 (Sample Preparation Reagent E to virus) showed cytopathic effect before any passaging was performed. Virus exposed to Sample Preparation Reagent E at 1:1 ratios did not result in cells with cytopathic effect, even after the third passage.

TABLE 39
Virus recovery of Nipah virus (Bangladesh isolate) mixed with
Sample Preparation Reagent E at various ratios (1:1, 5:1, 9:1)
and incubated at room temperature for 2, 5 and 10 minutes.

| | Incubation time | | | | | | | | |
|---|---|---|---|---|---|---|---|---|---|
| | 2 minutes | | | 5 minutes | | | 10 minutes | | |
| Nipah virus to Sample | CPE positive wells/total well number | | | | | | | | |
| Preparation Reagent E ratio | P1 | P2 | P3 | P1 | P2 | P3 | P1 | P2 | P3 |
| 1:1 | 0/3 | 0/3 | 0/3 | 0/3 | 0/3 | 0/3 | 0/3 | 0/3 | 0/3 |
| 5:1 | 3/3 | | | 3/3 | | | 3/3 | | |
| 9:1 | 3/3 | | | 3/3 | | | 3/3 | | |
| 1:1 PBS control | 3/3 | | | 3/3 | | | 3/3 | | |

Note:
Mixtures (100 μL) were added to individual wells of Vero cells (approximately 70% confluent) on 6-well plates. Plates were incubated for 7 days and then scored for the presence of Nipah virus cytopathic effect (CPE). Wells showing no evidence of Nipah virus CPE had 200 μL supernatant removed and added to fresh cells (blind passage) and plates, again incubated for 7 days before scoring for Nipah virus CPE. This blind passaging of negative samples was done for two additional passages (three 7 day incubations on Vero cells). Number indicates the number of CPE positive wells of the total of wells for each condition. Grey boxes mark where samples were not passaged due to the presence of CPE in the previous passage. P1-3: Passage 1 to 3. Phosphate buffered saline (PBS) was used as a negative control.

Additional viral recovery studies were performed at ratios 5:1, 2:1 and 1:1 (virus to Sample Preparation Reagent E) with Nipah virus (Bangladesh isolate and Malaysia isolate) and Hendra virus (Redlands/QLD isolate) (Table 40 and Table 41). A 1:1 ratio of Sample Preparation Reagent E to virus was needed for complete inactivation. A 1:2 ratio resulted in complete inactivation for 2 of 3 samples (1 of 2 samples for Nipah virus Malaysia isolate), with one sample from each treatment resulting in virus recovery in the first passage. A 1:5 ratio did not completely inactivate any tested virus sample. These results indicate a 1:1 ratio of Sample Preparation Reagent E to virus sample was needed to ensure complete inactivation of henipaviruses. This inactivation was rapid with it occurring in less than 2 minutes incubation at room temperature.

TABLE 40
Henipavirus recovery from samples incubated with and
without Sample Preparation Reagent E using different
ratios (sample to Sample Preparation Reagent E; 1:1,
2:1 and 5:1) at an incubation time of 10 minutes.

| Sample to Sample | | | |
|---|---|---|---|
| Preparation Reagent | CPE positive wells/total well number | | |
| E ratio | P1 | P2 | P3 |
| Nipah virus Bangladesh 5:1 | 3/3 | | |
| Nipah virus Bangladesh 2:1 | 1/3 | 1/3 | 1/3 |
| Nipah virus Bangladesh 1:1 | 0/3 | 0/3 | 0/3 |
| 1:1 PBS control | 3/3 | | |
| Nipah virus Malaysia 5:1 | 3/3 | | |
| Nipah virus Malaysia 2:1 | 1/2* | 1/2 | 1/2 |
| Nipah virus Malaysia 1:1 | 0/3 | 0/3 | 0/3 |
| 1:1 PBS control | 3/3 | | |
| Hendra virus 5:1 | 3/3 | | |
| Hendra virus 2:1 | 1/3 | 1/3 | 1/3 |
| Hendra virus 1:1 | 0/3 | 0/3 | 0/3 |
| 1:1 PBS control | 3/3 | | |

Note:
Mixtures (100 μL) were added to individual wells of Vero cells (approximately 70% confluent) on 6-well plates. Plates were incubated for 7 days and then scored for the presence of Nipah virus CPE. Wells showing no evidence of Nipah virus CPE had 200 μL supernatant removed and added to fresh cells (blind passage) and plates, again incubated for 7 days before scoring for Nipah virus CPE. This blind passaging of negative samples was done for two additional passages (three 7 day incubations on Vero cells). Number indicates the number of cytopathic effect (CPE) positive wells of the total of wells for each condition. Greyed out boxes mark where samples were not passaged due to the presence of CPE in the previous passage. P1-3: Passage 1 to 3.
*One tube was found to not contain Sample Preparation Reagent E when virus was added at PC4 and therefore was removed from the experiment.

TABLE 41
Henipavirus recovery from samples incubated with and without
Sample Preparation Reagent E using a 1:1 ratio (sample to Sample
Preparation Reagent E) at an incubation time of 2 minutes.

| Sample to Sample | | | |
|---|---|---|---|
| Preparation Reagent | CPE positive wells/total well number | | |
| E ratio | P1 | P2 | P3 |
| Nipah Bangladesh 1:1 | 0/3 | 0/3 | 0/3 |
| Nipah Malaysia 1:1 | 0/3 | 0/3 | 0/3 |
| Hendra 1:1 | 0/3 | 0/3 | 0/3 |
| Nipah Bangladesh 1:1 PBS control | 3/3 | | |
| Nipah Malaysia 1:1 PBS control | 3/3 | | |
| Hendra 1:1 PBS control | 3/3 | | |

Note:
Mixtures (100 μL) were added to individual wells of Vero cells (approximately 70% confluent) on 6-well plates. Plates were incubated for 7 days and then scored for the presence of Nipah virus CPE. Wells showing no evidence of Nipah virus CPE had 200 μL supernatant removed and added to fresh cells (blind passage) and plates, again incubated for 7 days before scoring for Nipah virus CPE. This blind passaging of negative samples was done for two additional passages (three 7 day incubations on Vero cells). Number indicates the number of cytopathic effect (CPE) positive wells of the total of wells for each condition. Greyed out boxes mark where samples were not passaged due to the presence of CPE in the previous passage. P1-3: Passage 1 to 3.
*One tube was found to not contain Sample Preparation Reagent E when virus was added at PC4 and therefore was removed from the experiment.

Inactivation and recovery studies with henipaviruses confirmed a 1:1 ratio Sample Preparation Reagent E to virus is able to inactivate Nipah virus (Bangladesh/Human/2004/Rajbari, R1 isolate: 5.54×10⁷ TCID₅₀/mL; Malaysia/Human/99 isolate: 2.13×10⁷ TCID₅₀/mL) and Hendra virus (Redand/QLD isolate: 9.5×10⁷ TCID₅₀/mL) in as little as 2 minutes.

### RT-PCR Testing of Nipah Virus RNA

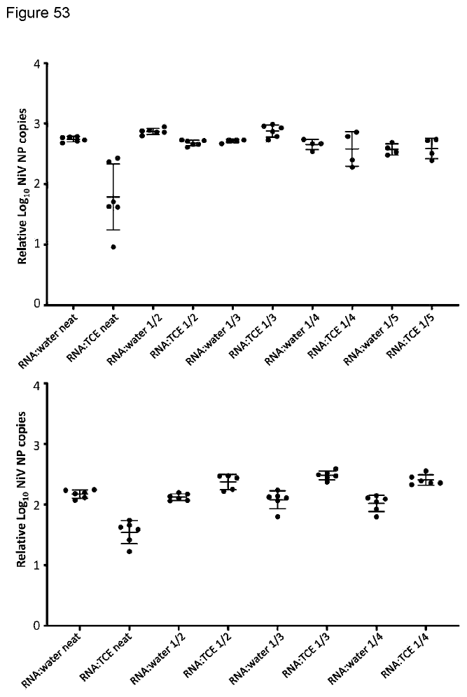

Sample Preparation Reagent E enables PCR testing of Nipah virus RNA. An RNA sample was mixed 1:1 with Sample Preparation Reagent E before serial dilution and addition to an RT-PCR (AgPath RT-PCR mix, Thermofisher). Without diluting the sample and adding 2 μL to the RT-PCR master mix, there was a significant reduction in the relative copy numbers calculated compared to a sample that was just diluted in a similar way with water. However, if the Sample Preparation Reagent E mixed sample was diluted, even as little as 1 in 2, before adding to the RT-PCR master mix, there was no effect from the Sample Preparation Reagent E on copy numbers calculated (FIG. 53).

### EXAMPLE 17: Sample Preparation Reagent can Inactivate Salmonella and Enable Sensitive PCR Detection

Sample preparation reagent was trialled to determine if it would inactivate Salmonella, a Gram-negative bacteria, and also enable detection of Salmonella by qPCR.

A variety of Sample preparation Reagent Formulations were trialled for their ability to inactivate Salmonella abony. In this experiment, Sample Preparation Reagent E was diluted to different percentages using 200 mM Tris pH 8.8. The percentage of Sample Preparation Reagent E required to inactivate S. abony was initially determined to be 25% or higher (Table 42), with the bacteria not inactivated at 17% Reagent E. Further testing identified bacteria could be inactivated at Sample Preparation Reagent concentrations of 21% or higher (Table 43), with the bacteria only partially inactivated at 17 and 18% Reagent E concentrations.

TABLE 42
Trialling different sample preparation reagent formulations
for their ability to inactivate S. abony and E. coli.

| | | Reagent E | | |
|---|---|---|---|---|
| | | percentage | | |
| Sample Preparation Reagent | | after | | |
| Formulation | | mixing | | |
| Reagent E | Tris | 1:1 with | Bacterial inactivation Results | |
| percentage | 200 mM | bacteria | | E. coli |
| % | % | % | S. abony | (control) |
| 100% | 0% | 50% | Inactivated | Inactivated |
| 50% | 50% | 25% | Inactivated | Inactivated |
| 33% | 67% | 17% | Not | Not |
| | | | inactivated | inactivated |
| 25% | 75% | 13% | Not | Not |
| | | | inactivated | inactivated |
| 20% | 80% | 10% | Not | Not |
| | | | inactivated | inactivated |
| 17% | 83% | 8% | Not | Not |
| | | | inactivated | inactivated |

TABLE 43
Trialling additional sample preparation reagent formulations for their ability to inactivate S. abony.

| | | Conditions for mixing | | | | | |
|---|---|---|---|---|---|---|---|
| Sample Preparation | | with S. abony culture | | | | | |
| Reagent Formulation | | | | Reagent E | Inactivation results | | |
| Reagent E | Tris | Volume | Volume | percentage | Bacterial | Log | |
| percentage | 200 mM | Reagent | bacteria | after mixing | growth | reduction | |
| (%) | (%) | (HL) | (HL) | (%) | (Cfu/mL) | from control | Conclusion |
| 100% | 0% | 25 | 25 | 50% | 0 | 8.40 | Inactivated |
| 33% | 67% | 70 | 10 | 29% | 0 | 8.40 | Inactivated |
| 25% | 75% | 70 | 10 | 22% | 0 | 8.40 | Inactivated |
| 20% | 80% | 70 | 10 | 18% | 1 × 10⁵ | 3.70 | Not inactivated |
| 33% | 67% | 50 | 10 | 28% | 0 | 8.40 | Inactivated |
| 25% | 75% | 50 | 10 | 21% | 0 | 8.40 | Inactivated |
| 20% | 80% | 50 | 10 | 17% | 2.7 × 10⁷ | 1.27 | Not inactivated |

| | | | | | | |
|---|---|---|---|---|---|---|
| Control (no Reagent E) | 0 | 10 | 0 | 5 × 10⁸ | N/A | Not inactivated |

Sample Preparation Regent E was also trialled for detection of cultured Salmonella by real-time PCR. Cultured S. abony (5.67×10⁸ Cfu/mL) was diluted in PBS and each dilution was mixed 1:1 with either Sample Preparation Reagent E or PBS (as a control). These were then tested by real-time PCR (1 μL/reaction) for detection of the Salmonella InvA gene (Table 44). Sample Preparation Reagent E could enable subsequent PCR detection of down to 2.8 Cfu/mL (3/3 replicates), which was 100× improved sensitivity compared to the PBS control (3/3 replicates at 283 Cfu/mL). In addition, the dilutions prepared using Sample Preparation Reagent E had improved Ct values (average improvement of 2.1) compared to PBS alone. This data shows that Sample Preparation Reagent E improves detection of cultured bacteria compared to no sample preparation treatment (PBS control).

TABLE 44
PCR detection of Salmonella abony cultures
prepared using Sample Preparation Reagent E.

| | | | | | | | Sample Preparation | |
|---|---|---|---|---|---|---|---|---|
| | | | | Salmonella:Sample | | | Reagent E | |
| Amount of | Salmonella:PBS | | | Preparation | | | improvement | |
| bacteria in | (1:1, control) | | | Reagent E (1:1) | | | compared to PBS | |
| sample | Av | Stdev | Replicates | Av | Stdev | Replicates | Ct | |
| Cfu/reaction | Ct | Ct | (/3) | Ct | Ct | (/3) | difference | |
| 283333 | 20.7 | 0.16 | 3 | 19.3 | 0.09 | 3 | 1.3 | Average |
| 28333 | 24.6 | 0.33 | 3 | 22.3 | 0.11 | 3 | 2.4 | 2.1 |
| 2833 | 28.1 | 0.31 | 3 | 25.8 | 0.06 | 3 | 2.3 | Stdev |
| 283 | 31.9 | 0.41 | 3 | 29.4 | 0.12 | 3 | 2.5 | 0.53 |
| 28 | 34.4 | 0.11 | 2 | 32.9 | 0.17 | 3 | Detected | |
| | | | | | | | 3/3 | |
| 2.8 | — | — | 0 | 36.2 | 0.76 | 3 | Detected | |
| | | | | | | | 3/3 | |
| 0.28 | — | — | 0 | 38.7 | 0.19 | 2 | — | |

Legend.
Av Ct—average cycle threshold value,
Stdev Ct—standard deviation of the cycle threshould value,
Replicates (/3)

Collectively, this data shows that Sample Preparation Reagent E can both inactivate Salmonella Abony, and enable sensitive PCR detection (down to 2.8 Cfu/μL).

### EXAMPLE 18: Sample Preparation Reagent can Inactivate Some Bacteria in Urine and Enable Sensitive PCR Detection of Spiked-Urine Samples

The ability of Sample Preparation Reagent E to a) inactivate and b) process spiked artificial urine for downstream molecular testing was assessed. For this, artificial urine was spiked with either a) E. coli, b) K. pneumoniae or c) E. hormaechei and then processed with either Sample Preparation Reagent E or 0.9% sterile saline (control), using a 1:1 ratio of spiked urine: processing reagent. After incubation of the mixtures at room temperature for 10 min, the processed samples were tested for bla_{OXA-48}, bla_{KPC} and blamp with either real-time PCR or the RPA-LFD assays.

Inactivation results show that while Sample Preparation Reagent E was able to inactivate some of the bacteria present, it was unable to inactivate all flora found in the artificial urine. This differed from previous testing where 0.5 McFarland standards of E. coli, K. pneumoniae or E. hormaechei could be fully inactivated when mixed 1:1 with Sample Preparation Reagent E and incubated for 10 min at room temperature. This suggests that the artificial urine contained microorganisms that were more robust and might need a longer incubation step or higher ratio of Sample Preparation Reagent E than used in this testing to be fully inactivated.

Testing the samples processed with Sample Preparation Reagent E with real-time PCR (Qiagen QuantiTect probe mastermix) and the RPA-LFD assays showed (Table 45) that both molecular diagnostic methods were compatible with Sample Preparation Reagent E, with real-time PCR being able to detect down to <10 CFU/μL of bacteria added to the artificial urine. Similar results were seen with the RPA-LFD assay, with the assays 5-fold less sensitive than real-time PCR, except for the bla_{OXA-48} RPA-LFD, which was as sensitive as real-time PCR.

TABLE 45
Summary of Sample Preparation Reagent E's ability to process spiked
artificial urine samples and use in downstream RPA-LFD and real-
time PCR reactions. Lowest detection limits are shown in table.

| Extraction | | | | |
|---|---|---|---|---|
| method | Gene target | RPA-LFD | | PCR |
| SPR | OXA-48 | 4.5 | CFU/μL | 4.5 CFU/μL |
| extracted | KPC | 22.1 | CFU/μL | 4.4 CFU/μL |
| (1:1) | IMP | 28.1 | CFU/μL | 5.6 CFU/μL |
| Not | OXA-48 | ND | | 4.5 CFU/μL |
| extracted | KPC | 88.2 | CFU/μL | 4.4 CFU/μL |

| | | | |
|---|---|---|---|
| (saline) | IMP | ND | 5.6 CFU/μL |
| Kit | OXA-48 | NT | 4.5 CFU/μL |
| extracted | KPC | NT | 4.4 CFU/μL |
| | IMP | NT | 5.6 CFU/μL |

SPR: Sample Preparation Reagent E,
ND: Not detected,
NT: Not tested

### EXAMPLE 19: Sample Preparation Reagent can Detect SARS-CoV-2 from Nasal and Saliva Swabs in a Range of Different Viral Transport Mediums

Sample Preparation Reagent E was tested for the ability to process samples for subsequent RT-qPCR detection, using SARS-CoV-2 spiked into (1) transport media alone, (2) transport media containing a saliva swab from a healthy participant, (3), transport media containing a nasal swab from a healthy participant.

Results indicated (Table 46) that Sample Preparation Reagent E was compatible for enabling RT-qPCR detection from samples consisting of transport medias alone (all tested), with most transport medias in the presence of a saliva swab, and in a select number of transport medias in the presence of a nasal swab.

These results demonstrate that Sample Preparation Reagent E can be used to process cultured SARS-CoV-2, and both saliva and nasal swabs collected in a range of different transport medias.

TABLE 46
Transport media compatibility testing results

| Transport media trialled | Media alone | Media + saliva swab | Media + nasal swab |
|---|---|---|---|
| Nuclease free water | PASS | PASS | Ct shift ~2 |
| (Invitrogen) | | | Reduces LOD by ½ log |
| RPMI 1640 + 2% FBS + antibiotics | PASS | PASS | Did not detect 25x LOD |
| (Thermofisher) | | | |
| Σ Virocult | PASS | PASS | PASS |
| (MWE Medical Wire) | | (Ct shift ~1.5) | (Ct shift ~3.5) |
| Σ Transwab + Liquid ames (blue | PASS | PASS | Did not detect 25x LOD |
| cap - ultra-fine swab, MWE) | (Ct shift ~1.5) | (Ct shift ~1.5) | |
| Σ Transwab + Liquid ames (orange | PASS | PASS | Ct shift ~1.7 |
| cap - standard swab, MWE) | | | Reduces LOD by ½ log |
| Transport Medium | PASS | PASS | PASS |
| (Vircell) | | | |
| Universal transport media | PASS | PASS | PASS |
| (Copan) | | | (Ct shift ~1.5) |
| Sodium Chloride 0.9% | PASS | PASS | PASS |
| (Baxter) | | | |
| Phosphate buffered saline pH 7.45 | PASS | PASS | Ct shift ~1.5, |
| (PBS; Gibco) | | | Reduces LOD by ½ log |
| Phosphate Buffer Solution | PASS | Ct shift ~2.8 | Did not detect 25x LOD |
| (Edwards) | | Reduces LOD by ½ | |
| | | log | |

LOD: limit of detection which was 1.5 log₁₀ CCID₅₀/mL or ~65 RNA copies/reaction; PASS: detected SARS-CoV-2 at LOD (3/3 replicates); Ct shift: Detection at LOD but with a noticeable shift in cycle threshold values (Ct) as indicated; Reduces LOD by ½ log: for trials that did not have consistent detection at LOD (3/3 replicates); Did not detect 25x LOD: For trials that did not detect a SARS-CoV-2 concentration 25 times the LOD (the highest concentration trialled).

### EXAMPLE 20: SARS-CoV-2 can be Detected in Samples Stored in Sample Preparation Reagent E at a Range of Incubation Temperatures and Times

Mock-infected SARS-CoV-2 samples were incubated with Sample Preparation Reagent E for a range of times and temperatures to determine the length of time samples could be stored in Reagent E and still enable sensitive detection of the viral RNA.

UV-inactivated SARS-CoV-2 was diluted in cell culture medium and exposed to Sample Preparation Reagent E (5:1 ratio, Sample: Reagent E). The Reagent E exposed samples were stored at room temperatures (22° C.), 4° C. and -20° C. for 10 min, 90 min, 5 hours, 24 hours, 72 hours, 1 week, and 2 weeks and 1 month. Each sample was then tested for presence of SARS-CoV-2 by RT-qPCR. Results (Table 47) indicated that SARS-CoV-2 could still be detected with high sensitivity after incubation at room temperature for 3 days, or two weeks at 4° C. and −20° C.

TABLE 47
Detection of SARS-CoV-2 after samples were incubated with Sample
Preparation Reagent E at a range of times and temperatures.

| | | Incubation temperature | | |
|---|---|---|---|---|
| Incubation time | | Room Temp | 4° C. | −20° C. |
| 10 | min | PASS | PASS | PASS |
| 90 | min | PASS | PASS | PASS |
| 5 | h | PASS^{w} | PASS | PASS |
| 24 | h | PASS | PASS | IMPROVED |
| | | (Ct shift ~2) | | |
| 72 | h | PASS | PASS | IMPROVED |
| | | (Ct shift ~2) | | |
| 1 | week | 25x LOD | PASS | PASS |
| | | (Ct shift ~4) | | |
| 2 | weeks | Did not detect | IMPROVED | IMPROVED |
| | | 25x LOD | | |
| 1 | month | Not tested | Did not detect | 25x LOD |

| | |
|---|---|
| 25x LOD | (Ct shift ~6) |

LOD: limit of detection which was 1.5 log₁₀ CCID₅₀/mL or ~65 RNA copies/reaction; PASS: detected SARS-CoV-2 at LOD (all replicates); Ct shift: Detection at LOD but with a noticeable shift in cycle threshold values (Ct) as indicated; 25x LOD: SARS-CoV-2 only detected at 25x the limit of detection. Did not detect 25x LOD: For trials that did not detect a SARS-CoV-2 concentration 25 times the LOD (the highest concentration trialled).
^{w}watch - only 1 replicate detected in one of the LOD dilutions tested. Improved: SARS-CoV-2 detection was improved (LOD 0.5 log₁₀CCID₅₀/mL; ~13 RNA copies/reaction).

In another configuration, Sample Preparation Reagent E was pre-diluted in 200 mM Tris pH 8.8 (1:5 ratio, Reagent E: Tris). This mixture (500 μL) was exposed to a human saliva swab (from a healthy individual). UV-inactivated SARS-CoV-2 was then diluted in the resultant mixture and incubated at either 4° C. or room temperature (22° C.) for 24 hours, 48 hours and 7 days. Each sample was then tested for presence of SARS-CoV-2 by RT-qPCR. Results (Table 48) indicated that SARS-CoV-2 could still be detected to the limit of detection after incubation at room temperature for 7 days.

TABLE 48
Stability of samples mixed with Sample Preparation Reagent E

| Sample incubation | | | |
|---|---|---|---|
| temp | 24 hours | 48 hours | 7 days |
| Fresh | PASS | PASS | PASS |
| 4 C. | PASS | PASS | PASS |
| RT | PASS | PASS | PASS |

PASS: detected SARS-CoV-2 at limit of detection (3/3 replicates) which was 1.5 log₁₀ CCID₅₀/mL or ~65 RNA copies/reaction.

These results demonstrate that samples containing SARS-CoV-2 and a compatible transport media that are exposed to Sample Preparation Reagent E can be stored at a range of temperatures for subsequent sensitive RT-qPCR detection after several days and even as long as two weeks in certain circumstance.

### EXAMPLE 21: Sample Preparation Reagent can be Used for Genetic Testing, Including Processing of Cells and Tissue for PCR and Mass Array Detection

Sample Preparation Reagent E was tested to see if it could prepare canine swab samples for mass array single nucleotide polymorphism (SNP) genetic testing.

Two canine swab samples were first tested for detection of the mammalian β-actin gene by PCR. Sample Preparation Reagent E (1 part) was pre-diluted with either 1 part water (1:2), or 5 parts water (1:5). Canine swabs were then added to 1.5 mL Eppendorf tubes containing 300 μL of either solution and incubated for 10 minutes at room temperature before being stored at 4° C. Samples were then tested for detection of the β-actin gene by PCR either undiluted (1×) or diluted 1/10 or 1/100 in water. A no-sample control was included to determine background-level detection of the mammalian β-actin gene (which could, for example, detect the operator's human DNA). Results (Table 49) demonstrated that a high level of mammalian β-actin gene could be detected in the canine swab samples (Ct values from 24-32) compared to the no-DNA control sample (Ct values >36). The gene was detected highest in the undiluted samples (Ct values 23-24), followed by the 1/10 dilution (Ct values 27-28), and then finally in the lowest dilution trialled (1/100; Ct values 31-32). Slightly improved Ct values were observed for samples that were prepared using the more dilute Sample Preparation Reagent E (1:5), suggesting that Sample Preparation Reagent E may have been slightly inhibitory to the PCR reaction.

TABLE 49
PCR testing of canine swab samples prepared using Sample Preparation
Reagent E, for detection of the mammalian β-actin gene.

| | Dilution in | | | | | | |
|---|---|---|---|---|---|---|---|
| | water | | | | | | |
| | (after | Swab A | | | Swab B | | |
| | sample | Ct | Ct | Replicates | Ct | Ct | Replicates |
| | prep) | Average | STDEV | (/2) | Average | STDEV | (/2) |
| 1 part | 1x | 24.400 | 0.437 | 2 | 24.588 | 0.004 | 2 |
| Reagent E | 1/10 | 28.231 | 0.450 | 2 | 28.689 | 0.021 | 2 |
| mixed with 1 | 1/100 | 31.769 | 0.379 | 2 | 32.174 | 0.312 | 2 |
| part water | No sample | 39.866 | — | 1 | 36.969 | — | 1 |
| 1 part | 1.00E+00 | 23.800 | 0.474 | 2 | 24.326 | 0.056 | 2 |
| Reagent E | 1.00E+01 | 27.658 | 0.002 | 2 | 28.478 | 0.070 | 2 |
| mixed with 5 | 1.00E+02 | 31.079 | 0.598 | 2 | 31.945 | 0.036 | 2 |
| parts water | No sample | 41.185 | — | 1 | 39.747 | 1.120 | 1 |

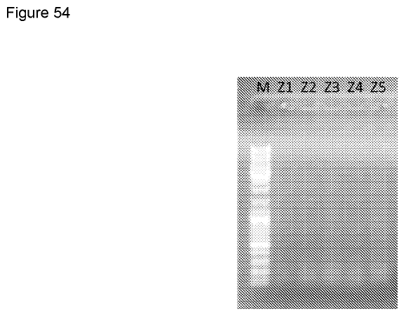

Samples were subsequently prepared for testing on two commonly used genotyping technologies being used commercially for Canine genotyping: (a) Bead-based Microarray Technology (Infinium Array, Illumina) on which probes bind to a complementary sequence in the sample DNA allowing allele identification, and (b) MassArray System (Agena Bioscience) which couples mass spectrometry with end-point PCR, enabling highly multiplexed reactions under universal cycling conditions to provide accurate, sensitive and rapid genetic analysis. Sample preparation reagent E (1 part) was pre-diluted with 5 parts of either (A) Water or (B) AE buffer (10 mM Tris-Cl, 0.5 mM EDTA). Canine swabs were then added to 1.5 mL Eppendorf tubes containing 300 μL of either solution and incubated for 10 minutes at room temperature before being stored at 4° C. Visualisation by gel electrophoresis confirmed DNA was extracted regardless of whether water or AE buffer was used as the diluent (FIG. 54). Two samples (Z3 and Z5) were chosen for external testing. Each was tested undiluted or diluted in water 1/2 or 1/5 to assess any inhibitory effects of Sample Preparation Reagent E. Also included was a swab collected at the same time as the other samples, from the same dog, but extracted using a standard magnetic bead-based DNA purification protocol. This sample was used as the comparator/control for analysis of performance (call rate and concordance measures) for each sample prepared with Sample Preparation Reagent E.

All samples prepared were tested for detection using the Illumina Infinium assay (which detects >10,000 SNPs) and the Agena MassArray (which detects 113 different SNPs). For the Illumina assay, 5 of the samples prepared using Sample Preparation Reagent E were 100% concordant with the control sample prepared using a standard DNA purification, with the most diluted sample (Z3 diluted 1/5 in water) giving near concordant values (99.4%). Call rate is an important Quality Control measure of DNA quality for the array, with greater than 0.95 (95%) commonly considered as industry standard for acceptance. All canine DNA extractions (control and the six Reagent E extracted and diluted samples) returned call rates exceeding 0.99 which demonstrates excellent input DNA quality. With the Agena assay, the two most diluted samples (Z3 and Z5 diluted 1 in 5) were ˜95% concordant with the sample prepared using a standard DNA purification (Table 50). It is well known that the MassArray technology is sensitive to salts, so the results confirm that dilutions can remove this issue while retaining DNA of sufficient quality/quantity for genotyping. Further dilutions are recommended for future optimisation if required.

These results demonstrate that Sample Preparation Reagent E is suitable for rapid sample preparation (without the need for expensive column- or magnetic bead-based DNA purification methods) from canine swab samples for subsequent genetic testing, using either PCR, Illumina assay, or the Agena assay.

TABLE 50
Mass array testing of canine swab samples prepared using Sample Preparation Reagent E

| | | | Illumina Assay | | Agena Assay | |
|---|---|---|---|---|---|---|
| | | Post- | | Concordance | | Concordance |
| | | extraction | | to Lab- | | to Lab- |
| | Sample | Dilution | Call | Extracted | Success | Extracted |
| Sample | Preparation | (in water) | Rate | DNA | Count | DNA |
| Z3 | Place swab into 1 | undiluted | 0.9969 | 100.00% | 5 | 4.40% |
| | part Reagent E | ½ | 0.9962 | 100.00% | 81 | 71.70% |
| | mixed with 5 parts | ⅕ | 0.9906 | 99.40% | 107 | 94.70% |
| | Water | | | | | |
| Z5 | Place swab into 1 | undiluted | 0.9975 | 100.00% | 5 | 4.40% |
| | part Reagent E | ½ | 0.9969 | 100.00% | 67 | 59.30% |
| | mixed with 5 parts | ⅕ | 0.9956 | 100.00% | 108 | 95.60% |
| | AE buffer | | | | | |
| Standard | | Undiluted | 0.9981 | 100.00% | 113 | 100.00% |
| DNA | | | | | | |
| purification | | | | | | |

Sample Preparation Reagent E was also tested for detection of genetic material from prawns.

Prawn pleopods (swimming legs) were removed from prawns and added to Sample Preparation Reagent E (3 μL per mg tissue) in a 1.5 mL Eppendorf tube. Each was mixed briefly with a pestle and incubated at 10 minutes at room temperature. After 10 minutes, the sample was diluted with either 5 volumes (1A and 1B) or 10 volumes (1C and 1D) sterile-grade water and mixed. Solutions were spun briefly and the supernatant collected in a separate tube and stored cold (4° C.), or at room Temperature (22° C.), 37° C., or 50° C. for 7 days. A positive control sample was prepared by extracting DNA using a standard laboratory (column-based) DNA extraction kit. All samples were diluted 1/50 in water for subsequent PCR detection and visualisation of genomic DNA extracted using agarose gel electrophoresis.

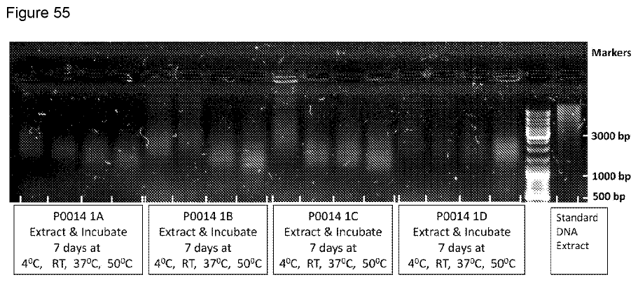

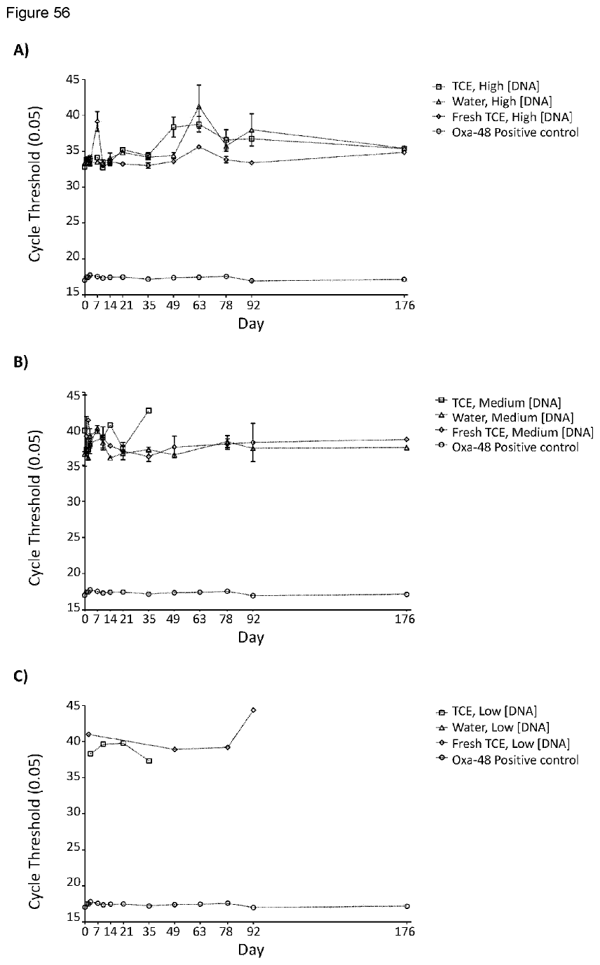

Gel electrophoresis demonstrated that after 7 days incubation at different temperatures, the DNA size range was approximately 1000-3000 bp fragments, with a small decrease in size observed as the storage temperature increased (FIG. 55). Presence of amplifiable DNA was confirmed using real-time PCR detection of β-actin. PCR testing indicated that the prawn β-actin gene could be detected in all samples, with no significant difference in Ct value regardless of the storage temperature of the sample (Table 51). The lack of detection in some of the replicates is most likely due to operator error, as there was no trend between samples on whether replicates were more likely to be detected at lower, higher, or moderate storage temperatures. Overall, the testing detected 37/40 replicates, resulting in a sensitivity of detection of 92% (with 95% confidence interval range from 79.61% to 98.43%).

TABLE 51
PCR testing of four Prawn samples prepared using Sample Preparation Reagent E.

| | Sample 1 | | | Sample 2 | | | Sample 3 | | | Sample 4 | | |
|---|---|---|---|---|---|---|---|---|---|---|---|---|
| | Av | Stdev | #Pos | Av | Stdev | #Pos | Av | Stdev | #Pos | Av | Stdev | #Pos |
| Storage Temp. | Ct | Ct | (/2) | Ct | Ct | (/2) | Ct | Ct | (/2) | Ct | Ct | (/2) |
| Minus 80° C. | 25.38 | 0.13 | 2 | 28.63 | 0.11 | 2 | 24.97 | 0.10 | 2 | 26.31 | 0.19 | 2 |
| +4° C. | 26.85 | 0.17 | 2 | 26.55 | 0.01 | 2 | 28.05 | — | 1 | 27.53 | 0.07 | 2 |
| Room temp (+22° C.) | 27.19 | 0.01 | 2 | 27.09 | 0.05 | 2 | 26.56 | 0.15 | 2 | 27.18 | 0.12 | 2 |
| +37° C. | 27.22 | 0.22 | 2 | 26.46 | 0.16 | 2 | 26.49 | 0.66 | 2 | 25.52 | — | 1 |
| +50° C. | 27.53 | 0.27 | 2 | 26.35 | 0.02 | 2 | 25.95 | 0.15 | 2 | 24.86 | — | 1 |
| Positive control | 26.41 | 0.09 | 2 | | | | | | | | | |
| (purified prawn DNA | | | | | | | | | | | | |
| diluted 1/1000) | | | | | | | | | | | | |

Table legend:
Av Ct—average cycle threshold value, Stdev Ct—standard deviation of the cycle threshold value, #Pos - (/2) number of positive replicates out of the total number replicates (two).

These results show that Sample Preparation Reagent E can be used for DNA genetic testing of prawn tissues by subsequent detection of β-actin genes via real-time PCR.

Finally, we trialled Sample Preparation Reagent E for detection of both human DNA and RNA in cultured human B cells. DNA detection targeted the RNAse P gene, with only 2 copies per cell. RNA detection targeted the glyceraldehyde-3-phosphate dehydrogenase (GAPDH) RNA transcript, known to be expressed at higher copies/cell, and targeted a genomic region interrupted by an intron to ensure only transcribed RNA was detected, and not genomic DNA.

A variety of sample preparation protocols were trialled to enable for subsequent RT-qPCR detection of both RNAse P and GAPDH, to determine the protocol with the most favourable Ct value and least dilution of sample (Table 52). The optimal protocol mixed 5 parts cells with 1 part Sample Preparation Reagent E, incubated for 5 minutes at room temperature, followed by a 1/2 dilution in nuclease free water before subsequent PCR testing.

TABLE 52
Optimal dilution of B cells mixed with Sample Preparation Reagent E for subsequent
RT-PCR detection of both RNAse P genomic DNA and GAPDH RNA transcripts.*

| Reagent E | | RNAse P genomic DNA PCR | | | GAPDH RNA transcript RT-PCR | | |
|---|---|---|---|---|---|---|---|
| dilution into B | Post-incubation | | | #Pos/ | | | #Pos/ |
| cells | diution (1/X) | Ct | Stdev | #Reps | Ct | Stdev | #Reps |
| None | None | 35.5 | 0.47 | 3 | | | |
| ⅙. | None | ND | | 0 | 24.49 | 1.93 | 3 |
| ¼. | None | ND | | 0 | 16.08 | FAIL | 1 |
| ½. | None | ND | | 1 | ND | | 1 |
| None | 2 | 35.4 | 0.73 | 3 | 22.42 | 0.19 | 3 |
| ⅙. | 2 | 28.7 | 0.43 | 3 | 21.26 | 0.43 | 3 |
| ¼. | 2 | 29.7 | 0.07 | 3 | 20.96 | 0.05 | 3 |
| ½. | 2 | ND | | 1 | 22.79 | 0.24 | 3 |
| None | 5 | 36.6 | 1.65 | 3 | 20.96 | 0.43 | 3 |
| ⅙. | 5 | 30.3 | 0.30 | 3 | 19.97 | 0.07 | 3 |
| ¼. | 5 | 30.1 | 0.10 | 3 | 23.27 | 1.54 | 3 |
| ½. | 5 | 30.7 | 1.20 | 3 | 22.94 | FAIL | 3 |

| | | | | |
|---|---|---|---|---|
| NTC | ND | 0 | ND | 0 |

*Sample Preparation Reagent E was diluted in cultured B cells (0.5 million cells in 50 μL PBS RNAse out buffer; SCHU IHW09013, Fred Hutch, Seattle, WA, USA) as indicated and incubated for 5 minutes at room temperature before dilution in nuclease free water as indicated. PCR was performed by adding 2.5 μL prepared and diluted sample to 7.5 μL master mix. RNAse P detection used the TaqMan RNAseP TaqMan ™ Copy Number Reference Assay (Thermofisher, Waltham, MA USA) mixed with the GoTaq Probe qPCR Master Mix with dUTP (Promega, Madison, WI, USA). GAPDH detection used the TagMan ™ RNAse P Gene expression assay (Thermofisher, Waltham, MA USA) with the GoTaq ® Probe qPCR Master Mix with dUTP (Promega) with the GoScript ™ RT Mix for 1-Step RT-qPCR (Promega, Madison, WI, USA). Optimal Reagent E procedure is highlighted in the row with the red borders.

Analytical sensitivity and limit of detection testing (Table 53) determined cells could be detected from 500 cell/μL to a limit of detection of 10 cells/μL when detecting of genomic DNA (RNAse P gene), or the equivalent of 50 copies/reaction. RNA transcripts (GAPDH) were detected from 500 cells/μL to a limit of detection of 0.25 cells/μL. Inter-assay variability was low, with an average cycle threshold (Ct) value standard deviation of less than 1.5 between trials, which represents less than 1/2 logo variability between tests (1 log 10 variability is approximately 3.3).

TABLE 53
Inter-assay variability and limit of RT/PCR detection of RNAse P (genomic DNA) and
GAPDH (RNA transcripts) in B cells processed using Sample Preparation Reagent E.

| RNAse P genomic DNA PCR test | | | | | | | GAPDH RNA transcript RT-PCR test | | | | | | |
|---|---|---|---|---|---|---|---|---|---|---|---|---|---|
| Cells/ | Ct | st | | | % | # | Cells/ | Ct | | | | % | # |
| μL | av | dev | #pos | #tot | pos | trials | μL | av | stdev | #pos | #tot | pos | trials |
| 500 | 28.7 | 0.01 | 6 | 6 | 100% | 2 | 500 | 21.1 | 1.51 | 26 | 26 | 100% | 6 |
| 50 | 32.6 | 0.62 | 21 | 21 | 100% | 5 | 50 | 23.6 | 0.00 | 6 | 6 | 100% | 2 |
| 20 | 34.2 | 0.84 | 20 | 20 | 100% | 1 | 5 | 27.8 | 0.94 | 26 | 26 | 100% | 6 |
| 10 | 36.6 | 0.53 | 51 | 52 | 98% | 3 | 0.05 | 35.2 | 1.00 | 63 | 64 | 98% | 3 |
| 5.0 | 37.4 | 0.5 | 21 | 24 | 88% | 2 | 0.25 | 32.9 | 0.19 | 52 | 55 | 95% | 4 |

*Cultured B cells (frozen and stored for less than 1 month) were serially diluted in PBS (24 μL) and mixed with 6 μL Sample Preparation Reagent E and incubated for 5 minutes at room temperature, before addition of 30 μL nuclease free water. Resultant dilutions (2.5 μL) were added to PCR reactions (7.5 μL) for detection of RNAse P (Genomic DNA) and GAPDH (RNA transcripts) as described in Table 52. The average cycle threshold (Ct) value was calculated from a series of experiments performed across multiple trials (# trials) and inter-assay variability was determined by calculating the standard deviation of the average Ct value across trials (stdev). The total number of replicates (#total) and the total number of positive replicates (#pos) was cumulated for all trials, and used to determine the percentage of replicates positive across all assays (% positive). Limit of detection was determined from the concentration of cells with >95% positive replicates across all trials. #Intra-assay variability (Ct value stdev within trial) reported due to only one trial of this concentration. Limit of detection is highlighted in the rows with red borders.

A protocol was designed to assess Sample Preparation Reagent E's capability of processing high, medium, and low concentrations of cells with a larger number of replicates. While the Ct value shifted slightly compared to the earlier experiments (due to a change in the way the cells were processed and stored ahead of time for testing larger replicate numbers) this testing confirmed earlier results (Table 54). Even with repeat testing, the analytical sensitivity of detection remained the same for both RNAse P (genomic DNA detection between 500 cells/μL and 10 cells/μL) and GAPDH (RNA transcripts limit of detection to 0.25 cells/μL).

TABLE 54
More than 1 month old frozen B cells: inter-assay variability and limit of RT/PCR detection of RNAse
P (genomic DNA) and GAPDH (RNA transcripts) after processing with Sample Preparation Reagent E.*

| RNAse P genomic DNA PCR test | | | | | | | | GAPDH RNA transcript RT-PCR test | | | | | | | |
|---|---|---|---|---|---|---|---|---|---|---|---|---|---|---|---|
| Cells/ | | | | | % | | Ct | Cells/ | Ct | | | | % | # | Ct |
| μL | Ct av | stdev | #pos | #tot | pos | # trials | shift | μL | av | stdev | #pos | #tot | pos | trials | shift |
| 500 | 30.6 | 0.868 | 60 | 60 | 100% | 12 | 1.8 | 500 | 23 | 1.139 | 60 | 60 | 100% | 12 | 1.6 |
| 50 | 33.1 | 0.470 | 60 | 60 | 100% | 12 | 0.5 | Not tested | | | | | | | |
| 20 | 34.4 | 0.434 | 60 | 60 | 100% | 12 | 0.2 | 5 | 31 | 1.482 | 60 | 60 | 100% | 12 | 2.9 |
| 10 | 35.7 | 0.566 | 58 | 60 | 97% | 12 | −0.8 | 0.25 | 36 | 1.566 | 59 | 60 | 98% | 12 | 3.0 |

*Cultured B cells frozen for more than one month were tested for sample processing with Sample Preparation Reagent E over a three week period, using the procedure as described in Table 52 and Table 53. “Ct shift” indicates the shift in average Ct values from these trials compared to the trials reported in Table 53. Limit of detection is highlighted in the row with the red borders.
Abbreviations: “undt,” undetected.

These results show that Sample Preparation Reagent E can be used to prepare cultured human B cells for genetic testing by RT-qPCR for both human DNA (RNase P) and human RNA (GAPDH).

Collectively, the canine swab, prawn tissue, and human B cell testing results show that Sample Preparation Reagent E is suitable for processing samples for genetic testing (either DNA or RNA).

### EXAMPLE 22: Sample Preparation Reagent E is Stable for Up to 23 Months when Stored at Temperatures Ranging from Minus 20C to 37C, and 3 Months when Stored at Minus 80 C and 45 C

Storage and shipping stability testing at different temperatures was performed on Sample Preparation Reagent E, with testing results summarised in Table 55. Cumulatively, results indicated the Sample Preparation Reagent was stable for at least 23 months when stored between −20° C. to 37° C., and 3 months when stored at 50° C. or −80° C.

TABLE 55
Storage stability of Sample Preparation Reagent E batches at different temperatures.

| | | | Testing performed | | | |
|---|---|---|---|---|---|---|
| Incubation | Incubation | Reagent E batch | SARS-CoV-2 | Dengue virus | K. pneumoniae | K. pneumoniae |
| temperature | time | tested | detection¹ | inactivation² | inactivation³ | detection⁴ |
| −80° C. | 1 month | E007 | PASS | Not tested | Not tested | Not tested |
| −80° C. | 3 months | E007 | Not tested | PASS | Not tested | Not tested |
| −20° C. | 23 months | PRE001 | Not tested | PASS | Not tested | Not tested |
| −20° C. | 27 months | PRE001 | Not tested | Not tested | PASS | PASS |
| 4° C. | 6 months | E007 | PASS | Not tested | Not tested | Not tested |
| RT | 23 months | PRE001 | PASS | PASS | PASS | PASS |
| 37° C. | 23 months | PRE001 | PASS | PASS | PASS | PASS |
| 50° C. | 3 months | E007 | PASS | PASS | Not tested | Not tested |

¹For SARS-CoV_2 detection, a pass indicates that after sample processing with 5 parts sample and 1 part Sample Preparation Reagent E, the virus was detected by RT-qPCR down to the limit of detection (3/3 replicates at 32 CCID₅₀/mL or 64 RNA copies/reaction) and Ct values were consistent for the test and reference sample (<±1.65 Ct value standard deviation).
²For Dengue virus inactivation, a pass indicates that after sample processing with 5 parts sample (DENV-1, ET00.243, 2 × 10⁶ TCID₅₀/mL in cell culture media) mixed with 1 part Sample Preparation Reagent E, the virus was inactivated with no virus detected by TCID₅₀ ELISA testing.
³For K. Pneumoniae inactivation, a pass indicates that after sample processing with 1 part sample (0.5 McFarland's standard, 9.33 × 10⁷ cfu/mL bacterial culture in saline) mixed with 1 part Sample Preparation Reagent E, the bacteria was inactivated, with no bacterial growth observed by bacterial culture testing.
⁴For K. Pneumoniae detection, a pass indicates that after sample processing with 1 part sample (0.5 McFarland's standard, 9.33 × 10⁷ of bacterial culture in saline) mixed with 1 part Sample Preparation Reagent E, the bacteria was detected by real-time PCR down to the limit of detection (a 10⁻⁴ dilution, 2/2 replicates) and Ct values were consistent for the test and reference sample (<±1.65 Ct value standard deviation).

### EXAMPLE 23: Sample Preparation Reagent E can be Made Using Chemicals from More than One Supplier

A further three batches (E008, E009 and E010) of Sample Preparation Reagent E were produced by BioCifer, to determine if some reagents from alternative suppliers could be used for manufacturing without affecting quality of the reagent. All batches passed testing (Table 56) indicating that performance of Sample Preparation Reagent E is unlikely to be affected by changing suppliers of raw materials.

TABLE 56
Sample Preparation Reagent E made using chemical from different suppliers

| | Specification/ | | | |
|---|---|---|---|---|
| Location | Quality test | E008 | E009 | E010 |
| Batch | Deviations | None | Used | Used food-grade (rather |
| production | | | alternative | than molecular biology |
| information | | | supplier for | grade) version of Betaine |
| | | | water. | |
| | pH | 14.39 | 14.50 | 14.52 |
| Testing | Detection of SARS-CoV-2 RNA¹ | PASS | PASS | PASS |
| | Inactivation of dengue virus.² | PASS | PASS | PASS |
| | Detection of Klebsiella | PASS | PASS | PASS |
| | pneumoniae³ | | | |
| | Inactivation of Klebsiella | PASS | PASS | PASS |
| | pneumoniae⁴ | | | |
| | Inactivation of Salmonella | PASS | PASS | PASS |
| | abony⁵ | | | |

¹For SARS-CoV_2 detection, a pass indicates that after sample processing with 5 parts sample and 1 part Sample Preparation Reagent E, the virus was detected by RT-qPCR down to the limit of detection (3/3 replicates at 32 CCID₅₀/mL or 64 RNA copies/reaction) and Ct values were consistent for the test and reference sample (<±1.65 Ct value standard deviation).
²For Dengue virus inactivation, a pass indicates that after sample processing with 5 parts sample (DENV-1, ET00.243, 2 × 10⁶ TCID₅₀/mL in cell culture media) mixed with 1 part Sample Preparation Reagent E, the virus was inactivated with no virus detected by TCID₅₀ ELISA testing.
³For K. Pneumoniae detection, a pass indicates that after sample processing with 1 part sample (0.5 McFarland's standard, 9.33 × 10⁷ of bacterial culture in saline) mixed with 1 part Sample Preparation Reagent E, the bacteria was detected by real-time PCR down to the limit of detection (a 10⁻⁴ dilution, 2/2 replicates) and Ct values were consistent for the test and reference sample (<±1.65 Ct value standard deviation).
⁴For K. Pneumoniae inactivation, a pass indicates that after sample processing with 1 part sample (0.5 McFarland's standard, 9.33 × 10⁷ cfu/mL bacterial culture in saline) mixed with 1 part Sample Preparation Reagent E, the bacteria was inactivated, with no bacterial growth observed by bacterial culture testing.
⁵For S. abony inactivation, a pass indicates that after sample processing with 1 part bacterial culture sample (5 × 10⁸ Cfu/mL) mixed with 1 part Sample Preparation Reagent E, the bacteria was inactivated, with no bacterial growth observed by bacterial culture testing.

### EXAMPLE 24: Sample Preparation Reagent E can Inhibit RNAse and DNase

A series of experiments were performed to demonstrate the ability of Sample Preparation Reagent E to inhibit both DNase and RNase.

For DNase inhibition testing, Sample Preparation Reagent E (or water) was spiked with 0, 7 or 14 units of DNase and known amounts of bla_{OXA-48} copies/μL added to the mixture to test the reagent's ability to inhibit DNase function. Mixtures were incubated for 10 minutes at 37° C. for optimal DNase function, followed by a DNase inactivation step at 75° C., after which 1 μL of each mixture was tested with a bla_{OXA-48} real-time PCR assay. Results suggest that the Sample Preparation Reagent E has some ability to protect the sample from DNase activity, which is shown by the successful amplification of as low as 135.8 copies of bla_{OXA-48} when up to 14 units of DNase were added to the Sample Preparation Reagent E (Table 57).

TABLE 57
Sample Preparation Reagent E testing for DNase inhibition.

| | | | DNase | | | | Copies added | | Ct difference |
|---|---|---|---|---|---|---|---|---|---|
| Mixture | SPR-E | DNase | Buffer | Water | Template | Total | to DNase | | from no |
| no. | (μl)* | (units) | (μl) | (μl) | (μl) | (μl) | reaction | Ct | DNase mixture |
| 1 | 10 | 0 | 2 | 7 | 1 | 20 | 1358.2 | 32.14 | — |
| 2 | 10 | 7 | 2 | 3.5 | 1 | 20 | 1358.2 | 31.23 | −0.91 |
| 3 | 10 | 14 | 2 | 0 | 1 | 20 | 1358.2 | 32.49 | 0.35 |
| 4 | 0 | 0 | 2 | 17 | 1 | 20 | 1358.2 | 28.66 | — |
| 5 | 0 | 7 | 2 | 13.5 | 1 | 20 | 1358.2 | no amp. | n/a |
| 6 | 0 | 14 | 2 | 10 | 1 | 20 | 1358.2 | no amp. | n/a |
| 7 | 10 | 0 | 2 | 7 | 1 | 20 | 135.8 | 33.61 | — |
| 8 | 10 | 7 | 2 | 3.5 | 1 | 20 | 135.8 | 33.16 | −0.45 |
| 9 | 10 | 14 | 2 | 0 | 1 | 20 | 135.8 | 35.82 | 2.21 |
| 10 | 0 | 0 | 2 | 17 | 1 | 20 | 135.8 | 31.92 | — |
| 11 | 0 | 7 | 2 | 13.5 | 1 | 20 | 135.8 | no amp. | n/a |
| 12 | 0 | 14 | 2 | 10 | 1 | 20 | 135.8 | no amp. | n/a |
| 13 | 10 | 0 | 2 | 7 | 1 | 20 | 13.6 | 37.5 | — |
| 14 | 10 | 7 | 2 | 3.5 | 1 | 20 | 13.6 | no amp. | n/a |
| 15 | 10 | 14 | 2 | 0 | 1 | 20 | 13.6 | no amp. | n/a |
| 16 | 0 | 0 | 2 | 17 | 1 | 20 | 13.6 | 34.89 | — |
| 17 | 0 | 7 | 2 | 13.5 | 1 | 20 | 13.6 | no amp. | n/a |
| 18 | 0 | 14 | 2 | 10 | 1 | 20 | 13.6 | no amp. | n/a |
| 19 | 10 | 0 | 2 | 7 | 1 | 20 | 1.36 | no amp. | — |
| 20 | 10 | 7 | 2 | 3.5 | 1 | 20 | 1.36 | no amp. | n/a |
| 21 | 10 | 14 | 2 | 0 | 1 | 20 | 1.36 | no amp. | n/a |
| 22 | 0 | 0 | 2 | 17 | 1 | 20 | 1.36 | no amp. | — |
| 23 | 0 | 7 | 2 | 13.5 | 1 | 20 | 1.36 | no amp. | n/a |
| 24 | 0 | 14 | 2 | 10 | 1 | 20 | 1.36 | no amp. | n/a |

*SPR-E = Sample Preparation Reagent E;
Ct = cycle threshold − Ct values are based on a threshold set at 0.05;
no amp. = no amplification

Further testing focused on 13.6 copies of bla_{OXA-48} with the aim to determine the highest amount of DNase (units) that could be added to the Sample Preparation Reagent E/water that would allow us to still detect the DNA sample added to the reaction. The same protocol as above was used for this, with the results showing that 13.6 copies of bla_{OXA-48} could still be detected even at the highest DNase amount (7 units) tested. In comparison, addition of DNase (any amount) to water resulted in no detection of the DNA sample. These results further suggest that the Sample Preparation Reagent E has some protective ability, even when only 13.6 copies of bla_{OXA-48} and up to 7 units of DNase are present in the reaction (Table 58).

TABLE 58
Additional Sample Preparation Reagent E testing for DNase inhibition.

| | | | DNase | | | | | | |
|---|---|---|---|---|---|---|---|---|---|
| Mixture | SPR-E | DNase | Buffer | Water | Template | Total | Copies added to | | Ct difference from |
| no. | (μl)* | (units) | (μl) | (μl) | (μl) | (μl) | DNAse reaction | Mean Ct | no DNase mixture |
| 1 | 10 | 7.00 | 2 | 3.50 | 1 | 20 | 13.6 | 33.46 | 0.61 |
| 2 | 10 | 3.50 | 2 | 5.25 | 1 | 20 | 13.6 | 33.19 | 0.35 |
| 3 | 10 | 1.75 | 2 | 6.13 | 1 | 20 | 13.6 | 33.29 | 0.44 |
| 4 | 10 | 0.88 | 2 | 4.81 | 1 | 20 | 13.6 | 32.73 | −0.12 |
| 5 | 10 | 0.44 | 2 | 5.91 | 1 | 20 | 13.6 | 32.82 | −0.03 |
| 6 | 10 | 0.22 | 2 | 6.45 | 1 | 20 | 13.6 | 32.93 | 0.08 |
| 7 | 10 | 0 | 2 | 7.00 | 1 | 20 | 13.6 | 32.84 | — |
| 8 | 0 | 7.00 | 2 | 13.50 | 1 | 20 | 13.6 | ND | NA |
| 9 | 0 | 3.50 | 2 | 15.25 | 1 | 20 | 13.6 | ND | NA |
| 10 | 0 | 1.75 | 2 | 16.13 | 1 | 20 | 13.6 | ND | NA |
| 11 | 0 | 0.88 | 2 | 14.81 | 1 | 20 | 13.6 | ND | NA |
| 12 | 0 | 0.44 | 2 | 15.91 | 1 | 20 | 13.6 | ND | NA |
| 13 | 0 | 0.22 | 2 | 16.45 | 1 | 20 | 13.6 | ND | NA |
| 14 | 0 | 0 | 2 | 17 | 1 | 20 | 13.6 | 33.24 | — |

*SPR-E = Sample Preparation Reagent E;
Ct = cycle threshold − Ct values are based on a threshold set at 0.05;
NA = not applicable,
ND = not detected

Similarly, RNase spiked into Sample Preparation Reagent E did not interfere with sample preparation of SARS-CoV-2 samples and subsequent downstream RT-qPCR detection of the E gene of SARS-CoV-2. Results indicated that even high concentrations of RNase A (100 μg/mL or less) did not interfere (Table 59). For control purposes, SARS-CoV-2 samples were spiked with 100 μg/mL RNase A before processing with unspiked Sample Preparation Reagent E, and this resulted in a complete impairment of E gene amplification (data not shown). These results indicate that Sample Preparation Reagent E is able to inhibit up to at least 100 μg/mL RNAse.

TABLE 59
Sample Preparation Reagent E tolerance for up to 100 μg/mL RNAse.

| Sample | log10 | | | | | | | | |
|---|---|---|---|---|---|---|---|---|---|
| preparation | CCID₅₀/ | Ct | Ct | Wells | Ct | Ct | | Diff from | |
| Reagent E | mL | Rpt 1 | Rpt 2 | detected | Avg | Stdev | Stdev <2.33¹ | PRE001I | Diff <1.65² |
| control | 2.9 | 33.61 | 30.70 | 2 | 32.15 | 2.06 | Acceptable | 0.00 | Acceptable |
| | 2.2 | 35.19 | 34.36 | 2 | 34.78 | 0.59 | Acceptable | 0.00 | Acceptable |
| | 1.5 | 37.66 | 37.89 | 2 | 37.78 | 0.17 | Acceptable | 0.00 | Acceptable |
| | 0.8 | n.d. | n.d. | 0 | — | — | Fail | — | Fail |
| 100 μg/mL | 2.9 | 31.79 | 33.10 | 2 | 32.45 | 0.93 | Acceptable | 0.29 | Acceptable |
| RNase | 2.2 | 34.88 | 37.10 | 2 | 35.99 | 1.57 | Acceptable | 1.21 | Acceptable |
| | 1.5 | 36.35 | 37.90 | 2 | 37.12 | 1.10 | Acceptable | −0.65 | Acceptable |
| | 0.8 | n.d. | 37.46 | 1 | 37.46 | — | Fail | — | Fail |

Analyzed RT-qPCR results detecting the E gene in UV-inactivated SARS-CoV-2 samples (measured in duplicate) diluted in RMI 1640 cell culture medium after sample preparation with RNase A-spiked (100 μg/mL) Sample Preparation Reagent E batch or unspiked Sample Preparation Reagent E batch (PRE001) as control at room temperature for 10 minutes using the iTaq Universal Probes One-Step Kit (BioRad). Avg: Average; Ct: Cycle threshold; diff: difference; Rpt: repeat; Stdev: Standard deviation; TCE: Sample Preparation Reagent E. 1. Standard deviation acceptance limits were <2.33, and had Ct-values reported for both replicates. 2. Difference from sample prep with PRE001 defined acceptable Ct value differences as <1.65.

### EXAMPLE 25: Stability of DNA and RNA Spiked into TNA-Cifer Reagent E to Trial Persistence of DNA or RNA Detection

Sample Preparation Reagent E was trialled for the length of time DNA and RNA would persist in the reagent.

Sample Preparation Reagent E was spiked with high (˜6,791 target copies/μL), medium (˜679 target copies/μL) and low (˜68 target copies/μl) of DNA, stored at 4° C. and tested at different time-points to determine how long DNA contamination would persist in the reagent. Of those, low amounts of DNA spiked into Sample Preparation Reagent E and stored at 4° C. were only intermittently detected up to day 35, suggesting that this amount was at the limit of detection and may have been fully degraded by day 49. Similarly, medium amounts of DNA spiked into Sample Preparation Reagent E and stored at 4° C. could be detected up to day 35, but appear to have been fully degraded by day 49. In contrast, high amounts of DNA spiked into Sample Preparation Reagent E and stored at 4° C. could be detected throughout the testing period (176 days), suggesting that high (>6,791 target copies/μL) amounts of DNA contamination can persist for at least 176 days in the Sample Preparation Reagent E without substantial degradation (FIG. 566).

To assess how long contaminating RNA would persist at 4° C. storage temperature, kit-purified SARS-CoV-2 RNA in Sample Preparation Reagent E was tested for detection of RNA after incubation for 10 minutes at room temperature (positive control) as well as 24 hours at 4° C. storage. Results demonstrated that concentrations equating to viral copies as high as 3.7 log ₁₀CCID₅₀/mL (5.3×10³ CCID₅₀/mL), or approximately 2580 RNA copies/μL, could be detected after 10 minutes incubation at room temperature.

These results also show that if TNA-Cifer is contaminated with RNA (for example, during manufacture), this contamination will be degraded after 24 hours storage at 4° C. (Table 60). Further testing demonstrated that TNA-Cifer contaminated with RNA and held for 24 hours at 4C was fully functional at processing SARS-CoV-2 (data not shown).

TABLE 60
Sample Preparation Reagent E was not affected by RNA contamination up to 2580 copies/uL.

| | Sample | | | | | | |
|---|---|---|---|---|---|---|---|
| | Preparation | | | | | | |
| | Reagent E | Equivalent | Equivalent | | | | Stdev |
| Experiment | Incubation | viral | RNA | #Pos/ | Ct | Ct | Acceptance |
| # | time | log₁₀CCID₅₀/mL | copies/μL | #Tot | avg | stdev | (>2.33) |
| Expt 1 | 10 minutes RT | 3.7 | 2580 | 4/4 | 24.2 | 0.24 | Acceptable |
| | 24 hours 4 C. | 3.7 | 2580 | 0/2 | Undet | Undet | Fail |
| Expt 2 | 10 minutes RT | 3.7 | 2580 | 4/4 | 26.6 | 0.67 | Acceptable |
| | 24 hours 4 C. | 3.7 | 2580 | 0/2 | Undet | Undet | Fail |

Avg: Average; Ct: Cycle threshold; diff: difference; detec.: detected; Exp: experiment; F: Fail; n.d.: not detected; #pos: number of replicates testing positive; #Tot: total number of replicates tested; Stdev: Standard deviation. 1. Standard deviation acceptance limits were <2.33, with Ct-values reported for both replicates.

Further studies were performed to assess the stability of column-purified RNA in the presence of Sample Preparation Reagent E at room temperature. Column-purified SARS-CoV-2 RNA was either mixed with Sample Preparation Reagent E or water in a ratio of 5 parts RNA: 1 part Reagent E or water (12,435 copies/reaction) and tested for detection of SARS-CoV-2 RNA after incubation for different time points. Results suggested that RNA was already degraded after 30 minutes (ct difference ˜5) and that degradation continued for 2 hours (ct difference ˜8) before being completed degraded by 4 hours (Table 61).

TABLE 61
Stability of RNA (hours) in the presence of TNA-Cifer at room temperature.

| | | | | | | RNA with TNA-Cifer | | | |
|---|---|---|---|---|---|---|---|---|---|
| | | RNA | RNA alone | | | | Ct | Replicates | Ct |
| | | copies/reaction | Ct Av | Stdev | Rep/3 | Ct Avg | Stdev | (/3) | diff |
| 30 | min | 12,435 | 28.2 | 0.1 | 3 | 32.9 | 0.1 | 3 | 4.8 |
| 1 | h | 12,435 | 28.3 | 0.8 | 3 | 33.7 | 0.5 | 3 | 5.4 |
| 2 | h | 12,435 | 26.5 | 0.0 | 3 | 34.6 | 0.3 | 3 | 8.0 |
| 4 | h | 12,435 | 26.7 | 0.1 | 3 | Fail | Fail | 0 | NA |
| 6 | h | 12,435 | 28.2 | 0.1 | 3 | Fail | Fail | 0 | NA |

These results indicate that while unprotected DNA is relatively stable, and could be detected from 35 days to 176 days depending on the concentration of DNA, unprotected RNA could not be left for long periods of time in the presence of TNA-Cifer without degradation, and that samples containing unprotected RNA should therefore be tested immediately after a short incubation period with Sample Preparation Reagent E.

Additional studies were performed to determine if RNA could be stabilised after incubation with TNA-Cifer Reagent E by the addition of a neutralising buffer, to prevent the high-pH hydrolysis of unprotected RNA.

Column-purified SARS-CoV-2 RNA (25 μL) was mixed with TNA-Cifer Reagent E (5 μL) for 10 minutes and then mixed with 3 μL of 1M Tris of either pH 7, pH 8, and pH 8.8. These reactions were incubated for 4 hours at room temperature before testing for detection of SARS-CoV-2 RNA (Table 62). Results show that RNA left with TNA-Cifer for 4 hours was degraded compared to RNA alone (Ct difference ˜ 8.6), whereas RNA that had been neutralised by the Tris buffers were well stabilised (Ct difference 0.9-2).

TABLE 62
Degradation of RNA in the presence of TNA-Cifer Reagent E
for 4 hours, and rescue from degradation by neutralisation.

| | RNA | | | Replicates | | Av Ct |
|---|---|---|---|---|---|---|
| | copies/reaction | Ct Avg | Ct Stdev | (/3) | Ct diff | diff |
| RNA only | 1,243,540 | 19.26 | 0.15 | 3 | NA | NA |
| control | 124,354 | 22.95 | 0.19 | 3 | NA | |
| | 12,435 | 27.74 | 0.11 | 3 | NA | |
| RNA + | 1,243,540 | 25.78 | 0.03 | 3 | 6.52 | 8.6 |
| TNA-Cifer | 124,354 | 31.75 | 0.14 | 3 | 8.80 | |
| | 12,435 | 38.17 | 1.05 | 3 | 10.43 | |
| Tris pH 7 | 1,243,540 | 20.09 | 0.01 | 3 | 0.83 | 0.9 |
| rescue | 124,354 | 24.32 | 0.12 | 3 | 1.38 | |
| | 12,435 | 28.26 | 0.37 | 3 | 0.51 | |
| Tris pH 8 | 1,243,540 | 20.64 | 0.09 | 3 | 1.38 | 2.2 |
| rescue | 124,354 | 26.29 | 0.03 | 3 | 3.34 | |
| | 12,435 | 29.75 | 0.15 | 3 | 2.01 | |
| Tris pH 8.8 | 1,243,540 | 21.88 | 0.04 | 3 | 2.62 | 2.2 |
| rescue | 124,354 | 25.62 | 0.02 | 3 | 2.67 | |
| | 12,435 | 29.17 | 0.02 | 3 | 1.42 | |

Ct Avg: Average cycle threshold value for all detected replicates; Ct Stdev: standard deviation of cycle threshould values for all detected replicates; #Rep (/3): number of replicates detected out of a total of three replicates tested; h: hours; Ct diff: difference between replicate cycle threshould values and the control cycle threshold values.

A follow up study was performed to determine how long unprotected RNA could be stabilised by neutralisation, after a 10 minute incubation with TNA-Cifer Reagent E. Column-purified SARS-CoV-2 RNA (25 μL) was mixed with TNA-Cifer Reagent E (5 μL) for 10 minutes and then mixed with 3 μL of 1M Tris of either pH 6, pH 7, and pH 8. These reactions were tested for detection of SARS-CoV-2 RNA at 4 hours, 2 days, 3 days and 7 days (Table 63). Results show that RNA left with TNA-Cifer was degraded compared to RNA alone, whereas RNA that had been subsequently neutralised by the Tris buffers were stabilised and could be detected even as long as seven days incubation at room temperature (average Ct-difference compared to RNA-alone from was ˜4-8).

TABLE 63
Degradation of RNA in the presence of TNA-Cifer Reagent E for 7 days, and rescue from degradation by neutralisation.

| | | | | | | | | | | | | | | Day 7 | |
|---|---|---|---|---|---|---|---|---|---|---|---|---|---|---|---|
| | | 4 h | | | Day 2 | | | Day 3 | | | Day 7 | | | Ct diff | |
| | RNA copies/ | Ct | Ct | #Rep | Ct | Ct | #Rep | Ct | Stdev | #Rep | Ct | Stdev | #Rep | Ct | Av Ct |
| Sample | reaction | Avg | Stdev | (/3) | Avg | Stdev | (/3) | Avg | Ct | (/3) | Avg | Ct | (/3) | diff | diff′ |
| RNA only | 1,243,540 | 18.5 | 0.1 | 3 | 18.5 | 0.1 | 3 | 18.3 | 0.3 | 3 | 18.7 | 0.1 | 3 | NA | NA |
| | 124,354 | 21.9 | 0.1 | 3 | 22.1 | 0.0 | 3 | 22.1 | 0.1 | 3 | 22.8 | 0.1 | 3 | NA | |
| | 12,435 | 25.1 | 0.3 | 3 | 25.1 | 0.2 | 3 | 25.0 | 0.1 | 3 | 25.6 | 0.2 | 3 | NA | |
| RNA + | 1,243,540 | 34.4 | Fail | 1 | 31.7 | 0.1 | 3 | 31.7 | 0.3 | 3 | Fail | Fail | 0 | Fail | Fail |
| TNA-Cifer | 124,354 | Fail | Fail | 0 | 37.0 | 1.3 | 3 | 35.7 | 0.8 | 3 | Fail | Fail | 0 | Fail | |
| | 12,435 | Fail | Fail | 0 | Fail | Fail | 0 | 48.5 | Fail | 0 | Fail | Fail | 0 | Fail | |
| Rescue | 1,243,540 | 19.1 | 0.1 | 3 | 19.2 | 0.1 | 3 | 19.1 | 0.1 | 3 | 19.3 | 0.1 | 3 | 0.6 | 4.8 |
| Tris pH 6 | 124,354 | 22.9 | 0.1 | 3 | 23.1 | 0.2 | 3 | 23.2 | 0.2 | 3 | 23.7 | 0.1 | 3 | 0.9 | |
| | 12,435 | 37.6 | 1.3 | 3 | 36.9 | 1.7 | 3 | 35.8 | 0.6 | 3 | 38.6 | Fail | 1 | 13.0 | |
| Rescue | 1,243,540 | 19.0 | 0.2 | 3 | 19.4 | 0.1 | 3 | 20.2 | 0.0 | 3 | 23.2 | 0.2 | 3 | 4.5 | 7.6 |
| Tris pH 7 | 124,354 | 22.4 | 0.2 | 3 | 23.0 | 0.1 | 3 | 23.8 | 0.1 | 3 | 27.1 | 0.2 | 3 | 4.2 | |
| | 12,435 | 38.8 | 1.8 | 2 | 37.4 | 0.1 | 3 | 37.0 | 1.2 | 3 | 39.7 | 0.6 | 1 | 14.1 | |
| Rescue | 1,243,540 | 19.2 | 0.0 | 3 | 19.4 | 0.1 | 3 | 19.4 | 0.1 | 3 | 20.0 | 0.1 | 3 | 1.3 | 7.2 |
| Tris pH 8 | 124,354 | 25.6 | 0.1 | 3 | 27.2 | 0.1 | 3 | 28.6 | 0.2 | 3 | 33.2 | 0.3 | 3 | 10.3 | |
| | 12,435 | 29.9 | 0.1 | 3 | 32.8 | 0.2 | 3 | 33.4 | 0.5 | 3 | 35.5 | 0.8 | 3 | 9.9 | |

Ct Avg: Average cycle threshold value for all detected replicates; Ct Stdev: standard deviation of cycle threshould values for all detected replicates; #Rep (/3): number of replicates detected out of a total of three replicates tested; h: hours; Ct diff: difference between replicate cycle threshould values and the control cycle threshold values.

A second follow up study was performed to confirm protection of RNA when exposed to Sample Preparation Reagent E followed by neutralisation, and also to determine if this would improve detection of unpurified RNA within a virus particle (e.g. SARS-CoV-2 virus). SARS-CoV-2 Virus or purified SARS-CoV-2 RNA (25 μL) was mixed with TNA-Cifer Reagent E (5 μL) for only 5 minutes and then mixed with 3 μL of 1M Tris of either pH 6, pH 7, and pH 8. These reactions were tested for detection of SARS-CoV-2 RNA at 4 hours, 2 days, 3 days and 7 days (Table 64). Results showed that Both RNA and virus left with TNA-Cifer was progressively degraded over 7 days compared to RNA alone, as anticipated. However, virus and RNA that had been neutralised by the Tris buffers were stabilised and could be detected even as long a seven days incubation at room temperature (average Ct-difference compared to control RNA or virus was ˜3-10).

TABLE 64
Degradation of purified RNA and RNA within SARS-CoV-2 virus in the presence of
TNA-Cifer Reagent E for 7 days, and rescue from degradation by neutralisation.

| Virus or | | | | | |
|---|---|---|---|---|---|
| RNA | Sample | 4 h | Day 1 | Day 2 | Day 3 |
| SARS-CoV- | TNA-Cifer + SARS- | PASS | PASS | PASS | PASS |
| 2 virus | CoV-2 | | | | |
| | (5 min control) | | | | |
| | COVID + TNA-Cifer | Pass | FAIL | FAIL | FAIL |
| | (no rescue) | Ct shift 4 | | | |
| | pH 6 rescue | Pass | 4x LOD | 40x LOD | 40x LOD |
| | | Ct shift 2 | Ct Shift 6 | Ct Shift 7 | Ct Shift 9 |
| | pH 8 rescue | PASS | Pass | Pass | Pass |
| | | | Ct shift 3 | Ct shift 4 | Ct shift 6 |
| Purified | RNA only control | Pass | PASS | PASS | PASS |
| RNA | | Ct shift 4 | | | |
| | RNA + TNA-Cifer | FAIL | FAIL | FAIL | FAIL |
| | (no rescue) | | | | |
| | pH 6 rescue | Pass | 10x reduced | 10x reduced | 10x reduced |
| | | Ct shift 2 | sensitivity | sensitivity | sensitivity |
| | | | Ct shift 3 | Ct shift 5 | Ct shift 10 |
| | pH 8 rescue | PASS | Pass | Pass | Pass |
| | | | Ct shift 3 | Ct shift 4 | Ct shift 3 |

Pass: all concentration of purified RNA (124354, 12435, and 1244 copies/reaction) or SARS-CoV-2 virus (estimated 2000, 200 and 50 RNA copies/reaction) were detected in all three replicates. Fail - no detection in any replicates. Ct shift: Average Ct values for all replicates and RNA copy numbers were shifted compared to the control; LOD: limit of detection of SARS-CoV-2 virus, which was 50 RNA copies/reaction; 4x LOD: Only replicates at 4 times the limit of virus detection were detected; 40x LOD: only replicates at 40x the limit of virus detection were detected. 10x reduced sensitivity: Purified RNA was only detected down to 12,435 copies (not 1,244 copies).

### EXAMPLE 26: Sample Processing Protocols Using Sample Preparation Reagent E or U

Liquid samples: For subsequent quantitative PCR (qPCR)/isothermal testing, the following liquid sample preparation protocol can be used.

### Requirements

Sample Preparation Reagent E or U
Downstream molecular test (e.g. qPCR, reverse-transcription qPCR (RT-qPCR) or isothermal amplification equivalent)·
Purified sample positive control (e.g., with a known qPCR or RT-qPCR cycle threshold)
Molecular grade water

### Protocol

1. Set up a series of mixtures containing different ratios of samples and Sample Preparation Reagent E or U, as suggested in the first part of Table 1.
   a. Cap tubes, and then mix samples by flicking with your finger.
2. Incubate the Sample/Sample Preparation Reagent E or U mixtures for 5 minutes at room temperature.
3. Immediately make two dilutions of the sample/reagent mixtures, as suggested by the second and third parts of Table 1. Recommended dilution volumes are:
   a. 1 in 2 (5 μL Sample/Reagent E or U into 5 μL of molecular grade water), and
   b. 1 in 5 (5 μL of Sample/Reagent E or U mix into 20 μL of molecular grade water).
   Note: Downstream amplification reaction buffer can be used for this dilution rather than water.
   c. Cap dilution tubes and mix by flicking.
4. Immediately place each Sample/Reagent E or U mixture prepared in step 1, as well as diluted mixtures (step 3), into separate tubes containing downstream molecular test reagents (e.g. 5 μL into a 15 μL qPCR or RT-qPCR mixture, for a final volume of 20 μL).
   a. Cap tubes and mix by flicking (do not vortex).
5. Run downstream molecular test, and record the results (e.g. in the final Table 1 below).
   a. If measuring cycle threshold (Ct) values, your ideal Sample/Sample Preparation Reagent E mixture is one that returns a cycle threshold (Ct) value as close as possible to the purified sample positive control

### Nasal Swab Samples in Transport Medium

Using Sample Preparation Reagent E, this protocol has been demonstrated effective for processing SARS-CoV-2 infected nasal swabs collected in a range of transport mediums, followed by subsequent RT-qPCR detection (e.g, from brands including Bioline, Bio-Rad, Qiagen & Thermofisher). In addition, mixing Sample Preparation Reagent E with cultured SARS-CoV-2 1 in 6 (i.e. 1 part Reagent E and 5 parts virus in cell culture medium) was demonstrated to inactivate SARS-CoV-2 within 5 minutes incubation at room temperature, providing an additional level of safety for operators.

### Protocol

1. Mix 4 μL Sample Preparation Reagent E with 20 μL nasal swab sample in viral transport medium.
2. Incubate at room temperature for 2-10 minutes.
3. Test directly in downstream molecular test.

For example, place 5 μL of Sample/Sample Preparation Reagent E mixture into a qPCR with final volume of 20 μL.

### Whole Blood Samples

Using Sample Preparation Reagent U, this protocol has been demonstrated effective at detecting Malaria by extracting Plasmodium falciparum nucleic acids from infected red blood cells, and subsequent detection using recombinase polymerase amplification.
1. Mix blood directly with Sample Preparation Reagent U (1:1 ratio): i.e. 25 μL blood+25 μL Reagent U.
Note: Optimization according to the Table 1 protocol should be performed to trial different volumes of blood/Sample Preparation Reagent U or E
2. Incubate at room temperature for 5 minutes.
3. Dilute 1 in 1 with molecular grade water (i.e. 10 μL blood/Sample Preparation Reagent E mix in 10 μL water).
Notes: (1). Optimization as per Table 1., protocol can be performed to trial different dilutions prior to downstream molecular test. (2). Instead of water, the diluent could be one that prepares the nucleic acid for downstream molecular testing (e.g., Magnesium acetate for recombinase polymerase amplification testing). (3) For subsequent PCR testing dilutions of 1:10, 1:100, and 1:1000 could also provide improvements, depending on the enzyme compatibility with the samples).
4. Place 2 μL into 8 μL of downstream molecular test (e.g. recombinase polymerase amplification).

### Tissue Samples

Using Sample Preparation Reagent U, this protocol has been demonstrated effective at extracting prawn hepatopancreatic parvovirus and genetic material from infected prawn tissue, followed by PCR and recombinase polymerase amplification.
1. Measure ˜ 40 μg of tissue and place into 50 μL Sample Preparation Reagent U or E.
Note: Optimization can be adapted to mirror Table 1 by adding different concentrations of Sample Preparation Reagent U or E diluted in molecular grade water. Different volumes of Reagent E can sometimes be helpful for genetic testing (e.g. 3 μL per mg tissue).
2. Homogenize with sterile pestle.
3. Incubate for 10 minutes at room temperature.
4. Dilute 1 in 5 with molecular grade water (i.e. 5 μL tissue/Sample Preparation Reagent E mix in 20 μL water).
Notes: (1). Optimization as per Table 1 protocol can be performed to trial different dilutions prior to downstream molecular test. (2). Instead of water, the diluent could be one that prepares the nucleic acid for downstream molecular testing (e.g., Magnesium acetate for recombinase polymerase amplification testing).
5. Test directly in downstream molecular test.
   For example, place 2 μL into 8 μL of for recombinase polymerase amplification mixtures.
   For example, place 5 μL of Sample/Sample Preparation Reagent E mixture into a qPCR with final volume of 20 μL.

### Mosquito Samples

Using Sample Preparation Reagent U, this protocol has been demonstrated effective at extracting Dengue virus or Wolbachia bacteria from infected mosquitoes, followed by recombinase polymerase amplification.
1. Place single mosquitoes into a microcentrifuge tube with ˜50 μL Sample Preparation Reagent E, or mosquito pools in 250 μL Sample Preparation Reagent U.
Note: Optimization can be adapted to mirror Table 1 by adding different concentrations of Sample Preparation Reagent U or E diluted in molecular grade water.
2. Homogenize with sterile pestle.
3. Incubate for 10 minutes at room temperature.
4. Dilute 1 in 5 with molecular grade water (i.e. 5 μL tissue/Sample Preparation Reagent E mix in 20 μL water).
Notes: (1). Optimization as per Table 1 protocol can be performed to trial different dilutions prior to downstream molecular test. (2). Instead of water, the diluent could be one that prepares the nucleic acid for downstream molecular testing (e.g., Magnesium acetate for recombinase polymerase amplification testing).
5. Place 2 μL into 8 μL of downstream molecular test (e.g. recombinase polymerase amplification).

### Dry Swab Samples

Protocol based on successful wet-swab protocol above.
1. Place dry swabs in ˜500 μL of 1 in 5 diluted Sample Preparation Reagent E in molecular grade water into a microcentrifuge tube.
Note: Optimization can be adapted to mirror Table 1 by adding different concentrations of Sample Preparation Reagent E or U diluted in molecular grade water. In step 1 a starting concentration of 1 in 5 dilution can be used due to the success of Sample Preparation Reagent E with wet swabs in the protocol above.
   2. Cut swab at a length that allows you to close the tube.
   3. Incubate for 5 minutes at room temperature.
   4. Test directly in downstream molecular test.
   For example, place 5 μL of sample/Sample Preparation Reagent E mixture into a qPCR or RT-qPCR reaction with final volume of 20 μL.
Note: Optimization as per Table 1 protocol can be performed to trial different dilutions prior to downstream molecular test.

### Plasma Samples

Using Sample Preparation Reagent E, this protocol has been demonstrated effective at detecting Dengue virus nucleic acids in mock-infected plasma (plasma spiked with high titer Dengue virus), and subsequent detection using recombinase polymerase amplification.
1. Mix plasma directly with Sample Preparation Reagent E (2:1 ratio): i.e. 10 μL plasma +5 μL Reagent E.
Note: Optimization according to the Table 1 protocol should be performed to trial different volumes of plasma/Sample Preparation Reagent U or E
2. Incubate at room temperature for 10 minutes.
3. Dilute 1 in 1 with molecular grade water (i.e. 5 μL plasma/Sample Preparation Reagent E mix in 5 μL water).
Notes: (1). Optimization as per Table 1 protocol can be performed to trial different dilutions prior to downstream molecular test. (2). Instead of water, the diluent could be one that prepares the nucleic acid for downstream molecular testing (e.g., Magnesium acetate for recombinase polymerase amplification testing).
4. Place 2 μL into 8 μL of downstream molecular test (e.g. recombinase polymerase amplification).

### Serum Samples

Using Sample Preparation Reagent E, this protocol has been demonstrated effective at detecting Dengue virus nucleic acids in mock-infected serum (serum spiked with high titer Dengue virus), and subsequent detection using recombinase polymerase amplification.
1. Mix serum directly with Sample Preparation Reagent E (5:1 ratio): i.e. 25 μL serum+5 μL Reagent E.
Note: Optimization according to the Table 1 protocol should be performed to trial different volumes of serum/Sample Preparation Reagent U or E
2. Incubate at room temperature for 10 minutes.
3. Dilute 1 in 1 with molecular grade water (i.e. 5 μL serum/Sample Preparation Reagent E mix in 5 μL water).
Notes: (1). Optimization as per Table 1 protocol can be performed to trial different dilutions prior to downstream molecular test. (2). Instead of water, the diluent could be one that prepares the nucleic acid for downstream molecular testing (e.g., Magnesium acetate for recombinase polymerase amplification testing).
4. Place 2 μL into 8 μL of downstream molecular test (e.g. recombinase polymerase amplification).

### Cell Culture Supernatant

Using Sample Preparation Reagent E, this protocol has been demonstrated effective at detecting Dengue virus and SARS-CoV-2 nucleic acids in cell culture supernatant, and subsequent detection using RT-qPCR or recombinase polymerase amplification.
1. Mix cell culture supernatant directly with Sample Preparation Reagent E (5:1 ratio): i.e. 25 μL cell culture supernatant+5 μL Reagent E.
Note: Optimization according to the Table 1 protocol should be performed to trial different volumes of cell culture media//Sample Preparation Reagent U or E
2. Incubate at room temperature for 10 minutes.
3. Test directly in downstream molecular test.
For example, place 5 μL into 15 μL of a real-time PCR mix. Or place 1 μL into 8 μL of downstream molecular test and start reaction with 1 μL Magnesium acetate for recombinase polymerase amplification testing.
Notes: (1). Optimization as per Table 1 protocol can be performed to trial different dilutions prior to downstream molecular test. (2). Instead of water, the diluent could be one that prepares the nucleic acid for downstream molecular testing (e.g., Magnesium acetate for recombinase polymerase amplification testing).

### Gram-Negative and Gram-Positive Bacterial Isolate Samples

Using Sample Preparation Reagent E, this protocol has been demonstrated effective with 0.5 McFarland standards of E. coli, K. pneumoniae and Enterobacter hormaechei, as well as cultures of Salmonella abony and Staphylococcus epidermidis, using bacteria suspended in solutions such as saline, PBS, water or urine.
1. Make a 0.5 McFarland standard solution of the respective isolate, then mix the standard solution 1:1 with Sample Preparation Reagent E.
2. Incubate the mixture for 10 minutes at room temperature.
3. Test directly in downstream molecular test.

E.g. add 1 μL of the mixture to 8 μL of a recombinase polymerase amplification mastermix (+1 μL Magnesium acetate if not already incorporated, to make a final volume of 10 μL), or 1 μL of the mixture to 19 μL of a real-time PCR mix.

1. A method of preparing nucleic acids for nucleic acid amplification from a sample, said method comprising
contacting a sample comprising nucleic acids with a composition comprising a quaternary ammonium compound, a denaturing agent and a precipitating agent to form a reaction sample.

2. The method according to claim 1, wherein contacting the sample with the composition renders an infectious agent in the sample non-infectious.

3. The method according to claim 1, wherein the quaternary ammonium compound is betaine.

4. The method according to claim 1, wherein the denaturing agent is selected from the group consisting of NaOH and KOH.

5. The method according to claim 1, wherein the precipitating agent is selected from the group consisting of isopropanol, ethanol, and methanol.

6-9. (canceled)

10. The method according to claim 1, wherein the composition comprises from about 0.078 M to about 2.5 M betaine.

11. The method according to claim 1, wherein the composition comprises from about 15 mM to about 1000 mM denaturing agent.

12. The method according to claim 1, wherein the composition comprises from about 10% to about 80% precipitating agent.

13-20. (canceled)

21. The method according to claim 1, wherein the nucleic acids are not separated from the reaction sample the prior to nucleic acid amplification.

22-34. (canceled)

35. A method of detecting nucleic acids in a sample comprising:
contacting a sample comprising nucleic acids with a composition comprising a quaternary ammonium compound, a denaturing agent and a precipitating agent to form a reaction sample;
optionally diluting the reaction sample; and
performing nucleic acid amplification on the sample.

36. The method according to claim 35, wherein contacting the sample with the composition renders an infectious agent in the sample non-infectious.

37. A sample preparation composition comprising a quaternary ammonium compound, a denaturing agent and a precipitating agent.

38. The composition according to claim 37, wherein the quaternary ammonium compound is betaine.

39. The composition according to claim 37, wherein the denaturing agent is selected from the group consisting of NaOH, and KOH.

40. The composition according to claim 37, wherein the precipitating agent is selected from the group consisting of isopropanol, ethanol, and methanol.

41-44. (canceled)

45. The composition according to claim 37, wherein the composition comprises from about 0.078 M to about 2.5 M betaine.

46. The composition according to claim 37, wherein the composition comprises from about 10% to about 80% precipitating agent.

47. The composition according to claim 37, wherein the composition comprises up to 80% precipitating agent.

48. The composition according to claim 37, wherein the composition comprises up to 80% isopropanol, up to 125 mM NaOH, and up to 0.16 M betaine.

49. The composition according to claim 37, wherein the composition comprises up to 80% isopropanol, up to 31 mM NaOH, and up to 0.31 M betaine.

50-55. (canceled)
